SEQUENCE LISTING (46 sequences)

SEQ ID NO: 1 - DNA, 284 bp, synthetic construct
  source 1..284: mol_type = other DNA
tggctttgttggaaataggctcaaaactaagctaaaactattcgttagcacacccattattctcccgttttataaagttgaccaaacttaaggtaaggctatgatttcaggcatcctagcatctcctggtaggacactcgtcgctttcggagttgaaaagttcattgctgagaaagttcagtaatacgtagtaccaagtaattcaatacttaatggccattagacggcgaaaaaagtcgtctaatgagctatattggtatactataattcgacagaataaaact

SEQ ID NO: 2 - DNA, 4166 bp, synthetic construct
  source 1..4166: mol_type = other DNA
catttcgcatagataggctccaaccctctgctatatttaagtgcaaatattatgtcgtaaagctaacgatttctgattattgagcattaactagtttgttaagcttatttgaaaaaagtgtttgacagttgaacactcgttgcctataatcgaaagtcgagttatcgagattttcaggagaggaagctcatatgtcgaatatagaacatggtctaagctttatcgacataatggtcttcgccatttatgtcgcaattatcattggggtcggactttgggtatctcgtgataaaaaaggcactcagaaaagtacggaagattatttcttggcgggaaaatctttgccttggtgggctgtcggtgcttcgctaattgctgcaaatatttctgcggaacaatttataggaatgtctggttcaggctattcaattggcttggctatcgcatcttatgaatggatgtcggcaataacattgattattgttggtaagtactttctacctattttcattgaaaaaggaatctataccattcctgaatttgttgaaaaacgcttcaataaaaaactaaaaacaattttggccgttttttggatttccttgtacatttttgtaaacctaacttcagtactgtatttaggtggtttggctctcgaaaccattttgggtattccgttgatgtactcaattctaggtcttgcgctgtttgcgttggtgtactcaatttatggtggtttatcggcagtagtatggaccgatgtcatccaagtgttcttcttagttttgggtggttttatgactacctacatggcagtgagctttattggtggtacggacggttggttcgctggggtgtctaaaatggtcgatgcagctcctggccactttgagatgatcttggatcaaagtaatccacaatacatgaaccttcctggtattgccgtattaattggtggtctttgggtagcaaacttatattactggggctttaaccagtacattattcaaagaacgcttgctgcaaaatcagtatcggaagctcagaaaggtattgtttttgcagcgtttttgaaacttatcgttccgtttctcgtggtattgccaggtattgccgcttacgttattacttcggacccacaactaatggcaagccttggtgatattgcagcaacaaaccttccaagtgctgctaatgcggataaagcatacccttggctaactcagttcttgcctgttggtgttaaaggtgttgtttttgcggctcttgctgctgcaattgtttcttcactagcatcaatgcttaactcaacagccactatcttcactatggatatttacaaagagtatatctctcctgactcaggtgaccacaagttggtgaatgttgggcgtactgcagctgtggtggcactaattattgcttgcctaattgccccaatgttaggtggtattggccaagcattccaatacatccaagaatatacaggtttagttagccctggtattttggctgtattcttacttggcttattctggaagaaaacaaccagtaaaggggctattattggtgtagtagcatcaataccatttgccttgttcttgaaatttatgccactttccatgccatttatggatcaaatgctatacacattattgtttacaatggttgttatcgcatttacaagtttgagcacatcaattaatgatgatgatcctaaaggtattagtgttacatcatcgatgtttgtaacagatcgaagctttaatatcgctgcttacggcataatgattgttttggcagtgttatatacattgttctgggtaaacgcagacgcagaaatcacactaatcattttcggtgtaatggcaggcgtaatcggtacaatcctactaatcagctacggtatcaaaaaactaatcaaaggtggcggtggtggcggtcatcaccatcatcaccatggtggcggtggcggtggctcagagcaatacgcattagtaggcgacatcggcggtactaacgcaagattggctctctgcgagttatctacgggcacgatttctgatatcgtcacttatcctgcagcagaatacgaatccctcgaagtagtaatgcgccagtacttggatcgtcatgataccccgatctcttcagcgtgtatcgccattgcatgtccagtaacaggtgactgggtaagtatgactaatcaccattgggaattctctatccaggagctaaaatcacaacttaacctacaagtactagaagtaatcaacgattttacggctgtgagttttgctattccaagcttgaaaccagaggaccgtgtccaaattgggggtggtgaagcgatcgcaaacaagccaatcgctgtgtatggtgctggtaccggcctgggtgttgcgcagctgatccacggaggcaacctatggcattcggttccgggcgagggcggacatgttgacttagcagcgtgtacacctgaagaagatgatcttattgtttacttgagaaacaagtttggtagagtctctgctgagcgttgtttatcaggccaaggtatccgtaacatttacgacttcgttgtttcccaccatggtgcagcacctgaagattacacaccagcaatgatcacagaaaaagctcttaaagcggagtgcaaagactgtgaacgcgcactgtatcttttttgtatccttatgggccgatttggaggcaatcttgcgctaactaacggcagtttcggcggtgtgtacattgccggtggtatcgtaccaaaagtccaggaactttttatcaagagcggcttccgtgttgctttcgaagacaagggtcgatttaaagaatacctgcagtcaatcccggtttttctaatcactcattcagaacctggtcttctaggtgccggtacatatattcgccaatctctcaacattagtatcaactaaggcccgcatgtagtcaaaagcctccggtcggaggcttttgactcttggtaaggagaagcttatttgaaaaaagtgtttgacagttgaacactcgttgcctataatcgaaagtcgagttatcgagattttcaggagctaaggaagctcatatgtcggaacagtatgccttggtaggggatatcgggggaaccaacgcgcgtttagctctttgtgagttatcgacaggaacgatcagcgatatcgtgacttaccctgctgccgaatatgaatcgctggaagtcgtcatgcgtcaataccttgatcgtcacgacactccgatctcatcggcctgtattgcgattgcttgtccggtaacgggggattgggtttccatgactaaccaccactgggagttctctatccaggaactaaaatcccaacttaaccttcaggttttggaagtcattaacgatttcactgcggtttcctttgctattccaagtttaaaacctgaggacagagttcagatcggtggcggtgaagcgatagccaataagccgatagccgtatacggagcgggaaccgggcttggtgtcgctcagctgattcatggcggaaacctatggcatagcgtaccaggagaaggtgggcatgttgacctggcggcttgtacccctgaagaagacgacttgattgtttacctacgtaacaagttcggtcgtgtgtctgcggaacgctgtttgtctggccaaggtatccgaaatatatacgactttgttgtgtcacaccatggtgccgcacctgaagactacaccccggcaatgatcactgagaaagcactcaaagcggaatgcaaggattgtgagcgcgcactctatctgttttgcattttgatggggcgttttggcggtaacctggcactgactaatggatcatttggtggtgtgtatattgctggcggtattgttcctaaagttcaagaactattcatcaagtccggctttcgagtggcttttgaagataaaggccgatttaaagagtaccttcaatccatccctgtgttcttaatcactcattccgaacctggcttgctgggcgcgggcacctacatcagacagtcattaaatatctctataaattaatatttaactctaacccgaattcataaattggattcgggattctgcttgccatcttcatttttgtaatcaattctccagttataacctgtactttcaaagt

SEQ ID NO: 3 - DNA, 284 bp, synthetic construct
  source 1..284: mol_type = other DNA
tcatggcttctttgctcttatcaataatgaaaatacccaccaacgatggctgagtgaacacctgctcagtactcccttttacttttagaagaagactgttatgaatacgacaactcgaatcatacctgcgaggacactcgtcgctttcggagattatgaaaaatatttgagtaaccggacctagccttttcctcagattctgaataagaacatcgtttataggaaatgaatgaaatggaaacaaaaaagcagagtaagaatactctgcttttaggtatatgaag

SEQ ID NO: 4 - DNA, 284 bp, synthetic construct
  source 1..284: mol_type = other DNA
caacattcagtgtcaagcctattgattgatcgattaaaaaagtgtttcattaaccaatacaccaaaaataactttatcattttgaatcggaaataacttgatgcaaaaaacaactcgtaccatgcctgcaaggacactcgtcgctttcggagactatgaagcctatctgagcgagcgcacataatctgtgcccgatattttagataaaacaaaggcgataccgagtatcgcctttgttatttcaaacggtgtctattttatttagcgcgaagtaatgattgggt

SEQ ID NO: 5 - DNA, 285 bp, synthetic construct
  source 1..285: mol_type = other DNA
attttttaaggatattgctcgcaatcttacctatgaagattgacttctatccattagaatttattcccccttacctaaaaaataaaaagagaaacgcccacatgtctgaagacaaagcagtaactcccgaaaggacactcgtcgctttcggaattgaaatgcctgaaaactacatggcagagtaagacaaagatttaaatgacataaaaaaatgggacgcgatggcgtcccatttttttatgtcatttttagtattaggcgctaaataaccgcttggtgtattca

SEQ ID NO: 6 - DNA, 2060 bp, synthetic construct
  source 1..2060: mol_type = other DNA
tgagagagaatcacgcgactttgaaaccctctggatgacttcttttgagtataaatttccaccagacgtgaattttattgcaacattaccttgatgtgcaaagcttatttgaaaaaagtgtttgacaaggtgcccgaaaccggttataatcctcgttcgagttatcgagattttcaggagctaaggaagtcatatgggttcaacgccaactgatctgccaggcgaagatgtcgctgataaccgttcaggcgtaggcggcggaatctctgatgtatacggtgaggacagcgcgacgcttgaccagcttgttaccccttgggtgacaagcgtagcctctggctacacgttaatgcgtgatccacgttacaacaaaggcctagcattcacggacaaagagcgagacgcacactacttaactggactcctaccacctgttatactatcacaagatgtccaagaaagaaaggttatgcataacctacgacagtatacagttccacttcagcgatacatggcactaatggacttacaagaacgcaacgagcgccttttttacaagctgctaatcgacaacgtagaagaacttcttccggtagtctacacgcctacagtaggcgaagcatgtcagaaatatggttctattttccgtaaaccacagggtctttatatcagtctaaacgaaaaaggaaaaatccttgaagtattaaaaaactggccacaaagaggcatccaagttatcgtggtaactgacggggaacgcattcttggtttgggcgatcttggttgtcaaggtatgggtatccctgtcggcaagctctcactttacaccgcgttaggcggtattagaccatcagcctgcctccctattacaattgacgtcggaacaaacaacgaaaaactattaaatgacgaattttatatcggccttaaacagcgtcgcgctactggtcaggagtacgcagaattccttcatgagttcatgtgtgcagttaaacagaattacggtgaaaaagtcctagtgcagtttgaagatttcgcgaaccataacgccttcgatctactttcaaagtattcggacagtcacttggtttttaatgatgacattcagggtacagcgtctgtagttttggctggcttgatcgctgcacaaaaagtactaggtaaaaagctagctgatcacacctttttatttctgggtgcaggtgaagctggtacaggtattgctgaattgatcgccctcaagatctccaaagagactggcgcaccgattacggagacgcgtaagaagatctggctcgttgattctaagggtctgatcgtatcttctcgcaaagaatcactgcaacattttaagcaaccgtgggctcatgaacataaaccggtgaaggaccttatcggcgcagtcaacgcaatcaaaccgactgtcttaatcggcacaagtggtgtaggccaaacatttactaaagaggtggtggaagcaatggcaactaacaacgaaaaaccattaatcttggcactatcaaatccaactagtcaagcggagtgtactgcggaacaagcatacacatggaccaaaggtagagcgatctttggtagcggttcaccttttgacccagtagtttacgatggcaaaacgtaccttccgggtcaggctaacaattgttatatctttcctggtctgggcttggggctaatcatgtccggcgcaattcgtgttagagacgatatgcttttggcggcgagtgaggcattagcagctcaagtaactgaagagcactatgctaacggactgatttatccaccgtttagtaacatccgtgaaatttccgcgaacatcgctgcctgtgttgcagctaagacatacgatcttggtctggcatctaacctacctcgtgctaaagatctcgttaaattcgcagagtcgtctatgtatagccccgtttaccgtaactaccgctaaatttaaccgcaaaagaaaaactcgcctaataattagcccctaacacataggggcttttgttttttaaacgtttttttatcatcctaaccgtattttggca

SEQ ID NO: 7 - DNA, 1278 bp, synthetic construct
  source 1..1278: mol_type = other DNA
gaatgaagggatcaaaaaagccgaccctaagatcggctttttataaacgtataaacgcttaaacgggcccctgaagtcagccccatacgatataagttgtaagcttatttgaaaaaagtgtttgacagttgaacactcgttgcctataatcgaaagtcgagttatcgagattttcaggagaggaagctcatatgaattcaccgcaaaacgtctctacgaaaaaggtcacagtcaccggcgcagctggtcaaatctcttattcacttttatggcgcatcgccaacggtgaagtattcggcactgacacgccagtagaactgaaacttctagaaatcccgcaagctcttggcggggcagagggtgttgctatggaacttctagattcggccttccccctcctacgaaacatcacgatcactgcggatgccaacgaggcattcgacggcgctaatgctgcatttttggtaggtgcgaaacctcgcggtaaaggcgaagagagagcagacttactggctaacaacggcaaaatttttggtcctcaaggtaaagctatcaacgacaacgccgcagatgacatccgtgtgctagttgttggaaacccagcgaacaccaacgcgttgatagcttcagctgcggcaccagatgttccagcatcaagattcaacgcaatgatgcgccttgatcataaccgtgcgatcagtcagctagcaactaagcttggccgtggttctgcagaatttaacaacatcgtggtatggggaaatcattccgcaacacaatttccagacatcacgtacgcaacagttggtggagaaaaggtcactgacttagttgatcacgattggtacgtagaagagtttattcctcgtgtggctaaccgtggcgctgaaatcattgaggtacgtggtaaatcatctgcagcttctgcagcatcctctgcgattgatcatatgagagattgggtacaaggtacagaggcgtggagcagtgcggcaattcctagcactggtgcatacggcattccggaaggcatctttgttggtctgccaacagtaagtcgaaacggtgaatgggaaatcgttgaaggcctagagatttcagattttcagagagcacgtatcgacgcgaatgctcaggaattacaggctgaacgcgaggcagtgcgcgacttgctctaagtagttctccttgagagtatttttttataaatgtatttgtagggtagtttttaaccaaccaagtaaagctataggaatcacaacttgattacaatcaatt

SEQ ID NO: 8 - DNA, 1826 bp, synthetic construct
  source 1..1826: mol_type = other DNA
cgcagtatcgagagtaacagcagaaacaccacaagaagtgaaagatctggttgagcacgtacgtgtaccagtaggtgcgggccaagctgtcgatcactaactgaaataaagaacaaaatagaaagctgggcttaattgtagggatatagctcacttgctccaagccgatgcctgtattcatgcatcggcttttttaagcttatttgaaaaaagtgtttgacagttgaacactcgttgcctataatcgaaagtcgagttatcgagattttcaggagaggaagctcatatgaacaactcaccacgtttgttttcttcagccagtgctgcactacaaaaatttcgcgcggagcgtgatacattcggtgatctccaagtgccagctgaccgatactggggcgcacaaactcaaagatctcttcaaaacttcgacatcggaggtccgactgaacgaatgccagagccactaattagagcattcggtgtattaaaaaaggccgcagcaacggtcaacatgacgtacggtctggatcctaaggttggcgaagcaattcaaaaggcagccgatgaagttattgatggttcacttatcgatcattttccacttgttgtctggcaaacaggttctggaacacagacaaaaatgaacgtgaacgaagtaatctcaaaccgcgcgatagagctcttaggcggtgaacttggctcaaaagctccggttcatcctaacgaccacgtaaacatgagtcaatcttcaaacgacacgttcccgacagctatgcatgtggcagcggttgtagagattcatggtcgtctcattccagctctaacgacgttgcgcgacgcgctgcaggcaaaatccgctgaatttgaacacatcatcaaaatcggacgcactcatttgcaggatgcgactcctctaaccctaggccaggaattttcgggttacactcaacagcttacatatgggatcgctagagtacagggcacgctagagcgcctttataatctcgcacaaggcggtactgcagtcggcactggtttgaacacgcgtaaaggctttgatgctaaagttgcagaagcaattgcaagcatcacgggcctgccttttaaaaccgcgccaaacaaattcgaagcactagcagcgcacgacgcattagtcgaggctcatggtgctttgaacaccgtggcatgttctcttatgaagatcgcgaatgatatccgttaccttggctccggtcctcgttgtggtctaggtgaactttcgttacccgagaacgaaccgggttcaagtatcatgccaggcaaggttaaccctacacaatgcgaggcaatgactatggtttgtgctcaggttatgggtaacaacacagcgatcagtgtagctggtagtaacggccagttcgaacttaacgtatttaaaccggtaatgatcaagaatttaatccaatcaatcagactaattagcgacgcgtctatctcttttactaaaaactgtgtagtcggcattgaggctaatgaaaaaaagattagctctatcatgaatgaatccctgatgcttgttactgccctgaacccacatattggctacgacaaggctgcaaaatgtgctaaaaaggcgcacaaagaagggacaaccctaaaagaagcagcactttctttaggttatctgacatcagaagagtttgatcagtgggtacgtccggaagatatgatcagcgccaaagactaagtcatcaaaatcctcgtaaatcacctatggttgataagagacttaggattgagaaccgaggagcacgtcatgaccagagactttcgtattgaaaccgaca

SEQ ID NO: 9 - DNA, 3137 bp, synthetic construct
  source 1..3137: mol_type = other DNA
tctcagcctaaagcgcgtctatacggtgaagctgcagaaaaagccgcagcagaagaagcagcgaagaacgcagaggagcaagcataagacactgttataaaagtcaaaagcctccgaccggaggcttttgactgggccctgaagtcagccccatacgatataagttgtaagcttatttgaaaaaagtgtttgacagttgaacactcgttgcctataatcgaaagtcgagttatcgagattttcaggagaggaagctcatatgaccagtaacgaacgaattctgcagccatttactttaccgaatgggactgaactgaaaaaccgtttgctaatggcgccaatgaccacctgcaccggttatttcgatggtaccgtcaccagcgagctcgttgaatactatcgcgcccgcgccggcagcatcgggactatcatcgttgaatgctgctttattgatgattatggtctggccttcccgggcgctattggcattgataacgatgaaaaaatcgccggactggcgaaaatcgccgaagcgataaaagcggaaggctcgaaagcgattctgcagatttaccacggcggccgcatggttgacccacagttgattggtggtcgccagccggtggcgccgagcgccattgccgcgccgcgcgacggcgccgccatgccgcgcgccctcagtggtgaagaagtcgaaggcatgattgccaaatttggcgacggcgtacgccgcgccattctcgccggtttcgacggcgtcgaaatccatggcgccaacacctatcttattcaacagttctattcgccgaactccaaccagcgtgacgatgagtggggcggcagccgtgacaaccgcgccagattcccgctggctgtactggacatcacgcataaaatggcccgtcagtacgctgacgatgcctttattatcggctatcgcttctcgccggaagagatggaagtcccggggatccgcttcgacgacaccatgtatctgctggaaaaactagccgcccgcggcgtcgattacctgcacttctcggttggcgcgacgctgcgcccgtctatcgtagataccagcgatccgacgccgctgattgaaaaatactgcgccatgcgttctgaaacgctggcgcaggtgccggtgatgggcgtcggcggggtggttaacgccgctgatgccgaacagggcctcgatcacggttatgatctgatggccgtcggccgcgcctgcatcgcctatccggattgggcttcacgtatcgccgccggcgaagagctggaactctttatcgacagcacccagcgcgaggcgctgcatattccggaaccgctgtggcgtttctcgctggtcgaagcgatgatccgcgacatgagcatgggcgacgcgaaatttaaaccggggatgtttgtcgaaaccgttcaggacgatgccaacgaactggtcattaacgtcagcctcgaaaacgatcgcattgcggatattgagctggccgcaagcccagtacagagcgttgaatttaccaccagcttcgaagagatccgcgaacgtattctgaccgccaacacgccgcacgtcgacgctatctccggcgcgactagccagagcgaagcggtgaaaaaagcggtgtccaaagcgatgctgaaatctagcaaagcgctggctgcggaagagggcggcaacgatgccgcgccgaaaagctatgacgtggtggtggtagggagcggcggcgccggtctggccgcggcgattcaggcccacgacgaaggcgccagcgtgctgatcgttgaaaaaatgccgactattggcggcaacaccatcaaagcttccgccgggatgaacgccgcggaaacccgcttccagcgcgtcaaaggtattcaggacagtaaagagttgttctatcaggaaaccctgaagggcggccacaacaaaaacaacccgcagctgctgcgccgtttcgttgaaaacgcgccgcaagccattgaatggttggcggaccgtgggattatgcttaacgatatcaccactaccggcgggatgagtatcgaccgtacccaccgtccgcgcgatggatcggcggttggcggctacctgattagcggtctggtgcgtaatatcaccaaacgcggtatcgatgtactgctggatacctcggtagaagagatcctgatgcgccacgacgaagtgagcggcgtgcgtcttatcaacgatgaacaagagatcatcgacgtgcagaccaaaagcatcatcgtggcaaccggcggcttcagcgccaacagcgcgatggtggtgaaatatcgtcctgacctcgaaggcttcgtgaccaccaaccataaaggggcgaccggtagcggtattgcgctgctggagcgcattggtgccggcaccgtggacatgggcgaaattcaaattcacccgaccgtcgagcagcagacctcgtacctgatttccgaatcgattcgcggcggcggcgctattctcgttaaccagcaggggaaccgcttcttcaacgagatggagacccgcgataaagtctctgcggcgattatcgcgctgccggaacactatgcttacatcgtcttcgacgagtatgtgcgggcgaaaaacaaagccgccgacgagtatatcgccaaaggcttcgtcactagcgccagctcgccgcgtgaactggcggagaagctggggatggattaccatgcgttcctcgctaccctggaatgctacaacggcgcggtggaaaaacagcacgatgaagcgtttggccgcacaaccgcgctgcgcgcgccgattaacgaaggtccgttccacgccattcgtattgccccaggggtgcaccacactatgggcggcgtgaccattaataccgatggcgaagtgctgaatgtggcgcagcagccgattcgcggcgcctacgccgcgggtgaagtggttggcggtatccatggcggtaaccgcatcggcggtaacgcggtcgcggatatcatcatcttcggtaccctcgcgggccatcaggcggcgaaacgtgccagaggataataggtaattgctgatgccgggctggcttagccgcccggcttatcccttctaaggagagcccatgaaaaaagctggctttatgccagctttttttgttttt

SEQ ID NO: 10 - DNA, 284 bp, synthetic construct
  source 1..284: mol_type = other DNA
tattctcggtgattatcggtcgtatctgttaaataaatgttaacttttgtacatttggacgctgaaattggttataacaataaatgctcaatggagctgagtgagcaagcccgtgaaagaaagaaagtcaaggacactcgtcgctttcggaatcaagtcgatgctggtaaatcgctcggtgtaatttactcgtagatgaaccagttgcagtgttgtgtaaagtaaaccccatcactgcgctgcaactgcataactcgagtagacgaatattaaaattgccgatt

SEQ ID NO: 11 - DNA, 6790 bp, synthetic construct
  source 1..6790: mol_type = other DNA
aggcatggtttcaaccatgcctttttgtatacaaaaaataattacttttgttgaaaattaactacatttttgtgtgtacagcgagggaagtggccgatataagttgagcatttattgatttagtttaaggtttgcaaaaaaattttagtttaacctgcaattgttgtttcaaaaggtagacgtttaaagcagtgacaatatttatggttttgtaaccctaaaaagtgattttaagactataaatattgttattcataagagtgtactgttcccctttatttctggggatagctggctcaacggcgaagcagtacgtatttttttctactaaaaaggtaggtatgtcatgtctgatatgaagcatgacgtagatgcactggaaactcaagaatggctacaagcacttgagtcagttgtacgtgaagaaggcgtagagcgtgctcagttcctacttgagcaagttctagataaagcacgtctagacggtgttgacatgccaactggaatcaccacgaactacatcaacacgattccagcagatcaagaaccagcttacccaggtgacacaacacttgagcgtcgtattcgttccatcattcgctggaacgcaatcatgatcgttctacgtgcatcgaagaaagacctagaactaggcggccacatggcgtctttccagtcttctgctgcattctacgaaacatgtttcaaccatttcttccgtgctccaaacgagaaggatggtggcgacctagtttactaccaaggtcacatttctccaggtatctactctcgtgcattcgttgaaggccgtctaactgaagagcagctagacaacttccgtcaagaagtagatggtaaaggtatcccgtcatacccacaccctaaactaatgcctgaattctggcagttcccaaccgtatctatgggtctaggtccgatctctgcgatctaccaagctcgtttccttaagtaccttgaaggccgcggcatgaaagatacttctgagcagcgcgtttacgcgttcctgggtgacggtgagatggatgagccagaatcacgtggtgctatctctttcgctgcgcgtgagaagctagacaacctatgtttcctaatcaactgtaaccttcagcgtctagacggccctgtaatgggtaacggtaagatcattcaagaacttgaaggtctgttcaaaggtgctggctggaacgtagttaaagtaatctggggtaacaactgggattctctactagctaaagatacaactggtaagcttctacagcttatgaacgaaacaatcgatggcgattaccaaacgttcaaagcgaaagacggtgcatacgtacgtgagcacttcttcggtaagtacccagaaacagctgcactagttgctgacatgactgatgacgaaatcttcgcacttaagcgtggtggtcacgagtcttctaagctatacgctgcatacaaaaacgcggcagacactaaaggccgtccaacggttatcctagctaagactgttaaaggttacggcatgggtgaagcggctgaaggtaagaacatcgcgcaccaagttaagaagatggacatgactcacgttctacacctacgtgatcgtctaggtctacaagatcttctaactgacgaagctgtgaaagagcttccgtacctgaaacttgaagaaggttcaaaagagtacgaatacctacacgctcgtcgtaaagcgctacacggttacacgcctcagcgtctaccgaacttcactcaagaactgatcgttcctgaactagaagaattcaagcctctactagaagagcagaagcgtgacatctcttctactatggcattcgtacgttcactgaacgttctgcttaagaacaagaacattggtaagaacatcgttcctatcattgctgacgaagcacgtacattcggtatggaaggtctattccgtcaaatcggtatttacaacccgcacggccagacttacacaccagaagaccgtggcgttgtttcttactacaaagaagcgacttcaggtcaggttctacaagaaggtatcaacgagctaggtgctatgtcttcatgggttgcagcagcaacttcatactcgacaaacgacctaccaatgattccgttctacatctactactctatgttcggtttccaacgtgttggcgacatggcgtggatggctggtgaccaacaagcacgtggtttcctactaggtgcaactgctggtcgtacgacactgaacggtgaaggtctgcagcacgaagatggtcactcgcacatcatggcgggtacagttcctaactgtatctcttacgatccgacattcgcttacgaagtagcggtaatcatgcaagacggtatccgtcgcatgtacggtgagcaagagaacgtgttctactacctaacgctaatgaacgagaactacgcaatgccagcaatgccagaaggcgctgaagaaggcattcgtaagggtatctacaagctagaaacttacactggttctaaaggtaaagttcagctaatgagctctggtactatcatgaacgaagtacgtaaagcagctcaaatccttagcgatgagtacggtgttgcatctgacgtttactctgtaacgtcattcaacgaagtaactcgcgacggtcaagcggcagagcgttacaacatgctacacccagaagcggaagcgcaagtaccatacatccaaacagtaatgggtactgagccagctatcgcagcaactgactacatgaagaactacgcagagcaggttcgtgcattcatccctgctgaatctttcaaagttcttggtactgatggcttcggtcgctcagacagccgtgaaaacctacgtcgtcacttcgaagtgaacgcaggttacgtagtagtagcagcgctaactgaactagcgaaacgtggcgaagttgagaagtctgtaattgctgaagcaattaagaaattcgacatcgacactgaaaaaacaaacccgctatacgcttaattaagaaggtagataagaaatggcaatcgaaattaatgtaccagacatcggtacggatgaggttgaagttactgagattcttgtaagcgttggcgacaaggttgaagaagagcagtctctgatcactgttgaaggcgataaagcttctatggaagttcctgcttctcaagcgggtatcgttaaagaaatcaaagttgcagaaggcgacaaggtttctactggttctctaatcatgattttcgaagccgagggtgcagctgaagctgcgcctgctcctgcggcggaagcagctccagcagcagctccagctccagcagcagcggcagaactgaaagaagttcacgtaccagatatcggtggtgacgaagttgaagttactgaaatcatggtagcaatcggcgacagcatcgaagaagagcaatctctgatcactgtagaaggcgacaaagcttctatggaagttcctgcaccattcgcaggtacgcttaaagaaatcaaagtagcagcaggcgataaagtatcgactggctctctaatcatggtattcgaagtagcaggttcaggcgctccagcagcagctcctgcggcagtagaagcaccagcggcagcagctccagcagcatctgcggcaaaagaagttaacgttccagatatcggtggcgacgaagtagaagttactgaaatcatggttgcagttggcgatacagtggaagaagagcaatctctaatcactgtagaaggcgacaaagcttctatggaagttcctgcaccattcgctggtactgttaaagaaatcaagattgcagctggcgacaaagtgtctactggctcactaatcatggtattcgaagtggcgggcgcagctccagctcctgcagcggctccagctcaagcggcagcacctgcagcagcggctcctaaagcagaagctccagcggcggcagctccagcagcgacaggcgatttcaaagagaacgacgagtacgctcacgcgtctccagttgttcgtcgtctagctcgtgagttcggcgtaaacctttctaaggttaaaggttctggtcgtaagagccgtatcctgaaagaagacgttcagaactacgtgaaagaagcgcttaagcgtcttgagtctggtgctgcagcatctggcaaaggcgacggtgcagctcttggtctactaccatggccaaaagtggacttcagcaagttcggtgagacagaagttcaacctctatctcgcattaagaagatttctggtgctaacctacaccgtaactgggtaatgatcccgcacgttacacagtgggataacgcagacatcacggctcttgaagcattccgtaaagagcaaaacgcgattgaagcgaagaaagacactggcatgaagatcactccactagtgttcatcatgaaagcagttgctaaagcacttgaagcgttcccggcgttcaactcttctctatcagaagacggtgagagcctaattctgaagaaatacgtgaacgttggtatcgcggtagatacgccaaacggtctggttgttcctgtatttaaagacgtgaacaagaaaggcatctacgagctatctgaagagctaatggctgtttctaagaaagctcgtgctggtaagctaactgcggctgacatgcaaggtggctgtttcactatctctagccttggcggcattggcggtactgcgtttactccaatcgtgaacgcgccagaagtgggtatcctaggtgtatctaagtcagagatgaaaccggtatggaacggtaaagagttcgaaccgcgtctacaacttccactgtctctatcatacgaccaccgtgtgatcgatggtgcggaaggtgcgcgtttcatcacttacctaaactcatgtctatctgacattcgtcgtctagttctttaatagaagttagatgtatgagaggcggcaatttagccgcctctgctaataataattactgaacaatttggttatcgcccttattcactatggggttttcacaaagttgagcatttattgatttagtttaaggtttgcaaaaaaattttagtttaacctgcaattgttgtttcaaaaggtagacgtttaaagcagtgacaatatttatggttttgtaaccctaaaaagtgattttaagactataaatattgttattcataagagtgtactgttcccctttatttctggggatagctggctcaacggcgaagcagtacgtatttttttctactaaaaaggtaggtatgtcatgagcaaagaaattaaagcccaagttgttgtacttggttctggtcctgctggttactctgcagcattccgttgtgcggatttaggtcttgaaactgtactagttgaacgttacagcactcttggcggtgtgtgtctgaacgtgggttgtatcccatctaaagcacttcttcacgtttctaaagtaattgaagaagcaaaagcaatggctgatcacggcgttgtatttggcgaaccgcaaactgacatcaacaaaatccgcatctggaaagaaaaagttgttaaccaacttactggcggcctaggtggcatggctaagatgcgtaacgtaactgtagttaacggttacggtaagtttactggtcctaactctatccttgtagaaggtgaaggcgagtcaactgttgttaacttcgacaacgcaatcgttgctgctggttctcgtccaatcaaactgccatttatcccgcatgaagacccacgtatttgggattctactgacgctcttgaactaaaagaagtgccagaaaaactacttatcatgggtggtggtatcatcggtctggaaatgggtacggtttaccactctctaggttctaaagttgaagttgttgagatgtttgatcaggtaatccctgcagcagacaaagatatcgttaaggtttacaccaagcgtatcaaagacaagttcaagctaatgctagaaacgaaagtaacagcagttgaagcgaaagaagacggtatctacgtttcaatggaaggtaagaaagcacctgctgaagcagagcgctacgatgctgttcttgttgctatcggtcgtgtaccaaacggcaaactgattgacggtgaaaaagcgggtctagaaatcgacgagcgcggcttcatcaacgttgacaagcaaatgcgtactaacgttcctcacatcttcgcgatcggtgatatcgttggtcaaccaatgcttgctcacaaaggtgtgcatgaaggtcacgtagcagctgaagttatctctggtaagaagcactactttgaccctaaagttatcccttcaatcgcatacactaaaccagaagtggcttgggttggtaagactgagaaagaagcgaaagacgaaggcatcaagtacgaagtggcaacattcccatgggctgcatcaggtcgtgcaatcgcatctgactgttcagacggtatgactaagctaatcttcgacaaagaaactcaccgcgtaatcggtggtgctatcgttggtactaacggtggtgaactacttggcgaaatcggtcttgcgatcgagatgggttgtgatgcagaagatatcgctctgactatccacgctcacccaactctacacgagtctgttggtctagctgctgaagtatttgaaggttctatcactgaccttccaaacaaaaaagcagttaagaagaagtaagtttctttactgattaaataataaaaaccgctgaacattcagcggtttttttgtttttaacagctaagtttcaaatacaaaaaagcctggcagaagccag

SEQ ID NO: 12 - DNA, 292 bp, synthetic construct
  source 1..292: mol_type = other DNA
ccaaaaaggtgatctagttactatttttatgtgctaggtaaagtatgatgtgccacataagtcaggacggattttccagcccaaaattctgatgaatggaagcttatttgaaaaaagtgtttgacaaggtgcccgaaaccggttataatcctcgttcgagttatcgagattttcaggagctaaggaagtcatatggtcatggtcgaactcttcgtggttctgctctttatctatttgggagcgagaatcggcggtatcggcatcggttttgccggtggtgcgggtgtcattg

SEQ ID NO: 13 - DNA, 284 bp, synthetic construct
  source 1..284: mol_type = other DNA
accaagattaggttttttctttcgtgttttttcactccgctgctaacctgcacgccactttgaagtgaactcagagtgcataactataaggagtgttctcatgaataccaagaaaccgatgtcgcttaccaggacactcgtcgctttcggagaagaagtgcatcttaaacgcgctgacgcttaaaccgtttacggttatcctgtctaaaacaaagccccgatggaaacatcggggctttgttcattgagaaaggtttattaagcaatcttaaaggcttttagtc

SEQ ID NO: 14 - DNA, 2005 bp, synthetic construct
  source 1..2005: mol_type = other DNA
tatccgcattggcgtatatatcttcaagagagctgtaacgtgcgcccatctctattgccagtgggttttcgtaaggatcatggcaaagaatttccatgccgttgtcgccgttagtacgccatggttctctctcgaaatcattgtgttctctaataccgtaatgcaacttaggtacaatgaatagataaatctgcaatacgggctctgtcataccaaaatgccattaacataggttttttacgtccccacgttataatcagattgctttgtcatcatcaactgactagcatatcccttctcttctttccttgttggcttctttatttattaggcgttctggcgatgtcagtaaaatggtgttctttacttgtatgttgtgcgttcgtgttcgaagcgcagggtgctgtcgatcttgttaaagtcgacaagtcgaagcgtcgaatgtacttgatggaagaaggcgctgtgataaaggagtatcgaattgcgctgggtgctaaccccaaaggtcataaacaagaagaaggagacaatcgcacaccagaaggtgattatactctggattacgtgatctatgactcggctttttaccgttccgtacatatcagctaccccgatcctattgatacgcttgaggcccatcgtcgcggtgtctaccctggtggcaacattaaaatccatggcttaaaaaatggagaaacccaagatccaagctttatacaaagttttgattggaccaatgggtgtatcgcgattactaacgaagagatggatgagtttattcaattggttagaaggggcacacctattaccattgagtggtagcagtatatccccctcatgatctaaaacagtcttttaccgaagaaaccgtgtagggtaatcactgaagatgccatctagaggtggcagatgtttgagtttatgtgggttgttgacggtatagcacatcactttatagccatgttgttggagttttttaatctgacgggagcgcacccaattgatgtttaagttacagctgtaagcgttgacttcttcaagcagcaattggtcttttctggtgagaaactcactcaatacacctaaacggtagcctctgcagtgctcaaagagcgctctaatcacatcatgactaaaactagaaagtaaaatactctctgagggcagagggctaccagaaagcactttcgctaataactttgcaactggctctgcgggatgacgatcaactttaacttcgatgtttaaattgaggttgtgctctgcggccagttcaagaagctcttccaacgtcatgatggtttcacccttaaactcatcactaaaccagctaccaaagtctaagcttttgagttcatccaaagtcagttcatctattcgcccttgtccgttactacagcggttgaccgtgtggtcatggcataccaccaaaatgttgtctttcgtcggctgaacatcgacttctacccatttcaaatcaaggtttattgcagccagaacgcttatctttgtgttttctgggtaagaccccgccacacctcggtgaccaattaacaatgtatccatgttgtcgttcttctcgtttgggttgaacccgatactaatcgattttaagtggtttggtttagtgggcatcaagcgaggtcgatgcaacaccttcatttacagcggtattcacaggaatacaaatatgttggacttttactctatcgcagctcatttttaaagtaacgactaatcttgcaagcgcagaacaatacccagaaacgcacggagttgagctgagcataagcctttatgcatgtcgaaaaatttgatgattggatggacttgaatcctgtattttgcagtgattttttagaattatcagggagagaaaaatggcgtactaacggcgacaactttggttacgtattaggcgagcatggtagtcattgttttacgccgaagagtttaatttctatcgcaggccaaccggcggattactactgtgatacacaca

SEQ ID NO: 15 - DNA, 846 bp, synthetic construct
  source 1..846: mol_type = other DNA
ttggtgccaaatattacaaatctataactatactagctattaattcgataataacaccctacaaatagttaaaataaccattatagaaagaggatacaggatggcgtactaacggcgacaaccctgcaaaagtcgtcattgtgggcggcggtgtcgttggcgcaaacgcagctcgtatggctgtgggccttcgtgcagacgtcactattcttgatcgtaatgtcgatactctgcgtaaactggacgaagaattccaaggccgcgctaaagtcgtttattctacagaagacgcgattgagaagcatgttgtagaagctgacctagtaattggcgcagttcttatcccgggtgcagctgcacctaaactggtgacaaaagaacacattgcgaagatgaagccaggcgcagcggtagttgatgtagctatcgaccaaggtggttgctttgaaacatcgcacgcaaccactcacgcagaaccgacatacatcgtcgatgaagtcgttcactactgcgtagcaaacatgccaggtgccgttgcccgtacttctacttttgcattgaacaacgcaacgttaccttacattttgaaactggcgaataaaggctaccaaaaagcccttctcgacgagaaaggtttccttgaaggactaaacgttattcacggcaaagtgacttgtaaagaagttgccgacagctttggtcttgaatatgtagaagcagagcaagcgattgcaatgttcaactaatgacaaaataattcatttttgtcacgttttgtttacaaaagcccactagcaggtgggctttttcctatagagcatttagatataaataataaccattact

SEQ ID NO: 16 - DNA, 962 bp, synthetic construct
  source 1..962: mol_type = other DNA
tgttattcataagagtgtactgttcccctttatttctggggatagctggctcaacggcgaagcagtacgtatttttttctactaaaaaggtaggtatgtcatggcgtactaacggcgacaaccctgacgcatacggccgtggtcgtatcatcggtgactaccgtcgcgttgcgctttacggtatcgacttcctaatgaaggacaaactagctcagttcacttctcttcaagagaaatttgagaacggcgaagaccttcacatgactatgcaacttcgtgaagaaattgcagagcaacaccgcgctctaggtcagatcaaacaaatggctgcgaaatacggtttcgatatttctcgccctgctgaaactgcacaagaagctatccaatggacttacttcggctacctagctgctgttaagtctcaaaacggtgctgcaatgtctctaggtcgtacttctacattcctagacgtgtacatcgagcgtgatatcgctgcaggtaagatcactgaagaccaagctcaagaaatgatcgaccacttcgtaatgaaactacgtatggttcgtttcctacgtactcctgagtacgatgagctattctctggcgacccaatctgggcaacagaatcaatgggtggtatgggtcttgacggtcgtacgctagtaacgcgttctaacttccgtttcctaaacagcctatacactatggggccttctccagagccaaacatcactgttctttggtctgaagcacttccagatggcttcaaacgtttctgtgcaaaagtatctatcgatacttcttctatccagtacgaaaacgacgatctaatgcgtccagacatggaatcagacgattacgctatcgcttgttgtgtatctccaatggttgttggtaagcaaatgcagttcttcggtgctcgtgcgaaccttgctaaaactatgctttacaccatcaacggc

SEQ ID NO: 17 - DNA, 284 bp, synthetic construct
  source 1..284: mol_type = other DNA
gaccaagattagtacgcacccttgctggtatcgactattcttgtgattaatttttcattctttaactgattttgatcagacgaataacaggtagtcatacatgtctaagctagttttagttttaaactgcaggacactcgtcgctttcggagctgaagacactgcacgcctagcaggtctttaatgcataaactggctagccaattggctagccagtttttcttccaaagagctttctttaggggctcagctcctattctttgaagatcatggaataagaattg

SEQ ID NO: 18 - DNA, 281 bp, synthetic construct
  source 1..281: mol_type = other DNA
taagtcgtttgtttgttaagtggctgataagcagcaaaaagagataagcaatagccttactaaaaagtttttaatatttgttaattttaggagaatccctatgcctgtaactaatatggctgaactagataggacactcgtcgctttcggaaaagctaaaaaagaaaaagcagacgcgtaagtaagcgttgttttgaacaaggttttcgctagcacgaaaccttaatcgggaaaagttaaaagccccagtcgaatgactggggctttttttatcttctttg

SEQ ID NO: 19 - DNA, 475 bp, synthetic construct
  source 1..475: mol_type = other DNA
atggcgtactaacggcgacaactttggttacgtattaggcgagcatggtagtcattgttttacgccgaagagtttaatttctatcgcaggccaaccggcggattactactgtgatacacacaatatcaaacgaattgatgcggatgagctgttggaggcggtgaaacaagccggctatgaaatatttcgtcgcaagaacaataccgtacacggcattgccgcgagtgtatttcggatagtgcaggcagttatcattaacgagcgctcggtgttgcctgtcggaacgatgataaaagggcaatatggcgtgaataaagtcgtactcagtttgccaacggttgtgggtaaaaatggcgcagactcggttcttactcatccatttagtggtgaagagctaaaaacgttaagagccatcagtgagaacttacgttcaatcgtaaagaacgttgcatcgtcaaccagtttagagtgttag

SEQ ID NO: 20 - DNA, 517 bp, synthetic construct
  source 1..517: mol_type = other DNA
ttagttgatcagttcattgcctgaaacttgcccagagaagaaggcttctacgttatttagtgtcaccgacgcgatgttgttgagcgcttcgtgagttaagaaagcttgatgacccgtgaacagcacgttatggcatgccgacagtcggcggaaaacatcgtctacgataatgtcgtttgatttatcctggaagaacaactctttttcattgtcgtacacatccaagcctagtgagccgattctgccttgttttagcgcttcgattgctgctacagaatccaatagctctccacgactcgtattaatgatcatcaccccatctttcattttcgaaaatgagtcagcattgaggaggtggtagttttctttactcatcgggcaatgcaaagtaataatatccgcattggcgtatatatcttcaagagagctgtaacgtgcgcccatctctattgccagtgggttttcgtaaggatcatggcaaagaatttccatgccgttgtcgccgttagtacgccat

SEQ ID NO: 21 - DNA, 646 bp, synthetic construct
  source 1..646: mol_type = other DNA
atggcgtactaacggcgacaaccctgcaaaagtcgtcattgtgggcggcggtgtcgttggcgcaaacgcagctcgtatggctgtgggccttcgtgcagacgtcactattcttgatcgtaatgtcgatactctgcgtaaactggacgaagaattccaaggccgcgctaaagtcgtttattctacagaagacgcgattgagaagcatgttgtagaagctgacctagtaattggcgcagttcttatcccgggtgcagctgcacctaaactggtgacaaaagaacacattgcgaagatgaagccaggcgcagcggtagttgatgtagctatcgaccaaggtggttgctttgaaacatcgcacgcaaccactcacgcagaaccgacatacatcgtcgatgaagtcgttcactactgcgtagcaaacatgccaggtgccgttgcccgtacttctacttttgcattgaacaacgcaacgttaccttacattttgaaactggcgaataaaggctaccaaaaagcccttctcgacgagaaaggtttccttgaaggactaaacgttattcacggcaaagtgacttgtaaagaagttgccgacagctttggtcttgaatatgtagaagcagagcaagcgattgcaatgttcaactaa

SEQ ID NO: 22 - DNA, 762 bp, synthetic construct
  source 1..762: mol_type = other DNA
atggcgtactaacggcgacaaccctgacgcatacggccgtggtcgtatcatcggtgactaccgtcgcgttgcgctttacggtatcgacttcctaatgaaggacaaactagctcagttcacttctcttcaagagaaatttgagaacggcgaagaccttcacatgactatgcaacttcgtgaagaaattgcagagcaacaccgcgctctaggtcagatcaaacaaatggctgcgaaatacggtttcgatatttctcgccctgctgaaactgcacaagaagctatccaatggacttacttcggctacctagctgctgttaagtctcaaaacggtgctgcaatgtctctaggtcgtacttctacattcctagacgtgtacatcgagcgtgatatcgctgcaggtaagatcactgaagaccaagctcaagaaatgatcgaccacttcgtaatgaaactacgtatggttcgtttcctacgtactcctgagtacgatgagctattctctggcgacccaatctgggcaacagaatcaatgggtggtatgggtcttgacggtcgtacgctagtaacgcgttctaacttccgtttcctaaacagcctatacactatggggccttctccagagccaaacatcactgttctttggtctgaagcacttccagatggcttcaaacgtttctgtgcaaaagtatctatcgatacttcttctatccagtacgaaaacgacgatctaatgcgtccagacatggaatcagacgattacgc

SEQ ID NO: 23 - DNA, 6292 bp, synthetic construct
  source 1..6292: mol_type = other DNA
agtcaaaagcctccgaccggaggcttttgactaagttgagcatttaaagcttatttgaaaaaagtgtttgacagttgaacactcgttgcctataatcgaaagtcgagttatcgagattttcaggagaggaagctcatatgtctgatatgaagcatgacgtagatgcactggaaactcaagaatggctacaagcacttgagtcagttgtacgtgaagaaggcgtagagcgtgctcagttcctacttgagcaagttctagataaagcacgtctagacggtgttgacatgccaactggaatcaccacgaactacatcaacacgattccagcagatcaagaaccagcttacccaggtgacacaacacttgagcgtcgtattcgttccatcattcgctggaacgcaatcatgatcgttctacgtgcatcgaagaaagacctagaactaggcggccacatggcgtctttccagtcttctgctgcattctacgaaacatgtttcaaccatttcttccgtgctccaaacgagaaggatggtggcgacctagtttactaccaaggtcacatttctccaggtatctactctcgtgcattcgttgaaggccgtctaactgaagagcagctagacaacttccgtcaagaagtagatggtaaaggtatcccgtcatacccacaccctaaactaatgcctgaattctggcagttcccaaccgtatctatgggtctaggtccgatctctgcgatctaccaagctcgtttccttaagtaccttgaaggccgcggcatgaaagatacttctgagcagcgcgtttacgcgttcctgggtgacggtgagatggatgagccagaatcacgtggtgctatctctttcgctgcgcgtgagaagctagacaacctatgtttcctaatcaactgtaaccttcagcgtctagacggccctgtaatgggtaacggtaagatcattcaagaacttgaaggtctgttcaaaggtgctggctggaacgtagttaaagtaatctggggtaacaactgggattctctactagctaaagatacaactggtaagcttctacagcttatgaacgaaacaatcgatggcgattaccaaacgttcaaagcgaaagacggtgcatacgtacgtgagcacttcttcggtaagtacccagaaacagctgcactagttgctgacatgactgatgacgaaatcttcgcacttaagcgtggtggtcacgagtcttctaagctatacgctgcatacaaaaacgcggcagacactaaaggccgtccaacggttatcctagctaagactgttaaaggttacggcatgggtgaagcggctgaaggtaagaacatcgcgcaccaagttaagaagatggacatgactcacgttctacacctacgtgatcgtctaggtctacaagatcttctaactgacgaagctgtgaaagagcttccgtacctgaaacttgaagaaggttcaaaagagtacgaatacctacacgctcgtcgtaaagcgctacacggttacacgcctcagcgtctaccgaacttcactcaagaactgatcgttcctgaactagaagaattcaagcctctactagaagagcagaagcgtgacatctcttctactatggcattcgtacgttcactgaacgttctgcttaagaacaagaacattggtaagaacatcgttcctatcattgctgacgaagcacgtacattcggtatggaaggtctattccgtcaaatcggtatttacaacccgcacggccagacttacacaccagaagaccgtggcgttgtttcttactacaaagaagcgacttcaggtcaggttctacaagaaggtatcaacgagctaggtgctatgtcttcatgggttgcagcagcaacttcatactcgacaaacgacctaccaatgattccgttctacatctactactctatgttcggtttccaacgtgttggcgacatggcgtggatggctggtgaccaacaagcacgtggtttcctactaggtgcaactgctggtcgtacgacactgaacggtgaaggtctgcagcacgaagatggtcactcgcacatcatggcgggtacagttcctaactgtatctcttacgatccgacattcgcttacgaagtagcggtaatcatgcaagacggtatccgtcgcatgtacggtgagcaagagaacgtgttctactacctaacgctaatgaacgagaactacgcaatgccagcaatgccagaaggcgctgaagaaggcattcgtaagggtatctacaagctagaaacttacactggttctaaaggtaaagttcagctaatgagctctggtactatcatgaacgaagtacgtaaagcagctcaaatccttagcgatgagtacggtgttgcatctgacgtttactctgtaacgtcattcaacgaagtaactcgcgacggtcaagcggcagagcgttacaacatgctacacccagaagcggaagcgcaagtaccatacatccaaacagtaatgggtactgagccagctatcgcagcaactgactacatgaagaactacgcagagcaggttcgtgcattcatccctgctgaatctttcaaagttcttggtactgatggcttcggtcgctcagacagccgtgaaaacctacgtcgtcacttcgaagtgaacgcaggttacgtagtagtagcagcgctaactgaactagcgaaacgtggcgaagttgagaagtctgtaattgctgaagcaattaagaaattcgacatcgacactgaaaaaacaaacccgctatacgcttaattaagaaggtagataagaaatggcaatcgaaattaatgtaccagacatcggtacggatgaggttgaagttactgagattcttgtaagcgttggcgacaaggttgaagaagagcagtctctgatcactgttgaaggcgataaagcttctatggaagttcctgcttctcaagcgggtatcgttaaagaaatcaaagttgcagaaggcgacaaggtttctactggttctctaatcatgattttcgaagccgagggtgcagctgaagctgcgcctgctcctgcggcggaagcagctccagcagcagctccagctccagcagcagcggcagaactgaaagaagttcacgtaccagatatcggtggtgacgaagttgaagttactgaaatcatggtagcaatcggcgacagcatcgaagaagagcaatctctgatcactgtagaaggcgacaaagcttctatggaagttcctgcaccattcgcaggtacgcttaaagaaatcaaagtagcagcaggcgataaagtatcgactggctctctaatcatggtattcgaagtagcaggttcaggcgctccagcagcagctcctgcggcagtagaagcaccagcggcagcagctccagcagcatctgcggcaaaagaagttaacgttccagatatcggtggcgacgaagtagaagttactgaaatcatggttgcagttggcgatacagtggaagaagagcaatctctaatcactgtagaaggcgacaaagcttctatggaagttcctgcaccattcgctggtactgttaaagaaatcaagattgcagctggcgacaaagtgtctactggctcactaatcatggtattcgaagtggcgggcgcagctccagctcctgcagcggctccagctcaagcggcagcacctgcagcagcggctcctaaagcagaagctccagcggcggcagctccagcagcgacaggcgatttcaaagagaacgacgagtacgctcacgcgtctccagttgttcgtcgtctagctcgtgagttcggcgtaaacctttctaaggttaaaggttctggtcgtaagagccgtatcctgaaagaagacgttcagaactacgtgaaagaagcgcttaagcgtcttgagtctggtgctgcagcatctggcaaaggcgacggtgcagctcttggtctactaccatggccaaaagtggacttcagcaagttcggtgagacagaagttcaacctctatctcgcattaagaagatttctggtgctaacctacaccgtaactgggtaatgatcccgcacgttacacagtgggataacgcagacatcacggctcttgaagcattccgtaaagagcaaaacgcgattgaagcgaagaaagacactggcatgaagatcactccactagtgttcatcatgaaagcagttgctaaagcacttgaagcgttcccggcgttcaactcttctctatcagaagacggtgagagcctaattctgaagaaatacgtgaacgttggtatcgcggtagatacgccaaacggtctggttgttcctgtatttaaagacgtgaacaagaaaggcatctacgagctatctgaagagctaatggctgtttctaagaaagctcgtgctggtaagctaactgcggctgacatgcaaggtggctgtttcactatctctagccttggcggcattggcggtactgcgtttactccaatcgtgaacgcgccagaagtgggtatcctaggtgtatctaagtcagagatgaaaccggtatggaacggtaaagagttcgaaccgcgtctacaacttccactgtctctatcatacgaccaccgtgtgatcgatggtgcggaaggtgcgcgtttcatcacttacctaaactcatgtctatctgacattcgtcgtctagttctttaatagaagttctagcataaccccttggggcctctaaacgggtcttgaggggttttttggcttagccacaagcttatttgaaaaaagtgtttgacagttgaacactcgttgcctataatcgaaagtcgagttatcgagattttcaggagaggaagctcatatgagcaaagaaattaaagcccaagttgttgtacttggttctggtcctgctggttactctgcagcattccgttgtgcggatttaggtcttgaaactgtactagttgaacgttacagcactcttggcggtgtgtgtctgaacgtgggttgtatcccatctaaagcacttcttcacgtttctaaagtaattgaagaagcaaaagcaatggctgatcacggcgttgtatttggcgaaccgcaaactgacatcaacaaaatccgcatctggaaagaaaaagttgttaaccaacttactggcggcctaggtggcatggctaagatgcgtaacgtaactgtagttaacggttacggtaagtttactggtcctaactctatccttgtagaaggtgaaggcgagtcaactgttgttaacttcgacaacgcaatcgttgctgctggttctcgtccaatcaaactgccatttatcccgcatgaagacccacgtatttgggattctactgacgctcttgaactaaaagaagtgccagaaaaactacttatcatgggtggtggtatcatcggtctggaaatgggtacggtttaccactctctaggttctaaagttgaagttgttgagatgtttgatcaggtaatccctgcagcagacaaagatatcgttaaggtttacaccaagcgtatcaaagacaagttcaagctaatgctagaaacgaaagtaacagcagttgaagcgaaagaagacggtatctacgtttcaatggaaggtaagaaagcacctgctgaagcagagcgctacgatgctgttcttgttgctatcggtcgtgtaccaaacggcaaactgattgacggtgaaaaagcgggtctagaaatcgacgagcgcggcttcatcaacgttgacaagcaaatgcgtactaacgttcctcacatcttcgcgatcggtgatatcgttggtcaaccaatgcttgctcacaaaggtgtgcatgaaggtcacgtagcagctgaagttatctctggtaagaagcactactttgaccctaaagttatcccttcaatcgcatacactgagccagaagtggcttgggttggtaagactgagaaagaagcgaaagacgaaggcatcaagtacgaagtggcaacattcccatgggctgcatcaggtcgtgcaatcgcatctgactgttcagacggtatgactaagctaatcttcgacaaagaaactcaccgcgtaatcggtggtgctatcgttggtactaacggtggtgaactacttggcgaaatcggtcttgcgatcgagatgggttgtgatgcagaagatatcgctctgactatccacgctcacccaactctacacgagtctgttggtctagctgctgaagtatttgaaggttctatcactgaccttccaaacaaaaaagcagttaagaagaagtaa

SEQ ID NO: 24 - DNA, 1428 bp, synthetic construct
  source 1..1428: mol_type = other DNA
atgagcaaagaaattaaagcccaagttgttgtacttggttctggtcctgctggttactctgcagcattccgttgtgcggatttaggtcttgaaactgtactagttgaacgttacagcactcttggcggtgtgtgtctgaacgtgggttgtatcccatctaaagcacttcttcacgtttctaaagtaattgaagaagcaaaagcaatggctgatcacggcgttgtatttggcgaaccgcaaactgacatcaacaaaatccgcatctggaaagaaaaagttgttaaccaacttactggcggcctaggtggcatggctaagatgcgtaacgtaactgtagttaacggttacggtaagtttactggtcctaactctatccttgtagaaggtgaaggcgagtcaactgttgttaacttcgacaacgcaatcgttgctgctggttctcgtccaatcaaactgccatttatcccgcatgaagacccacgtatttgggattctactgacgctcttgaactaaaagaagtgccagaaaaactacttatcatgggtggtggtatcatcggtctggaaatgggtacggtttaccactctctaggttctaaagttgaagttgttgagatgtttgatcaggtaatccctgcagcagacaaagatatcgttaaggtttacaccaagcgtatcaaagacaagttcaagctaatgctagaaacgaaagtaacagcagttgaagcgaaagaagacggtatctacgtttcaatggaaggtaagaaagcacctgctgaagcagagcgctacgatgctgttcttgttgctatcggtcgtgtaccaaacggcaaactgattgacggtgaaaaagcgggtctagaaatcgacgagcgcggcttcatcaacgttgacaagcaaatgcgtactaacgttcctcacatcttcgcgatcggtgatatcgttggtcaaccaatgcttgctcacaaaggtgtgcatgaaggtcacgtagcagctgaagttatctctggtaagaagcactactttgaccctaaagttatcccttcagttgtttacactgagccagaagtggtatgggttggtaagactgagaaagaagcgaaagacgaaggcatcaagtacgaagtggcaacattcccatgggctgcatcaggtcgtgcaatcgcatctgactgttcagacggtatgactaagctaatcttcgacaaagaaactcaccgcgtaatcggtggtgctatcgttggtactaacggtggtgaactacttggcgaaatcggtcttgcgatcgagatgggttgtgatgcagaagatatcgctctgactatccacgctcacccaactctacacgagtctgttggtctagctgctgaagtatttgaaggttctatcactgaccttccaaacaaaaaagcagttaagaagaagtaa

SEQ ID NO: 25 - DNA, 84 bp, synthetic construct
  source 1..84: mol_type = other DNA
atgatttcaggcatcctagcatctcctggtaggacactcgtcgctttcggagttgaaaagttcattgctgagaaagttcagtaa

SEQ ID NO: 26 - DNA, 1397 bp, synthetic construct
  source 1..1397: mol_type = other DNA
gggccttaatgttgcggtgcgggatcgttatgtgtttcctgcgccacctcaatcgccggtttgttctgcaacacggtccagataaccagcgcacctaacaggtcgaacactgccagaactgcgaacagcgggctgaagccgatggtgtcagccagtgcaccgacaaccagcgcaaacagcgtacttgccagccatgcggacatcccggttaaaccgtttgccgttgccacttcgttacgaccaaacacgtcagaagagagcgtaatcagcgcgccagacagtgcctggtgggcaaaaccaccgatacacagcagcataattgcgacatacgggttggtgaacagaccgatcatacccgggccaatcatcagcacagcacccagcgttacgaccatcttacgggaaacgatcaggttcacaccaaaccaacgctggaacagcggcggcaggtaaccaccgaggatacaaccgaggtcagcaaacagcatcggcatccaggcgaacatcgcgatctctttcaggttaaagccgtaaactttaaacatgaacagcgggatccacgcgttaaaagtaccccaggccggttctgccaggaaacgcggcagcgcgataccccagaactgacggttacgcaggatctgaccaacggacattttcttcgccgtgctcacctggtgctgggcttcctgaccattaataatatagtcgcgttcttcatcggtcagatgcttctggtcgcgcggatgtttatagaaaatcagccatgccatcgcccagataaagctcaatgcaccggagatgataaatgccatctgccagctgtgcattacgattgcccataccaccagcggcggcgcaatcatcgcaccaatagaagaacctacgttaaagtaacctactgcgatggaacgctctttcgccgggaaccattcggagctggctttcagacccgccgggatcatcgctgcttccgcggcaccgactgcaccacgagcaatcgccaggccaccccaactaccggccagcgcggttgcaccacagaacacggcccacagtacagcaaacattgcataaccgattttcgtacccagtacatccagtacatagcccgctaccggttgcatgaccgtataagcagcagaataggctgcgatgatataggaatactgttgggtggagatgtttaactcttccatcagagttggcgcagctgccgccacagtgttacgcgtcaggtaaccaagcacggtgccaagcgtcaccagtgcgatcatataccaacgtaaccctttaattttacgcatatgacttccttagctcctgaaaatctcgataactcgaacgaggattataaccggtttcgggcaccttgtcaaacacttttttcaaataagctt

SEQ ID NO: 27 - DNA, 1065 bp, synthetic construct
  source 1..1065: mol_type = other DNA
aagcttatttgaaaaaagtgtttgacaaggtgcccgaaaccggttataatcctcgttcgagttatcgagattttcaggagctaaggaagtcatatgtcagagcaatacgcattagtaggcgacatcggcggtactaacgcaagattggctctctgcgagttatctacgggcacgatttctgatatcgtcacttatcctgcagcagaatacgaatccctcgaagtagtaatgcgccagtacttggatcgtcatgataccccgatctcttcagcgtgtatcgccattgcatgtccagtaacaggtgactgggtaagtatgactaatcaccattgggaattctctatccaggagctaaaatcacaacttaacctacaagtactagaagtaatcaacgattttacggctgtgagttttgctattccaagcttgaaaccagaggaccgtgtccaaattgggggtggtgaagcgatcgcaaacaagccaatcgctgtgtatggtgctggtaccggcctgggtgttgcgcagctgatccacggaggcaacctatggcattcggttccgggcgagggcggacatgttgacttagcagcgtgtacacctgaagaagatgatcttattgtttacttgagaaacaagtttggtagagtctctgctgagcgttgtttatcaggccaaggtatccgtaacatttacgacttcgttgtttcccaccatggtgcagcacctgaagattacacaccagcaatgatcacagaaaaagctcttaaagcggagtgcaaagactgtgaacgcgcactgtatcttttttgtatccttatgggccgatttggaggcaatcttgcgctaactaacggcagtttcggcggtgtgtacattgccggtggtatcgtaccaaaagtccaggaactttttatcaagagcggcttccgtgttgctttcgaagacaagggtcgatttaaagaatacctgcagtcaatcccggtttttctaatcactcattcagaacctggtcttctaggtgccggtacatatattcgccaatctctcaacattagtatcaactaa

SEQ ID NO: 28 - DNA, 1078 bp, synthetic construct
  source 1..1078: mol_type = other DNA
ttagagcaagtcgcgcactgcctcgcgttcagcctgtaattcctgagcattcgcgtcgatacgtgctctctgaaaatctgaaatctctaggccttcaacgatttcccattcaccgtttcgacttactgttggcagaccaacaaagatgccttccggaatgccgtatgcaccagtgctaggaattgccgcactgctccacgcctctgtaccttgtacccaatctctcatatgatcaatcgcagaggatgctgcagaagctgcagatgatttaccacgtacctcaatgatttcagcgccacggttagccacacgaggaataaactcttctacgtaccaatcgtgatcaactaagtcagtgaccttttctccaccaactgttgcgtacgtgatgtctggaaattgtgttgcggaatgatttccccataccacgatgttgttaaattctgcagaaccacggccaagcttagttgctagctgactgatcgcacggttatgatcaaggcgcatcattgcgttgaatcttgatgctggaacatctggtgccgcagctgaagctatcaacgcgttggtgttcgctgggtttccaacaactagcacacggatgtcatctgcggcgttgtcgttgatagctttaccttgaggaccaaaaattttgccgttgttagccagtaagtctgctctctcttcgcctttaccgcgaggtttcgcacctaccaaaaatgcagcattagcgccgtcgaatgcctcgttggcatccgcagtgatcgtgatgtttcgtaggagggggaaggccgaatctagaagttccatagcaacaccctctgccccgccaagagcttgcgggatttctagaagtttcagttctactggcgtgtcagtgccgaatacttcaccgttggcgatgcgccataaaagtgaataagagatttgaccagctgcgccggtgactgtgacctttttcgtagagacgttttgcggtgaattcatatgagcttcctctcctgaaaatctcgataactcgactttcgattataggcaacgagtgttcaactgtcaaacacttttttcaaataagctt

SEQ ID NO: 29 - DNA, 1724 bp, synthetic construct
  source 1..1724: mol_type = other DNA
cgatataagttgtaagcttatttgaaaaaagtgtttgacagttgaacactcgttgcctataatcgaaagtcgagttatcgagattttcaggagaggaagctcatatgactgacctaaacaaactcgtcaaggaacttaatgacctaggccttacagatgttaaggaaattgtatataaccccagttacgagcaacttttcgaggaggaaactaaacctggcttggagggttttgataaagggacgctaaccactcttggcgcagttgccgtcgatacggggatttttacaggacgttcaccgaaggataaatatatcgtttgtgatgaaactacgaaggacacagtatggtggaacagcgaagctgcgaaaaacgataacaaaccaatgacgcaagagacttggaagagtttgagagaattagtggctaaacaactttctggtaaaagactattcgtggtagagggttactgcggcgcaagtgaaaaacaccgcatcggtgtgcgtatggtcactgaagtggcatggcaggcgcattttgtaaaaaacatgtttatccgaccaacagatgaagagcttaaaaatttcaaggcggattttaccgtgctaaacggtgctaaatgtactaacccaaactggaaagaacagggcctcaacagtgaaaactttgtcgcttttaatattaccgaaggtattcagcttatcggcggcacttggtacggcggtgaaatgaaaaagggtatgttttcaatgatgaactacttcctgccattaaaaggtgtagcttctatgcattgttcggcaaacgtaggaaaagacggtgacgtagctattttcttcggcctatctggtacgggtaaaacaacgctttcgaccgatcctaaacgccaattaatcggtgatgacgagcacggttgggatgaaagcggcgtatttaacttcgaaggtggctgttacgcgaagaccatcaaactatcgaaagaagccgagcctgatatctacaacgctatccgccgcgatgcgctgctagaaaacgtaacggtacgtaacgatggttctatcgactttgatgatggttctaagacagagaacactcgtgtttcttacccgctttaccacattgaaaacatcgttaagccagtatcgaaaggcggtcatgcgaataaagtgatcttcctgtctgctgacgcgtttggtgtgcttcctccagtatcgaaactgacaccagagcaaaccaagtaccacttcctgtctggctttactgccaaactagcaggtacagagcgcggtatcactgagccgacgccaactttctctgcgtgttttggcgctgcgttcctaacgcttcacccaactaagtacgcagaagtactggttaaacgtatggaagcagcaggtgcagaagcttacctggttaacactggctggaatggtagcggtaaacgtatctcaattcaggatacacgcggcatcattgatgcgatcctagatggctcaattgaagatgcaccaactaagcatatcccaatcttcaaccttgaagttcctacttcactaccaggtgttgatccaagcattcttgatccacgtgatacttacgttgatccactgcaatgggaaagcaaagcgaaagatctagcagaacgctttatcaataactttgataaatacacagacaacgcagaaggtaaatctctggttgctgctggtccacagcttgactaa

SEQ ID NO: 30 - DNA, 6119 bp, synthetic construct
  source 1..6119: mol_type = other DNA
ttacgaccgaaggaccaagtcgttttggtaaagagagcgaaaacgtgaataacgctcagacagtcctgcttcacgctcgacattcggttgataaacgcgatgaatttccggcttcttacatacttcatccaccgattttcctgacgccaggcaagcaagtcgagccgcccccaacgccccacccgtttcaccacctttgtgagtaatcgttggcaagtgaagaatatctgcaagtaactgcgcccagaacgagctacgtgcaccaccaccaaccaatgagcactgttcgatgtgcgtaccgctttcttgcaacactcgcaaaccatcagcaataccaaagctcaccccttcgatcaccgcatagccaagtaaggcgcgattggtggaatgggtcatactgtggaacatacccgtcgcttccggatcgttatgtggcgtacgctcaccagacagatacggtaagaaaattggcgcttttgctttctcttcttcactaagctgctcaatttcagccagcaactccacttccgttgtgcctactatgcggcagaaccattgaaggcagctggcagcacttagcatgacactcatctgatgccatcgttgtggcaaaacatgacagaaggcgtgtacggcagactcaggtgcggggcgatatttctcgttgaccacaaacagtacgccagaagtgccaagggatataaacgcatcacccggattgaccgcaccaacaccgattgcgcttacggcattatcaccaccgccaccagcgacgatgacagatggacttagtccccatttgtgggccatttcagcggataaagaagccgatacttcacagccctcgaccagttcaggcatttgttcccgcgttagctggcatttgtcgagcagagaatctgaccagtcacgcttagcgacatccaaccataacgtgcctgcagaatccgacatgtcggaaacttttttgcccgtcattttgaagcgaagataatctttcggcagcagcactgtgctaatgcgggaaaagttttcaggttcatgcttgcgaacccaaattaacttgggagccgtaaatcctggcatcgcaaggttaccagcaatttcatgcagatccggagcagccgcttcaagttcggtgcactcttcgtgacaacgtgtgtcgttccacaaaatagctggacgaatcacatcattggcctcatccagaagcactgcgccatgcatctgtccagacaaaccaatggctttaattgctccccaatgctcagtgcatttctcacgcagcgccccgatcaaatagttcgttgcttcccaccactcttgcggtgcctgttccgaccaatgaagatgcgggcgttgaatcgtcagtggtgcgctgtgtgaagcgaccgtttcaccgtgttcatcaatcaccaacgctttcacttctgaggtgccgagatctatacctagatacatgcccattccttattgaatcattgagtaagctgctgcaactttttctcgcagcaatgtttcaaaatccgcgcgactagctagttcaccaaacaaagcgacatcttgcgcgtaagcggcgattgggtcttcagaatcaaacatgctgtgcacggcattttcatcaagaatcccatcctgatactcataaggcagtaaacctttgtgccactggtgcataaagacaaagaacagcgcaggcagcatcgcagtcgcatctggtgtaacacctcgctgatagcattcaatcatcgtcggcgtaatcatggccgggattttagaaaacccatccgcagcgacacgctggttggtgtctttaatgtatgggttggtaaagcgctctaacaccacctcaagataggtcggcagatcgataccattgtcaccaaggcaaggaatcacatcctgactgacgtaatcgtgcgcaatcttgtagataaaatccgtcaaagtactttcgtgaataaatgactggccaactaatgttccggcccaagcgatacagctgtgcgaagcattcaagattcggattttcgcctcttcgtaaggaataaccgactccaccatttccacgcctacattttccagtgcaggtcgaacgtctttgaaactgtcttcaattacccactgaatgaaagtttcacccatcactggcgcgttatcttcaatacctgtttgtgctttaatccgatatggcaggtcttcagcgggacgaggtgtaatacgatccaccatggtgttaggacttgtcgtattttccttcacccattgcaacacgtcagccttaccagtcagagtcaggaactccattagaccatctcggaatcgctcaccattgtgacgcacgttgtcacagttcagcagtgtcactggtccagcactatccgccatacggcgctcaaggattttcgtcaccgtgccgtaaattgtttggtggccaccgctcagatcagcttttaggactgcgttatcaacatccagtgagtgatcggttcttaagtagtacccgccttctgtgaccgtaaaggcgatcacttttgtctgcgcttttgcaccttcttcaatcagcggcgccaaatcttcctgccacgggagcaacgtctgaatagatttgatcacctcatattcacgctcccctgctggactgaccgtctctaacacatactcaccattctgcgcacggagagtttcaacggttgattctgcatcattgcgaatgttacctgccgcgatatgccagctttggtcacccgtttctagcaatttgtgcaggtaccaggcttggtgcgcacggtgaaatgaacccagaccgatgtgaagccacgtatattggtttttcattatatttcctcgtttttgtttgatggcttaactctaattgatcaaattaccaaataaagagcgatggatcacaaatgaacaaaagaccgataattggttttttgttcatttgtgagtaattgattgctattgcactcacgcgaagttagagagaagagtggctagagacgatagttttaaaaccgataggacactcgtcgctttcggagcaacagcgagatggttattaacgcaataagcgaattttcagtttaagccgtagatctcttcaggtgtcgcccgcgccccgttaaaccagactttcaattgtccgcataacgctatcaatttgcggctcaattgaaagtctgcatcaagacaacaacgttttctcattagaaacacgagaagttaatgtgtttaattaatacggtagaaacgtttagccaaattagtgtgttagcctttctatcaacgcttgttgatattcaggtaatttcgaacgagagacggattgaaatgctgatggcgttaggtaagtcatgcctgcatgtagcgcactcgctttcattggtaagaacacatcttctagtttaacgcctattctgcccgattccccgtaggtacttgctccaccagccgcagtcgtcaccaacagcatttcttttccctgccataatccaggtcctacactcccccaaacatcattcatgtaagctttcatcatcggagtaatattgaaccaatgggttggaaacataaatacaatgcgatcttgttctctcgttagctcacgctccttttgtgcatcgatggcgcgagtatcaaaaccataaatagtctctaggtttcggactgtgactccttcaacactttcagccgcttgctgcatccccttggtcatcactgacctttctggatagggatgtgagacaataaccagcgtttttatatccgttttatgaacctgctctgcagcggacacaacattgatgcatgtaagccacagaacttgcaaggcgagaaaaaatccagcctgcttaatgacgctattccatttcatttgcgctgttcctaaaattctatctttccacagttgttgtaaaaaaaattaagcccagcgagctgagcttaattttattgatgccaagattttattgaccagcaacgtattgctcgtcagtcactttctctttccagataacatttttgccatctttcgatgctgtaacaacaaggtgtgtcattggtgcatctggggtcgcgccgtgccagtgatcaatacccggtgggcaccaaacgctctcgttttcgctaaagcgaatcactttaccatctcgggtaccagtaagtgccacaccagaagtgacaatcatatgctggcctgcaggatgcatgtgccacgcggtacgagcgcctttttcgaatgtcacataagcagcgctgtaattcgcgacgtcattttcagggaatagcatgtccacttttacatttcctgtgaacaaatcttccggtccgttaaaggatttaagatcttgtgcgcgatacgcttgctggctgcctgccgcatcgtcagcagcattggtagttgaaaccattgccgtcgatgaaaccatccctgccagtaacgttaatatgagtttatttttcataatcgaattccctgttatttatttaggaccgagtccagaacttgttgtgcgttgttagccacgtcttcaccaacttgcttttcaatgatggtgatgaattcttttagctcatcaacggttacatcgttattcatactaatgctgtaatgagctgccaattggctgtttacacctggtatattggatagagtcgcaatcgtagcgacttcacgttgtttccaagtcagcacatcacgagcaaaaatgtcaccaaacaaatgagatttcaggtattcatcaatttgaggagagaaatcaaataacggcccttttacttcaacaccgatgagtttagtttggttttcagcaccaaattcaaggctggttttatctgtcggtagtggcgatgactctggaccaacgacgtcttcgattccattgctctttctttcttcaagaacctgcataaatgcagccaatgcattcaggctacgagggaaaccagcatatgcgtatagctgacccagcacttccttggtttcattaatggttaaacctgcgtccaagccttcgttcaagcttacttttagcttctcgatttcaccactggcggcaaacgctgcgatcggaataatcgccttctctttagtatcaaaaccttctgccgccgttgcggtattcatagtcaataccgctagcaaactcgccacaaaaatggaccatttactgaaatatctgcctatatgaaacatttgtgtctccctattccctttgcatttttgagtcttcaatcgcactctctaaatataaagttatcgcaaaatttgcatactactttgcctagaagagtttaattattgcctaatcttctattaagcaaactccactagaattgagatataaataaaaacatgatgttacatttggaaacaataataacgcgaccatttacttgactattaaaatgtaaaaactactcaataaacaattaatcgttattcgtactagctagtcatttaaatgtagaagtaaacatcgtctatttaattaaatagtaataatcaggcgtaatttcatttttatctcggcccgcttattaacgtagaaatgcctgttaatctttttgattaacaggcatttatttacaaaatccaatgtattattcaataacctctatagtgactttcattgaaccaccagaagtaaagcgcgataaatcgccgtcaactttcccgaggttaatcaatccagatgcatagccaaagttattataaaacacgactaagttcccccaaggtgcgtagtaagaaatgtctcccgctttggaactcacgccagctggcgccccttccttggttaacttcgatggtaggtaggctattttttcagtactggcgtaatcttccaactctaaagttaatggtagttgggcgataaaatcttgtgtgctcggactatcatttaacgtcgcagtgacggtgcgatcatcaaatacaaaacgaatcttcattgtactctccccggtaataactccgctctcggtcacgttgccggaagcgaaacaaatttggctaataagtaatgcgggcaacactgtaagcgtggataaaatggttttgtgccacttctttaacaacatagtagtgattcccttttagttaagtgacagtt

SEQ ID NO: 31 - DNA, 6623 bp, synthetic construct
  source 1..6623: mol_type = other DNA
gtgagcgaaaacagcgttaaaatcgcacatttaacattcattcagtctactataacccgtatggggcaaaattcttttctgctaaaggggtggtgtattacactaattgccgctctatttgcgttatctccgagttcatctaacaagtcgtttgctcttatctcttacatgccattatttttattctggttgatggatgcttattatctttcgctcgaacgttcatacatccagctttataacctagtggcaaagaatgaggttaaaagtgatgcacttaccttagatttagaacaagtaggcggtggagagagtttgttcaatacggctttttccaaaactctgttggtgttttatggaagtgtgatttctatagttttatttttgatgtttggagttttgggctaatgagtagtgaaagctgtgattatttaatatccctatcttttgctggtgaagatagagagtatgtcgatgctgttgcctctgggttaaagaataatggtatatcagttttttacgacaagtatgagcaagcctctctctggggtaaggatctttaccagcacctaacagacgtttatcaaaatagatcccgctacgttgtaatgtttatttccgagcattatgctaaaaaactttggacctcacacgaaaaacgaatggctcaggcgagagcatttaaagaaagtagggagtacatattacccgtaaagtttgatgacactattattcctgatattccagacacaactggttatttggatgcccgagaatttgtgccagatgagatagttaagttcattaaacaaaaactgcaagatgacgggataacaataacaaaacaaaagtcactgaccaatgagttattaaaatcgacagcagaagagctagtaaatttaattagagaagaactaatccagcacgaacagaaaaccgagagccttaatagtccaattcggtcccttcattctgaagctgagttttccaaactttgggaggcgcggactaagagacaacatcaattaaaaaagtggttattaaatagtttttctaacaaatatcaatcgaaatccatattaataaaaaatgaattaatacaaaggcttaatttaaatgaaagagatgtttatttcgatgcagtgtacgaaaaccctgtaaacccgctaggtatatcagaagttgcggcagacctagaaaaattaagtatgatgctggatacaaacgaatgagccatttgaagtcaataaataaaattcctttcaaagagttacttatatgtggaagtagagtttatgaaacgcaaaaaattattgaatacaaagatatagagccaattgtaattgcgaatgggataaagccaagaatatggctaacggtattagttgaaaatggcgattcgttcgctttggtagatgactccagagcgaagcatgaatccgttatttgcaatgttacaactagtaacgtggaaatttatgtagatgaccattttatccttaaaggcaccagatcgagaacggagcgatttcatatacatcacctagacttaagaagcttaggttttagtgtatacggttcggatgaatctggcttgtatgcgaatggagtatctttaaattctatctcagctagaggcggtcgttgccttatcaagttaggttaatttacgcttgctgggtatccattcggcggctttcgttcgatggccgccacgaaacggtaagacgttttcttttgcaggatagaccttaggttggctgatgtgaccatacatttcgccaaactaaaaatgttcgtcacgcaaagctttctaattccttcgggctgaactcacgacggtgtggtgacaatggcgcgtttctcgatgattttaaacctagactaacgacggccattgggtaatcaaaccaacgctacttggctcttcagaaaaaagagcagggaagtcatgaagttagctccacaagtcgtcagtaaacattctagtgaatctaagttttagtgaccgcgtaacttaatgtcaatgataaatcaacaagattcaggtagatgtgggatctgaatatggcgtgtattctcagtctggttttttcagctatatttatcttgaatatcacatttaacaagatgggctagacaatgaaactagcatattacgtaagcgatatagctcaagataatagcgaccacgaggtccataatagtggtgaccatgaagtccataactcgggttgtcctcattttgatagaatccaatccaagacttatttaggcttatttgataattgtgctgaagcagtcattgaagcaaagaaaaaatacgaccagtcaaatggttgttattggtgttgtaacgaatgccacatgtcacactagttaggaacgggaactttctacaaacaaatcgcgcacgcacattctggtagtaagctcccaatagaaatgaaggcgacacttgagaaacaccaagagccatacgcagcaggaacttggtgcaagtgacgccgccaataactccggattaaagttaaagcgctttgagtttataagaagcagcaaacaacatttcactgaacgccagtaagtcggacagtcagaaatgttgagagcgtatctacacaatgagaactctagaaaatcagagcgtaagaaagtacggctcaattagaatgctcaggcgaattagtagcagaacttcaagaacgccaacttgaactgataaaccgagcgaattttagttcaaggatatagttgctgtaggcaagcaatagaaaatcaatccagctattcggttacttgaatgagccttaacgctgtccatcgaacaatgtgagcaagcagactgggtttattcgcggcaatgtgaagatccttcccctattttgtctaatagcataaaagaaaaagccacctatcacgcgcggcgtaataggtggctttgtaattcaacggggcccctgaagtcagccccatacgatataagttgtaagcttatttgaaaaaagtgtttgacagttgaacactcgttgcctataatcgaaagtcgagttatcgagattttcaggagctaaggaagctcatatggattttttgtccgattttttgactaaatttggttcacagttacagtccccgacgctcggctttttaattggcggtattgtcatcgcagccttcggtagccgacttacaatcccagatgcagtatacaagttcatcgtttttatgctgttaatcaaagtcggtctttcaggcggtatcgcaattcgtaataccaacatcacggagatgctactccctgcgttatttgctgtactaatgggcatcctaatcgtatttatcgggcgttttaccttagcaaaactgccaggtatcagaacggtagacgcagtggcaactgcaggcctattcggcgcagtgagtggttcgacccttgctgctggaatcacggtcatggaaggccaaggtgttttctacgaaccgtgggcagcggcactttatccttttatggacattcccgccctggtgacagcgattgttgtagcttcactttataagtcaaaacagcgcgaggttgaagcagatgacttcagcaaacaaccagtagcagctggtgaatactctggtgagcctgtttacccaacaacgcgtcaagaatacctgggtcaaaaacgtggtaaggctactaacagagttgaaatttggccaattgttaaggaatcactacagggttctgccctatcagcattgcttctcggacttgctctaggtttgcttactcgaccagaaagtgtctttgagagtttctacgagccactcttccgtggttttctttctatcttgatgctggtaatggggatggaagcttggtctagacttggcgaactgcgcaaagttgctcaatggtacgctgtctatgcgtttattgcgccgctactccatggatttattgcattcggtctaggcatgatcgcacactatgtaacaggcttcagtcctggtggtgttgccctattagcaattatcgcggcgtcttcaagcgacatctctggcccgcctactttacgcgctggaattccgtcggctaacccttctgcttacatcggtagttctactgcaatcggtacaccagtagcgatcgcaatcggcataccactttttatcggccttgcgcaagcaacaatgggcggctaaggcccgcatgtagtcaaaagcctccggtcggaggcttttgactcttggtaaggagtagtgttattccacggtaactgcttttgccaggttacgacaagatgaataagcaagatgccgagcctacgtgcatggtgtcgaacgtaacaaaagaatcagtgtaagaacttaatcggcgtttaaagctgatgactggtttttaaaaaattggacgtcgtggtgaagttgaattcatggtccaggcaagaaagcggataaaacctttaaagaagcaaaagccaaatacggtgtaattaagctgacatcagcactgttaatcttttaaccctttagtgcgcattatgtatattatgttaaataaagcataacgctacgccaaatctttactcgtttagcctcctatccctctcttatttcaatttcatctaggggtttgaggctttcattaaacataatggctatttaccataaaatacttaatatagatcttaataatctatcgattgttaagatctatattaaggctaatcattttctacaaacacttgaatggcttcatagatgtcttgtctacggactggtttggaaatgaagctgtccataccggcttctaagcatttgtcacggtcactttctaaagcgtgtgctgttaatgcgatgattggaatcgtgatgcctttttctcggatcaagcgcgttgcggtaatgccgtccatgactggcatcgatacatccataagtataatgtcgattgagtcgctgttatcttccagaaatgccagtgcttcttcgccatggcttgagatgtgaacgttatgtcctaacttgttgagaattaatttaatcacgagctggtttgaatgtgtatcttcaaccacaagtaaatttaacgctttgactgggctgtttgcttcgatttttacaggacgattctgatgaagcctagtcaataccggaatctttattgtgaatgacgagcccacgtgttctttactatcaactatgatggttccgcgcatcatctctactaaatgtttggtgatagccaggcctaatcctgttcctccgaaacgtctggttatcgatctgtcgccttgatgaaaagccgtgaataaacgagcttgtttggcttttgatataccaatgcccgtgtcagagacggaaattattagctcgtctttctgttccaaaaccgaaacgctcactgtaccgctgtcggtgaatttaatcgcgttaccgataagattaaataaaacctgagataacctggtcgaatcaatccaatactgtttgtcttgcggtatgcggcaatcaagattgaagttgaggtttttgctaatagctatttgtttttgctgggaaatgacaaaagtcacagcgtcacttaaattggtccactgttcatgtaactgaaagctacccgactcgattttagagagatccaggatgtcacttatgatggcgagaagcagttccgcagagctctccatttgacacagcgcttcgaactgctcgttggataatgttgattgccttaatatatcgagcatacccagaacggaattaagcggcgttcggatttcatggctcatcatggccagaaactcggacttagattggtttgcgagttcagcttcttcgcgtgctttagctagctctgttgtacgtacatcaacgatacgttgtaacttctctttggtctcgatgtctattaccgctcgctctattagagggcgaaagcgtcgaagtgtttctttgttttcaatactgaagtgccctttttgtgctccaattaataggattatattttgagttacttcagagcgaatgccagtcagaatgacagagttcacgtggttcttgacgaatatatttaagttagagaactcgcccaaccttaatggttcgaacagtaatacacattcaccatttagcgctcgctccatggtgttaccgtgtaaccaattaaccgtgtcgaatacgctgttcgttgatattaacgttttgaaagattgcctgttatcgtcacgagtaataacgataaagtcttcaaagttaatgtatttttttaagaccgtatttaagccagagaagatttcattgcggtttttggcttcgctcatggctgatattgccgaaagaatggctttgttctcgtcagcgagcaatttttctctatgcttggcttgttgtaactcaaccaatgcttcctgcaaagcttcttgttggtaggtctctaaatatttatctttcataaggcttatggaatatcgctgatgagatcattaggttcccatgtccattttcgccaccaataaatcttccttgttcgccaaaggtaaatgggcatataaatggcaggtgttccagttcttggttgatttgttcacaaacgctgttcatgtcttgcttcaagtgcaacatgggcc

SEQ ID NO: 32 - DNA, 84 bp, synthetic construct
  source 1..84: mol_type = other DNA
ttaaccagtgttacgcatacctgccgcgatacctccgaaagcgacgagtgtcctgcttttgagtgcggcgtatttctcgttcat

SEQ ID NO: 33 - DNA, 1711 bp, synthetic construct
  source 1..1711: mol_type = other DNA
aagcttatttgaaaaaagtgtttgacagttgaacactcgttgcctataatcgaaagtcgagttatcgagattttcaggagaggaagctcatatgagttttcttgtagaaaaccaactattggcgctcgtcgttattatgacagtcggtcttttgctgggccgaattaagatctttggttttagactgggtgtagctgctgtgctttttgttggtttagcattaagcacgatcgaaccagatatctcagttccgtcgctcatttacgttgtgggtctatccctttttgtatacaccatcggtctagaagctggtccaggctttttcacttccatgaaaactacaggcttacgcaacaacgctttaacgctcggcgcaatcatagctacgacagccttagcatgggctctaatcactgtcttaaacatcgatgcagcttctggcgcgggtatgcttacgggtgcgttaactaatacacctgcaatggcggcggtagttgatgcgcttccgtctcttatcgatgataccggccagctgcatctgattgcagaacttccggtcgtagcgtatagtcttgcatatccactaggtgtattgattgtcattcttagtattgcaattttttctagtgtattcaaagtggatcataacaaggaagcggaggaagctggcgtagcagttcaagaacttaaaggtagacgtatccgtgttactgtcgcagacctgcctgcacttgagaacatcccagaactccttaacctgcatgtaatcgtttcgcgcgttgagcgtgatggggaacagtttatcccattgtacggtgaacacgctagaatcggtgacgttcttacagtagtcggtgccgacgaggagctaaaccgagcagaaaaagcaatcggtgagcttatcgacggcgatccatactcaaacgttgaactggactacagacgtatttttgtatcaaatactgcggtagtgggtacgccgctctctaaactgcaacctctatttaaagatatgctaattacacgtatccgccgcggtgacaccgacctagttgcttcatctgatatgacacttcaactaggcgatcgtgttcgcgttgtagcaccagctgagaaactgcgtgaagcgacacaattactgggagacagctacaagaaactatcagacttcaacctcttaccactcgcagcaggcctaatgatcggtgtgcttgtgggaatggttgaattcccacttccgggaggctcctcactcaaattgggcaacgcaggaggccctttagttgtggcactattgctaggcatgattaaccgtactggcaaattcgtgtggcaaattccttacggtgctaatttggctttacgccagctaggcatcaccttgtttctagctgccattggtacgtctgcgggtgcaggttttagaagcgctatctcagaccctcaaagtctaactatcatcggctttggggcgcttctaacacttttcatctctatcacggtcctgttcgtaggccataagttaatgaagatcccgttcggtgagacagccggtatccttgcaggcactcagacgcacccagcagtcttgtcatatgtatctgatgcttcacgtaacgaactacccgcaatgggatacacttctgtatatcctttggcaatgatcgctaaaatccttgcagcccagacactactttttttactcatttaa

SEQ ID NO: 34 - DNA, 1671 bp, synthetic construct
  source 1..1671: mol_type = other DNA
atggaactggttatttctctattacagcaaatgtgtgtttacttagtactggcgtacatgctgagtaaaacacccattatattgcctctgttgagcatctcttctcgcttaagccaccgcttaatttgttacgtacttttctcgggcttctgtattctcggcacctattttggtctgcatattaacgatgcaattgccaatactcgtgctatcggtgcagtcatgggcgggctatttggtggccccgtggttggtttcgcggtaggacttactggtggtattcaccgctacacactcggtggttttaccgatctggcttgtgcaatctcaaccacggctgaaggggttatcggtggcctgcttcacgtatacctaatcaaacgcaacaaaggtgccctgctatttaacccaagcgtcgtgttcagcgtgacttttgtggctgaagttgtacaaatgatcttactgctcgcggtcgcaaaaccgttcgaccaggcttatgaactggtatcagcgattgctgctccgatgatcattgccaactcgtttggtgctgctctgttcatgagcattcttcaagaccgaaaaaccatttttgaaaagtactcggcaaccttttcacgccgagcgttaaccattgccgaccgctcagtgggcattttaagtaacggttttaacactgaaaacgcagaaaaaatcgcccgtattatttatgaagagacaaaagtaggcgcggtcgcgattaccgatcaggagaaaatcctcgcctttgttggtatcggtgatgatcaccacaaacccaacaccccgatttcatcgcaaagtaccttagactcgatggaaaagaacgacattatttatctggatggctccgagcgcccttatcagtgctccattgcgaaagactgtaaactgggttcagcgcttattattcgacttagagcgggcaaggaagtaatcggtactatcaaactctacgagccgaagcgaaaactgttttcgacggcaaacatgtcgatggcagaagggattgcacaactgttatccagccagattttatatggagattatcagcaacaacaaaccttattagctcaggcagaaatcaaattgctgcacgcgcaggttaatccacatttcttgtttaatgcgctcaataccatcagcgcgattacacgacgtgacccggataaagctcgagaactgatccaaaacttatcgcacttcttccgcagtaacttaaaacagaacatcaacacggtaacgctaaaagaagagcttgctcatgtgaattcttacctgagtattgaaaaggctcgttttactgaccgactcgaagtcgagatagatattgaaccagaattactggacatcaagttaccaagctttacacttcagcctttggtagaaaatgccattaaacacggcgtctctaatatgctggaaggcggcaaggtgagaatatacagccaagcgcatccacaaggtcatctgattaccgtggaagacaatgcaggcagttttgagccacctaaagacaaccactctggtttaggtatggagattgtcgacaaacgtctcacaaatcagtttgggcgtgattcagcgctaaaaattgcgtgcgagaagcatcaatttactaaaatgagttttattattcccacaacttcgtaa

SEQ ID NO: 35 - DNA, 9520 bp, synthetic construct
  source 1..9520: mol_type = other DNA
acccacaaccaataaatccggttgccattctttgatctgtttttcgatgtcgtcccagtttggaataccatcattggctttaaaagccttgagcggagaggcggtaccggtaatttcctggccaatcgcgctgccgatgcttttggttccaaaatcaaaagccataattgtacgtgacataaggtgtctcaaatctcaatctaggggaacagtctgccttatcaggcatgtcctgcatcagaagaaagctgactcggatcgatccccagtttttctactgcttttttccaacggtctgtaattggagtatcaaagatgatttctggcgtagcttctatggtcagccaagagttttcaactaactcattttccagctgccctgcgctccaacctgcgtagcccaaagcaaccaaatagtcgcttggctcagcttcggtccccaacacagtcagaatgtctcgcgaggttgtcactgcaagctcatcagtcatctgaatactggattcatagtaatctttcggtttatgcagaataaagccacgatcttctgagatcgggccgccgttatatacaggccggtcaaggcttgcttcgaacaaacgcgggtgtacaggttgtactttgacttgcttaagcatgttacccacggtgatatcgactggcgcattgattatcaaccccattgcgccttcttcgttgtgctcgcagacgtagatcacgctattctggaaatagggatctttcattccaggcatggcaactaaaaaatggtttgtgaggttcatattcatactgatccttaggactcagaagtgcacttaagcaaattggcaatatatgcatgaatttgcttaagtttgcatttagctctctactataactttactatatatttagggtctgttgacctttcgcgctgatttttgcagctgtttgtgggttctttatacaaggcagaggctttgaaatgtagttggcctacctgataagccgataacgcagtagaaagtatccacaaacgctgcccgaagggttcggctaaaagcgctttactctttgttgagaggtattttgcttagaatgactaggctataaacctctcgccgcgattaaaacgcttttatctcgaacaaaaattaaccacgcaagataaacagaccctatttattcgccttttactcggcgttcaatggcatccattagtttgcccgtaattgagatatcaaatgctgcttcgatttcacgaatacaggtcgggctggttacgttgatttctgtcagcttatcgccaatcacatcaagacccacaaaaataagacctttttctttcagagttggtgctacggtttgggcaattttaatgtctgtttcacttagaggacgagcttcacccgtgccgcccgccgcaaggttaccgcgcgtttcgcctttcgccggaatacgtgccagacagtaaggcattggctcgccgtcaaccacgagaatgcgcttgtcaccattgctgatatcaggcacgaatgtttgtgccatcgcgtaatgctgaccatggttagtcagcgtttcgatgatcactgatacgtttggatcgttctcatttacgcggaaaatagacgcaccgcccataccgtcaagaggctttaagatcacatcgccgtgttcttcacggaatgctttaatcttttcagctttacgggtaacgatagtggttggagtcaattcagggaaccatgctgtaaacaacttctcattgcagtcacgaaggctctgaggtttgttgacgatcagtgtgccttgctcttctgcacgctcaaggatgtacgttgcgtagatgtattcagtgtcaaacggaggatctttacgcatcagaaccgcgtctaactcagatagctcgatagtttgctctgatttgaattcgtaccagccgtttggatcttcttttagctcaaccactttggtgtcggcaatggccacaccttggtcgaggtgaagatcattcatttccatgtagtggatttcgtagccgcggcgttgcgcttcaagcatcatggcaaagctagagtctttcttgatgttaatggacgaaattgggtccattacaataccgagtttgatcattatttttctccgttttaaccaagatcgccgaaacgaacttgtaaagctgtaattgcggtaagagccgctgtttcggtgcgaagtacacgcggaccgagaagcgtctcttcaaattggtactcgcgcgtcatatcgatttcttcagctgacaaaccgccttcagggccaatcagcaggcgcactttttcaactggtgtaggcagggtgttgatcgagtatttggcacgagggtgaaggttgagcttaagtccatcgtactcttctttgctccactcttccaagctcatgattgggcgaatttctggaacgatgttacgtccacactgctcgcaagcactgatagcaatcttctgccattgggccagtttcttctcaaatcgtttttgatcgagctttacgccacaacgttcagaaataaggggagtgatggtatttactccgagttcgactgacttctgaatggtgaactccatcttgtcgcctcgtgaaatcacctgtcctaggtgaagatccaaaggggattcaatgctgttctctacgcgctcagagatgtctacgaggacattctttttgctgacttctgcgataactgcgggaaactctgcaccactaccgtcaaataggagaacttcctgaccttccttcatacgaagtacgcggccaatatggccagcggcgtcgtcacttaaagcgagtgtaccaagttggtgaatggtttctggatgataaattcgagggatacgcatgagatttccagcaagagaataaacttagttcctaacatggatgctttccatcgaaaaaacaaggggcaagaggcgtttcaattggagtaaatgaaacgcctcaaacagaaagattagcgattgtcgcgattggtgaggattttagttacggggccttgacaattaatcatcggctcgtataatgtgtggaattgtgagcggataacaatttcacaaaggaggtgcggccgcatgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccgtggaattcttggtcgatgaaaagggcaaccacgtcttcatcgaaatgaacccacgtatccaggttgagcacaccgtgactgaagaagtcaccgaggtggacctggtgaaggcgcagatgcgcttggctgctggtgcaaccttgaaggaattgggtctgacccaagataagatcaagacccacggtgcagcactgcagtgccgcatcaccacggaagatccaaacaacggcttccgcccagataccggaactatcaccgcgtaccgctcaccaggcggagctggcgttcgtcttgacggtgcagctcagctcggtggcgaaatcaccgcacactttgactccatgctggtgaaaatgacctgccgtggttccgactttgaaactgctgttgctcgtgcacagcgcgcgttggctgagttcaccgtgtctggtgttgcaaccaacattggtttcttgcgtgcgttgctgcgggaagaggacttcacttccaagcgcatcgccaccggattcattgccgatcacccgcacctccttcaggctccacctgctgatgatgagcagggacgcatcctggattacttggcagatgtcaccgtgaacaagcctcatggtgtgcgtccaaaggatgttgcagctcctatcgataagctgcctaacatcaaggatctgccactgccacgcggttcccgtgaccgcctgaagcagcttggcccagccgcgtttgctcgtgatctccgtgagcaggacgcactggcagttactgataccaccttccgcgatgcacaccagtctttgcttgcgacccgagtccgctcattcgcactgaagcctgcggcagaggccgtcgcaaagctgactcctgagcttttgtccgtggaggcctggggcggcgcgacctacgatgtggcgatgcgtttcctctttgaggatccgtgggacaggctcgacgagctgcgcgaggcgatgccgaatgtaaacattcagatgctgcttcgcggccgcaacaccgtgggatacaccccgtacccagactccgtctgccgcgcgtttgttaaggaagctgccagctccggcgtggacatcttccgcatcttcgacgcgcttaacgacgtctcccagatgcgtccagcaatcgacgcagtcctggagaccaacaccgcggtagccgaggtggctatggcttattctggtgatctctctgatccaaatgaaaagctctacaccctggattactacctaaagatggcagaggagatcgtcaagtctggcgctcacatcttggccattaaggatatggctggtctgcttcgcccagctgcggtaaccaagctggtcaccgcactgcgccgtgaattcgatctgccagtgcacgtgcacacccacgacactgcgggtggccagctggcaacctactttgctgcagctcaagctggtgcagatgctgttgacggtgcttccgcaccactgtctggcaccacctcccagccatccctgtctgccattgttgctgcattcgcgcacacccgtcgcgataccggtttgagcctcgaggctgtttctgacctcgagccgtactgggaagcagtgcgcggactgtacctgccatttgagtctggaaccccaggcccaaccggtcgcgtctaccgccacgaaatcccaggcggacagttgtccaacctgcgtgcacaggccaccgcactgggccttgcggatcgtttcgaactcatcgaagacaactacgcagccgttaatgagatgctgggacgcccaaccaaggtcaccccatcctccaaggttgttggcgacctcgcactccacctcgttggtgcgggtgtggatccagcagactttgctgccgatccacaaaagtacgacatcccagactctgtcatcgcgttcctgcgcggcgagcttggtaaccctccaggtggctggccagagccactgcgcacccgcgcactggaaggccgctccgaaggcaaggcacctctgacggaagttcctgaggaagagcaggcgcacctcgacgctgatgattccaaggaacgtcgcaatagcctcaaccgcctgctgttcccgaagccaaccgaagagttcctcgagcaccgtcgccgcttcggcaacacctctgcgctggatgatcgtgaattcttctacggcctggtcgaaggccgcgagactttgatccgcctgccagatgtgcgcaccccactgcttgttcgcctggatgcgatctctgagccagacgataagggtatgcgcaatgttgtggccaacgtcaacggccagatccgcccaatgcgtgtgcgtgaccgctccgttgagtctgtcaccgcaaccgcagaaaaggcagattcctccaacaagggccatgttgctgcaccattcgctggtgttgtcaccgtgactgttgctgaaggtgatgaggtcaaggctggagatgcagtcgcaatcatcgaggctatgaagatggaagcaacaatcactgcttctgttgacggcaaaatcgatcgcgttgtggttcctgctgcaacgaaggtggaaggtggcgacttgatcgtcgtcgtttcctaaccggcagttaaagtctttaaaaagtatgactttatccattctatcactcagatattagctttcgtaataagcaagttaataataacaaaagaaatgttggtcaatatttcctattgagacttaattgaactgaggattaggaaagctggacaccagtgaagctaagaggctgacgacatttttgacacgaatacacggcttgtttgcgcagcactttgttgtgacggcgaatagaaaggggatatgtcgtgcagcgacatcggtattcgaaggttttgccttgaacggaagagacttcgaaactatgtgtggttttggccggaacgttaaatacggattccattacggtttgccactctttgccatgaggacggacgcgaccgtaaacctgatatgtgattaagtgagcgacctcatgcggtatcacttcttgcaagaacgcttgggtgttttctttaaataaaaccgggttaaggcgaatctcattgagctgaaggtacgcttttcctgctgctttacctcggactttaaaggtcaggctgggaatagggaatgaacgcttaaaagcatgttcagctaatgcgatatagtgcgccatcacctgttttgctttgtagcttagttcaatgtccattcgttcctctcaagttcaataaaaagcccccgcagttgcgagggccggatcatagcatcactgaattaaggtgtgtgttttttcttaatcgttgcatggtaggcgtgccaagtaccgtagccgagaataggcatagtaaacagcataccgattccgtaggtggcgaaaccaattaagatacccccacagatgattgccgcccacacgatcattgctggaatgttggatttgaccgcattaaagctggtgaataccgccgtcatcatgtcaactctgcgctccatcattaatggaatagagaaggcagaaatgctgaataccacgcttgctagtacaaaaccaatgagtgacccaatcactaagaagggcaagaactctgtcagcggtgcaccttgaacggaagggtacaatgcatggagaagtgcggcgattcgcatccagaaaatcatacaaaccgcaagcagaaccgcaaatgcccattgagagcttgagttacgacctatcgctttcattgagtggaacaggcgagctttgtggccgcgctctcgttcccaactggcatcatacaaacctagggctaaaaatggcccaataagcatatagacgatcaaactcggcatcacgacaaggtgcgtcccttgccattgcacaagcaatacgatgccaatcgctgctgccatgaagcacagtccataaaaggcactaatgagtggcatacgcacgaagtcatgaagccctagcgcgagccagtggaaaggcgcgctaagattgaccgtgttgcacggaatggttctagcgtattcgttatcagggacttcctttcgtttactatggaagtcggatggattgactgttcgaggcataactcctccttggcttattcctacacagttgtctgtcaaatgacttgtacccatcggagtaaataattgttcattctaacgcgttcaaatactcgataattccgcgacaatgtttgactggaaaataattatttatcaaaacattacgcgttaatttcaggcgttttgactacgctattcctgacagtttgcttacttggattggtactaacccctataagttttaaaagcataaaggatgtttagctaacgttttccccaaggttgctgtcgtcacactagcttacatccacgttgttacggaaacgcaatttaacaatattttaactattggcggagttgggggaatttacaactatttgcttaaaatttgttcttagtgggtggggctggaggatagagaaggaggggagtcgggggaaacaaaaagcccccaccataaggcgagggctttggaaattcagagcaagctcgattacttgatgccagcgaattcgcgaagaagtgctgctttatcagtcgcttcccatgggaactcttcacgaccaaagtggccgtatgctgctgtcttcttgtagattggctgcagaaggttaagcatttcttgcagaccgtatggacgtaggtcgaagaactgacgtactgcttcgatgatgatgtcgtgagatactttctcagtaccaaacgtttccaccatgattgacgttggatctgccacaccgatagcgtaagaaagttggatttcacaacggtcagccataccagctgcaacgatgtttttcgctacgtaacgcgctgcgtatgctgcactacggtcaacttttgatggatctttacctgagaacgcaccaccaccgtgacgagccgcaccgccgtaagtatctacgataatcttacgaccagttagaccacagtcacccattggaccaccgattacgaaacggccagttgggttgatgaagaagttagtctctttgttgatccactctgaaggtagaactggcttgatgatctcttccattaccgcttcacgcagttcaggagttgagattgaatcacagtgctgagttgatagaacaacagcatcgatacctacgatcttaccttggtcgtattggaacgttacctgagatttcgcatctggacgtaaccaaggaagtgtaccgttcttacgtacttctgcttgtttttcaacaagacggtgagagtaagtaattggagctggcattaaaacttcagtttcgttacatgcgtaaccgaacatgatgccttggtcaccggcgccttgctcttttggatccgctttatcaacaccttggttgatatctggtgactgtttaccaattgtgtttagtactgcacaagagttcgcatcaaaacccatatctgagtgtacgtagccgatttcacgaacggtttcacgagttagctcttcaatgtcaacccatgctgaagtcgtgatttcaccacccaccattaccataccggttttaacgtaagtctcacat

SEQ ID NO: 36 - DNA, 7373 bp, synthetic construct
  source 1..7373: mol_type = other DNA
gaatatcagtgcctttaaagctggcactaaattcatttgctatcgcaccaggggcacagagtaggctccctgccaataaccaagcacttttgcttaaccaatgtttcacgtatttctccctgcacttacgctgatagcctaagtattacaattggatatagatttcgtactgctggccttcacgctcaacagtcagattcagctcttgcggatcgttcatgattcgcatcaattcagctgaagaagaaggatcactcaagtcgatcccattaagttgtactgcaatatcaccagactctaagcccacatcattaaataatgcagcgtcccgacccggagcaactcggtaacccacaatgtcatcaccattcttaaccggtgaaatgctgatgtatttgaaaacacttttcggatcttgagcaatttcctctcggatttgctccaatttgtcacccgctacagactctggttgtgctgcctgagcacgtcttaattgaggcgattcagataacttgttgtattcaatcccctgcatcatcagggtttcgtcacggccttggttttcgatgatcacacggtccaccaatacggctttcagtgtcgcacgtgtgccttcgatttcttcaccgataccatacgttgactgtttacctttattagcaatcaccgctaagctggtttgcacctgattactggccactgcccccacgagagttaagtttagacgagttttaggcgcatctttgacaactggttgttcgatgatgacaggtttttgttcagagtattgaccgaataagtgagcattttgaagggcattgaaatcaatcgcgtctttgttattgccagcaataacgttacctgctgaaggccgccatgggctgatatcagccgtttgtgggatcgctaaccacactatctttccgagcagccacccggctagcgcgataaataaacaagtaaacagcaaactaagcttggtttgaatcgcgtagttattggatttcatctgagccagtagcgggctaccagataatccagcactcagctctattcgcttcactaaattgattcctttctttcgatgtaggtcgaactggctattaaaaaaacatcgactctatctttctaaaatgacagtaaaattactaataaacaaatagcagtgaactatatcacaacagatatggcatacctttttcttcttcagtttttgccaactcccttgaaaaaattagaccttggctccatctgtaggtgaataatccatcactcactgcagttaatggcagtggttagatagcgtagtgttctatgagttccgttaatgaagccgttaggctagataaatggctttgggccgccagattttataaaacccgctccatcgctcgtaacatggtcgatggcggtaaagtccactataatggccaacgaagcaaaccgagtaaaatcgttgaagtgggcgccgaaattaaacttcgtcaaggcaatgaagagaaaacggtgatcattgagataatctccgatcagcgtaagggcgcaccaatcgcacagactctgtatcgcgaaactaatgaaagtatagcgaaacgtgaggaacacgcgacacaacgtaaacttaatgcccacaacccaagtccggaacgccgcccagataagaagcagcgccgagacatcattaagttcaaacaccagtaaaaagccggaataccaacgaggaaatcactccatggcaaataatgttttaaaccgctacctatttgaagacctttcagttcgtggcgagttggtacaattggacgaagcgtaccagcgtattatctccagcaaggaatatccggcggcagtacaaaaactgttaggcgagctactcgtatcaaccacacttttaaccgcgacgcttaagttcgaaggctcaatcactattcagattcaaggcgacggcccggtttctcttgctgtgatcaatggtgaccacaaacaaagaattcgtggtgttgcgcgctgggaaggcgatattgccgatgaggcgagccttcacgatatgatgggtaaaggtcatatggtgatcaccatcgaacctaaagtaggcgagcgctaccaaggtgttgttggcttagaaggtgacaatcttgcagaagttctggaaggctacttcgctaactctgagcagctaaaaacgcgtctttggatccgtactggtgaattcgaaggtcaaccgcatgctgcgggtatgctgattcaggtaatgccggatggtactggctctcctgatgactttgaacatttggagcaactgactgacacagtaaaagaggaagagctgttcggtttagaagccaatgaactactttaccgtctttacaaccaggacaaagtacgtctttacgaacctcaaccagtttcattccagtgcggctgttctcgtgaacgcagtggtgctgcgatcgtcactgttgagaaagcggaaatttatgacattttagccgaggttggatcagtttcgttacattgtgattactgtggcacaacttatagcttcgatgaagctgaggttactgcgctctacgaacaagcttcaacggacaaaaaaacgctgcattaattttcatagcttaccaagcacgtaattcccccttcaaaaggccagctcaatgctggcctttttgtgacctaagccccgtaaaccctagcttttaccgtaattttagttacggggccctgaagtcagccccatacgatataagttgtaagcttatttgaaaaaagtgtttgacagttgaacactcgttgcctataatcgaaagtcgagttatcgagattttcaggagaggaagctcatatgactgacctaaacaaactcgtcaaggaacttaatgacctaggccttacagatgttaaggaaattgtatataaccccagttacgagcaacttttcgaggaggaaactaaacctggcttggagggttttgataaagggacgctaaccactcttggcgcagttgccgtcgatacggggatttttacaggacgttcaccgaaggataaatatatcgtttgtgatgaaactacgaaggacacagtatggtggaacagcgaagctgcgaaaaacgataacaaaccaatgacgcaagagacttggaagagtttgagagaattagtggctaaacaactttctggtaaaagactattcgtggtagagggttactgcggcgcaagtgaaaaacaccgcatcggtgtgcgtatggtcactgaagtggcatggcaggcgcattttgtaaaaaacatgtttatccgaccaacagatgaagagcttaaaaatttcaaggcggattttaccgtgctaaacggtgctaaatgtactaacccaaactggaaagaacagggcctcaacagtgaaaactttgtcgcttttaatattaccgaaggtattcagcttatcggcggcacttggtacggcggtgaaatgaaaaagggtatgttttcaatgatgaactacttcctgccattaaaaggtgtagcttctatgcattgttcggcaaacgtaggaaaagacggtgacgtagctattttcttcggcctatctggtacgggtaaaacaacgctttcgaccgatcctaaacgccaattaatcggtgatgacgagcacggttgggatgaaagcggcgtatttaactttgaaggcggttgttacgctaaaaccatcaacctctctcaagagaacgaaccagatatctacggcgcaatccgtcgtgacgcacttttagagaacgtcgtagttcgagcagacggttccgttgactttgacgacggttcaaaaacagagaatactcgtgtttcatatccgatttaccacatcgacaacatcgttcgtcctgtatctaaagccggccatgcaacaaaagtgatcttcctgtctgctgacgcgtttggtgtgcttcctccagtatcgaaactgacaccagagcaaaccaagtaccacttcctgtctggctttactgccaaactagcaggtacagagcgcggtatcactgagccgacgccaactttctctgcgtgttttggcgctgcgttcctaacgcttcacccaactaagtacgcagaagtactggttaaacgtatggaagcagcaggtgcagaagcttacctggttaacactggctggaatggtagcggtaaacgtatctcaattcaggatacacgcggcatcattgatgcgatcctagatggctcaattgaagatgcaccaactaagcatatcccaatcttcaaccttgaagttcctacttcactaccaggtgttgatccaagcattcttgatccacgtgatacttacgttgatccactgcaatgggaaagcaaagcgaaagatctagcagaacgctttatcaataactttgataaatacacagacaacgcagaaggtaaatctctggttgctgctggtccacagcttgactaatgttgtgcgaaaacccggtcgagagtgacttggttaccaaaatattctacaagcccctcttttgaggggctttttattgaatcccacccaagattgatacaagctaaagcagacttccatactttacgtaggttctggcaaggatgaaaacagcgcttcgaattctaattctgttacttgtattactgattgctatccctgcaaccgttattgcgctgcttaccacttcctacgctaaccaaacctgggcctttatttcagagcacctgaacttgcctattcaggcagaaaaggtgcactacgactttccttatcacctcaccgtgcaaggcattcgaacgaatagcgaagatatatccagcattgagcaagtcgatgtatggctaaacccagatgtgcgccgcgatggtaagtggattgtcgatagcttacttatcaatggcgtgagcttaaaacaaggactgccggatttcgccaatctagaatctgtccagttccaccaagtggcggttaaaaatctcgattacgcgaacaaacagttagtgatcaatgggctgaatgtacaaatccaatcaccagactggcaaaacaactcgtatgtactgccttatggcgagattcagttgtctgctgcgcaaatctactggaatggcgaagctttcgataatgtgttgcttgatatggattatcgaccagaaaacagcactctctatggtacgtcgtttaaatggagagacagtctgatctctggtcagggtgagcaataccctcaaggctggtctttggtcaatgtcaccgtcgataagctgaaaatgaacaatgcccagctgcaatcgctgctcgctaagccatggaatgccatacctttcaaaattaaccacattaatagtctcgacctgttaaacgctgacattgaatggggcgactggcattggcaaaacctggaactttcgctcgaggatgcttcacttccgctatcactttggcagacacaagctcagatctctttgcaagcagacagcgttagctttcaggaacaaactgcaatagagccacgtttaagcgcggtaatgacgcctagtcagattgaactgaaagaactctacctggactggcaacaaggccgtgttcaaatttcaggacagttccaaccaacccaatggcaaatagacaatgcctcaatttatggcttgaaatgggcaatgaagcctgacgaaagttcagactggtggcaagcggcaacaaaagcactaaacgaagtgacgataaatcagctcgacattgaacacagccaaatcatacaaatatcacgacagccatattggcaggtttcgggcctgaatttggaaggtcgtcaacttgacctcaaacgcctagggacgcactggtcggtgtggaatgggaacctggacgttagcgtcgtcaacgctagctatgatcaggtgattgcctctcacgctgcgctttcaactcaaagtgacaatggcctttggcagttgactcgtttatttgcgccattagagcaaggctatatcgaaggttacgggcagattgatgtcagcaccaccagccaaccatggacgctgaacatcaatgcagatggcattcctctgcgcctctttcactcatacctacctaaagtattaaccgtagaaggcttgtccgatctgaatcttgatctccaaggcctggctggcgatcataatatgctggcgtacagcttaaccggagatatcgaagccaacttccgtgacacgatgttgctctctcaagccgatcagtcactgaagtcgatcacattctctcctgtccgtctgcaagcgcgacgtggtgaggtttcaatccaacccatcaccatttcgggaagcgacattaaaggcaaaatcagtggtgcgtttgatctggctaacaacccattaagtgggcttgagtatcagctagaagagcagtgtggcgtcattagcggcgatattttcagccatgagccgacgaaaaatgactgtatcaagcccgctgagatcgaaggacaggagtcaacaaaagagactcaacaaccagagttaacagcaggagaagcgcaacctacagctcctatcgcggaaatcaatatggagctgcaggaagaagagttggttgaagagatagtggaagaagatgaactaacaataaatgctgttgccgacgaagcactaacagctgagtaattgcgaataattagggattagcatgtaagtcccgacaaaatcgacagctggcgtgtcaccactataaatcaccacatgaatcacgattcgcgctttacgacccgatttcagccgatccaagtcaccactgatgccatcaagtgatgtgctggcgacagggttttgctcaacgggttggcgataacgaatggaactgtccgcgagaacaatatcaccgtgcaagccacgctctttaagtagtaaccaggtcattccccacccagtcaatgtcgccaaggtaaaagcagagccagcaaacatggtattgtgagggttaaggttagggttaagctgggcgcagcattcaaattgataacccgtgtattgattaatcttaatccccattttgtcactgatcgggatttgctcttcccatcgcttctggagctccgtacaccactcagggcggcgaagaacattagccatcgggtctaagggcttaaccatttgttggtggcggaccgggccacgctcatcagtcagctcaccacggcgctcaaaaccacttttctcatagaacgcaatcgcgtcttcacgggcattacataccaagcgtttcgcaccttcctggcgcgcaagtgactccagtgccaccaacactaatgaacccatgcctttgccacgacgattactttttaccgccatgtaacgaatctgaccgtcat

SEQ ID NO: 37 - protein, 886 aa, Vibrio natriegens
MSDMKHDVDALETQEWLQALESVVREEGVERAQFLLEQVLDKARLDGVDMPTGITTNYINTIPADQEPAYPGDTTLERRIRSIIRWNAIMIVLRASKKDLELGGHMASFQSSAAFYETCFNHFFRAPNEKDGGDLVYYQGHISPGIYSRAFVEGRLTEEQLDNFRQEVDGKGIPSYPHPKLMPEFWQFPTVSMGLGPISAIYQARFLKYLEGRGMKDTSEQRVYAFLGDGEMDEPESRGAISFAAREKLDNLCFLINCNLQRLDGPVMGNGKIIQELEGLFKGAGWNVVKVIWGNNWDSLLAKDTTGKLLQLMNETIDGDYQTFKAKDGAYVREHFFGKYPETAALVADMTDDEIFALKRGGHESSKLYAAYKNAADTKGRPTVILAKTVKGYGMGEAAEGKNIAHQVKKMDMTHVLHLRDRLGLQDLLTDEAVKELPYLKLEEGSKEYEYLHARRKALHGYTPQRLPNFTQELIVPELEEFKPLLEEQKRDISSTMAFVRSLNVLLKNKNIGKNIVPIIADEARTFGMEGLFRQIGIYNPHGQTYTPEDRGVVSYYKEATSGQVLQEGINELGAMSSWVAAATSYSTNDLPMIPFYIYYSMFGFQRVGDMAWMAGDQQARGFLLGATAGRTTLNGEGLQHEDGHSHIMAGTVPNCISYDPTFAYEVAVIMQDGIRRMYGEQENVFYYLTLMNENYAMPAMPEGAEEGIRKGIYKLETYTGSKGKVQLMSSGTIMNEVRKAAQILSDEYGVASDVYSVTSFNEVTRDGQAAERYNMLHPEAEAQVPYIQTVMGTEPAIAATDYMKNYAEQVRAFIPAESFKVLGTDGFGRSDSRENLRRHFEVNAGYVVVAALTELAKRGEVEKSVIAEAIKKFDIDTEKTNPLYA

SEQ ID NO: 38 - protein, 629 aa, Vibrio natriegens
MAIEINVPDIGTDEVEVTEILVSVGDKVEEEQSLITVEGDKASMEVPASQAGIVKEIKVAEGDKVSTGSLIMIFEAEGAAEAAPAPAAEAAPAAAPAPAAAAELKEVHVPDIGGDEVEVTEIMVAIGDSIEEEQSLITVEGDKASMEVPAPFAGTLKEIKVAAGDKVSTGSLIMVFEVAGSGAPAAAPAAVEAPAAAAPAASAAKEVNVPDIGGDEVEVTEIMVAVGDTVEEEQSLITVEGDKASMEVPAPFAGTVKEIKIAAGDKVSTGSLIMVFEVAGAAPAPAAAPAQAAAPAAAAPKAEAPAAAAPAATGDFKENDEYAHASPVVRRLAREFGVNLSKVKGSGRKSRILKEDVQNYVKEALKRLESGAAASGKGDGAALGLLPWPKVDFSKFGETEVQPLSRIKKISGANLHRNWVMIPHVTQWDNADITALEAFRKEQNAIEAKKDTGMKITPLVFIMKAVAKALEAFPAFNSSLSEDGESLILKKYVNVGIAVDTPNGLVVPVFKDVNKKGIYELSEELMAVSKKARAGKLTAADMQGGCFTISSLGGIGGTAFTPIVNAPEVGILGVSKSEMKPVWNGKEFEPRLQLPLSLSYDHRVIDGAEGARFITYLNSCLSDIRRLVL

SEQ ID NO: 39 - protein, 475 aa, Vibrio natriegens
MSKEIKAQVVVLGSGPAGYSAAFRCADLGLETVLVERYSTLGGVCLNVGCIPSKALLHVSKVIEEAKAMADHGVVFGEPQTDINKIRIWKEKVVNQLTGGLGGMAKMRNVTVVNGYGKFTGPNSILVEGEGESTVVNFDNAIVAAGSRPIKLPFIPHEDPRIWDSTDALELKEVPEKLLIMGGGIIGLEMGTVYHSLGSKVEVVEMFDQVIPAADKDIVKVYTKRIKDKFKLMLETKVTAVEAKEDGIYVSMEGKKAPAEAERYDAVLVAIGRVPNGKLIDGEKAGLEIDERGFINVDKQMRTNVPHIFAIGDIVGQPMLAHKGVHEGHVAAEVISGKKHYFDPKVIPSVVYTEPEVVWVGKTEKEAKDEGIKYEVATFPWAASGRAIASDCSDGMTKLIFDKETHRVIGGAIVGTNGGELLGEIGLAIEMGCDAEDIALTIHAHPTLHESVGLAAEVFEGSITDLPNKKAVKKK

SEQ ID NO: 40 - protein, 432 aa, Escherichia coli
MRKIKGLRWYMIALVTLGTVLGYLTRNTVAAAAPTLMEELNISTQQYSYIIAAYSAAYTVMQPVAGYVLDVLGTKIGYAMFAVLWAVFCGATALAGSWGGLAIARGAVGAAEAAMIPAGLKASSEWFPAKERSIAVGYFNVGSSIGAMIAPPLVVWAIVMHSWQMAFIISGALSFIWAMAWLIFYKHPRDQKHLTDEERDYIINGQEAQHQVSTAKKMSVGQILRNRQFWGIALPRFLAEPAWGTFNAWIPLFMFKVYGFNLKEIAMFAWMPMLFADLGCILGGYLPPLFQRWFGVNLIVSRKMVVTLGAVLMIGPGMIGLFTNPYVAIMLLCIGGFAHQALSGALITLSSDVFGRNEVATANGLTGMSAWLASTLFALVVGALADTIGFSPLFAVLAVFDLLGALVIWTVLQNKPAIEVAQETHNDPAPQH

SEQ ID NO: 41 - protein, 589 aa, Vibrio parahaemolyticus
MSNIEHGLSFIDIMVFAIYVAIIIGVGLWVSRDKKGTQKSTEDYFLAGKSLPWWAVGASLIAANISAEQFIGMSGSGYSIGLAIASYEWMSAITLIIVGKYFLPIFIEKGIYTIPEFVEKRFNKKLKTILAVFWISLYIFVNLTSVLYLGGLALETILGIPLMYSILGLALFALVYSIYGGLSAVVWTDVIQVFFLVLGGFMTTYMAVSFIGGTDGWFAGVSKMVDAAPGHFEMILDQSNPQYMNLPGIAVLIGGLWVANLYYWGFNQYIIQRTLAAKSVSEAQKGIVFAAFLKLIVPFLVVLPGIAAYVITSDPQLMASLGDIAATNLPSAANADKAYPWLTQFLPVGVKGVVFAALAAAIVSSLASMLNSTATIFTMDIYKEYISPDSGDHKLVNVGRTAAVVALIIACLIAPMLGGIGQAFQYIQEYTGLVSPGILAVFLLGLFWKKTTSKGAIIGVVASIPFALFLKFMPLSMPFMDQMLYTLLFTMVVIAFTSLSTSINDDDPKGISVTSSMFVTDRSFNIAAYGIMIVLAVLYTLFWVNADAEITLIIFGVMAGVIGTILLISYGIKKLIKGGGGGGHHHHHH

SEQ ID NO: 42 - protein, 323 aa, Vibrio natriegens
MSEQYALVGDIGGTNARLALCELSTGTISDIVTYPAAEYESLEVVMRQYLDRHDTPISSACIAIACPVTGDWVSMTNHHWEFSIQELKSQLNLQVLEVINDFTAVSFAIPSLKPEDRVQIGGGEAIANKPIAVYGAGTGLGVAQLIHGGNLWHSVPGEGGHVDLAACTPEEDDLIVYLRNKFGRVSAERCLSGQGIRNIYDFVVSHHGAAPEDYTPAMITEKALKAECKDCERALYLFCILMGRFGGNLALTNGSFGGVYIAGGIVPKVQELFIKSGFRVAFEDKGRFKEYLQSIPVFLITHSEPGLLGAGTYIRQSLNISIN

SEQ ID NO: 43 - protein, 328 aa, Corynebacterium glutamicum
MNSPQNVSTKKVTVTGAAGQISYSLLWRIANGEVFGTDTPVELKLLEIPQALGGAEGVAMELLDSAFPLLRNITITADANEAFDGANAAFLVGAKPRGKGEERADLLANNGKIFGPQGKAINDNAADDIRVLVVGNPANTNALIASAAAPDVPASRFNAMMRLDHNRAISQLATKLGRGSAEFNNIVVWGNHSATQFPDITYATVGGEKVTDLVDHDWYVEEFIPRVANRGAEIIEVRGKSSAASAASSAIDHMRDWVQGTEAWSSAAIPSTGAYGIPEGIFVGLPTVSRNGEWEIVEGLEISDFQRARIDANAQELQAEREAVRDLL

SEQ ID NO: 44 - protein, 539 aa, synthetic construct
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTINLSQENEPDIYGAIRRDALLENVVVRADGSVDFDDGSKTENTRVSYPIYHIDNIVRPVSKAGHATKVIFLSADAFGVLPPVSKLTPEQTKYHFLSGFTAKLAGTERGITEPTPTFSACFGAAFLTLHPTKYAEVLVKRMEAAGAEAYLVNTGWNGSGKRISIQDTRGIIDAILDGSIEDAPTKHIPIFNLEVPTSLPGVDPSILDPRDTYVDPLQWESKAKDLAERFINNFDKYTDNAEGKSLVAAGPQLD

SEQ ID NO: 45 - protein, 539 aa, synthetic construct
MTDLNKLVKELNDLGLTDVKEIVYNPSYEQLFEEETKPGLEGFDKGTLTTLGAVAVDTGIFTGRSPKDKYIVCDETTKDTVWWNSEAAKNDNKPMTQETWKSLRELVAKQLSGKRLFVVEGYCGASEKHRIGVRMVTEVAWQAHFVKNMFIRPTDEELKNFKADFTVLNGAKCTNPNWKEQGLNSENFVAFNITEGIQLIGGTWYGGEMKKGMFSMMNYFLPLKGVASMHCSANVGKDGDVAIFFGLSGTGKTTLSTDPKRQLIGDDEHGWDESGVFNFEGGCYAKTIKLSKEAEPDIYNAIRRDALLENVTVRNDGSIDFDDGSKTENTRVSYPLYHIENIVKPVSKGGHANKVIFLSADAFGVLPPVSKLTPEQTKYHFLSGFTAKLAGTERGITEPTPTFSACFGAAFLTLHPTKYAEVLVKRMEAAGAEAYLVNTGWNGSGKRISIQDTRGIIDAILDGSIEDAPTKHIPIFNLEVPTSLPGVDPSILDPRDTYVDPLQWESKAKDLAERFINNFDKYTDNAEGKSLVAAGPQLD

SEQ ID NO: 46 - protein, 370 aa, Synechococcus elongatus
MDFLSDFLTKFGSQLQSPTLGFLIGGIVIAAFGSRLTIPDAVYKFIVFMLLIKVGLSGGIAIRNTNITEMLLPALFAVLMGILIVFIGRFTLAKLPGIRTVDAVATAGLFGAVSGSTLAAGITVMEGQGVFYEPWAAALYPFMDIPALVTAIVVASLYKSKQREVEADDFSKQPVAAGEYSGEPVYPTTRQEYLGQKRGKATNRVEIWPIVKESLQGSALSALLLGLALGLLTRPESVFESFYEPLFRGFLSILMLVMGMEAWSRLGELRKVAQWYAVYAFIAPLLHGFIAFGLGMIAHYVTGFSPGGVALLAIIAASSSDISGPPTLRAGIPSANPSAYIGSSTAIGTPVAIAIGIPLFIGLAQATMGG

(19) United States
(12) Patent Application Publication
(10) Pub. No.: US 2025/0297291 A1
(43) Pub. Date: Sep. 25, 2025

(54) BIO-BASED PRODUCTION OF SUCCINIC ACID USING VIBRIO NATRIEGENS

(71) Applicant: General Biological Corporation, New Haven, CT (US)

(72) Inventors: David Kendall Sozanski, Oxford, CT (US); Kyle Phillip Mohler, New Haven, CT (US); Abhinav Godavarthi, New Haven, CT (US); Timothy J. Hanly, New Haven, CT (US)

(21) Appl. No.: 19/083,007

(22) Filed: Mar. 18, 2025

Related U.S. Application Data
(60) Provisional application No. 63/567,141, filed on Mar. 19, 2024.

(51) Int. Cl. C12P 7/46; C12N 9/02; C12N 9/04; C12N 9/12; C12N 15/74

(52) U.S. Cl. CPC C12P 7/46; C12N 9/0006; C12N 9/0008; C12N 9/12; C12N 9/1205; C12N 15/74; C12Y 101/01037; C12Y 102/05001; C12Y 207/01002; C12Y 207/11013; C12N 2800/101

(57) ABSTRACT

This disclosure provides methods and genetically engineered strains of Vibrio natriegens, specifically developed for the bio-based production of succinate. Capitalizing on the rapid growth kinetics and highly efficient carbon metabolism of V. natriegens, this disclosure provides an environmentally friendly, scalable, and cost-effective alternative to traditional petrochemical methods for succinate production.

### CROSS-REFERENCE TO RELATED APPLICATIONS

This application claims the benefit of U.S. provisional patent application No. 63/567,141, filed Mar. 19, 2024, which is incorporated herein by reference in its entirety.

### REFERENCE TO SEQUENCE LISTING SUBMITTED ELECTRONICALLY

The content of the electronically submitted sequence listing (Name: 5625_0020001_ST26_SequenceListing.xml; Size: 120,048 bytes; and Date of Creation: Mar. 17, 2025), filed with the application, is incorporated herein by reference in its entirety.

### BACKGROUND OF THE DISCLOSURE

Succinic acid (also known as amber acid, butanedioic acid), a critical platform chemical, finds extensive applications in various industries including polymer, pharmaceutical, and food sectors, owing to its utility as a versatile precursor for synthesizing a range of high-value products. Traditionally, succinic acid production has predominantly relied on petrochemical processes, specifically through the hydrogenation of maleic anhydride, which is derived from butane. While these conventional methods have been the mainstay of commercial succinic acid supply for many years, they are increasingly recognized for their significant environmental, economic, and societal drawbacks, highlighting the imperative for sustainable production alternatives.

The petrochemical synthesis of succinic acid is heavily dependent on finite fossil fuel resources, leading to considerable environmental concerns such as high carbon emissions and ecological impacts associated with resource extraction and processing. Economic vulnerabilities arise from the direct correlation between petrochemical succinic acid production and fluctuating oil prices, introducing supply chain instabilities. Furthermore, the operational complexities involved in separating by-products, managing hazardous reagents, and adhering to strict environmental regulations pose challenges to the viability and public acceptance of petroleum-based succinic acid production methods. In light of these challenges, there is a growing interest in the development of bio-based processes for succinic acid production. Microbial fermentation offers a renewable, environmentally benign, and economically viable alternative, aligning with global sustainability objectives and addressing the limitations inherent in petrochemical methods. The present disclosure introduces an innovative bio-based process for producing succinic acid (via succinate) utilizing the bacterium Vibrio natriegens.

V. natriegens, a gram-negative facultative anaerobe indigenous to estuarine environments, is renowned for its exceptionally rapid growth rate, achieving doubling times of under 10 minutes. See Weinstock, M. T., et al., “Vibrio natriegens as a fast-growing host for molecular biology,” Nature Methods, 13 (10): 849-851 (2016). This organism's remarkable growth is underpinned by its highly efficient metabolic and nutrient uptake systems, enabling it to metabolize a diverse array of carbon sources effectively. The elevated metabolic turnover rate of V. natriegens is conducive to high-yield bio-production. Additionally, the genetic tractability of this species allows for sophisticated metabolic engineering to enhance succinate synthesis. These characteristics render V. natriegens an exemplary candidate for industrial-scale bio-production applications.

This disclosure capitalizes on the unique metabolic properties of V. natriegens to offer an efficient, scalable, and sustainable pathway for succinate production. This novel bio-process and the genetically optimized strains of V. natriegens described herein are designed to achieve superior succinate yields and productivity, while simultaneously reducing carbon emissions and diminishing reliance on non-renewable fossil fuels. This approach represents a significant stride towards realizing the potential of renewable chemical production.

### SUMMARY OF THE DISCLOSURE

This disclosure pertains to novel methods and genetically engineered strains of Vibrio natriegens, specifically developed for the bio-based production of succinate. Capitalizing on the rapid growth kinetics and highly efficient carbon metabolism of V. natriegens, this disclosure offers an environmentally friendly, scalable, and cost-effective alternative to traditional petrochemical methods for succinate production.

The core of the disclosure lies in the strategic modification of V. natriegens to significantly enhance succinate yields over wild-type V. natriegens. This is achieved through the integration of alternative substrate uptake mechanisms and carbon utilization strategies, which collectively amplify anaplerotic flux towards essential metabolic precursors. Additionally, the disclosure focuses on optimizing anaerobic metabolism in V. natriegens to effectively redirect cellular resources and intermediary metabolites into the reductive tricarboxylic acid (rTCA) pathway. A critical component of this strategy involves the suppression of competing metabolic pathways that otherwise lead to the generation of terminal metabolic byproducts.

In essence, the genetically optimized V. natriegens strains, coupled with the process innovations introduced in this disclosure, establish a scalable and economically viable platform for the renewable production of succinate. This aligns with global sustainability objectives, offering a greener alternative to conventional production methods. Key features of the disclosure include: i) engineered modifications to V. natriegens to enhance substrate uptake and increase flux through glycolytic and anaplerotic pathways, thereby boosting the availability of precursors for succinate synthesis; ii) metabolic pathway engineering of the core metabolic processes in V. natriegens, tailored to achieve high succinate yield, particularly during continuous culture under oxygen-limited conditions; and iii) strategic elimination of pathways leading to carbon and electron leakage into metabolic byproducts, ensuring a focused redirection of these resources towards the enhanced production of succinate.

The present disclosure provides a method of producing succinic acid, the method comprising: culturing a plurality of non-naturally occurring Vibrio natriegens cells comprising one or more genetic disruptions, wherein the one or more genetic disruptions increase the production of succinate as compared to the production of succinate from a V. natriegens cell that does not comprise the one or more genetic disruptions; and wherein under anaerobic conditions, the plurality of non-naturally occurring V. natriegens cells produce succinic acid from a glucose substrate at a rate of at least 0.48 g_{Succ} g_{CDW}⁻¹ h⁻¹.

In some aspects, the plurality of V. natriegens cells comprise 6 or more genetic disruptions.

In some aspects, the plurality of V. natriegens cells comprise a mutation to a native phosphoenolpyruvate carboxykinase (pck) gene. In some aspects, the V. natriegens cells comprise a pck fusion comprising a V. natriegens pck sequence and an A. succinogenes pck sequence operatively linked to a constitutive promoter.

In some aspects, the plurality of V. natriegens cells comprise a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter.

In some aspects, the plurality of V. natriegens cells comprise a deletion of an L-lactate dehydrogenase gene (lldH).

In some aspects, the plurality of V. natriegens cells comprise a deletion of a D-lactate dehydrogenase gene (dldH).

In some aspects, the plurality of V. natriegens cells comprise a deletion of an alanine dehydrogenase gene (alD).

In some aspects, the plurality of V. natriegens cells comprise a deletion of a pyruvate formate lyase gene (pflB).

In some aspects, the plurality of V. natriegens cells comprise a deletion of each of the lldH, dldH, alD, and pflB genes.

In some aspects, the plurality of V. natriegens cells comprise a deletion of a pts gene.

In some aspects, the plurality of V. natriegens cells comprise a native glucokinase gene (glk) operably linked to a constitutive synthetic promoter.

In some aspects, the plurality of V. natriegens cells overexpress a native glucokinase gene (glk).

In some aspects, the plurality of V. natriegens cells comprise a heterologous sodium-dependent glucose transporter protein (vsGLT) from Vibrio parahaemolyticus.

In some aspects, the plurality of V. natriegens cells comprise a mutation to the native E1 and E2 subunits of a pyruvate dehydrogenase complex (aceEF) gene.

In some aspects, the mutation comprises a replacement of the native promoter of the aceEF gene with a synthetic promoter.

In some aspects, wherein the plurality of V. natriegens cells comprise a mutation to a native E3 subunit of a pyruvate dehydrogenase complex (lpdA) gene. In some aspects, the mutation comprises an E354K mutation and a replacement of the native promoter of the lpdA gene with a synthetic promoter.

In some aspects, the plurality of V. natriegens cells comprise a heterologous E. coli hexuronate transporter (exuT) gene, wherein the exuT gene is operably linked to a constitutive synthetic promoter.

In some aspects, the plurality of V. natriegens cells comprise a deletion of a PN96_RS22390 gene.

In some aspects, the plurality of V. natriegens cells comprise: a deletion of each of the lldH, dldH, alD, and pflB genes; and a pck fusion comprising a V. natriegens pck sequence and an A. succinogenes pck sequence operatively linked to a constitutive promoter.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene; a deletion of a PN96_RS22390 gene.

The present disclosure provides a method of producing succinic acid, comprising culturing a plurality of genetically modified Vibrio natriegens cells in the presence of a carbohydrate, wherein the genetically modified V. natriegens cells enable production of succinate in the culture during the V. natriegens growth phase. In some aspects, the plurality of V. natriegens cells overexpress the E1 and E2 subunits of the pyruvate dehydrogenase complex (aceEF). In some aspects, the plurality of V. natriegens cells overexpress a dihydrolipoamide dehydrogenase gene (lpdA).

The present disclosure provides a method of producing succinic acid, comprising culturing a population of genetically modified Vibrio natriegens cells in a two-phase cultivation for a period of greater than 24 hours. In some aspects, the two-phase cultivation is for a period of less than 7 days. In some aspects, each phase of the two-phase cultivation takes place in the same vessel.

In some aspects, the culturing does not comprise a concentration step.

In some aspects, the plurality of V. natriegens cells comprise 6 or more genetic disruptions.

In some aspects, the plurality of V. natriegens cells comprise a mutation to a native phosphoenolpyruvate carboxykinase (pck) gene. In some aspects, the V. natriegens cells comprise a pck fusion comprising a V. natriegens pck sequence and an A. succinogenes pck sequence operatively linked to a constitutive promoter.

In some aspects, the plurality of V. natriegens cells comprise a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter.

In some aspects, the plurality of V. natriegens cells comprise a deletion of an L-lactate dehydrogenase gene (lldH).

In some aspects, the plurality of V. natriegens cells comprise a deletion of a D-lactate dehydrogenase gene (dldH).

In some aspects, the plurality of V. natriegens cells comprise a deletion of an alanine dehydrogenase gene (alD).

In some aspects, the plurality of V. natriegens cells comprise a deletion of a pyruvate formate lyase gene (pflB).

In some aspects, the plurality of V. natriegens cells comprise a deletion of each of the lldH, dldH, alD, and pflB genes.

In some aspects, the plurality of V. natriegens cells comprise a deletion of a ptsI gene.

In some aspects, the plurality of V. natriegens cells comprise a native glucokinase gene (glk) operably linked to a constitutive synthetic promoter.

In some aspects, the plurality of V. natriegens cells overexpress a native glucokinase gene (glk).

In some aspects, the plurality of V. natriegens cells comprise a heterologous sodium-dependent glucose transporter protein (vsGLT) from Vibrio parahaemolyticus.

In some aspects, the plurality of V. natriegens cells comprise a mutation to the native E1 and E2 subunits of a pyruvate dehydrogenase complex (aceEF) gene.

In some aspects, the mutation comprises a replacement of the native promoter of the aceEF gene with a synthetic promoter.

In some aspects, wherein the plurality of V. natriegens cells comprise a mutation to a native E3 subunit of a pyruvate dehydrogenase complex (lpdA) gene. In some aspects, the mutation comprises an E354K mutation and a replacement of the native promoter of the lpdA gene with a synthetic promoter.

In some aspects, the plurality of V. natriegens cells comprise a heterologous E. coli hexuronate transporter (exuT) gene, wherein the exuT gene is operably linked to a constitutive synthetic promoter.

In some aspects, the plurality of V. natriegens cells comprise a deletion of a PN96_RS22390 gene.

In some aspects, the plurality of V. natriegens cells comprise: a deletion of each of the lldH, dldH, alD, and pflB genes; and a pck fusion comprising a V. natriegens pck sequence and an A. succinogenes pck operatively linked to a constitutive promoter.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; and a deletion of a PN96_RS22390 gene.

In some aspects, the rate of succinic acid production of the population of genetically modified V. natriegens cells is from about 0.48 to about 2.00 g_{Succ} g_{CDW}⁻¹ h⁻¹.

In some aspects, the method is performed in a bioreactor.

In some aspects, the rate of succinic acid production is measured over a two hour period.

The present disclosure provides an engineered Vibrio natriegens cell comprising one or more genetic disruptions, wherein the one or more genetic disruptions increase the production of succinate as compared to the production of succinate from a V. natriegens cell that does not comprise the one or more genetic disruptions, wherein the V. natriegens cell produces succinic acid from a glucose substrate at a rate of at least 0.48 g_{Succ} g_{CDW}⁻¹ h⁻¹. In some aspects, the rate of succinic acid production of the cell is from about 0.48 to about 2.00 g_{Succ} g_{CDW}⁻¹ h⁻¹.

In some aspects, the cell comprises 6 or more genetic disruptions.

In some aspects, the cell comprises a mutation to a native phosphoenolpyruvate carboxykinase (pck) gene. In some aspects, the cell comprises a pck fusion comprising a V. natriegens pck sequence and an A. succinogenes pck sequence operatively linked to a constitutive promoter.

In some aspects, the cell comprises a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter.

In some aspects, the cell comprises a deletion of an L-lactate dehydrogenase gene (lldH).

In some aspects, the cell comprises a deletion of a D-lactate dehydrogenase gene (dldH).

In some aspects, the cell comprises a deletion of an alanine dehydrogenase gene (alD).

In some aspects, the cell comprises a deletion of a pyruvate formate lyase gene (pflB).

In some aspects, the cell comprises a deletion of each of the lldH, dldH, alD, and pflB genes.

In some aspects, the cell comprises a deletion of a ptsI gene.

In some aspects, the cell comprises a native glucokinase gene (glk) operably linked to a constitutive synthetic promoter.

In some aspects, the cell overexpresses a native glucokinase gene (glk).

In some aspects, the cell comprises a heterologous sodium-dependent glucose transporter protein (vsGLT) from Vibrio parahaemolyticus.

In some aspects, the cell comprises a mutation to the native E1 and E2 subunits of a pyruvate dehydrogenase complex (aceEF) gene. In some aspects, the mutation comprises a replacement of the native promoter of the aceEF gene with a synthetic promoter.

In some aspects, the cell comprises a mutation to a native E3 subunit of a pyruvate dehydrogenase complex (lpdA) gene. In some aspects, the mutation comprises an E354K mutation and a replacement of the native promoter of the lpdA gene with a synthetic promoter.

In some aspects, the cell comprises a heterologous E. coli hexuronate transporter (exuT) gene, wherein the exuT gene is operably linked to a constitutive synthetic promoter.

In some aspects, the cell comprises a deletion of a PN96_RS22390 gene.

In some aspects, the cell comprises: a deletion of each of the lldH, dldH, alD, and pflB genes; and a pck fusion comprising a V. natriegens pck sequence and an A. succinogenes pck sequence operatively linked to a constitutive promoter.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; and a deletion of a PN96_RS22390 gene.

The present disclosure provides a bioreactor comprising an engineered V. natriegens cell as disclosed herein.

In some aspects, the rate of succinic acid production in the bioreactor is at least 0.48 g_{Succ} g_{CDW}⁻¹ h⁻¹.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DISCLOSURE OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings.

FIG. 1A depicts the lldH KO Sequence [SEQ ID NO: 19] and the dldH KO Sequence [SEQ ID NOs: 20].

FIG. 1B depicts the alD KO Sequence [SEQ ID NO: 21].

FIG. 1C depicts the pflB KO Sequence [SEQ ID NO: 22].

FIG. 1D depicts the pC100-aceEF pC100-lpdA Sequence [SEQ ID NO: 23] and the lpdA E354K Sequence [SEQ ID NO: 24].

FIG. 1E depicts the ptsI KO Sequence [SEQ ID NO: 25].

FIG. 1F depicts the pC75-EcExuT Sequence [SEQ ID NO: 26].

FIG. 1G depicts the pC75-VnGLK-VpSGLT Sequence [SEQ ID NO: 27].

FIG. 1H depicts the pC100-CgMDH Sequence [SEQ ID NO: 28].

FIG. 1I depicts the pC100-AsVnPCK hybrid-2 Sequence [SEQ ID NO: 29].

FIG. 1J depicts the PN96_RS22390 KO Sequence [SEQ ID NO: 30].

FIG. 1K depicts the pC25-sbtA Sequence [SEQ ID NO: 31].

FIG. 1L depicts the pC100-AsVnPCK hybrid-1 Sequence [SEQ ID NO: 36].

FIG. 2 depicts a graphical overview of genetically modified pathways. This is a summary of genetic modifications and associated pathways in the engineered V. natriegens succinate bio-production strain. Further illustrated are the primary pathway reactions, mediating enzymes, and associated substrates underlying the biochemical conversion of glucose to succinate. Specific genetic modifications that facilitate the conversion from glucose substrate, to intermediary metabolites, and ultimately to succinate are provided and associated with a detailed description of the relevant aspects of the disclosure in the subsequent sections of this disclosure. Gene deletions are demarcated by a delta symbol (4) before the gene name (e.g., ΔptsI). Heterologous or native gene overexpression is demarcated by a “+” symbol before the relevant gene name.

FIGS. 3A and 3B depict the anaerobic growth of a wild-type V. natriegens strain (GBS004), an engineered strain as previously described in the literature (GBS027), and an engineered strain of the present invention (GBS802), as measured by optical density (OD600). Exponentially growing aerobic precultures of each strain were diluted to an OD600 of 0.05 and grown in 96-well microplate format in the following medium: 22 g/L NaCl, 4.7 g/L MgCl₂, 0.3 g/L KCl, 5 g/L Yeast Extract, 20 g/L Glucose, 100 mM MOPS buffer, 100 mM NaHCO₃, pH adjusted to 7.0 and sterile filtered. Cultures were grown for 20 hours in a BioTek synergy microplate reader in an anaerobic chamber at 37° C., and OD600 values for each strain were measured at various timepoints. FIG. 3A depicts the OD600 values for each strain over the course of the 20-hour growth period. FIG. 3B depicts the final OD600 value for each strain, measured at 20 hours post-inoculation.

FIG. 3C depicts the concentrations of glucose and succinate following a 3-hour fermentation using wild-type, GBS027, and GBS802 V. natriegens strains, i.e., the levels of glucose consumed and succinate produced during the fermentation. Exponentially growing aerobic precultures of the WT strain, the GBS027 strain, and the GBS802 strain were adjusted to an OD600 of 5.0 and grown in 96-well microplate format in the following medium: 22 g/L NaCl, 4.7 g/L MgCl₂, 0.3 g/L KCl, 5 g/L Yeast Extract, 20 g/L Glucose, 100 mM MOPS buffer, 100 mM NaHCO₃, pH adjusted to 7.0 and sterile filtered. Cultures were grown for 3 hours in an anaerobic chamber at 37° C. The concentrations of succinate and glucose present in each culture were measured by HPLC.

FIG. 3D depicts the results of a two-phase fermentation experiment comparing the abilities of the GBS027 and GBS802 strains to produce succinate. On average, the final succinate concentration produced by each strain during the two-phase fermentation was 62.3 g/L for GBS802 and 10.1 g/L for GBS027.

FIG. 3E depicts the results of a single-phase fermentation experiment comparing the abilities of the GBS027 and GBS802 strains to produce succinate. On average, the final succinate concentration produced by each strain during the single-phase fermentation was 52.6 g/L for GBS802 and 2.0 g/L for GBS027.

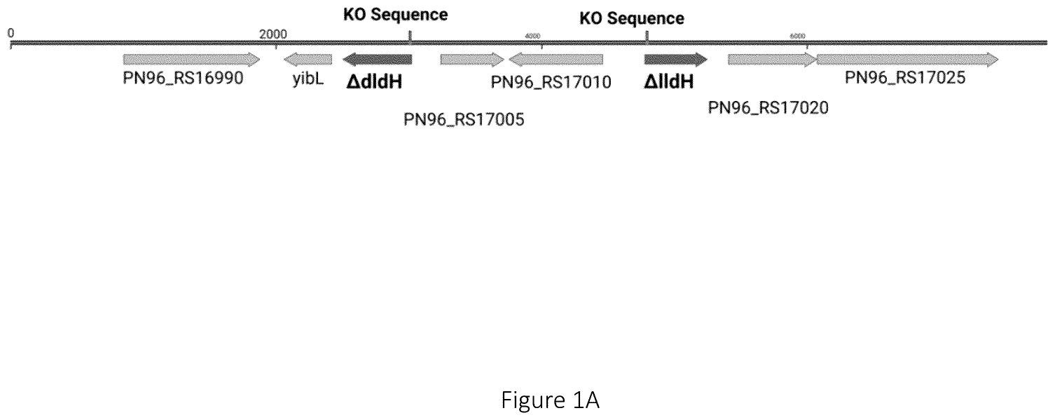

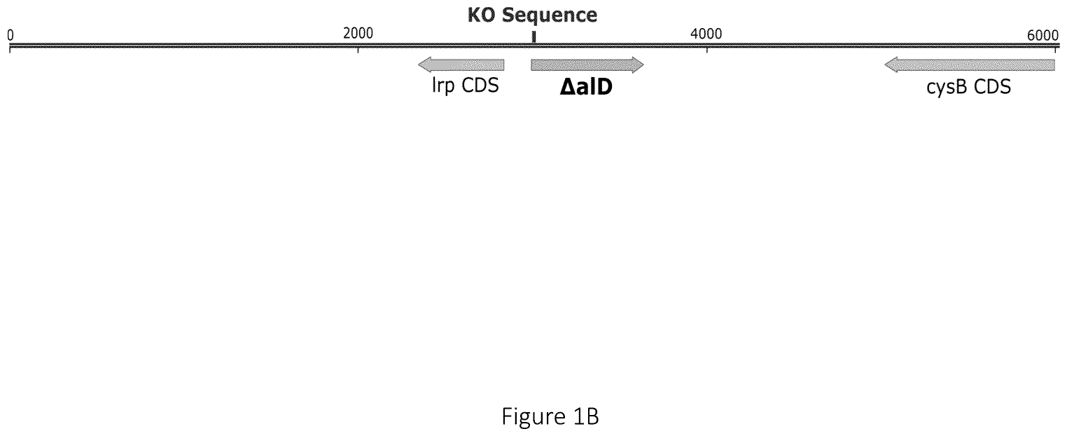

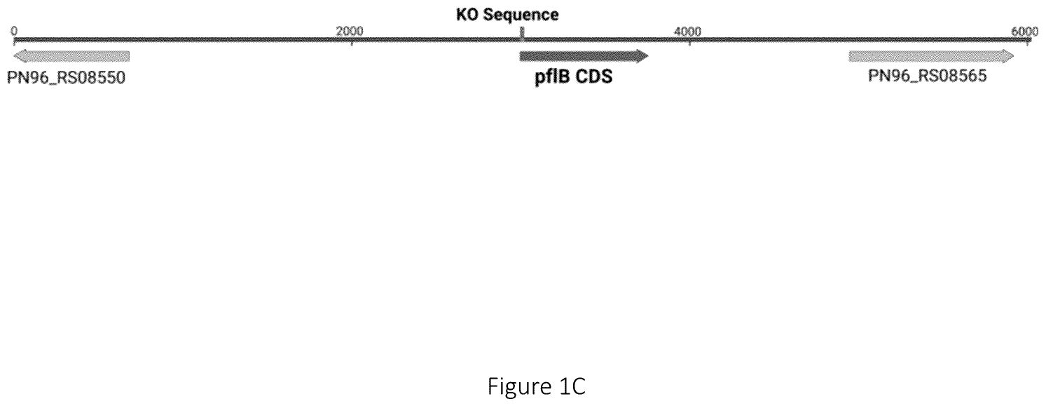

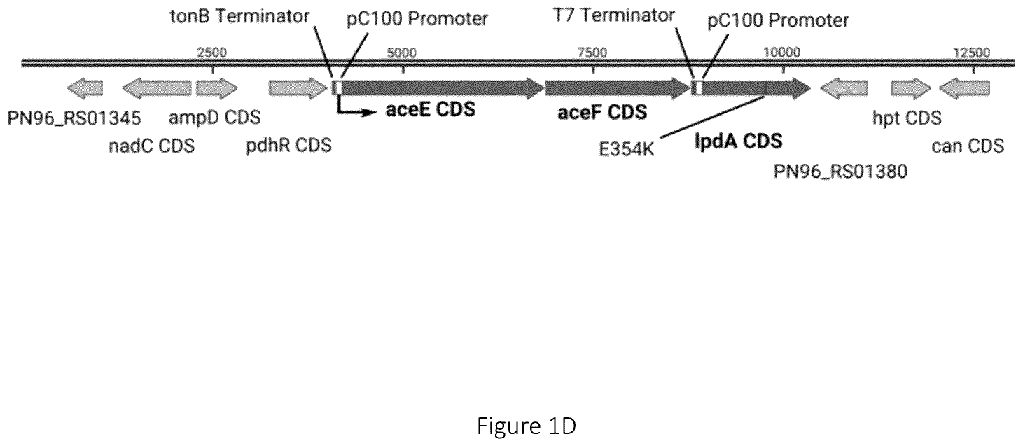

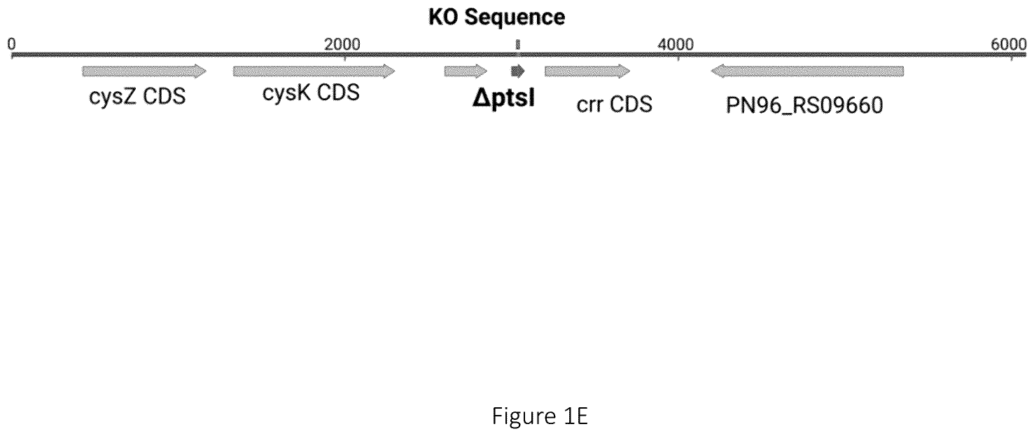

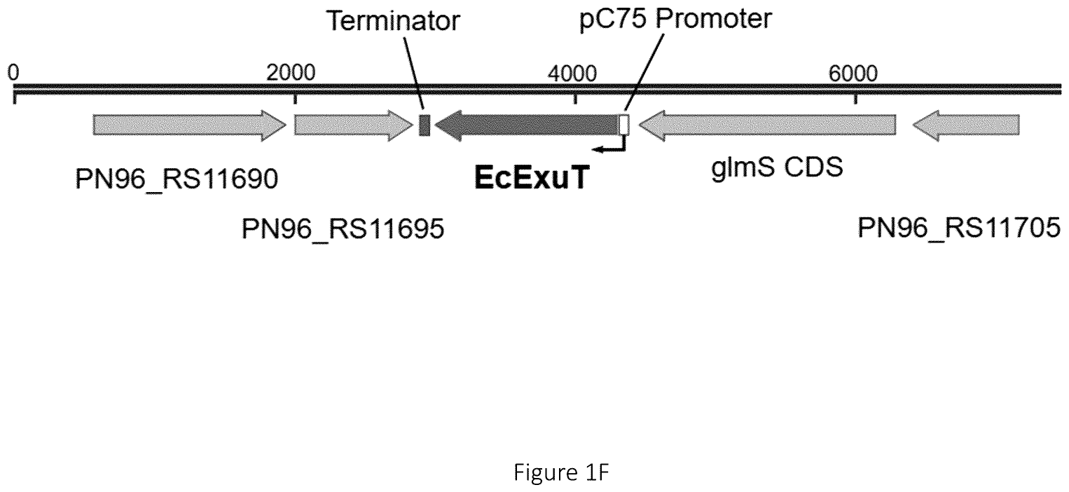

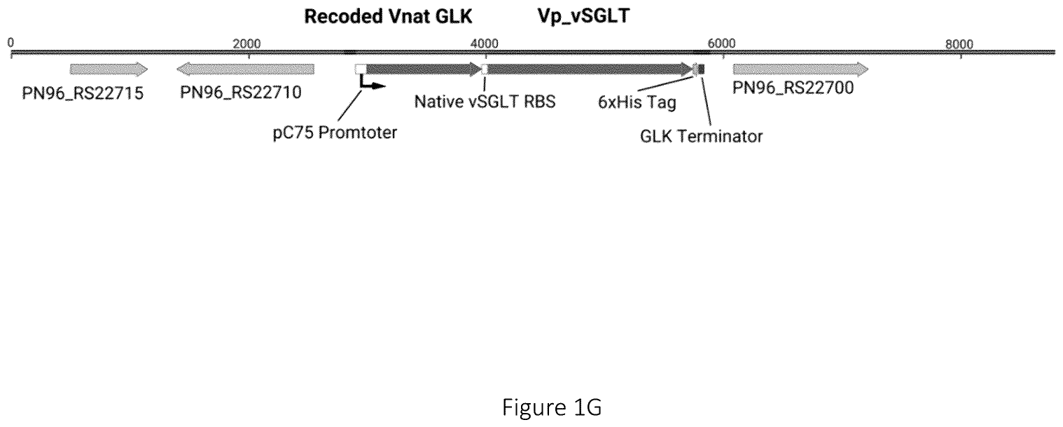

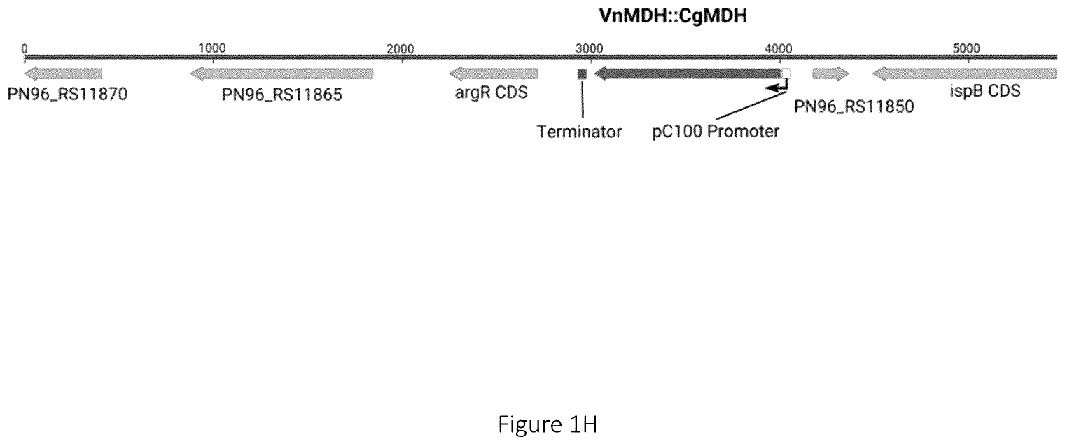

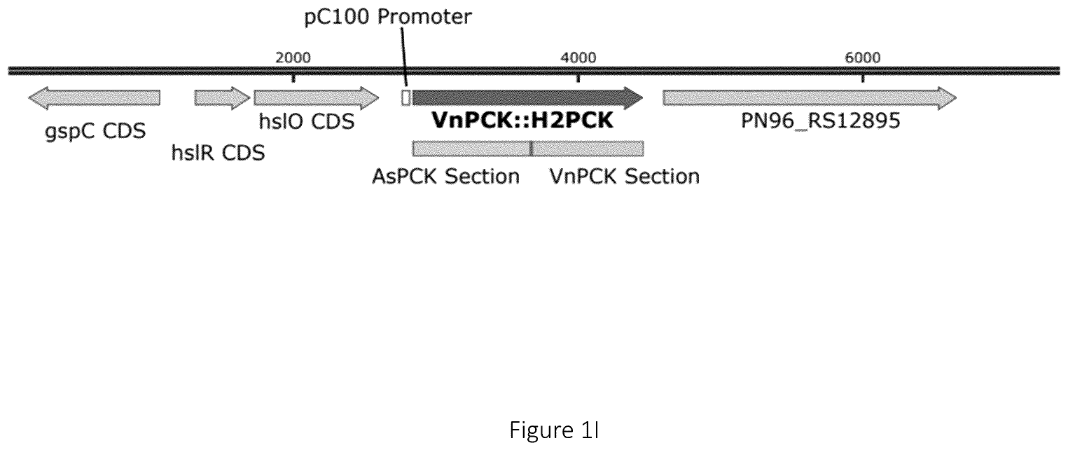

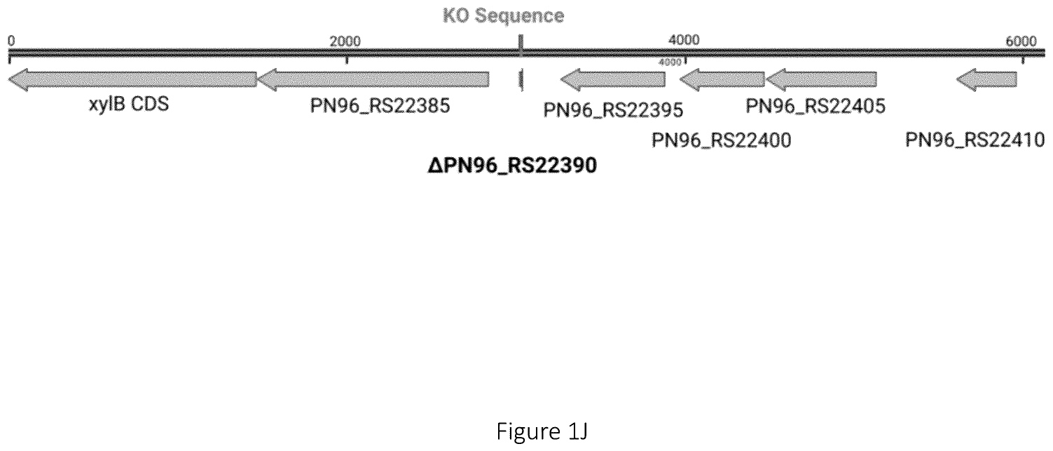

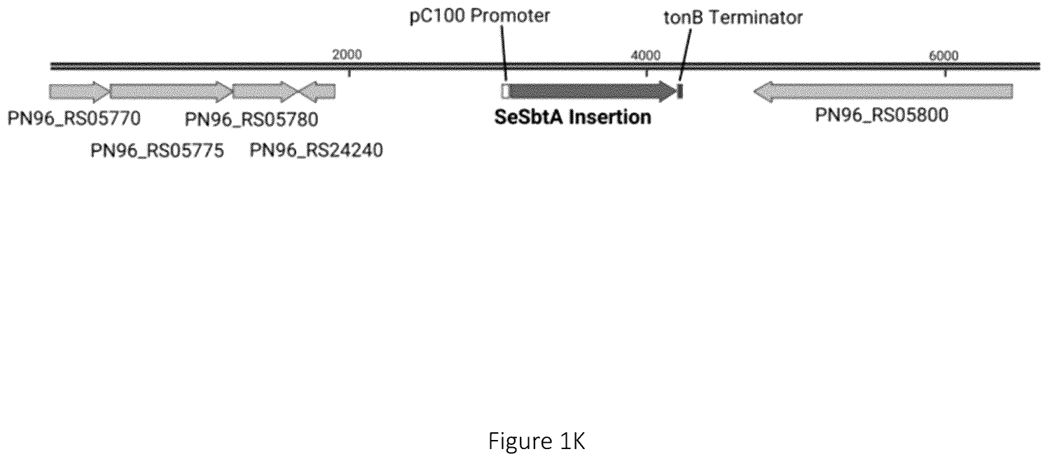

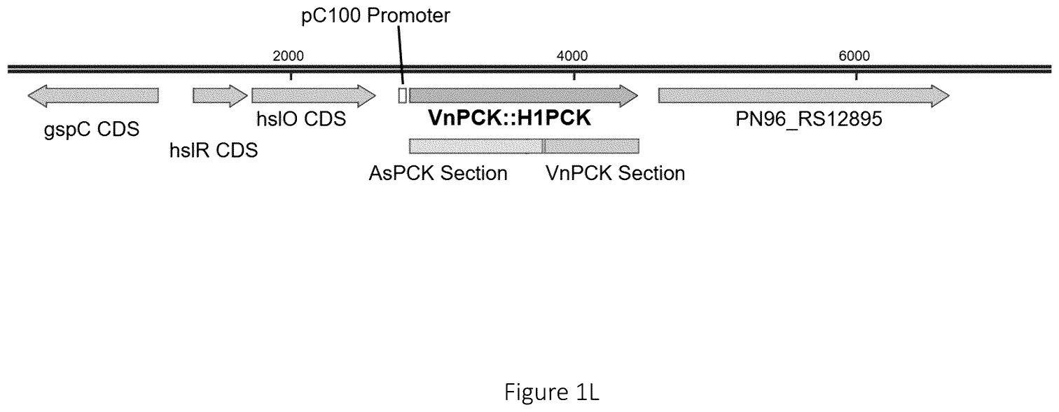

### DETAILED DESCRIPTION OF THE DISCLOSURE

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Definitions

All patents and publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

The practice of the technology described herein will employ, unless indicated specifically to the contrary, conventional methods of chemistry, biochemistry, organic chemistry, molecular biology, bioinformatics, microbiology, recombinant DNA techniques, genetics, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, N.Y. 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

Before describing the present disclosure in detail, it is to be understood that this disclosure is not limited to specific compositions or process steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, the singular forms “a,” “an” and “the” include plural referents unless the content clearly dictates otherwise. Thus, for example, reference to “a protein” includes a mixture of two or more proteins, and the like.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. As used herein, the terms “includes,” “including,” “includes,” “including,” “contains,” “containing,” “have,” “having,” and any variations thereof, are intended to cover a non-exclusive inclusion, such that a process, method, product-by-process, or composition of matter that includes, includes, or contains an element or list of elements does not include only those elements but can include other elements not expressly listed or inherent to such process, method, product-by-process, or composition of matter. Similarly, “comprise,” “comprises,” “comprising” “include,” “includes,” and “including” are interchangeable and not intended to be limiting.

Units, prefixes, and symbols are denoted in their Systeme International de Unites (SI) accepted form. Numeric ranges are inclusive of the numbers defining the range. Unless otherwise indicated, amino acid sequences are written left to right in amino to carboxy orientation. The headings provided herein are not limitations of the various aspects, which can be had by reference to the specification as a whole. Accordingly, the terms defined immediately below are more fully defined by reference to the specification in its entirety.

Other than in the operating examples, or where otherwise indicated, all numbers expressing quantities of ingredients or reaction conditions used herein should be understood as modified in all instances by the term “about.” The term “about” when used to described aspects of the disclosure, in connection with percentages means ±1%, ±2%, ±3%, ±4%, ±5%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

For the recitation of numeric ranges herein, each intervening number there between with the same degree of precision is explicitly contemplated. For example, for the range of 6-9, or 6 to 9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “plurality” is intended to mean a population of two or more different members. Pluralities can range in size from small, medium, large, to very large. The size of small plurality can range, for example, from a few members to tens of members. Medium sized pluralities can range, for example, from tens of members to about 100 members or hundreds of members. Large pluralities can range, for example, from about hundreds of members to about 1000 members, to thousands of members and up to tens of thousands of members. Very large pluralities can range, for example, from tens of thousands of members to about hundreds of thousands, a million, millions, tens of millions and up to or greater than hundreds of millions of members. Therefore, a plurality can range in size from two to well over one hundred million members as well as all sizes, as measured by the number of members, in between and greater than the above exemplary ranges.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

As used herein, the terms “ug” and “uM” are used interchangeably with “μg” and “μM,” respectively.

“Promoter” as used herein refers to a nucleic acid sequence that regulates expression of a transcriptional unit. A “promoter region” is a regulatory region capable of binding RNA polymerase in a cell and initiating transcription of a downstream (3′ direction) coding sequence. Within the promoter region will be found a transcription initiation site (conveniently defined by mapping with nuclease S1), as well as protein binding domains (consensus sequences) responsible for the binding of RNA polymerase such as the putative-35 region and the Pribnow box. The term “operably linked” when describing the relationship between two DNA regions simply means that they are functionally related to each other and they are located on the same nucleic acid fragment. A promoter is operably linked to a structural gene if it controls the transcription of the gene and it is located on the same nucleic acid fragment as the gene.

The term “mutation” is used herein as a general term and includes changes of both single base pair and multiple base pairs. Such mutations may include substitutions, frame-shift mutations, deletions, insertions and truncations.

The term “substrate” as used herein refers to a substance or compound that is converted or suitable for conversion into another compound (e.g., a product) by the action of at least one enzyme. The term includes not only a single compound but also combinations comprising more than one compound.

Nucleic acid sequences may be “introduced” into a cell by protoplast fusion, transfection, transduction, transformation, electroporation or any other suitable method known in the art. A nucleic acid sequence introduced into a eukaryotic or prokaryotic cell may be integrated into a chromosome or may be maintained as an episome.

As used herein, “codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome.

The term “heterologous” polynucleotide as used herein means any polynucleotide that is introduced into a host cell by laboratory techniques, and includes polynucleotides that are removed from a host cell, subjected to laboratory manipulation, and then reintroduced into a host cell. When “heterologous” is used with reference to a nucleic acid or polypeptide, the term refers to a sequence that is not normally expressed and secreted by an organism (e.g., a “wild-type” organism). In some embodiments, the term encompasses a sequence that comprises two or more subsequences which are not found in the same relationship to each other as normally found in nature, or is recombinantly engineered so that its level of expression, or physical relationship to other nucleic acids or other molecules in a cell, or structure, is not normally found in nature. For example, a heterologous nucleic acid is typically recombinantly produced, having two or more sequences from unrelated genes arranged in a manner not found in nature (e.g., a nucleic acid open reading frame (ORF) of the invention operatively linked to a promoter sequence inserted into an expression cassette, such as a vector).

As used herein, a “heterologous enzyme” is used in reference to an enzyme that is encoded by a heterologous gene. However, it is also contemplated herein that a heterologous gene can encode an endogenous or homologous enzyme. As used herein, the term “heterologous gene” refers to a gene that occurs in a form not found in a parental strain of the host cell. Thus, in some embodiments, a heterologous gene is a gene that is derived from a species that is different from the species of the host cell expressing the gene. In some embodiments, a heterologous gene is a modified version of a gene that is endogenous to the host cell (e.g., an endogenous gene subjected to manipulation and then introduced or transformed into the host cell). For example, in some embodiments, a heterologous gene has an endogenous coding sequence, but has modifications in the promoter sequence. Similarly, in other embodiments, a heterologous gene encodes the same amino acid sequence as an endogenous gene, but has modifications in codon usage and/or to noncoding regions (e.g., introns), and/or combinations thereof. In some embodiments, the heterologous gene is a gene that has been modified to overexpress a gene product of interest.

The term “overexpression” as used herein refers to any state in which a gene is caused to be expressed at an elevated rate or level as compared to the endogenous expression rate or level for that gene. In some embodiments, “overexpression” includes an elevated translation rate or level of the gene compared to the endogenous translation rate or level for that gene. In some embodiments, overexpression includes an elevated transcription rate or level of the gene compared to the endogenous transcription rate or level for that gene. It is intended that the term encompass overexpression of endogenous, as well as heterologous proteins.

The term “variant” or “mutant” as used interchangeably herein refer to a polypeptide sequence or polynucleotide sequence encoding a polypeptide, said sequence comprising one or more modifications relative to a corresponding wild-type enzyme (or other specified reference sequence) or the wild-type polynucleotide (or other specified reference sequence) such as substitutions, insertions, deletions, and/or truncations of one or more specific amino acid residues or of one or more specific nucleotides or codons in the polypeptide or polynucleotide. In some embodiments, reference to a variant at an amino acid residue refers to a substitution of the amino acid residue for another amino acid residue. Mutagenesis and directed evolution methods are well known in the art for creating variants. See, e.g., U.S. Pat. Nos. 7,783,428; 6,586,182; 6,117,679; and Ling, et al., 1999, “Approaches to DNA mutagenesis: an overview,” Anal. Biochem., 254 (2): 157-78; Smith, 1985, “In vitro mutagenesis,” Ann. Rev. Genet., 19:423-462; Carter, 1986, “Site-directed mutagenesis,” Biochem. J., 237:1-7; Minshull, et al., 1999, “Protein evolution by molecular breeding,” Current Opinion in Chemical Biology, 3:284-290.

The term “inactivated” as applied to a gene refers to any genetic modification that decreases or eliminates the expression of the gene and/or the functional activity of the corresponding gene product (mRNA and/or protein). The term encompasses complete or partial inactivation, genetic disruption, suppression, deletion, interruption, blockage, promoter alterations, antisense RNA, dsRNA, or down-regulation of a gene. This can be accomplished, for example, by gene “knockout,” inactivation, mutation (e.g., insertion, deletion, point, or frameshift mutations that disrupt the expression or activity of the gene product), or by use of inhibitory RNAs (e.g., sense, antisense, or RNAi technology). A deletion may encompass all or part of a gene's coding sequence. The term “knockout” refers to the deletion of most (at least about 95%, at least about 96%, at least about 97%, at least about 98%, or at least about 99%) or all (100%) of the coding sequence of a gene. In some embodiments, any number of nucleotides can be deleted, from a single base to an entire piece of a chromosome.

As used herein, the term “bioreactor” refers to an enclosed or isolated system or vessel for containment of a microorganism and a biomass material in which a cell culture medium can be contained and internal conditions of which can be controlled during the culturing period, e.g., pH and temperature. The “bioreactor” may preferably be configured for anaerobic growth of the microorganism.

As used herein, the term “culturing” refers to growing a population of microbial cells under suitable conditions using any suitable medium (e.g., liquid, solid, or semi-solid media).

As used herein, the term “fusion,” when referring to a gene, refers to a DNA fragment in which two or more genes are fused in a single reading frame to encode two or more proteins that are fused together via one or more peptide bonds. As used herein, the term “fusion protein” refers to a protein or polypeptide encoded by a fusion gene and it may be used interchangeably with the term “fusion gene product.”

As used herein, the term “upstream” and “downstream” refer to the position of an element of nucleotide sequence. “Upstream” signifies an element that is more 5′ than the reference element. “Downstream” signifies an element that is more 3′ than the reference element.

Various aspects of the disclosure are described in further detail in the following subsections.

### II. Methods of the Disclosure

The present disclosure relates to a method for the enhanced production of succinic acid in a genetically modified strain of Vibrio natriegens. This method involves a series of targeted genetic modifications aimed at augmenting the supply of precursor metabolites for the reductive tricarboxylic acid (rTCA) pathway and amplifying the anaerobic fermentative flux through this pathway. The rTCA pathway in this context includes the sequential enzymatic conversion of oxaloacetate to malate by malate dehydrogenase (Mdh), the transformation of malate to fumarate by fumarase (Fum), and the subsequent production of succinate from fumarate by fumarate reductase (Frd).

The present disclosure provides a method of producing succinic acid, the method comprising growing a plurality of non-naturally occurring Vibrio natriegens cells comprising one or more genetic disruptions, wherein the one or more genetic disruptions increase a production of succinate as compared to a production of succinate from a Vibrio natriegens cell that does not comprise the one or more genetic disruptions; and using the plurality of non-naturally occurring Vibrio natriegens cells to produce succinic acid from a glucose substrate at a rate of from about 0.5 to about 5 g_{Succ} g_{CDW}⁻¹ h⁻¹.

In some embodiments, a carbohydrate substrate involved in the production of succinate from the plurality of non-naturally occurring Vibrio natriegens cells comprise complex carbohydrates. In some embodiments, the complex carbohydrates are pentoses, hexoses, other complex carbohydrates, or a combination thereof. In some embodiments, the complex carbohydrates comprise sucrose, arabinose, xylose, chitin, lignocellulosic hydrolysates, or a combination thereof.

In some embodiments, the plurality of Vibrio natriegens comprise a mutation to a native phosphoenolpyruvate carboxykinase (pck) gene. In some embodiments, the Vibrio natriegens cells comprise a pck from A. succinogenes operatively linked to a constitutive promoter. In some embodiments, the plurality of Vibrio natriegens cells comprise a mutation to a native malate dehydrogenase (mdh) gene. In some embodiments, the mutation comprises a replacement of the native malate dehydrogenase (mdh) gene with an mdh gene from a C. glutamicum, further wherein the mdh gene from the C. glutamicum is operatively linked to a constitutive promoter. In some embodiments, the plurality of Vibrio natriegens cells comprise a mutation to a native fumC gene. In some embodiments, the mutation comprises a replacement of the native fumC gene with a fumR fumarase gene from a Rhizopus oryzae, further wherein the fumR fumarase gene from the Rhizopus oryzae is operatively linked to a constitutive promoter. In some embodiments, the plurality of Vibrio natriegens cells comprise one or more mutations to one or more native fumarate reductase genes (frdBCD). In some embodiments, the mutation comprises a replacement of the native frdBCD genes with a fumarate reductase from a Trypanasoma brucei, further wherein the fumarate reductase from the Trypanasoma brucei is operatively linked to one or more constitutive promoters. In some embodiments, the plurality of Vibrio natriegens cells comprise a mutation to a native mae gene. In some embodiments, the mutation comprises a replacement of the native mae gene with an NADPH-dependent malic enzyme from a Arabidopsis thaliana, further wherein the NADPH-dependent malic enzyme from the Arabidopsis thaliana is operatively linked to a constitutive promoter. In some embodiments, the plurality of Vibrio natriegens cells comprise a deletion of an maeB gene. In some embodiments, the plurality of Vibrio natriegens cells comprise a deletion of an L-lactate dehydrogenase gene (lldH). In some embodiments, the plurality of Vibrio natriegens cells comprise a deletion of a D-lactate dehydrogenase gene (dldH). In some embodiments, the plurality of Vibrio natriegens cells comprise a deletion of an alanine dehydrogenase gene (alD). In some embodiments, the plurality of Vibrio natriegens cells comprise a deletion of a pyruvate formate lyase gene (pflB). In some embodiments, the plurality of Vibrio natriegens cells comprise a deletion of a pyruvate formate lyase gene (pflB). In some embodiments, the plurality of Vibrio natriegens cells comprise a deletion of an alanine dehydrogenase gene (alD). In some embodiments, the plurality of Vibrio natriegens cells comprise a deletion of a native maeA gene. In some embodiments, the plurality of Vibrio natriegens cells comprise a deletion of a succinate dehydrogenase gene (sdh). In some embodiments, the plurality of Vibrio natriegens cells comprise a deletion of a ptsI gene, a component of a phosphoenolpyruvate-dependent sugar phosphotransferase system. In some embodiments, the plurality of Vibrio natriegens cells overexpress a native glucokinase gene (glk) as compared to an expression of a native glucokinase gene (glk) from a plurality of Vibrio natriegens cells that does not comprise the one or more genetic disruptions. In some embodiments, the plurality of Vibrio natriegens cells overexpress a native C4-dicarboxylate exporter gene (dcuA) as compared to an expression of a native C4-dicarboxylate exporter gene (dcuA) from a plurality of Vibrio natriegens cells that does not comprise the one or more genetic disruptions. In some embodiments, the plurality of Vibrio natriegens cells comprise a deletion of a native C4-dicarboxylate importer gene (dctA). In some embodiments, the plurality of Vibrio natriegens cells comprise a deletion of a native acetate kinase gene (ackA). In some embodiments, the plurality of Vibrio natriegens cells comprise a deletion of a native aldehyde-alcohol dehydrogenase gene (adhE). In some embodiments, the plurality of Vibrio natriegens cells comprise a deletion of two methylglyoxal synthase genes, mgsA1 and mgsA2.

The present disclosure also provides a method of producing succinic acid, comprising culturing a plurality of genetically modified Vibrio natriegens cells in the presence of a carbohydrate, wherein genetic modifications of the genetically modified Vibrio natriegens cells enable a coupled growth of microorganisms and production of succinate in the culture. In some embodiments, the plurality of Vibrio natriegens cells comprise a deletion of a gene encoding an regulator of a pyruvate dehydrogenase complex (pdhR). In some embodiments, the plurality of Vibrio natriegens cells overexpress E1 and E2 subunits of the pyruvate dehydrogenase complex (aceEF) as compared to an expression of E1 and E2 subunits of the pyruvate dehydrogenase complex (aceEF) from a plurality of Vibrio natriegens cells that does not comprise the genetic modifications. In some embodiments, the plurality of Vibrio natriegens cells overexpress a mutant copy of a dihydrolipoamide dehydrogenase gene (lpdA) as compared to an expression of a mutant copy of a dihydrolipoamide dehydrogenase gene (lpdA) from a plurality of Vibrio natriegens cells that does not comprise the genetic modifications.

The present disclosure also provides a method of producing succinic acid, comprising culturing a population of genetically modified Vibrio natriegens cells in a single-phase cultivation for a period of greater than 24 hours. In some embodiments, the population of genetically modified Vibrio natriegens cells comprise a pck from A. succinogenes operatively linked to a constitutive promoter. In some embodiments, the population of genetically modified Vibrio natriegens cells comprise a mutation to a native malate dehydrogenase (mdh) gene. In some embodiments, the mutation comprises a replacement of the native malate dehydrogenase (mdh) gene with an mdh gene from a C. glutamicum, further wherein the mdh gene from the C. glutamicum is operatively linked to a constitutive promoter. In some embodiments, the population of genetically modified Vibrio natriegens cells comprise a mutation to a native fumC gene. In some embodiments, the mutation comprises a replacement of the native fumC gene with a fumR fumarase gene from a Rhizopus oryzae, further wherein the fumR fumarase gene from the Rhizopus oryzae is operatively linked to a constitutive promoter. In some embodiments, the population of genetically modified Vibrio natriegens cells comprise one or more mutations to one or more native fumarate reductase genes (frdBCD). In some embodiments, the mutation comprises a replacement of the native frdBCD genes with a fumarate reductase from a Trypanasoma brucei, further wherein the fumarate reductase from the Trypanasoma brucei is operatively linked to one or more constitutive promoters. In some embodiments, the population of genetically modified Vibrio natriegens cells comprise a mutation to a native mae gene. In some embodiments, the mutation comprises a replacement of the native mae gene with an NADPH-dependent malic enzyme from a Arabidopsis thaliana, further wherein the NADPH-dependent malic enzyme from the Arabidopsis thaliana is operatively linked to a constitutive promoter. In some embodiments, the population of genetically modified Vibrio natriegens cells comprise a deletion of an maeB gene. In some embodiments, the population of genetically modified Vibrio natriegens cells comprise a deletion of an L-lactate dehydrogenase gene (lldH). In some embodiments, the population of genetically modified Vibrio natriegens cells comprise a deletion of a D-lactate dehydrogenase gene (dldH). In some embodiments, the population of genetically modified Vibrio natriegens cells comprise a deletion of an alanine dehydrogenase gene (alD). In some embodiments, the population of genetically modified Vibrio natriegens cells comprise a deletion of a pyruvate formate lyase gene (pflB). In some embodiments, the population of genetically modified Vibrio natriegens cells comprise a deletion of a pyruvate formate lyase gene (pflB). In some embodiments, the population of genetically modified Vibrio natriegens cells comprise a deletion of an alanine dehydrogenase gene (alD). In some embodiments, the population of genetically modified Vibrio natriegens cells comprise a deletion of a native maeA gene. In some embodiments, the population of genetically modified Vibrio natriegens cells comprise a deletion of a succinate dehydrogenase gene (sdh). In some embodiments, the population of genetically modified Vibrio natriegens cells comprise a deletion of a ptsG gene, a component of a phosphoenolpyruvate-dependent sugar phosphotransferase system. In some embodiments, the population of genetically modified Vibrio natriegens cells comprise a deletion of a ptsI gene, a component of a phosphoenolpyruvate-dependent sugar phosphotransferase system. In some embodiments, the population of genetically modified Vibrio natriegens cells overexpress a native glucokinase gene (glk) as compared to an expression of a native glucokinase gene (glk) from a plurality of Vibrio natriegens cells that does not comprise the one or more genetic disruptions. In some embodiments, the population of genetically modified Vibrio natriegens cells overexpress a native C4-dicarboxylate exporter gene (dcuA) as compared to an expression of a native C4-dicarboxylate exporter gene (dcuA) from a plurality of Vibrio natriegens cells that does not comprise the one or more genetic disruptions. In some embodiments, the population of genetically modified Vibrio natriegens cells comprise a deletion of a native C4-dicarboxylate importer gene (dctA). In some embodiments, the population of genetically modified Vibrio natriegens cells comprise a deletion of a native acetate kinase gene (ackA). In some embodiments, the population of genetically modified Vibrio natriegens cells comprise a deletion of a native aldehyde-alcohol dehydrogenase gene (adhE). In some embodiments, the population of genetically modified Vibrio natriegens cells comprise a deletion of two methylglyoxal synthase genes, mgsA1 and mgsA2. In some embodiments, the population of genetically modified Vibrio natriegens cells comprise a deletion of a gene encoding an regulator of a pyruvate dehydrogenase complex (pdhR). In some embodiments, the population of genetically modified Vibrio natriegens cells overexpress E1 and E2 subunits of the pyruvate dehydrogenase complex (aceEF) as compared to an expression of E1 and E2 subunits of the pyruvate dehydrogenase complex (aceEF) from a plurality of Vibrio natriegens cells that does not comprise the genetic modifications. In some embodiments, the population of genetically modified Vibrio natriegens cells overexpress a mutant copy of a dihydrolipoamide dehydrogenase gene (lpdA) as compared to an expression of a mutant copy of a dihydrolipoamide dehydrogenase gene (lpdA) from a plurality of Vibrio natriegens cells that does not comprise the genetic modifications. In some embodiments, a length of cultivation of the population of genetically modified Vibrio natriegens cells is greater than 100 hours, 1000 hours, or 10,000 hours. In some embodiments, a rate of succinic acid production of the population of genetically modified Vibrio natriegens cells is from about 0.5 to about 5 g_{Succ} g_{CDW}⁻¹ h⁻¹. In some embodiments, a rate of succinic acid production of the population of genetically modified Vibrio natriegens cells is at least about 1.33 g_{Succ} g_{CDW}⁻¹ h⁻¹.

The present disclosure provides a method of producing succinic acid, the method comprising: culturing a plurality of non-naturally occurring Vibrio natriegens cells comprising one or more genetic disruptions, wherein the one or more genetic disruptions increase the production of succinate as compared to the production of succinate from a V. natriegens cell that does not comprise the one or more genetic disruptions; and wherein under anaerobic conditions, the plurality of non-naturally occurring V. natriegens cells produce succinic acid from a glucose substrate at a rate of at least 0.48 g_{Succ} g_{CDW}⁻¹ h⁻¹.

In some aspects, under anaerobic conditions, the plurality of non-naturally occurring V. natriegens cells produce succinic acid from a glucose substrate at a rate of at least 0.75 g_{Succ} g_{CDW}⁻¹ h⁻¹. In some aspects, under anaerobic conditions, the plurality of non-naturally occurring V. natriegens cells produce succinic acid from a glucose substrate at a rate of at least 1.00 g_{Succ} g_{CDW}⁻¹ h⁻¹. In some aspects, under anaerobic conditions, the plurality of non-naturally occurring V. natriegens cells produce succinic acid from a glucose substrate at a rate of at least 1.25 g_{Succ} g_{CDW}⁻¹ h⁻¹. In some aspects, under anaerobic conditions, the plurality of non-naturally occurring V. natriegens cells produce succinic acid from a glucose substrate at a rate of at least 1.50 g_{Succ} g_{CDW}⁻¹ h⁻¹. In some aspects, under anaerobic conditions, the plurality of non-naturally occurring V. natriegens cells produce succinic acid from a glucose substrate at a rate of at least 1.75 g_{Succ} g_{CDW}⁻¹ h⁻¹. In some aspects, under anaerobic conditions, the plurality of non-naturally occurring V. natriegens cells produce succinic acid from a glucose substrate at a rate of at least 2.00 g_{Succ} g_{CDW}⁻¹ h⁻¹.

In some aspects, under anaerobic conditions, the plurality of non-naturally occurring V. natriegens cells produce succinic acid from a glucose substrate at a rate of about 0.48 g_{Succ} g_{CDW}⁻¹ h⁻¹. In some aspects, under anaerobic conditions, the plurality of non-naturally occurring V. natriegens cells produce succinic acid from a glucose substrate at a rate of about 0.75 g_{Succ} g_{CDW}⁻¹ h⁻¹. In some aspects, under anaerobic conditions, the plurality of non-naturally occurring V. natriegens cells produce succinic acid from a glucose substrate at a rate of about 1.00 g_{Succ} g_{CDW}⁻¹ h⁻¹. In some aspects, under anaerobic conditions, the plurality of non-naturally occurring V. natriegens cells produce succinic acid from a glucose substrate at a rate of about 1.25 g_{Succ} g_{CDW}⁻¹ h⁻¹. In some aspects, under anaerobic conditions, the plurality of non-naturally occurring V. natriegens cells produce succinic acid from a glucose substrate at a rate of about 1.50 g_{Succ} g_{CDW}⁻¹ h⁻¹. In some aspects, under anaerobic conditions, the plurality of non-naturally occurring V. natriegens cells produce succinic acid from a glucose substrate at a rate of about 1.75 g_{Succ} g_{CDW}⁻¹ h⁻¹. In some aspects, under anaerobic conditions, the plurality of non-naturally occurring V. natriegens cells produce succinic acid from a glucose substrate at a rate of about 2.00 g_{Succ} g_{CDW}⁻¹ h⁻¹.

In some aspects, under anaerobic conditions, the plurality of non-naturally occurring V. natriegens cells produce succinic acid from a glucose substrate at a rate of between 0.48 g_{Succ} g_{CDW}⁻¹ h⁻¹ to 0.75 g_{Succ} CDW 1 h⁻¹. In some aspects, under anaerobic conditions, the plurality of non-naturally occurring V. natriegens cells produce succinic acid from a glucose substrate at a rate of between 0.75 g_{Succ} g_{CDW}⁻¹ h⁻¹ to 1.00 g_{Succ} g_{CDW}⁻¹ h⁻¹. In some aspects, under anaerobic conditions, the plurality of non-naturally occurring V. natriegens cells produce succinic acid from a glucose substrate at a rate of between 1.00 g_{Succ} g_{CDW}⁻¹ h⁻¹ to 1.25 g_{Succ} g_{CDW}⁻¹ h⁻¹. In some aspects, under anaerobic conditions, the plurality of non-naturally occurring V. natriegens cells produce succinic acid from a glucose substrate at a rate of between 1.25 g_{Succ} g_{CDW}⁻¹ h⁻¹ to 1.50 g_{Succ} g_{CDW}⁻¹ h⁻¹. In some aspects, under anaerobic conditions, the plurality of non-naturally occurring V. natriegens cells produce succinic acid from a glucose substrate at a rate of between 1.50 g_{Succ} g_{CDW}⁻¹ h⁻¹ to 1.75 g_{Succ} g_{CDW}⁻¹ h⁻¹. In some aspects, under anaerobic conditions, the plurality of non-naturally occurring V. natriegens cells produce succinic acid from a glucose substrate at a rate of between 1.75 g_{Succ} g_{CDW}⁻¹ h⁻¹ to 2.00 g_{Succ} g_{CDW}⁻¹ h⁻¹.

In some aspects, the plurality of V. natriegens cells comprise 6 or more genetic disruptions. In some aspects, the plurality of V. natriegens cells comprise 8 or more genetic disruptions. In some aspects, the plurality of V. natriegens cells comprise 10 or more genetic disruptions. In some aspects, the plurality of V. natriegens cells comprise 12 or more genetic disruptions. In some aspects, the plurality of V. natriegens cells comprise 14 or more genetic disruptions. In some aspects, the plurality of V. natriegens cells comprise 16 or more genetic disruptions. In some aspects, the plurality of V. natriegens cells comprise 18 or more genetic disruptions. In some aspects, the plurality of V. natriegens cells comprise 20 or more genetic disruptions. In some aspects, the plurality of V. natriegens cells comprise 22 or more genetic disruptions. In some aspects, the plurality of V. natriegens cells comprise 24 or more genetic disruptions. In some aspects, the plurality of V. natriegens cells comprise 26 or more genetic disruptions.

In some aspects, the plurality of V. natriegens cells comprise a mutation to a native phosphoenolpyruvate carboxykinase (pck) gene. In some aspects, the V. natriegens cells comprise a pck fusion comprising a V. natriegens pck sequence and an A. succinogenes pck sequence operatively linked to a constitutive promoter. In some aspects, the pck fusion encodes a polypeptide about 85%, 90%, 95%, 96%, 97%, 98%, 99% or 100% identical to the amino acid sequence of SEQ ID NO: 45. In some aspects, the pck fusion encodes a polypeptide comprising the amino acid sequence of SEQ ID NO: 45.

In some aspects, the plurality of V. natriegens cells comprise a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter. In some aspects, the mdh gene from a C. glutamicum encodes a polypeptide about 85%, 90%, 95%, 96%, 97%, 98%, 99% or 100% identical to the amino acid sequence of SEQ ID NO: 43. In some aspects, the mdh gene from a C. glutamicum encodes a polypeptide comprising the amino acid sequence of SEQ ID NO: 43.

In some aspects, the plurality of V. natriegens cells comprise a deletion of an L-lactate dehydrogenase gene (lldH).

In some aspects, the plurality of V. natriegens cells comprise a deletion of a D-lactate dehydrogenase gene (dldH).

In some aspects, the plurality of V. natriegens cells comprise a deletion of an alanine dehydrogenase gene (alD).

In some aspects, the plurality of V. natriegens cells comprise a deletion of a pyruvate formate lyase gene (pflB).

In some aspects, the plurality of V. natriegens cells comprise a deletion of each of the lldH, dldH, alD, and pflB genes.

In some aspects, the plurality of V. natriegens cells comprise a deletion of a ptsI gene.

In some aspects, the plurality of V. natriegens cells comprise a native glucokinase gene (glk) operably linked to a constitutive synthetic promoter. In some aspects, the glk gene encodes a polypeptide about 85%, 90%, 95%, 96%, 97%, 98%, 99% or 100% identical to the amino acid sequence of SEQ ID NO: 42. In some aspects, the glk gene encodes a polypeptide comprising the amino acid sequence of SEQ ID NO: 42.

In some aspects, the plurality of V. natriegens cells overexpress a native glucokinase gene (glk).

In some aspects, the plurality of V. natriegens cells comprise a heterologous sodium-dependent glucose transporter protein (vsGLT) from Vibrio parahaemolyticus. In some aspects, the vsGLT gene encodes a polypeptide about 85%, 90%, 95%, 96%, 97%, 98%, 99% or 100% identical to the amino acid sequence of SEQ ID NO: 41. In some aspects, the vsGLT gene encodes a polypeptide comprising the amino acid sequence of SEQ ID NO: 41.

In some aspects, the plurality of V. natriegens cells comprise a mutation to the native E1 and E2 subunits of a pyruvate dehydrogenase complex (aceEF) gene. In some aspects, the aceEF gene comprises a nucleotide sequence that encodes a polypeptide about 85%, 90%, 95%, 96%, 97%, 98%, 99% or 100% identical to the amino acid sequences of SEQ ID NOs: 37 and/or 38. In some aspects, the aceEF gene comprises a nucleotide sequence that encodes a polypeptide comprising the amino acid sequences of SEQ ID NO: 37 and/or 38.

In some aspects, the mutation comprises a replacement of the native promoter of the aceEF gene with a synthetic promoter.

In some aspects, wherein the plurality of V. natriegens cells comprise a mutation to a native E3 subunit of a pyruvate dehydrogenase complex (lpdA) gene. In some aspects, the mutation comprises an E354K mutation and a replacement of the native promoter of the lpdA gene with a synthetic promoter. In some aspects, the lpdA gene encodes a polypeptide about 85%, 90%, 95%, 96%, 97%, 98%, 99% or 100% identical to the amino acid sequence of SEQ ID NO: 39. In some aspects, the lpdA gene encodes a polypeptide comprising the amino acid sequence of SEQ ID NO: 39.

In some aspects, the plurality of V. natriegens cells comprise a heterologous E. coli hexuronate transporter (exuT) gene, wherein the exuT gene is operably linked to a constitutive synthetic promoter. In some aspects, the exuT gene encodes a polypeptide about 85%, 90%, 95%, 96%, 97%, 98%, 99% or 100% identical to the amino acid sequence of SEQ ID NO: 40. In some aspects, the exuT gene encodes a polypeptide comprising the amino acid sequence of SEQ ID NO: 40.

In some aspects, the plurality of V. natriegens cells comprise a deletion of a PN96_RS22390 gene.

In some aspects, the plurality of V. natriegens cells comprise a heterologous S. elongatus (PCC7002) Sodium-Dependent Bicarbonate Transporter (sbtA) gene. In some aspects, the sbtA gene encodes a polypeptide about 85%, 90%, 95%, 96%, 97%, 98%, 99% or 100% identical to the amino acid sequence of SEQ ID NO: 46. In some aspects, the sbtA gene encodes a polypeptide comprising the amino acid sequence of SEQ ID NO: 46.

In some aspects, the plurality of V. natriegens cells comprise: a deletion of each of the lldH, dldH, alD, and pflB genes; and a pck fusion comprising a V. natriegens pck sequence and an A. succinogenes pck sequence operatively linked to a constitutive promoter.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; and a deletion of a PN96_RS22390 gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; a deletion of a PN96_RS22390 gene; and a heterologous S. elongatus (PCC7002) Sodium-Dependent Bicarbonate Transporter (sbtA) gene.

In some aspects, the plurality of V. natriegens cells comprise one or more modifications selected from Table D. In some aspects, the mutations are relative to a wild-type V. natriegens genome (e.g., a P. Baumann 111 strain, ATCC #14048).

In some aspects, the plurality of V. natriegens cells comprise a modification, wherein the modification comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 1-36. In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one of SEQ ID NOs: 1-36.

In some aspects, the plurality of V. natriegens cells comprise a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22.

In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-22. In some aspects, the plurality of V. natriegens cells comprise a plurality of modifications, wherein each of the plurality of modifications comprises the nucleotide sequence of each of SEQ ID NOs: 19-22.

In some aspects, the plurality of V. natriegens cells comprise a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29.

In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-22 and 29. In some aspects, the plurality of V. natriegens cells comprise a plurality of modifications, wherein each of the plurality of modifications comprises the nucleotide sequence of each of SEQ ID NOs: 19-22 and 29.

In some aspects, the plurality of V. natriegens cells comprise a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28.

In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 7, 19-24, and 29. In some aspects, the plurality of V. natriegens cells comprise a plurality of modifications, wherein each of the plurality of modifications comprises the nucleotide sequence of each of SEQ ID NOs: 7, 19-24, and 29. In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-24, 28, and 29. In some aspects, the plurality of V. natriegens cells comprise a plurality of modifications, wherein each of the plurality of modifications comprises the nucleotide sequence of each of SEQ ID NOs: 19-24, 28, and 29.

In some aspects, the plurality of V. natriegens cells comprise a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or 25, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2 or 27, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26.

In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 1, 2, 7, 19-24, 26, and 29. In some aspects, the plurality of V. natriegens cells comprise a plurality of modifications, wherein each of the plurality of modifications comprises the nucleotide sequence of each of SEQ ID NOs: 1, 2, 7, 19-24, 26, and 29. In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-29. In some aspects, the plurality of V. natriegens cells comprise a plurality of modifications, wherein each of the plurality of modifications comprises the nucleotide sequence of each of SEQ ID NOs: 19-29.

In some aspects, the plurality of V. natriegens cells comprise a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or 25, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2 or 27, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30.

In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 1, 2, 7, 19-24, 26, 29, and 30. In some aspects, the plurality of V. natriegens cells comprise a plurality of modifications, wherein each of the plurality of modifications comprises the nucleotide sequence of each of SEQ ID NOs: 1, 2, 7, 19-24, 26, 29, and 30. In some aspects, the plurality of V. natriegens cells comprise a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-30. In some aspects, the plurality of V. natriegens cells comprise a plurality of modifications, wherein each of the plurality of modifications comprises the nucleotide sequence of each of SEQ ID NOs: 19-30.

The present disclosure provides a method of producing succinic acid, comprising culturing a plurality of genetically modified Vibrio natriegens cells in the presence of a carbohydrate, wherein the genetically modified V. natriegens cells enable production of succinate in the culture during the V. natriegens growth phase. In some aspects, the plurality of V. natriegens cells overexpress the E1 and E2 subunits of the pyruvate dehydrogenase complex (aceEF). In some aspects, the plurality of V. natriegens cells overexpress a dihydrolipoamide dehydrogenase gene (lpdA).

The present disclosure provides a method of producing succinic acid, comprising culturing a population of genetically modified Vibrio natriegens cells in a two-phase cultivation for a period of greater than 24 hours. In some aspects, the two-phase cultivation is for a period of greater than 48 hours. In some aspects, the two-phase cultivation is for a period of greater than 3 days. In some aspects, the two-phase cultivation is for a period of greater than 4 days. In some aspects, the two-phase cultivation is for a period of greater than 5 days. In some aspects, the two-phase cultivation is for a period of greater than 6 days. In some aspects, the two-phase cultivation is for a period of greater than 7 days.

In some aspects, the two-phase cultivation is for a period of less than 7 days. In some aspects, the two-phase cultivation is for a period of less than 6 days. In some aspects, the two-phase cultivation is for a period of less than 5 days. In some aspects, the two-phase cultivation is for a period of less than 4 days. In some aspects, the two-phase cultivation is for a period of less than 3 days. In some aspects, the two-phase cultivation is for a period of less than 2 days.

In some aspects, the two-phase cultivation is for a period of between 1 and 7 days. In some aspects, the two-phase cultivation is for a period of between 1 and 6 days. In some aspects, the two-phase cultivation is for a period of between 1 and 5 days. In some aspects, the two-phase cultivation is for a period of between 1 and 4 days. In some aspects, the two-phase cultivation is for a period of between 1 and 3 days. In some aspects, the two-phase cultivation is for a period of between 1 and 2 days. In some aspects, the two-phase cultivation is for a period of between 2 and 7 days. In some aspects, the two-phase cultivation is for a period of between 3 and 6 days. In some aspects, the two-phase cultivation is for a period of between 4 and 5 days.

In some aspects, each phase of the two-phase cultivation takes place in the same vessel. In some aspects, the vessel is a bioreactor.

In some aspects, the culturing does not comprise a concentration step. In some aspects, the culturing does comprise a concentration step.

In some aspects, following the culturing and production steps, the method further comprises a purification step.

In some aspects, the plurality of V. natriegens cells comprise a mutation to a native phosphoenolpyruvate carboxykinase (pck) gene. In some aspects, the V. natriegens cells comprise a pck fusion comprising a V. natriegens pck sequence and an A. succinogenes pck sequence operatively linked to a constitutive promoter. In some aspects, the pck fusion encodes a polypeptide about 85%, 90%, 95%, 96%, 97%, 98%, 99% or 100% identical to the amino acid sequence of SEQ ID NO: 45. In some aspects, the pck fusion encodes a polypeptide comprising the amino acid sequence of SEQ ID NO: 45.

In some aspects, the plurality of V. natriegens cells comprise a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter. In some aspects, the mdh gene from a C. glutamicum encodes a polypeptide about 85%, 90%, 95%, 96%, 97%, 98%, 99% or 100% identical to the amino acid sequence of SEQ ID NO: 43. In some aspects, the mdh gene from a C. glutamicum encodes a polypeptide comprising the amino acid sequence of SEQ ID NO: 43.

In some aspects, the plurality of V. natriegens cells comprise a deletion of an L-lactate dehydrogenase gene (lldH).

In some aspects, the plurality of V. natriegens cells comprise a deletion of a D-lactate dehydrogenase gene (dldH).

In some aspects, the plurality of V. natriegens cells comprise a deletion of an alanine dehydrogenase gene (alD).

In some aspects, the plurality of V. natriegens cells comprise a deletion of a pyruvate formate lyase gene (pflB).

In some aspects, the plurality of V. natriegens cells comprise a deletion of each of the lldH, dldH, alD, and pflB genes.

In some aspects, the plurality of V. natriegens cells comprise a deletion of a ptsI gene.

In some aspects, the plurality of V. natriegens cells comprise a native glucokinase gene (glk) operably linked to a constitutive synthetic promoter. In some aspects, the glk gene encodes a polypeptide about 85%, 90%, 95%, 96%, 97%, 98%, 99% or 100% identical to the amino acid sequence of SEQ ID NO: 42. In some aspects, the glk gene encodes a polypeptide comprising the amino acid sequence of SEQ ID NO: 42.

In some aspects, the plurality of V. natriegens cells overexpress a native glucokinase gene (glk).

In some aspects, the plurality of V. natriegens cells comprise a heterologous sodium-dependent glucose transporter protein (vsGLT) from Vibrio parahaemolyticus. In some aspects, the vsGLT gene encodes a polypeptide about 85%, 90%, 95%, 96%, 97%, 98%, 99% or 100% identical to the amino acid sequence of SEQ ID NO: 41. In some aspects, the vsGLT gene encodes a polypeptide comprising the amino acid sequence of SEQ ID NO: 41.

In some aspects, the plurality of V. natriegens cells comprise a mutation to the native E1 and E2 subunits of a pyruvate dehydrogenase complex (aceEF) gene. In some aspects, the aceEF gene comprises a nucleotide sequence that encodes a polypeptide about 85%, 90%, 95%, 96%, 97%, 98%, 99% or 100% identical to the amino acid sequences of SEQ ID NOs: 37 and/or 38. In some aspects, the aceEF gene comprises a nucleotide sequence that encodes a polypeptide comprising the amino acid sequences of SEQ ID NO: 37 and/or 38.

In some aspects, the mutation comprises a replacement of the native promoter of the aceEF gene with a synthetic promoter.

In some aspects, wherein the plurality of V. natriegens cells comprise a mutation to a native E3 subunit of a pyruvate dehydrogenase complex (lpdA) gene. In some aspects, the mutation comprises an E354K mutation and a replacement of the native promoter of the lpdA gene with a synthetic promoter. In some aspects, the lpdA gene encodes a polypeptide about 85%, 90%, 95%, 96%, 97%, 98%, 99% or 100% identical to the amino acid sequence of SEQ ID NO: 39. In some aspects, the lpdA gene encodes a polypeptide comprising the amino acid sequence of SEQ ID NO: 39.

In some aspects, the plurality of V. natriegens cells comprise a heterologous E. coli hexuronate transporter (exuT) gene, wherein the exuT gene is operably linked to a constitutive synthetic promoter. In some aspects, the exuT gene encodes a polypeptide about 85%, 90%, 95%, 96%, 97%, 98%, 99% or 100% identical to the amino acid sequence of SEQ ID NO: 40. In some aspects, the exuT gene encodes a polypeptide comprising the amino acid sequence of SEQ ID NO: 40.

In some aspects, the plurality of V. natriegens cells comprise a deletion of a PN96_RS22390 gene.

In some aspects, the plurality of V. natriegens cells comprise a heterologous S. elongatus (PCC7002) Sodium-Dependent Bicarbonate Transporter (sbtA) gene. In some aspects, the sbtA gene encodes a polypeptide about 85%, 90%, 95%, 96%, 97%, 98%, 99% or 100% identical to the amino acid sequence of SEQ ID NO: 46. In some aspects, the sbtA gene encodes a polypeptide comprising the amino acid sequence of SEQ ID NO: 46.

In some aspects, the plurality of V. natriegens cells comprise: a deletion of each of the lldH, dldH, alD, and pflB genes; and a pck fusion comprising a V. natriegens pck sequence and an A. succinogenes pck sequence operatively linked to a constitutive promoter.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; and a deletion of a PN96_RS22390 gene.

In some aspects, the plurality of V. natriegens cells comprise: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; a deletion of a PN96_RS22390 gene; and a heterologous S. elongatus (PCC7002) Sodium-Dependent Bicarbonate Transporter (sbtA) gene.

In some aspects, the rate of succinic acid production of the population of genetically modified V. natriegens cells is from about 0.48 to about 2.00 g_{Succ} g_{CDW}⁻¹ h⁻¹. In some aspects, the rate of succinic acid production of the population of genetically modified V. natriegens cells is from about 0.50 to about 2.00 g_{Succ} g_{CDW}⁻¹ h⁻¹. In some aspects, the rate of succinic acid production of the population of genetically modified V. natriegens cells is from about 0.75 to about 1.75 g_{Succ} g_{CDW}⁻¹ h⁻¹. In some aspects, the rate of succinic acid production of the population of genetically modified V. natriegens cells is from about 1.00 to about 1.50 g_{Succ} g_{CDW}⁻¹ h⁻¹. In some aspects, the rate of succinic acid production of the population of genetically modified V. natriegens cells is from about 1.00 to about 1.75 g_{Succ} g_{CDW}⁻¹ h⁻¹. In some aspects, the rate of succinic acid production of the population of genetically modified V. natriegens cells is from about 1.25 to about 2.00 g_{Succ} g_{CDW}⁻¹ h⁻¹. In some aspects, the rate of succinic acid production of the population of genetically modified V. natriegens cells is greater than about 2.00 g_{Succ} g_{CDW}⁻¹ h⁻¹.

In some aspects, the method is performed in a bioreactor. In some aspects, the bioreactor is an anaerobic bioreactor.

In some aspects, the rate of succinic acid production is measured over a two hour period. In some aspects, the rate of succinic acid production is measured over a three hour period. In some aspects, the rate of succinic acid production is measured over a four hour period. In some aspects, the rate of succinic acid production is measured over a five hour period. In some aspects, the rate of succinic acid production is measured over a six hour period. In some aspects, the rate of succinic acid production is measured over a seven hour period. In some aspects, the rate of succinic acid production is measured over an eight hour period. In some aspects, the rate of succinic acid production is measured over a nine hour period. In some aspects, the rate of succinic acid production is measured over a ten hour period. In some aspects, the rate of succinic acid production is measured over an eleven hour period. In some aspects, the rate of succinic acid production is measured over a twelve hour period.

This disclosure delineates a novel approach for the biological production of succinic acid and related high-value intermediary metabolites in V. natriegens, facilitated by a comprehensive and methodical genetic engineering strategy. The disclosure capitalizes on the distinctive metabolic capabilities of V. natriegens, repurposing intermediates of the tricarboxylic acid cycle as precursors for a diverse array of chemicals and materials of industrial significance. The approach encompasses: 1.) the enhancement of glycolytic and anaplerotic fluxes to bolster the availability of metabolites for the rTCA pathway, 2.) the strategic redirection of cellular resources and intermediary metabolites into the rTCA pathway, and 3.) the suppression of competing fermentative pathways that otherwise divert carbon and electron flux away from the rTCA pathway. This disclosure thus presents a significant advancement in the field of industrial biotechnology, offering a novel and efficient route for the production of succinic acid and other valuable compounds.

1.) Enhancing Glycolytic and Anaplerotic Flux Towards rTCA Pathway Metabolites

### Enhancing Glycolytic Flux by Improving Glucose Substrate Uptake Rate and Efficiency:

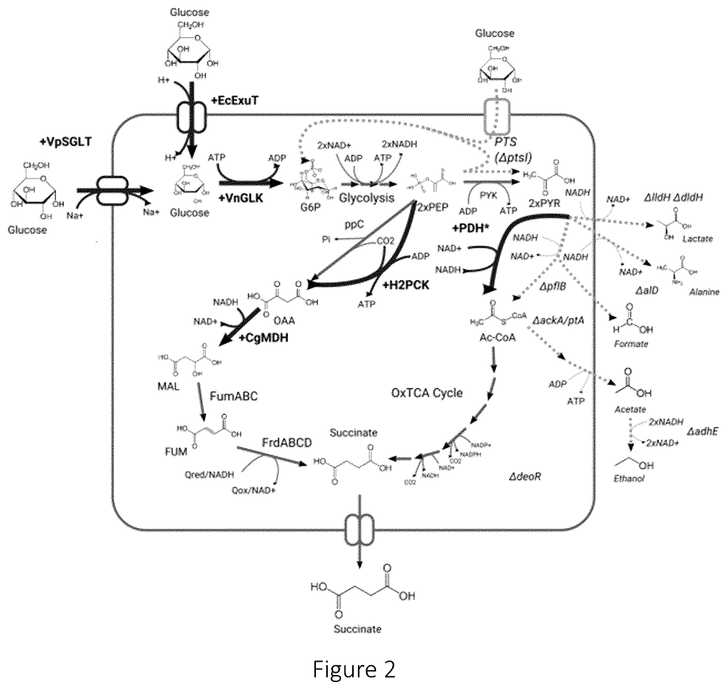

The present aspect of the disclosure (visually summarized in FIG. 2) pertains to a method for augmenting glycolytic flux in the marine bacterium Vibrio natriegens, achieved by enhancing the rate and efficiency of glucose substrate uptake. Vibrio natriegens is characterized by its remarkable substrate flexibility and uptake capabilities, attributable to its expansive genome that encodes a diverse array of transporters, catabolic enzymes, and regulatory elements. This genetic repertoire enables Vibrio natriegens to rapidly adapt to and utilize over 70 different carbon sources, including a variety of sugars, amino acids, carboxylic acids, and polysaccharides. Notably, the organism exhibits an extraordinary capacity for substrate uptake, facilitated by an abundance of ABC transport systems, allowing for the rapid import and catabolism of multiple substrates simultaneously. This capability, combined with swift enzyme kinetics and rapid cell division, positions Vibrio natriegens as a highly efficient scavenger and metabolizer of substrates. See Hoffart, E., et al., “High Substrate Uptake Rates Empower Vibrio natriegens as Production Host for Industrial Biotechnology,” Applied and Environmental Microbiology, 83 (22): e01614-17 (2017).

In aerobic and anaerobic conditions, Vibrio species predominantly employ the phosphoenolpyruvate-dependent phosphotransferase system (PTS) for glucose import. See Kotrba, P., et al., “Bacterial phosphotransferase system (PTS) in carbohydrate uptake and control of carbon metabolism,” Journal of Bioscience and Bioengineering, 92 (6): 502-517 (2001). This system, comprising a multi-protein symporter, integrates glucose transport with phosphorylation. The PTS involves general proteins such as Enzyme I (EI) and HPr, alongside glucose-specific components like EIIBC^{Glc} (encoded by the ptsG gene) and EIIA^{Glc}. EIIBC^{Glc}, an integral membrane protein, facilitates glucose entry into the cell while concurrently phosphorylating it, resulting in the intracellular formation of glucose-6-phosphate. This process involves a sequential transfer of phosphate from phosphoenolpyruvate (PEP) to glucose, mediated by the aforementioned PTS components.

As PEP becomes limiting under anaerobic or oxygen-limited conditions (i.e., during the anaerobic production of succinate), the disclosure incorporates the use of non-PTS transporters, including the galactose/glucose-Na+ symporter vSGLT. An aspect of this disclosure involves the implementation of a heterologous vSGLT symporter, which utilizes sodium influx to facilitate glucose transport into the cell, modified to enable substrate channeling via translational fusion to a substrate-modifying enzyme. Specifically, this instantiation enables constitutive, rapid glucose uptake via facilitated diffusion across all conditions, particularly under anoxic conditions or when PEP availability is restricted.

Furthermore, the disclosure encompasses a novel approach for intracellular glucose phosphorylation. A modified glucokinase (Glk) is employed operably linked to a constitutive synthetic promoter, and a heterologous sodium-dependent glucose transporter protein (vsGLT) from Vibrio parahaemolyticus is introduced. See Xie, Z., et al., “Characterization of the Vibrio parahaemolyticus Na+/Glucose Cotransporter: A Bacterial Member of the Sodium/Glucose Transporter (Sglt) Family,” Journal of Biological Chemistry, 275 (34): 25959-25964 (2000). These modifications ensures continuous PTS-independent glucose phosphorylation to glucose-6-phosphate, thereby sustaining carbon flux into glycolysis and subsequent metabolic pathways underlying fermentative metabolism.

Collectively, these advancements in glucose uptake and phosphorylation mechanisms significantly enhance the glycolytic flux in the non-naturally occurring Vibrio natriegens disclosed herein, thereby optimizing carbon metabolism and facilitating the efficient production of succinate, and other fermentative products, under varying oxygen conditions.

This aspect of the disclosure pertains to a specific genetic modification strategy designed to, for example, optimize glucose import in V. natriegens. One objective is to bypass the native phosphoenolpyruvate (PEP)-dependent phosphotransferase system (PTS) in favor of alternative non-PTS import pathways. This modification is intended, for example, to conserve PEP availability for the engineered succinate production pathway, thereby enhancing overall metabolic efficiency.

Specifically, this aspect of the disclosure involves:

The targeted deletion of the ptsI gene, which encodes the general Enzyme I (EI) component of the phosphotransferase system (PTS). The primary function of EI in the PTS is to initiate the process of PEP binding and subsequent de-phosphorylation. By removing the ptsI gene, the disclosure effectively impedes the initial step of PEP utilization within the PTS pathway.

This genetic modification is a component of the disclosure, as it addresses the need to efficiently manage intracellular PEP levels while ensuring a steady influx of glucose for metabolic processes. By diverting PEP away from the PTS and preserving it for succinate production, this aspect of the disclosure contributes to the overall effectiveness and productivity of the bio-based succinate production process.

This genetic alteration prevents the consumption of PEP by the glucose PTS, thereby conserving this critical metabolite, facilitating an increased anapleurotic flux towards the reductive tricarboxylic acid (rTCA) pathway by preventing the initial phosphotransferase step of the PTS, broadly eliminating PEP consumption across all PTSs. The conservation of PEP achieved through this modification effectively doubles the amount of PEP available for conversion into key rTCA intermediary metabolites. These intermediates are essential for the downstream production of succinate.

The genomic integration of vSGLT and glk, encoding the Vibrio parahaemolyticus galactose/glucose transporter (Vp_vSGLT) and the Vibrio natriegens glucokinase (Glk), respectively, delineates a sophisticated genetic modification strategy aimed at facilitating efficient non-PTS-mediated glucose uptake in Vibrio natriegens. The modification strategy includes the creation of a translational fusion between Vp_vSGLT and Glk to enable rapid and efficient glucose import and phosphorylation. Vp_vSGLT facilitates the diffusion of glucose across the cell membrane, driven by the sodium ion gradient. Subsequently, Glk phosphorylates the imported glucose to glucose-6-phosphate, utilizing ATP as the phosphate donor, as described by the reaction:
Glucose+ATP→Glucose· [symbol] -Glucose-6-phosphate+ADP

An additional aspect of this disclosure involves constructing a synthetic operon containing recoded version of the native V. natriegens glk locus under regulation of a synthetic constitutive promoter, pC75 and glk ribosome binding site, and downstream of the recoded V. natriegens glk, a heterologous sodium-dependent glucose transporter protein (vsGLT) from Vibrio parahaemolyticus. This modification ensures, for example, a stable intracellular pool of free, cytosolic Glk. In conjunction with the ptsI gene deletion, this aspect of the disclosure guarantees efficient import and phosphorylation of glucose substrates under oxygen-limited conditions while minimizing PEP-mediated glucose phosphorylation. As a result, the conservation of the precursor metabolite PEP is achieved, promoting additional carbon flux through the engineered rTCA pathway and thereby enhancing the production of key intermediary metabolites in the V. natriegens succinate production strain.

The targeted deletion of the mgsA1 and mgsA2 genes, which encode paralogs of the enzyme methylglyoxal synthase (MgsA), for example, to prevent catalysis of the glycolytic intermediate dihydroxyacetone phosphate. Methylglyoxal synthase is responsible for the conversion of the glycolytic intermediate dihydroxyacetone phosphate (DHAP) into methylglyoxal and inorganic phosphate. The enzymatic reaction catalyzed by MgsA involves the cleavage of the C2-C3 bond in DHAP, resulting in the release of phosphate and the formation of an enediol intermediate. This intermediate subsequently tautomerizes to form methylglyoxal, as described by the reaction:
DHAP+MgsA→DHAP·MgsA [symbol] MgsA+Methylglyoxal+Pi

The deletion of mgsA genes in this disclosure, for example, is designed to prevent the diversion of DHAP and mitigate the inhibitory effects on glycolytic enzymes. By eliminating the pathways that lead to methylglyoxal synthesis, this modification ensures uninterrupted glycolytic flux towards the rTCA pathway. This is particularly advantageous during periods of transient nutritional stress, which are common in large-scale bioproduction processes. These modifications represent a broadly applicable component of the metabolic engineering strategy of V. natriegens, ensuring optimal carbon flux through glycolysis and, in general, a more consistent and efficient pathway for the high-yield synthesis of fermentative products, including succinate.

Enhancing Anaplerotic Flux Towards Intermediary Metabolites of the rTCA Pathway:

This aspect of the disclosure (visually summarized in FIG. 2) focuses on augmenting the anaplerotic flux towards intermediary metabolites of the rTCA pathway in V. natriegens, a component for the bio-based production of succinic acid. The TCA cycle, a fundamental metabolic pathway in V. natriegens, is instrumental in the synthesis of various intermediates, including citrate, α-ketoglutarate, succinate, fumarate, and malate. These intermediates serve as precursors for the synthesis of a wide array of chemicals and materials with significant industrial value.

V. natriegens exhibits a unique metabolic versatility, enabling the efficient utilization of the rTCA pathway for central carbon metabolism and biosynthetic processes. Unlike the conventional oxidative TCA cycle, the rTCA cycle, functioning in the reverse direction, is characterized by its ability to fix carbon dioxide (CO₂) (e.g., CO₂ fixation by phosphoenolpyruvate carboxykinase) and consume NADH, thereby regenerating NAD⁺. Some exemplary genetic modification employed in the disclosure included the implementation of key heterologous enzymatic reactions mediating the carboxylation of phosphoenolpyruvate (PEP) to oxaloacetate by phosphoenolpyruvate carboxykinase (PEPCK) which enhance biosynthesis of succinate in the engineered V. natriegens strain disclosed herein.

This aspect of the disclosure, for example, pertains to the strategic utilization of native and non-native enzymes to enhance anaplerotic reactions, thereby optimizing carbon flux through the rTCA cycle to maximize succinate yield. The implementation of these genetic modifications is designed to create a more rapid, robust, and carbon-efficient metabolic pathway, contributing to the goals of sustainable and high-yield succinate production through bio-based processes.

Key components of this strategy involve the manipulation of the generation and flux of precursor intermediary metabolites within the rTCA cycle to bolster succinate production, specifically:

The gene pck, encoding phosphoenolpyruvate carboxykinase (PEPCK), is deleted, for example, by substitution of the native nucleotide coding sequence with a pck fusion comprising a V. natriegens pck sequence and an A. succinogenes pck codon-optimized coding sequence which is heterologously overexpressed using a modified synthetic constitutive promoter to facilitate the generation of oxaloacetate and ATP while preventing the reverse reaction and consumption of oxaloacetate by the native pck. PEPCK typically catalyzes the nucleoside triphosphate (NTP)-dependent conversion of oxaloacetate (OAA) to phosphoenolpyruvate (PEP), marking the initial step in the anabolic gluconeogenesis pathway in most organisms. By deleting the pck gene, this disclosure aims to inhibit the conversion of OAA to PEP, thereby preventing the diversion of OAA away from the rTCA pathway. However, in succinate producing microorganisms like A. succinogenes, M. succiniciproducens, and A. succiniciproducens, PEPCK enables the fixation of CO₂ through anaplerotic conversion of PEP to OAA coupled to the generation of ATP, as defined by the reaction:
PEP+CO₂+ADP [symbol] Oxaloacetate+ATP

This two-step reaction is initiated through binding of PEP, ADP, and CO₂ to the active site of PEPCK to form a ternary complex. CO₂ first reacts with PEP in a carboxylation reaction, facilitated by a divalent metal ion (M²⁺, typically Mg²⁺) cofactor bound to PEPCK, resulting in unstable carboxyphosphate (carboxyP) and enolpyruvate transient intermediates,
PEP+CO₂+PEPCK [symbol] PEPCK·PEP·CO₂ [symbol] PEPCK+CarboxyP+Enolpyruvate

In the second step, the carboxyphosphate intermediate is very unstable and spontaneously decarboxylates to generate free CO₂ and inorganic phosphate (Pi). Pi is transferred to ADP bound in the active site, regenerating ATP, while the released CO₂ simultaneously carboxylates the highly-reactive enolpyruvate intermediated, forming OAA, as defined by the reaction:
CarboxyP+ADP+PEPCK [symbol] PEPCK·CarboxyP·ADP [symbol] PEPCK+OAA+ATP

An exemplary embodiment of this component of the disclosure includes a genetic module comprising a pck fusion comprising a V. natriegens pck sequence and an A. succinogenes pck codon-optimized coding sequence for constitutive expression in V. natriegens mediated by the pC100 synthetic transcriptional promoter and designed for chromosomal integration at the native V. natriegens pck locus.

The present disclosure provides for a pck fusion comprising a V. natriegens pck sequence and an A. succinogenes pck sequence. In some aspects, the A. succinogenes pck codon-sequence is a codon-optimized coding sequence (e.g., codon-optimized for expression in V. natriegens). In some aspects, the pck fusion expression is mediated by a synthetic transcriptional promoter. In some aspects, the pck fusion expression is mediated by a pC100 synthetic transcriptional promoter. In some aspects the pck fusion is designed for chromosomal integration at the native V. natriegens pck locus. In some aspects, the pck fusion increases succinic acid product of a microorganism. In some aspects, the microorganism is an engineered V. natriegens. In some aspects, the microorganism is an engineered Mannheimia succiniciproducens. In some aspects, the microorganism is an engineered Actinobacillus succinogenes. In some aspects, the microorganism is an engineered Anaerobiospirillum succiniciproducens. In some aspects, the microorganism is an engineered Corynebacterium glutamicum. In some aspects, the microorganism is an engineered Escherichia coli.

In some aspects, the pck fusion comprises a polynucleotide about 75%, 80%, 85%, 86%, 87%, 88%, 89%, 90%, 91%, 92%, 93%, 94%, 95%, 96%, 97%, 98%, 99% or 100% identical to the nucleotide sequence of SEQ ID NO: 29. In some aspects, the pck fusion comprises a polynucleotide comprising the nucleotide sequence of SEQ ID NO: 29. In some aspects, the pck fusion comprises a polynucleotide about 75%, 80%, 85%, 86%, 87%, 88%, 89%, 90%, 91%, 92%, 93%, 94%, 95%, 96%, 97%, 98%, 99% or 100% identical to the nucleotide sequence of SEQ ID NO: 36. In some aspects, the pck fusion comprises a polynucleotide comprising the nucleotide sequence of SEQ ID NO: 36.

In some aspects, the pck fusion encodes a polypeptide about 85%, 90%, 95%, 96%, 97%, 98%, 99% or 100% identical to the amino acid sequence of SEQ ID NO: 44. In some aspects, the pck fusion encodes a polypeptide comprising the amino acid sequence of SEQ ID NO: 44. In some aspects, the pck fusion encodes a polypeptide about 85%, 90%, 95%, 96%, 97%, 98%, 99% or 100% identical to the amino acid sequence of SEQ ID NO: 45. In some aspects, the pck fusion encodes a polypeptide comprising the amino acid sequence of SEQ ID NO: 45.

This strategic intervention in the metabolic pathway of V. natriegens ensures the metabolic flux of OAA towards the rTCA pathway to support the production of intermediary metabolites for downstream succinate synthesis. Thus, deletion of the native V. natriegens PEPCK coupled to heterologous overexpression of a PEPCK fusion with favorable reaction kinetics from a succinate producing microorganism, for example, is aligned with the overarching objective of the present disclosure by enhancing anaplerosis and the CO₂-fixing capacity of V. natriegens, thereby increasing the overall efficiency and yield of the bio-based succinate production process in the engineered V. natriegens strain.

The targeted deletion of the maeB gene, which encodes malic enzyme MaeB, prevents the undesirable conversion of malate to pyruvate. In V. natriegens, the malic enzymes MaeA and MaeB, which are NADH- and NADPH-dependent respectively, play a role in mediating the directional flux of central carbon metabolism. These enzymes typically catalyze the decarboxylation of malate to pyruvate, thereby facilitating the conversion of TCA cycle intermediates into glycolytic metabolites. This enzymatic process forms a critical link between the TCA cycle and glycolysis, two central pathways in carbon metabolism. The deletion of the maeB gene in this aspect of the disclosure is designed to inhibit the conversion of malate to pyruvate.

By inhibiting the decarboxylation of malate to pyruvate, this aspect of the disclosure ensures the retention of key intermediates within the rTCA pathway. This approach not only optimizes the metabolic flux towards succinate production but also prevents carbon loss through the generation of CO₂—in alignment with the overarching objective of the disclosure to develop a more effective and sustainable method for the bio-based production of succinate.

Concomitantly with deletion of the maeB gene above, for example, the native V. natriegens gene maeA, encoding malic enzyme MaeA, is deleted by substitution with the non-native genetic element At_NADP-ME2*, encoding a mutant allele of the malic enzyme NADP-ME2 from Arabidopsis thaliana, specifically engineered for heterologous overexpression to facilitate the conversion of pyruvate to malate. The A. thaliana malic enzyme (ME) exhibits a ˜20 to 30-fold higher affinity for pyruvate (Kₘ=0.54 mM) compared to the native microbial MEs, which is advantageous for driving the reverse reaction pathway. This enhanced affinity enables the NADPH-dependent fixation of carbon dioxide onto pyruvate, generating malate and NADP⁺, as described by the reaction:
Pyruvate+CO₂+NADPH+H⁺ [symbol] Malate+NADP+

The reaction mechanism involves the initial binding of pyruvate and CO₂ to the ME active site, along with the divalent metal ion (Mn²⁺ or Mg²⁺) and NADPH cofactors. The metal ion aids in the addition of CO₂ to the enol form of pyruvate, forming a carboxy-pyruvate intermediate:
Pyruvate+CO₂+NADPH+H⁺+ME [symbol] ME·Pyr·CO₂·NADPH [symbol] Carboxy−Pyr+ME

Subsequently, this intermediate tautomerizes to enol-pyruvate, allowing the transfer of a hydride from NADPH to form malate and regenerate NADP+:

Enol-Pyr+NADPH+H⁺+ME [symbol] -ME·Enol−Pyr·NADPH [symbol] Malate+NADP++ME

Sufficient intracellular concentrations of both pyruvate and CO₂ substrates are ideally maintained to drive the desired reverse flux by mass action. This flux is additionally facilitated by genomic integration of a genetic module codon-optimized for expression in V. natriegens comprising a pC75 synthetic transcriptional promoter driving constitutive heterologous expression of the modified ME, NADP-ME2*. Overexpression of the mutant NADP-ME2* enzyme with a Cys490Ser substitution, significantly enhances pyruvate carboxylation activity, increasing reverse reaction efficiency between 50-70% relative to unmodified NADP-ME2.

The native V. natriegens MEs, MaeA and MaeB, predominantly catalyze the forward malate-to-pyruvate reaction. By deleting these genes and introducing a modified non-native ME with favorable kinetics for the reverse reaction, for example, this aspect of the disclosure effectively facilitates the conversion of pyruvate to malate, thus enhancing anaplerosis and CO₂-fixing capacity, and ultimately increasing succinate yields.

### 2.) Redirecting Cellular Resources and Metabolites Towards Succinate Production

This aspect of the disclosure focuses on the strategic redirection of cellular resources and intermediary metabolites in the engineered Vibrio natriegens strain to optimize succinate yields (as illustrated in FIG. 2). The approach involves a series of targeted metabolic engineering interventions designed to complement previously described aspects of the disclosure involving the overexpression of enzymes to increase the supply of essential metabolic precursors, pivotal to the synthesis of succinate through the engineered rTCA pathway. Further enhancing the redirection of metabolic flux, the expression, composition, and kinetic properties of downstream enzymes are modified to promote the forward flow of carbon through the rTCA pathway and prevent pathway inactivation, effectively ‘pulling’ carbon towards the synthesis of succinate. This is complemented by genetic modification and strategic deletion of enzymes associated with competing metabolic pathways to minimize the diversion of carbon and electron flux away from the targeted production of succinate. Additionally, this aspect of the disclosure addresses the optimization of intracellular energy pools and redox state within the engineered cells by leveraging modifications to increase the supply of ATP and, crucially, reducing power within the cell. This rebalancing is essential to compensate for the increased flux through the engineered rTCA pathway and any potential system-level dysregulation that might arise from the deletion of metabolic byproduct pathways.

In summary, this aspect of the disclosure presents a holistic approach to metabolic engineering in V. natriegens by coordinating genetic modifications and system-level adjustments aimed at redirecting metabolic flux and maximizing the efficiency and yield of succinate production.

This disclosure delineates a comprehensive metabolic engineering strategy aimed at optimizing succinate yields in an engineered strain of V. natriegens. The core of this strategy involves a series of targeted interventions designed to manipulate crucial branch points of carbon and electron flux within the cell, thereby facilitating a preferential redirection of intermediary metabolites towards succinate synthesis.

Aspects involve the manipulation of biochemical reactions mediating intermediary metabolite flux within the engineered rTCA cycle to optimize central carbon metabolism for the overproduction of succinate, specifically:

The gene mdh, encoding malate dehydrogenase (Mdh), is deleted, for example, by substitution of the native nucleotide coding sequence with a coding sequence for the Corynebacterium glutamicum Mdh homologue to facilitate enhanced generation of malate and NAD⁺. In V. natriegens, malate dehydrogenase catalyzes the reversible, NADH-dependent reduction of oxaloacetate (OAA) to malate, a key intermediate in the rTCA pathway. The reaction can be represented as:
OAA+NADH+H⁺ [symbol] Malate+NAD⁺

The active site of Mdh binds oxaloacetate and NADH to form a ternary enzyme-substrate-cofactor complex. It contains an arginine residue that facilitates the abstraction of a proton and catalyzes the hydride transfer from NADH to oxaloacetate, resulting in the formation of malate and oxidized NAD+. This reaction is crucial for the efficient conversion of OAA to malate and can be further define as:
OAA+NADH+H⁺+CgMdh [symbol] MDH·OAA·NADH [symbol] CgMdh+Malate+NAD⁺

This aspect of the disclosure involves the genomic integration of a codon-optimized synthetic genetic element encoding Mdh from C. glutamicum (Cg_Mdh) at the native V. natriegens mdh locus. Heterologously overexpression of Cg_Mdh driven by the modified synthetic constitutive promoter, pC100, is proposed due to its potential advantages in optimizing malate supply for downstream succinate production.

Compared to the native V. natriegens Mdh, the C. glutamicum Mdh exhibits favorable enzyme kinetics and allosteric activation by TCA intermediates, which are expected to increase flux through the engineered rTCA pathway. See Ahn, J. H., et al., “Enhanced succinic acid production by Mannheimia employing optimal malate dehydrogenase,” Nature Communications, 11 (1): 1970 (2020). This enhanced activity is particularly beneficial, for example, in the context of a genetically modified V. natriegens strain with increased OAA production, mediated by the heterologous expression of PEPCK to prevent metabolic bottlenecks or feedback inhibition caused by the accumulation of OAA.

The genes fumA and fumB and fumC, which encode the fumarase isoenzymes A, B, and C, respectively, are deleted, for example, and replaced with non-native genetic elements to eliminate native V. natriegens fumarase activity. Each of these fumarase isoenzymes exhibits distinct characteristics and regulatory mechanisms, adapting to varying cellular conditions. FumA functions primarily under aerobic conditions, FumB is active under anaerobic conditions, and FumC is upregulated under oxidative stress. All three isozymes of endogenous fumarase are highly efficient in catalyzing the undesirable hydration of fumarate to malate.

To prevent this activity, the native V. natriegens fumC gene is substituted with the non-native genetic element Ro_fumR, for example, encoding the fumarase enzyme (FumR) from Rhizopus oryzae, an organism known for its significant capacity for fumarate production. The kinetic properties of FumR, with Kₘ values for malate and fumarate of 0.46 mM and 3.07 mM, respectively, indicate a significantly higher substrate affinity for malate over fumarate. Additionally, the forward reaction conversion of fumarate to malate is inhibited by fumarate concentrations exceeding 2 mM, reinforcing the reverse enzyme activity, as defined by the reaction:
Malate+Ro_FumR [symbol] Ro_FumR·Malate [symbol] Ro_FumR+Fumarate+H₂O

This aspect of the disclosure prevents the accumulation of malate as a metabolic intermediate and enhances rTCA flux towards succinate production through genomic integration of a codon-optimized synthetic genetic element encoding a fumarase enzyme from R. oryzae (Ro_FumR) at the native V. natriegens fumC locus. Heterologously overexpression of Ro_FumR driven by the modified synthetic constitutive promoter, pC100, for example, enables the efficient conversion of malate to fumarate.

FumR from R. oryzae possesses several unique features that are advantageous for succinate production. The enzyme maintains robust activity under diverse conditions, with optimal activity at 30° C. and pH 7.2, and demonstrates stability below 45° C. across a broad pH range. Notably, FumR's activity is metal-independent and oxygen-agnostic, providing consistent catalytic activity for the conversion of malate to fumarate. These properties are particularly beneficial in fluctuating bioreactor conditions, allowing the engineered V. natriegens strain to maintain efficient rTCA flux regardless of oxygen levels or environmental changes. By integrating these unique properties of R. oryzae FumR into V. natriegens, this aspect of the disclosure further optimizes the engineered rTCA pathway for increased succinate yields, leveraging the enhanced enzymatic efficiency and favorable reaction kinetics of FumR.

The genes frdB, frdC, and frdD, encoding fumarate reductase complex subunits B, C, and D, respectively, are deleted, for example, by substitution with a non-native genetic element to remove the native V. natriegens fumarate reductase complex. This replacement aims to remove the native V. natriegens fumarate reductase activity. In V. natriegens, the fumarate reductase complex (FRDc) is a membrane-bound complex that participates in anaerobic respiratory metabolism, interacting with the bacterial electron transport chain under anaerobic conditions. The complex comprises four subunits: FrdA, FrdB, FrdC, and FrdD. The molecular mechanism of FRDc involves the reduction of fumarate to succinate, with electrons transferred from reduced quinone (QRd) molecules in the membrane to the iron-sulfur clusters of FrdB, and then to the FrdA subunit where fumarate reduction occurs. The reaction can be represented as:
Q_{Rd}+FrdB+FrdA+Fumarate [symbol] FrdB·Q_{Rd}·FrdA·Fum [symbol] FrdB+FrdA+Q_{Qx}+Succinate

While FrdA, the largest subunit, contains the active site for fumarate to succinate conversion, it also plays a non-canonical role in flagellar motor polarity regulation. Therefore, the frdA gene is retained to maintain this function. The iron-sulfur-cluster-dependent mechanism of FRDc is vulnerable to inactivation by molecular oxygen. This aspect of the disclosure addresses this vulnerability by replacing the native V. natriegens FRDc with a functionally homologous, oxygen-tolerant enzyme to facilitate flux through the rTCA pathway in the presence of oxygen.

The genes sdhA, sdhB, sdhC, and sdhD, encoding subunits A, B, C, and D of the succinate dehydrogenase complex (SDHc), respectively, may be deleted. This genetic intervention is designed to inhibit the conversion of succinate to fumarate, thereby enhancing succinate yield in the engineered strain. In its native state, the plasma membrane-bound SDHc plays a pivotal role in the electron transport chain during aerobic respiratory metabolism. The SdhA subunit, as the primary component of the complex, contains the active site for the oxidation of succinate to fumarate. The SdhB subunit, anchored in the membrane, is integral for electron transfer, containing iron-sulfur clusters that facilitate this process. These electrons, derived from the oxidation of succinate, are transferred to the quinone pool in the bacterial membrane. The smaller subunits, SdhC and SdhD, contribute to anchoring the complex to the plasma membrane. Collectively, the SDHc complex is responsible for the dehydrogenation of succinate, particularly in the presence of oxygen, as defined by:
Q_{Qx}+SdhB+SdhA+Succinate [symbol] SdhB·Q_{Qx}·SdhA·Suc [symbol] SdhB+SdhA+Q_{Rd}+Fumarate

The deletion of the SDHc in the context of the engineered V. natriegens succinate production strain, for example, represents a significant metabolic engineering strategy. By removing the complex, this aspect of the disclosure aims to prevent the oxidation of succinate back to fumarate through the oxidative TCA pathway. This intervention is crucial for improving succinate yield, as it ensures the accumulation of succinate rather than its conversion into other metabolites.

The transcriptional promoters for the genes, aceE, aceF, and lpdA encoding the pyruvate dehydrogenase, dihydrolipoyl transacetylase, and dihydrolipoyl dehydrogenase components, respectively, of the pyruvate dehydrogenase complex are substituted (e.g., the promoters for aceEF and lpdA) with a synthetic constitutive promoter to facilitate the production of Acetyl-CoA under micro- or anaerobic conditions. The pyruvate dehydrogenase complex (PDHc) plays a role in central carbon metabolism by catalyzing the irreversible, oxidative decarboxylation of pyruvate to acetyl-CoA. This reaction connects glycolysis to the TCA cycle by generating acetyl-CoA, a key precursor for many biosynthetic processes, as defined by the reaction:
Pyruvate+CoA+NAD⁺ [symbol] Acetyl-CoA+CO₂+NADH+H⁺

The reaction catalyzed by the PDH complex occurs in three sequential steps mediated by the E1 (AceE), E2 (AceF), and E3 (LpdA) component enzymes. The E1 enzyme AceE catalyzes the decarboxylation and reductive acetylation of pyruvate using a thiamine pyrophosphate (TPP) cofactor to form CO₂ and a hydroxyethyl-TPP intermediate. The acetyl group then transfers to the sulfhydryl of a lipoamide cofactor covalently bound to the E2 enzyme AceF, while TPP is reduced to THPP, as defined by the reaction:
Pyruvate+TPP+AceE [symbol] Hydroxyethyl-TPP+CO₂+AceE [symbol] Acetyl-AceF+THPP

Subsequently, the E2 enzyme AceF catalyzes the transfer of the acetyl group from its lipoamide cofactor to coenzyme A (CoA), forming acetyl-CoA, as defined by the reaction:
Acetyl-AceF+CoA [symbol] Acetyl-CoA+Dihydrolipoamide-AceF

And finally, the E3 enzyme LpdA re-oxidizes the dihydrolipoamide prosthetic group on E2 using NAD⁺ as an electron acceptor, regenerating the lipoamide for reuse in the cycle, as defined by the reaction:
Dihydrolipoamide-AceF+NAD⁺+LpdA [symbol] Lipoamide-AceF+NADH+H⁺+LpdA

Under anaerobic conditions, PDHc activity in V. natriegens is typically downregulated and the production of acetyl-CoA is predominantly mediated by the pyruvate-formate lyase fermentative pathway. However, this pathway represents a significant source of carbon waste, including the co-production of formate and, as such, has been removed from the succinate production strain disclosed herein. To reestablish acetyl-CoA synthesis, this aspect of the disclosure aims to increase PDHc activity in the absence of oxygen through deletion of PdhR, for example, and substitution of the native PDHc subunit promoters with the native promoter sequence from the anaerobically-induced promoter from the pflB gene (pflBp). Additionally, the lpdA gene is modified, for example, with a single amino acid substitution (E354K) to eliminate NADH-dependent feedback inhibition in the mutant enzyme, lpdA*. This alteration aims to, for example, enhance acetyl-CoA supply for the engineered reductive TCA cycle, compensating for the loss of pyruvate-formate lyase due to the deletion of pflB.

Elevated PDHc flux is expected to alter fermentation profiles, directing more pyruvate into acetyl-CoA synthesis. Critically, this aspect of the disclosure is a primary contributor to the restoration of anaerobic growth in the absence of PflB by re-enabling acetyl-CoA dependent pathways. Additionally, the increased production of NADH (from the abrogation of allosteric regulation in the mutant LpdA*) provides, for example, additional reducing power for the engineered rTCA pathway. This extra reducing power allows the engineered V. natriegens strain to maintain redox homeostasis while increasing rTCA pathway flux driven by enhanced activity from NADH-consuming pathway constituents.

The gene aspA, encoding aspartate ammonia-lyase, is deleted, for example, to prevent the conversion of fumarate to aspartic acid. Aspartate ammonia-lyase, or aspartase, is a lyase that catalyzes the reversible amination and deamination between aspartate and fumarate, participating in amino acid metabolism and in the urea cycle. In the reverse direction, ammonia is fixed onto fumarate to synthesize aspartate. This reaction requires a divalent metal ion cofactor and proceeds via an unstable succinimide intermediate, as defined by the reaction:
AspA+Fumarate+NH₃ [symbol] AspA·Fumarate·NH₃ [symbol] AspA+Aspartate

Under anaerobic conditions, deletion of aspA prevents the conversion of fumarate to aspartate so more fumarate may be channeled towards fumarate reductase, increasing flux through the engineered rTCA pathway and further improving succinate yield.

In conjunction with deletion of the gene aspA, for example the promoter of the of the polycistronic operon containing aspA and the gene dcuA, encoding a C4-dicarboxylate transporter, are replaced to enable constitutive expression from dcuA (i.e., ΔaspA1::pC75-dcuA). The dcuA and dcuB genes, encoding aerobically and anaerobically expressed C4-dicarboxylate transporter proteins, respectively, are primarily involved in the facilitated diffusion of C4-dicarboxylic acids (e.g., succinate) across the bacterial inner membrane. Functioning bidirectionally with antiporter activity coupled to movement of aspartate across the membrane, DcuA and DcuB play pivotal roles in balancing the intracellular concentrations of these key intermediary metabolites. Removing oxygen-mediated repression and enabling constitutive expressing of dcuA, for example, doubles the cell's C4-dicarboxylic acids transport capacity during anaerobic growth. This, in turn, facilitates more efficient export of succinate from the cell, alleviating succinate-based feedback inhibition and establishing a metabolic gradient that favors the continuous conversion of fumarate to succinate that pulls metabolic flux through the engineered rTCA pathway, enhancing the overall efficiency of succinate production.

The gene dctA, encoding the C4-dicarboxylate transporter, DctA, is deleted to prevent re-import of extracellular succinate. DctA specifically binds C4-dicarboxylate TCA cycle intermediates like succinate, fumarate, and malate and facilitates import under aerobic conditions. Under microaerobic or anaerobic conditions, deletion of dctA prevents reimport and catabolism of succinate in a strain optimized for succinate production.

3.) Eliminating Fermentative Pathways that Divert Carbon and Electron Flux from rTCA

To redirect more carbon flux towards succinate production, the engineered V. natriegens strain is modified to reduce carbon waste by eliminating major competing fermentative pathways that divert intermediates away from the reductive TCA cycle (as depicted in FIG. 2). As a facultative anaerobe, V. natriegens natively utilizes mixed-acid fermentation pathways under oxygen-limited conditions to maintain redox balance and energy production. These pathways branch off glycolysis and acetyl-CoA metabolism, converting pyruvate, acetyl-CoA and other intermediates into various byproducts including lactate, ethanol, acetate, and formate. However, these fermentative byproducts represent carbon loss that limits succinate yield. Therefore, targeted gene deletions are introduced to block lactate, ethanol, acetate and formate production pathways. Disrupting these alternative electron sinks conserves more pyruvate and acetyl-CoA to feed oxaloacetate synthesis and flux through the engineered reductive TCA cycle.

This aspect of the disclosure eliminates competing pathways that form undesirable fermentative byproducts to redirect carbon and electrons towards enhanced succinate generation, specifically:

The genes lldH and dldH are deleted to prevent conversion of pyruvate to lactate. Lactate production provides an alternative NADH oxidizing route that allows glycolytic flux to proceed under oxygen-limited conditions in V. natriegens. The enzymes L-lactate dehydrogenase (LldH) and D-lactate dehydrogenase (DldH) catalyze the interconversion between pyruvate and L- or D-lactate isomers, respectively, while regenerating NAD⁺ from NADH as defined by the reaction:
Pyruvate+NADH+H⁺ [symbol] DL-Lactate+NAD⁺

These pathways serve as electron sinks that prevent glycolytic stalling when respiratory electron transport is not available to re-oxidize NADH. However, lactate synthesis represents inefficient carbon usage that limits succinate production. Therefore, lldH and dldH are deleted to block both L- and D-lactate fermentation pathways, removing these NADH oxidation routes. This aspect of the disclosure is enabled by additional genetic modifications described herein that provide alternative mechanisms to maintain redox balance. Thus, eliminating lactate production prevents pyruvate loss and conserves this key precursor for oxaloacetate and succinate synthesis through the engineered rTCA pathway.

The gene ald is deleted to prevent conversion of pyruvate to alanine. The enzyme alanine aminotransferase, encoded by ald, catalyzes the conversion of pyruvate and glutamate to alanine and α-ketoglutarate in V. natriegens, as defined by the reaction:
Pyruvate+Glutamate+NAD⁺ [symbol] L-Alanine+α-Ketoglutarate+NADH+H⁺

While this reaction consumes excess pyruvate, alanine accumulation is not beneficial for succinate production since it represents a metabolic dead end. Alanine does not contribute to NADH oxidation or ATP generation through substrate-level phosphorylation. Therefore, the ald gene is deleted to in this aspect of the disclosure to eliminate alanine formation as an alternative overflow route for pyruvate disposal. This alanine synthesis pathway is a minor pathway in V. natriegens and deletion does not result in alanine auxotrophy in the engineered V. natriegens strain. Deletion of this additional non-essential competing pathway aims to conserve pyruvate for conversion to precursor metabolites oxaloacetate and malate, facilitating carbon flux through the rTCA pathway towards succinate generation.

The gene pflB is deleted to prevent conversion of pyruvate to formate and acetyl-CoA. The enzyme pyruvate-formate lyase (PFL), encoded by pflB, catalyzes the conversion of pyruvate and CoA to acetyl-CoA and formate during anaerobic glucose metabolism in V. natriegens, as defined by the reaction:
Pyruvate+CoA [symbol] Acetyl-CoA+Formate

Although this reaction does not directly contribute to NADH oxidation or ATP generation through substrate-level phosphorylation, catalysis of the reaction products (i.e., formate and acetyl-CoA) provides an additional electron sink for NADH oxidation and allows continued glycolytic flux under oxygen-limited conditions. However, formate production represents carbon loss that limits succinate yields. Therefore, pflB is deleted to eliminate the ability of V. natriegens to synthesize formate as a fermentative byproduct. This is expected to prevent pyruvate catabolism through the PFL pathway, potentially causing pyruvate accumulation and metabolic defects if glycolytic rate outpaces alternative pyruvate utilization routes. To mitigate these effects, the engineered strain incorporates additional pathways for pyruvate catabolism and optimizes alternative acetyl-CoA production pathways to maintain proper redox balance and central carbon metabolism following pflB deletion. Beneficially, eliminating formate production aims to conserve pyruvate for conversion to precursor metabolites oxaloacetate and malate, facilitating carbon flux through the rTCA pathway towards succinate generation.

The gene ackA is deleted to prevent conversion of acetyl-phosphate to acetate. In the engineered V. natriegens strain lacking PFL activity due to deletion of pflB, a modified PDHc catalyzes the anaerobic production of acetyl-CoA. This provides acetyl-CoA as a substrate for downstream energy-yielding enzymatic reactions. One reaction pathway, in particular, mediates the primary conversion of acetyl-CoA to acetate in a two-step pathway involving phosphotransacetylase (Pta) and acetate kinase (AckA). Pta, encoded by pta, transfers the acetyl group from acetyl-CoA to inorganic phosphate, forming acetyl phosphate (1). Then AckA, encoded by ackA, converts acetyl phosphate and ADP to acetate and ATP via substrate-level phosphorylation (2), as defined by the reactions:
(1) Acetyl-CoA+Pi [symbol] Acetyl-phosphate+CoA
(2) Acetyl-phosphate+ADP [symbol] Acetate+ATP

Although this acetate pathway generates ATP, excessive flux through this pathway can cause accumulation of acetate which can perturb intracellular pH. Additionally, acetate production does not directly contribute to NADH oxidation important for redox balance. Retaining pta allows the conversion of acetyl-CoA to acetyl-phosphate, thus preserving an important metabolic component of regulatory protein acetylation pathways. However, to reduce carbon loss and enhance succinate yields, the ackA gene is deleted to prevent the conversion of acetyl-phosphate to acetate and, more broadly, this overflow pathway for acetyl-CoA during anaerobic growth of the genetically modified strain. While this would reduce carbon lost to acetate, it also reduces downstream ATP generation mediated by the substrate-level phosphorylative activity of AckA. However, the loss of this pathway is compensated through upregulation of alternative ATP-generating reactions (e.g., constitutively expressed As_PckA) to maintain proper adenylate pool balance, energy metabolism, and growth.

The gene adhE is deleted to prevent conversion of acetyl-CoA to ethanol. The bifunctional enzyme aldehyde/alcohol dehydrogenase (AdhE), encoded by adhE, contains two distinct catalytic domains that, together, catalyze the conversion of acetyl-CoA to ethanol and regenerate 2 NAD⁺ from 2 NADH, as defined by the reaction:
Acetyl-CoA+2 NADH+2H⁺ [symbol] Ethanol+CoA+2 NAD⁺

First, the N-terminal aldehyde dehydrogenase domain catalyzes the NADH-dependent reduction of acetyl-CoA (Ac-CoA) to acetaldehyde (Ac-aldehyde) through a thiohemiacetal intermediate formed between the active site Cys and acetyl-CoA. Hydride transfer from NADH reduces this intermediate to acetaldehyde, as defined by the reaction:
Ac-COA+NADH+H⁺+AdhE [symbol] AdhE·Ac-COA·NADH [symbol] Ac-aldehyde+CoA+NAD⁺+AdhE

The C-terminal alcohol dehydrogenase domain then catalyzes the NADH-dependent reduction of acetaldehyde to ethanol through general base-catalyzed protonation of the carbonyl oxygen followed by hydride transfer from NADH to the carbonyl carbon, as defined by the reaction:
Ac-aldehyde+NADH+H⁺+AdhE [symbol] AdhE·Ac-aldehyde·NADH [symbol] Ethanol+NAD⁺+AdhE

Natively, this acetyl-CoA conversion pathway is a primary mechanism in V. natriegens to maintain redox balance and produce ethanol as a fermentation product under oxygen-limited conditions. However, as with acetate, ethanol synthesis from acetyl-CoA represents an additional source of carbon waste that limits succinate yields. Therefore, the adhE gene is deleted to eliminate ethanol formation. This is expected to disrupt redox homeostasis and potentially inhibit glycolysis due to NADH accumulation, while also increasing secretion of overflow metabolites like pyruvate. The loss of this pathway is compensated through upregulation of alternative NADH oxidation routes by optimizing dehydrogenase expression and activity (e.g., constitutively expressed Cg_MDH) at key reduction steps along the rTCA pathway. Similar to deleting ackA, deleting adhE aims to prevent carbon and electron loss from acetyl-CoA metabolism and, instead, redirect carbon and electron flux into the engineered rTCA pathway for enhanced succinate synthesis.

In certain aspects of the methods provided herein, the final yield of succinate on the carbon source is at least 10%, at least 20%, at least 30%, at least 40%, at least 50%, or greater than 50% of the theoretical yield. In certain aspects, the cells provided herein are capable of converting at least 80% or at least 90% by weight of a carbon source to succinate. The concentration, or titer, of succinate will be a function of the yield as well as the starting concentration of the carbon source. In certain aspects, the concentration may reach at least 1-3, at least 5, at least 10, at least 20, at least 30, at least 40, at least 50, or greater than 50 g/L at some point during the fermentation, and preferably at the end of the fermentation.

### III. Compositions of the Disclosure

The present disclosure provides an engineered Vibrio natriegens cell comprising one or more genetic disruptions, wherein the one or more genetic disruptions increase the production of succinate as compared to the production of succinate from a V. natriegens cell that does not comprise the one or more genetic disruptions, wherein the V. natriegens cell produces succinic acid from a glucose substrate at a rate of at least 0.48 g_{Succ} g_{CDW}⁻¹ h⁻¹. In some aspects, the rate of succinic acid production of the cell is from about 0.48 to about 2.00 g_{Succ} g_{CDW}⁻¹ h⁻¹.

In some aspects, under anaerobic conditions, the V. natriegens cell produces succinic acid from a glucose substrate at a rate of at least 0.75 g_{Succ} g_{CDW}⁻¹ h⁻¹. In some aspects, under anaerobic conditions, the V. natriegens cell produces succinic acid from a glucose substrate at a rate of at least 1.00 g_{Succ} g_{CDW}⁻¹ h⁻¹. In some aspects, under anaerobic conditions, the V. natriegens cell produces succinic acid from a glucose substrate at a rate of at least 1.25 g_{Succ} g_{CDW}⁻¹ h⁻¹. In some aspects, under anaerobic conditions, the V. natriegens cell produces succinic acid from a glucose substrate at a rate of at least 1.50 g_{Succ} g_{CDW}⁻¹ h⁻¹. In some aspects, under anaerobic conditions, the V. natriegens cell produces succinic acid from a glucose substrate at a rate of at least 1.75 g_{Succ} g_{CDW}⁻¹ h⁻¹. In some aspects, under anaerobic conditions, the V. natriegens cell produces succinic acid from a glucose substrate at a rate of at least 2.00 g_{Succ} g_{CDW}⁻¹ h⁻¹.

In some aspects, under anaerobic conditions, the V. natriegens cell produces succinic acid from a glucose substrate at a rate of about 0.48 g_{Succ} g_{CDW}⁻¹ h⁻¹. In some aspects, under anaerobic conditions, the V. natriegens cell produces succinic acid from a glucose substrate at a rate of about 0.50 g_{Succ} g_{CDW}⁻¹ h⁻¹. In some aspects, under anaerobic conditions, the V. natriegens cell produces succinic acid from a glucose substrate at a rate of about 0.75 g_{Succ} g_{CDW}⁻¹ h⁻¹. In some aspects, under anaerobic conditions, the V. natriegens cell produces succinic acid from a glucose substrate at a rate of about 1.00 g_{Succ} g_{CDW}⁻¹ h⁻¹. In some aspects, under anaerobic conditions, the V. natriegens cell produces succinic acid from a glucose substrate at a rate of about 1.25 g_{Succ} g_{CDW}⁻¹ h⁻¹. In some aspects, under anaerobic conditions, the V. natriegens cell produces succinic acid from a glucose substrate at a rate of about 1.50 g_{Succ} g_{CDW}⁻¹ h⁻¹. In some aspects, under anaerobic conditions, the V. natriegens cell produces succinic acid from a glucose substrate at a rate of about 1.75 g_{Succ} g_{CDW}⁻¹ h⁻¹. In some aspects, under anaerobic conditions, the V. natriegens cell produces succinic acid from a glucose substrate at a rate of about 2.00 g_{Succ} g_{CDW}⁻¹ h⁻¹.

In some aspects, under anaerobic conditions, the V. natriegens cell produces succinic acid from a glucose substrate at a rate of between 0.48 g_{Succ} g_{CDW}⁻¹ h⁻¹ to 0.75 g_{Succ} g_{CDW}⁻¹ h⁻¹. In some aspects, under anaerobic conditions, the V. natriegens cell produces succinic acid from a glucose substrate at a rate of between 0.75 g_{Succ} g_{CDW}⁻¹ h⁻¹ to 1.00 g_{Succ} g_{CDW}⁻¹ h⁻¹. In some aspects, under anaerobic conditions, the V. natriegens cell produces succinic acid from a glucose substrate at a rate of between 1.00 g_{Succ} g_{CDW}⁻¹ h⁻¹ to 1.25 g_{Succ} g_{CDW}⁻¹ h⁻¹. In some aspects, under anaerobic conditions, the V. natriegens cell produces succinic acid from a glucose substrate at a rate of between 1.25 g_{Succ} g_{CDW}⁻¹ h⁻¹ to 1.50 g_{Succ} g_{CDW}⁻¹ h⁻¹. In some aspects, under anaerobic conditions, the V. natriegens cell produces succinic acid from a glucose substrate at a rate of between 1.50 g_{Succ} g_{CDW}⁻¹ h⁻¹ to 1.75 g_{Succ} g_{CDW}⁻¹ h⁻¹. In some aspects, under anaerobic conditions, the V. natriegens cell produces succinic acid from a glucose substrate at a rate of between 1.75 g_{Succ} g_{CDW}⁻¹ h⁻¹ to 2.00 g_{Succ} g_{CDW}⁻¹ h⁻¹.

In some aspects, the cell comprises 6 or more genetic disruptions. In some aspects, the cell comprises 8 or more genetic disruptions. In some aspects, the cell comprises 10 or more genetic disruptions. In some aspects, the cell comprises 12 or more genetic disruptions. In some aspects, the cell comprises 14 or more genetic disruptions. In some aspects, the cell comprises 16 or more genetic disruptions. In some aspects, the cell comprises 18 or more genetic disruptions. In some aspects, the cell comprises 20 or more genetic disruptions. In some aspects, the cell comprises 22 or more genetic disruptions. In some aspects, the cell comprises 24 or more genetic disruptions. In some aspects, the cell comprises 26 or more genetic disruptions.

In some aspects, the cell comprises a mutation to a native phosphoenolpyruvate carboxykinase (pck) gene. In some aspects, the cell comprises a pck fusion comprising a V. natriegens pck sequence and an A. succinogenes pck sequence operatively linked to a constitutive promoter. In some aspects, the pck fusion encodes a polypeptide about 85%, 90%, 95%, 96%, 97%, 98%, 99% or 100% identical to the amino acid sequence of SEQ ID NO: 45. In some aspects, the pck fusion encodes a polypeptide comprising the amino acid sequence of SEQ ID NO: 45.

In some aspects, the cell comprises a mutation to a native malate dehydrogenase (mdh) gene. In some aspects, the mutation comprises a replacement of the native malate dehydrogenase (mdh) gene with an mdh gene from a C. glutamicum. In some aspects, the mdh gene from the C. glutamicum is operatively linked to a constitutive promoter. In some aspects, the mdh gene from a C. glutamicum encodes a polypeptide about 85%, 90%, 95%, 96%, 97%, 98%, 99% or 100% identical to the amino acid sequence of SEQ ID NO: 43. In some aspects, the mdh gene from a C. glutamicum encodes a polypeptide comprising the amino acid sequence of SEQ ID NO: 43.

In some aspects, the cell comprises a deletion of an L-lactate dehydrogenase gene (lldH).

In some aspects, the cell comprises a deletion of a D-lactate dehydrogenase gene (dldH).

In some aspects, the cell comprises a deletion of an alanine dehydrogenase gene (alD).

In some aspects, the cell comprises a deletion of a pyruvate formate lyase gene (pflB).

In some aspects, the cell comprises a deletion of each of the lldH, dldH, alD, and pflB genes.

In some aspects, the cell comprises a deletion of a ptsI gene.

In some aspects, the cell comprises a native glucokinase gene (glk) operably linked to a constitutive synthetic promoter. In some aspects, the glk gene encodes a polypeptide about 85%, 90%, 95%, 96%, 97%, 98%, 99% or 100% identical to the amino acid sequence of SEQ ID NO: 42. In some aspects, the glk gene encodes a polypeptide comprising the amino acid sequence of SEQ ID NO: 42.

In some aspects, the cell overexpresses a native glucokinase gene (glk).

In some aspects, the cell comprises a heterologous sodium-dependent glucose transporter protein (vsGLT) from Vibrio parahaemolyticus. In some aspects, the vsGLT gene encodes a polypeptide about 85%, 90%, 95%, 96%, 97%, 98%, 99% or 100% identical to the amino acid sequence of SEQ ID NO: 41. In some aspects, the vsGLT gene encodes a polypeptide comprising the amino acid sequence of SEQ ID NO: 41.

In some aspects, the cell comprises a mutation to the native E1 and E2 subunits of a pyruvate dehydrogenase complex (aceEF) gene. In some aspects, the mutation comprises a replacement of the native promoter of the aceEF gene with a synthetic promoter. In some aspects, the aceEF gene comprises a nucleotide sequence that encodes a polypeptide about 85%, 90%, 95%, 96%, 97%, 98%, 99% or 100% identical to the amino acid sequences of SEQ ID NOs: 37 and/or 38. In some aspects, the aceEF gene comprises a nucleotide sequence that encodes a polypeptide comprising the amino acid sequences of SEQ ID NO: 37 and/or 38.

In some aspects, the cell comprises a mutation to a native E3 subunit of a pyruvate dehydrogenase complex (lpdA) gene. In some aspects, the mutation comprises an E354K mutation and a replacement of the native promoter of the lpdA gene with a synthetic promoter. In some aspects, the lpdA gene encodes a polypeptide about 85%, 90%, 95%, 96%, 97%, 98%, 99% or 100% identical to the amino acid sequence of SEQ ID NO: 39.

In some aspects, the lpdA gene encodes a polypeptide comprising the amino acid sequence of SEQ ID NO: 39.

In some aspects, the cell comprises a heterologous E. coli hexuronate transporter (exuT) gene, wherein the exuT gene is operably linked to a constitutive synthetic promoter. In some aspects, the exuT gene encodes a polypeptide about 85%, 90%, 95%, 96%, 97%, 98%, 99% or 100% identical to the amino acid sequence of SEQ ID NO: 40. In some aspects, the exuT gene encodes a polypeptide comprising the amino acid sequence of SEQ ID NO: 40.

In some aspects, the cell comprises a deletion of a PN96_RS22390 gene.

In some aspects, the cell comprises a heterologous S. elongatus (PCC7002) Sodium-Dependent Bicarbonate Transporter (sbtA) gene. In some aspects, the sbtA gene encodes a polypeptide about 85%, 90%, 95%, 96%, 97%, 98%, 99% or 100% identical to the amino acid sequence of SEQ ID NO: 46. In some aspects, the sbtA gene encodes a polypeptide comprising the amino acid sequence of SEQ ID NO: 46.

In some aspects, the cell comprises: a deletion of each of the lldH, dldH, alD, and pflB genes; and a pck fusion comprising a V. natriegens pck sequence and an A. succinogenes pck sequence operatively linked to a constitutive promoter.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; and a mutation to a native malate dehydrogenase (mdh) gene.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; and a heterologous E. coli hexuronate transporter (exuT) gene.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; and a deletion of a PN96_RS22390 gene.

In some aspects, the cell comprises: a deletion of each of the lldH, dldH, alD, and pflB genes; a pck fusion comprising a V. natriegens pck sequence and an A. succinogenes pck sequence operatively linked to a constitutive promoter; a mutation to the native E1 and E2 subunits of a pyruvate dehydrogenase complex (aceEF) gene; a mutation to a native E3 subunit of a pyruvate dehydrogenase complex (lpdA) gene; a mutation to a native malate dehydrogenase (mdh) gene; a native glucokinase gene (glk) operably linked to a constitutive synthetic promoter; a deletion of a ptsI gene; a heterologous sodium-dependent glucose transporter protein (vsGLT) from Vibrio parahaemolyticus; a heterologous E. coli hexuronate transporter (exuT) gene; a deletion of a PN96_RS22390 gene; and a heterologous S. elongatus (PCC7002) Sodium-Dependent Bicarbonate Transporter (sbtA) gene.

In some aspects, the cell comprises one or more modifications selected from Table D. In some aspects, the mutations are relative to a wild-type V. natriegens genome (e.g., a P. Baumann 111 strain, ATCC #14048).

In some aspects, the cell comprises a modification, wherein the modification comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 1-36. In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to one of SEQ ID NOs: 1-36.

In some aspects, the cell comprises a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22.

In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-22. In some aspects, the cell comprises a plurality of modifications, wherein each of the plurality of modifications comprises the nucleotide sequence of each of SEQ ID NOs: 19-22.

In some aspects, the cell comprises a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29.

In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-22 and 29. In some aspects, the cell comprises a plurality of modifications, wherein each of the plurality of modifications comprises the nucleotide sequence of each of SEQ ID NOs: 19-22 and 29.

In some aspects, the cell comprises a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28.

In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 7, 19-24, and 29. In some aspects, the cell comprises a plurality of modifications, wherein each of the plurality of modifications comprises the nucleotide sequence of each of SEQ ID NOs: 7, 19-24, and 29. In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-24, 28, and 29. In some aspects, the cell comprises a plurality of modifications, wherein each of the plurality of modifications comprises the nucleotide sequence of each of SEQ ID NOs: 19-24, 28, and 29.

In some aspects, the cell comprises a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or 25, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2 or 27, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26.

In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 1, 2, 7, 19-24, 26, and 29. In some aspects, the cell comprises a plurality of modifications, wherein each of the plurality of modifications comprises the nucleotide sequence of each of SEQ ID NOs: 1, 2, 7, 19-24, 26, and 29. In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-29. In some aspects, the cell comprises a plurality of modifications, wherein each of the plurality of modifications comprises the nucleotide sequence of each of SEQ ID NOs: 19-29.

In some aspects, the cell comprises a plurality of modifications, wherein the plurality of modifications comprise a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 19, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 20, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 21, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 22, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 29, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 23, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 7 or 28, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or 25, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2 or 27, a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 26, and a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 30.

In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 1, 2, 7, 19-24, 26, 29, and 30. In some aspects, the cell comprises a plurality of modifications, wherein each of the plurality of modifications comprises the nucleotide sequence of each of SEQ ID NOs: 1, 2, 7, 19-24, 26, 29, and 30. In some aspects, the cell comprises a plurality of modifications, wherein each of the plurality of modifications comprises a nucleotide sequence about 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each of SEQ ID NOs: 19-30. In some aspects, the cell comprises a plurality of modifications, wherein each of the plurality of modifications comprises the nucleotide sequence of each of SEQ ID NOs: 19-30.

The present disclosure provides a bioreactor comprising an engineered V. natriegens cell as disclosed herein. In some aspects, the bioreactor is an anaerobic bioreactor.

In some aspects, the rate of succinic acid production in the bioreactor is at least 0.48 g_{Succ} g_{CDW}⁻¹ h⁻¹. In some aspects, the rate of succinic acid production in the bioreactor is at least 0.75 g_{Succ} g_{CDW}⁻¹ h⁻¹. In some aspects, the rate of succinic acid production in the bioreactor is at least 1.00 g_{Succ} g_{CDW}⁻¹ h⁻¹. In some aspects, the rate of succinic acid production in the bioreactor is at least 1.25 g_{Succ} g_{CDW}⁻¹ h⁻¹. In some aspects, the rate of succinic acid production in the bioreactor is at least 1.50 g_{Succ} g_{CDW}⁻¹ h 1. In some aspects, the rate of succinic acid production in the bioreactor is at least 1.75 g_{Succ} g_{CDW}⁻¹ h⁻¹. In some aspects, the rate of succinic acid production in the bioreactor is at least 2.00 g_{Succ} g_{CDW}⁻¹ h⁻¹.

In some aspects, the rate of succinic acid production is measured over a two hour period. In some aspects, the rate of succinic acid production is measured over a three hour period. In some aspects, the rate of succinic acid production is measured over a four hour period. In some aspects, the rate of succinic acid production is measured over a five hour period. In some aspects, the rate of succinic acid production is measured over a six hour period. In some aspects, the rate of succinic acid production is measured over a seven hour period. In some aspects, the rate of succinic acid production is measured over an eight hour period. In some aspects, the rate of succinic acid production is measured over a nine hour period. In some aspects, the rate of succinic acid production is measured over a ten hour period. In some aspects, the rate of succinic acid production is measured over an eleven hour period. In some aspects, the rate of succinic acid production is measured over a twelve hour period.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1. Engineering of Modified V. Natriegens Strains for Anaerobic Succinate Production

A V. natriegens strain engineered to produce succinic acid from glucose under anaerobic conditions has previously been described. See Thoma et al., “Metabolic engineering of Vibrio natriegens for anaerobic succinate production,” Microbial Biotechnology 15:1671-1684 (2021). However, this strain is impractical for industrial use for at least two reasons: (i) the strain is unable to grow anaerobically, requiring a two-phase cultivation; and (ii) per-cell productivity is low, requiring a large volume of aerobic preculture concentrated to very high density to achieve viable volumetric productivity.

A V. natriegens strain of the present disclosure was designed and engineered to overcome these limitations. Table A lists the genetic modifications present in the V. natriegens strain previously described in the literature (“GBS027”) and a V. natriegens strain of the present disclosure (“GBS802”). An “X” in the table indicates that the modification is present in the strain. A “−” in the table indicates that the modification is not present in the strain.

TABLE A
Genetic modifications of V. natriegens strains of Example 1

| | | Strain | |
|---|---|---|---|
| Gene | Modification | GBS027 | GBS802 |
| lldH | Deletion | X | X |
| dldH | Deletion | X | X |
| alD | Deletion | X | X |
| pflB | Deletion | X | X |
| aceEF | Replacement of native promoter with | — | X |
| | insulated synthetic promoter (pC100) | | |
| lpdA | Replacement of native promoter with | — | X |
| | insulated synthetic promoter (pC100) | | |
| lpdA | E354K mutation | — | X |
| ptsI | Deletion | — | X |
| exuT | Integration of E. coli hexuronate | — | X |
| | transporter under constitutive synthetic | | |
| | promoter (pC75) | | |
| VpSGLT | Integration of V. parahaemolyticus | — | X |
| | sodium-dependent glucose transporter | | |
| | under constitutive synthetic promoter | | |
| | (pC75) | | |
| GLK | Placement of native GLK under | — | X |
| | constitutive synthetic promoter (pC75) | | |
| MDH | Replacement of native gene with C. | — | X |
| | glutamicum MDH under constitutive | | |
| | promoter (pC100) | | |
| PCK | Replacement of native gene with V. | — | X |
| | natriegens/A. succinogenes PCK fusion | | |
| | under constitutive promoter (pC100) | | |
| PN96_RS22390 | Deletion | — | X |
| sbtA | Insertion of S. elongatus sodium- | — | X |
| | dependent bicarbonate transporter at | | |
| | vacant locus under weak pC25 | | |
| | constitutive promoter | | |
| PYC | Insertion of C. glutamicum pyruvate | X | — |
| | carboxylase under synthetic constitutive | | |
| | promoter at DNS endonuclease locus | | |

The GBS027 and GBS802 strains were generated and subsequently evaluated according to the following methods:

### Genetic Engineering of V. Natriegens Strains

To generate the GBS027 and GBS802 strains, chromosomal modifications to a wild-type V. natriegens (ATCC 14048) were made using mutant constructs generated using standard techniques of molecular biology, e.g., agarose gel electrophoresis and PCR amplification, were applied as described in the literature. See Sambrook, J., & Russell, R. W., Molecular Cloning: A Laboratory Manual, 3rd edn., Cold Spring Harbor, NY: Spring Harbor Laboratory Press (2001). Briefly, amplification primers were designed to amplify each 3 Kb homology arm from the genomic target site and to include any DNA cargo between the homology arms. Overlapping sequence between neighboring fragments was included to introduce homology for downstream fragment assembly. Target fragments were then amplified via PCR using amplification primers and run on a 0.8% TBE agarose gel to confirm target fragment size and purity. Correct target fragments were purified using a PCR Clean-Up Kit (Qiagen). Purified fragments were then diluted and mixed in equal ratios in a PCR tube on ice. An equal volume of 2× NEBuilder HiFi Assembly Master Mix was added to each assembly reaction and mixed. Assembly reactions were incubated in a thermocycler at 50° C., then heat inactivated by incubation at 94° C. Finally, the resulting cassettes were PCR amplified and introduced into the V. natriegens genome via tfoX-mediated natural transformation as described below.

V. natriegens strains harboring tfoX plasmids were induced to competence by growing overnight in LBv2+100 μg/mL carbenicillin+100 μM IPTG in a shaking incubator at 30° C. Overnight cultures were then diluted directly into 350 μL of Instant Ocean medium (28 g/L; Aquarium Systems Inc.) supplemented with 100 μM IPTG. Cassettes (>50 ng) were then added and reactions were incubated statically at 30° C. for 5 hours. After incubation, 1 mL of LBv2 was added to each reaction tube and reactions were outgrown at 30° C. with shaking (250 rpm) for ˜1-2 hrs. Following outgrowth, reactions were inoculated into fresh growth media containing the appropriate selection antibiotic and plated onto agar plates appropriate for selection.

Following the selection step, targeted genomic sites were amplified and isolated, and inserts were verified by sequencing as described in the literature. See Hoffart et al., “High substrate uptake rates empower Vibrio natriegens as production host for industrial biotechnology,” Appl Environ. Microbiol. 83:1-10 (2017), Dalia, A. B., et al., “Characterization of undermethylated sites in Vibrio cholerae,” J. Bacteriol. 195:2389-2399 (2013) and Dalia, T. N., et al., “Multiplex Genome Editing by Natural Transformation (MuGENT) for Synthetic Biology in Vibrio natriegens,” ACS Synth. Biol. 6 (9): 1650-1655 (2017).

### Comparative Anaerobic Growth of Engineered V. Natriegens Strains

The abilities of the engineered GBS027 and GBS802 V. natriegens strains to grow under anaerobic conditions were evaluated. Exponentially growing aerobic precultures of a wild type V. natriegens strain (GBS004), the GBS027 strain, and the GBS802 strain were diluted to an OD600 of 0.05 and grown in 96-well microplate format in the following medium: 22 g/L NaCl, 4.7 g/L MgCl₂, 0.3 g/L KCl, 5 g/L Yeast Extract, 20 g/L Glucose, 100 mM MOPS buffer, 100 mM NaHCO₃, pH adjusted to 7.0 and sterile filtered. Cultures were grown for 20 hours in a BioTek synergy microplate reader in an anaerobic chamber at 37° C.

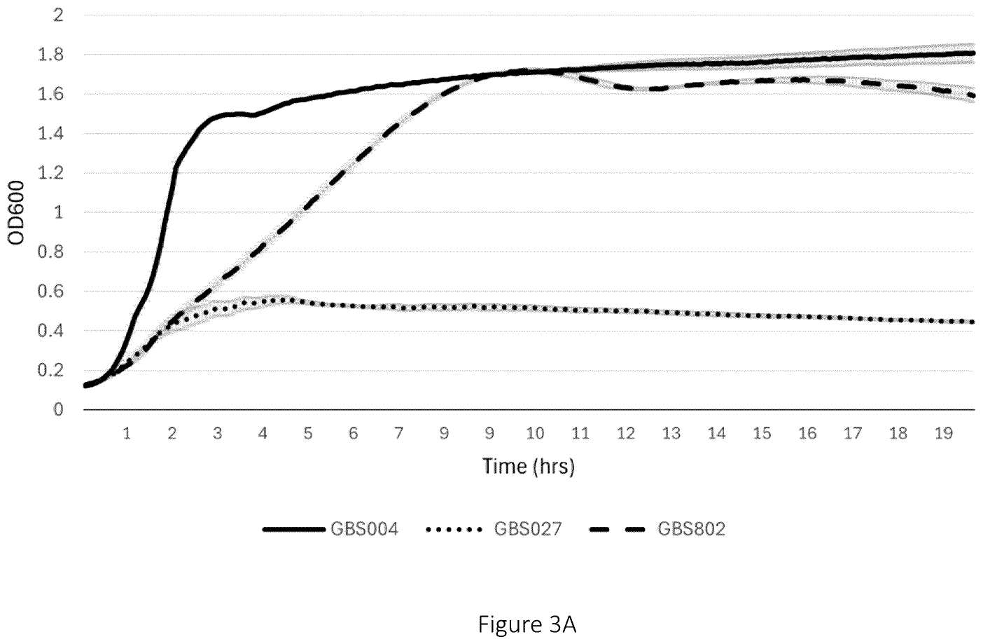

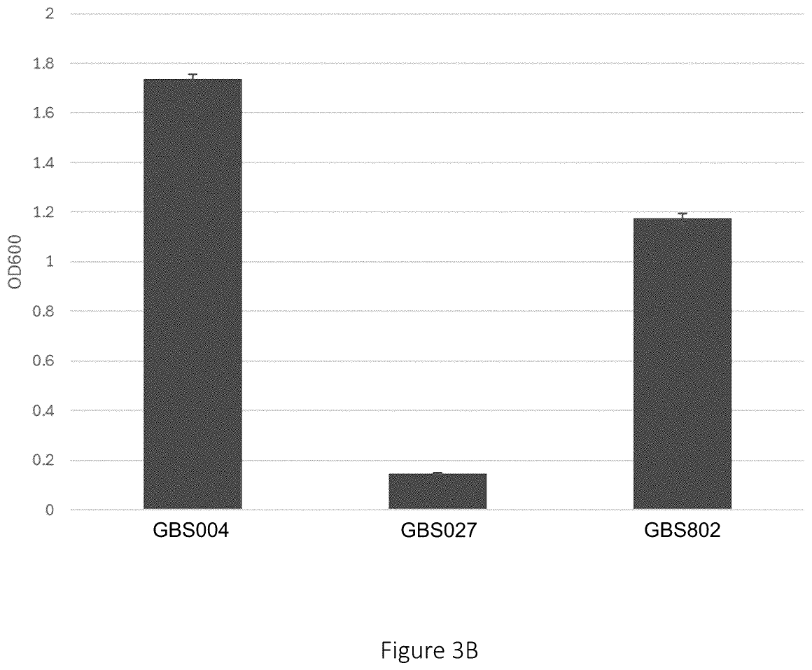

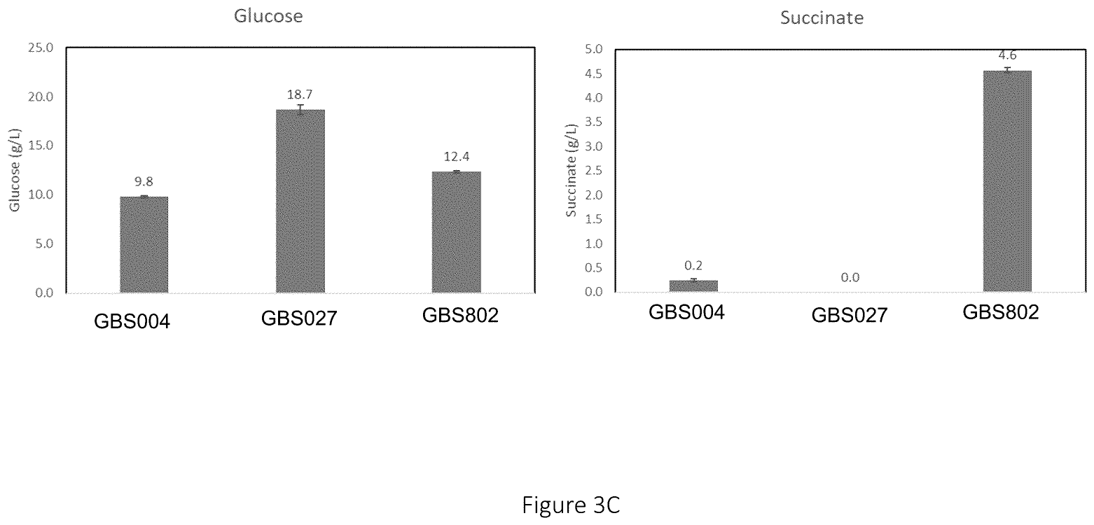

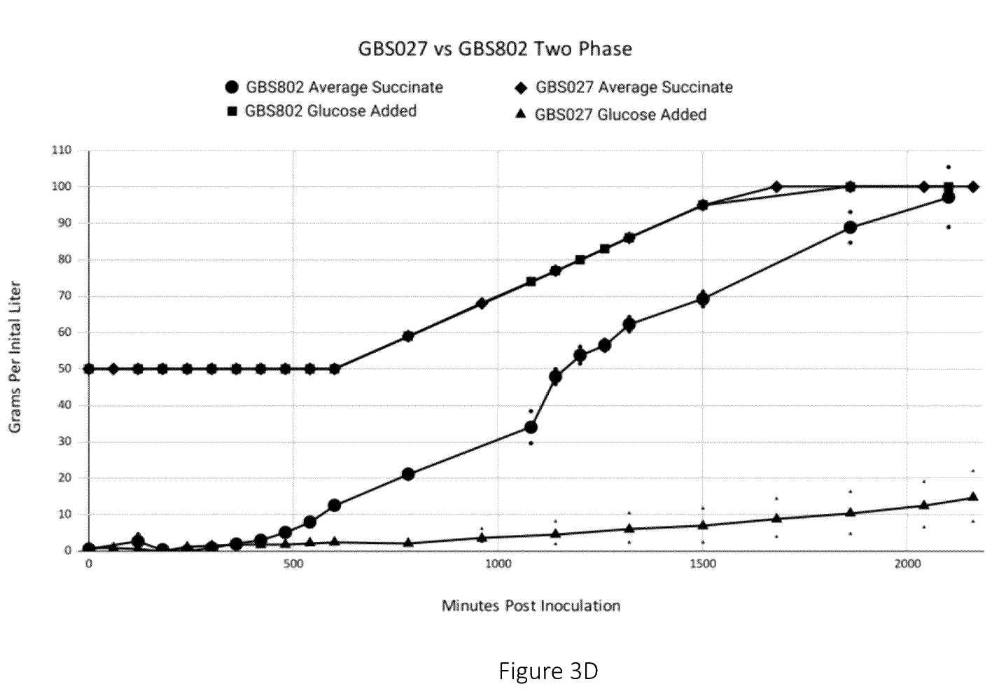

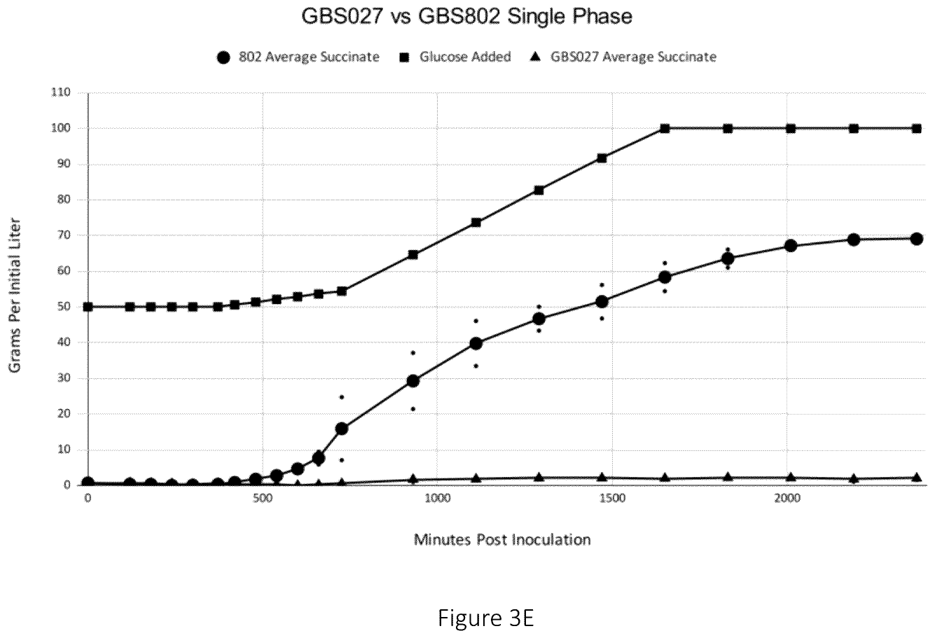

As shown in FIGS. 3A and 3B, both the wild type strain and the GBS802 strain grew anaerobically, having final OD600 readings of ˜1.8 and ˜1.2, respectively, at 20 hours post-inoculation. The GBS027 strain exhibited poor anaerobic growth, having a final OD600 of less than 0.2 at 20 hours post-inoculation.

### Comparative Succinate Production of Engineered V. Natriegens Strains

The abilities of the engineered GBS027 and GBS802 V. natriegens strains to produce succinate were evaluated.

In one experiment, exponentially growing aerobic precultures of the WT strain, the GBS027 strain, and the GBS802 strain were adjusted to an OD600 of 5.0 and grown in 96-well microplate format in the following medium: 22 g/L NaCl, 4.7 g/L MgCl₂, 0.3 g/L KCl, 5 g/L Yeast Extract, 20 g/L Glucose, 100 mM MOPS buffer, 100 mM NaHCO₃, pH adjusted to 7.0 and sterile filtered. Cultures were grown for 3 hours in an anaerobic chamber at 37° C. The concentrations of succinate and glucose present in each culture were measured by HPLC. The results of this experiment are shown in FIG. 3C.

In a two-phase fermentation experiment, bioreactors were prepared with the following medium: 22 g/L NaCl, 4.7 g/L MgCl₂, 0.3 g/L KCl, 10 g/L yeast extract and autoclaved. Following sterilization, 250 mL of 500 g/L sterile glucose solution was added to each bioreactor, bringing the final volume to 2.5 L and the glucose concentration to 50 g/L. Each bioreactor was inoculated with 1 mL of exponentially growing cultures of either the GBS027 or the GBS802 strain with an OD600 of 5.0 and started with 1VVM of air sparging. Bioreactors were maintained at 37° C., and bioreactor pH was maintained at 7.0 by addition of 50% NH₄OH. Five hours following inoculation, a linear feed of 3 mL/hour of sterile 1M NaHCO₃ was started. Eight hours following inoculation, the feed was increased to 20 mL/hour and maintained at that level for the duration of the run. Nine hours following inoculation, all sparging was stopped. Ten hours following inoculation, a linear feed of sterile 500 g/L glucose was started at 15 mL/hour and continued until a total of 250 g of glucose had been added to the fermentation (100 g/L of initial volume). The succinate concentration for each strain was measured at various points during the fermentation (see FIG. 3D). On average, the final succinate concentration produced by each strain was 62.3 g/L for GBS802 and 10.1 g/L for GBS027.

In a single-phase fermentation experiment, bioreactors were prepared with the following medium: 22 g/L NaCl, 4.7 g/L MgCl₂, 0.3 g/L KCl, 10 g/L yeast extract and autoclaved. Following sterilization, 250 mL of 500 g/L sterile glucose solution was added each bioreactor, bringing the final volume to 2.5 L and the glucose concentration to 50 g/L. Each bioreactor was inoculated with 1 mL of exponentially growing cultures of either the GBS027 or the GBS802 strain with an OD600 of 5.0 and started with 1VVM of air sparging. Bioreactors were maintained at 37° C., and bioreactor pH was maintained at 7.0 by addition of 50% NH₄OH. Five hours following inoculation, a linear feed of 5 mL/hour of sterile 1M NaHCO₃ and 375 g/L glucose (in the same solution) was started. Twelve hours following inoculation, the feed was increased to 20 mL/hour and continued until a total of 250 g of glucose was added to the fermentation (100 g/L of initial volume). The succinate concentration for each strain was measured at various points during the fermentation (see FIG. 3E). On average, the final succinate concentration produced by each strain was 52.6 g/L for GBS802 and 2.0 g/L for GBS027.

In the Thoma et al. study (“Metabolic engineering of Vibrio natriegens for anaerobic succinate production,” Microbial Biotechnology 15:1671-1684 (2021)), several different numbers are presented corresponding to V. natriegens succinate productivity on a g_{Succ} g_{CDW}⁻¹ h⁻¹ basis, which correspond to different experimental methods and methods of calculation. The most prominently featured succinate production measurement is 1.33 g_{Succ} g_{CDW}⁻¹ h⁻¹ (see, Thoma et al., Abstract). This number was produced “in test tubes containing 50 ml VN minimal medium with 27.5 mM glucose” (see, Thoma et al., FIG. 3) and the number represents the rate of succinate secretion “within the first 2 h” (see, Thoma et al., p. 1676). Such an experiment is not representative of an industrial production setting, given the very low glucose concentration utilized, which results in a low final succinate concentration, and the low biomass concentration of cells (1.4 g_{CDW} 1⁻¹). Thoma et al.'s later experiments were performed at high cell density in a bioreactor, requiring several precultures to obtain a starting biomass concentration of about 18 g_{CDW} 1-1, which is necessitated by the low productivity of this strain, and are quite impractical to implement at scale. These experiments yielded an average productivity of 0.48 g_{Succ} g_{CDW}⁻¹ h⁻¹ over a period of 7 hours, with a maximum productivity of 1.15 g_{Succ} g_{CDW}⁻¹ h⁻¹ over a 1 hour period (see, Thoma et al., Table 1).

In the experiments described herein, and in contrast to Thoma et al., cells were grown in the same vessel where the anaerobic succinate production occurred. Biomass concentration at the start of production phase was 3.92 g_{CDW}⁻¹ h⁻¹. When averaged over the production phase (the relevant comparison to Thoma et al.'s reactor experiments), the productivity of the cells described herein was 0.96 g_{Succ} g_{CDW}⁻¹ h⁻¹ over a period of 27 hours. The maximum productivity over a 2 hour period of the cells described herein was 2.52 g_{Succ} g_{CDW}⁻¹ h⁻¹ (see Example 1 and FIG. 3D).

The results of the two- and single-phase fermentation experiments are reproduced below in Table B:

TABLE B
Average succinate production rates, titers, and
yields from two- and single-phase fermentations

| | GBS802, | GBS027, | GBS802, | GBS027, |
|---|---|---|---|---|
| | two- | two- | single- | single- |
| | phase | phase | phase | phase |
| Titer | 62.3 | 10.1 | 52.6 | 2.0 |
| (g/L) | | | | |
| Rate | 2.77 | 0.40 | 1.96 | 0.06 |
| (g/L/h) | | | | |
| Yield | 0.972 | * | 0.690 | * |
| (gSuc/gGluc) | | | | |

* Glucose consumption was so low that yield could not be accurately computed.

TABLE C
Mutations in Vibrio natriegens cells described herein.

| Mutation | Type of Mutation | Mutation Category |
|---|---|---|
| L-lactate dehydrogenase | Deletion | Yield/elimination of side |
| (lldH) | | products |
| D-lactate | Deletion | Yield/elimination of side |
| dehydrogenase (dldH) | | products |
| Alanine dehydrogenase | Deletion | Yield/elimination of side |
| (alD) | | products |
| Pyruvate-formate lyase | Deletion | Yield/elimination of side |
| (pflB) | | products |
| Phosphoenolpyruvate | Replacement of native gene a pck | Productivity (anaplerotic |
| carboxykinase (pck) | fusion comprising a V. natriegens | flux), yield (incorporation of |
| | pck sequence and an A. | inorganic carbon), Growth |
| | succinogenes pck sequence | (ATP generation) |
| | operatively linked to a constitutive | |
| | promoter | |
| Phosphoenolpyruvate | deletion | Growth (conservation of |
| carboxylase | | ATP) |
| Malate dehydrogenase | Replacement of native gene with | Productivity, Growth (redox |
| (mdh) | gene from C. glutamicum | balance) |
| Fumarase (fumR) | Replacement of native fumC with | Productivity |
| | gene from R. oryzae | |
| Fumarate reductase | Replacement of native frdBCD | Productivity, Growth (Redox |
| (FRD) | with soluble FRD from T. bruceii | balance) |
| Succinate | Deletion | Yield (prevents conversion of |
| dehydrogenase | | succinate back to fumarate) |
| Malic Enzyme (MAE) | Replacement of native MaeA with | Productivity (anaplerosis), |
| | NADPH-dependent malic enzyme | Yield (inorganic carbon |
| | from A. thaliana | incorporation), Growth |
| | | (redox balancing) |
| ptsI | Deletion | Productivity (Conserves PEP |
| | | for anaplerotic flux instead of |
| | | glucose uptake) |
| glk | Overexpression of native gene | Productivity (Conserves PEP |
| | | for anaplerotic flux instead of |
| | | glucose uptake) |
| dcuA | Overexpression | Productivity (enhances |
| | | succinate export) |
| dctA | deletion | yield/productivity (prevents |
| | | succinate import) |
| ackA | deletion | Yield (formation of side |
| | | products) |
| adhE | deletion | Yield (formation of side |
| | | products) |
| mgsA1 | deletion | Yield (formation of side |
| | | products) |
| mgsA2 | deletion | Yield (formation of side |
| | | products) |
| aceEF | Promoter change/overexpression | Growth (production of acetyl- |
| | | coA without pflB) |
| lpdA | Promoter change/overexpression | Growth (production of acetyl- |
| | | coA without pflB, |
| | | maintaining redox balance) |
| lpdA | Mutation of protein (E354K | Growth (elimination of |
| | mutation) | NADH-dependent feedback |
| | | inhibition) |
| E. coli hexuronate | Integration of heterologous gene | Growth (importation of |
| transporter (exuT) | | glucose without ptsI) |
| Vibrio | Integration of heterologous gene | Growth (importation of |
| parahaemolyticus | | glucose without ptsI) |
| Sodium-dependent | | |
| Glucose Transporter | | |
| (VpSGLT) | | |
| PN96_RS22390 | Deletion | Derepression of native xylB |
| (Transcriptional | | |
| repressor deoR) | | |
| S. elongatus (PCC7002) | Insertion of heterologous gene | Productivity (increases |
| Sodium-Dependant | | intracellular bicarbonate to |
| Bicarbonate Transporter | | boost flux through PCK) |
| sbtA | | |

TABLE D
Listing of Exemplary Modifications to V. natriegens Genes

| SEQ | | |
|---|---|---|
| ID NO: | Description | Sequence |
| 1 | ΔptsI | TGGCTTTGTTGGAAATAGGCTCAAAACTAAGCTAAAACTATTCG |
| | (±100 bp) | TTAGCACACCCATTATTCTCCCGTTTTATAAAGTTGACCAAACTT |
| | | AAGGTAAGGCTATGATTTCAGGCATCCTAGCATCTCCTGGTAGG |
| | | ACACTCGTCGCTTTCGGAGTTGAAAAGTTCATTGCTGAGAAAGT |
| | | TCAGTAATACGTAGTACCAAGTAATTCAATACTTAATGGCCATT |
| | | AGACGGCGAAAAAAGTCGTCTAATGAGCTATATTGGTATACTAT |
| | | AATTCGACAGAATAAAACT |
| 2 | glk::pC100- | CATTTCGCATAGATAGGCTCCAACCCTCTGCTATATTTAAGTGCA |
| | Vp_vSGLT- | AATATTATGTCGTAAAGCTAACGATTTCTGATTATTGAGCATTAA |
| | rGlk_pC25 | CTAGTTTGTTAAGCTTATTTGAAAAAAGTGTTTGACAGTTGAAC |
| | (±100 bp) | ACTCGTTGCCTATAATCGAAAGTCGAGTTATCGAGATTTTCAGG |
| | | AGAGGAAGCTCATATGTCGAATATAGAACATGGTCTAAGCTTTA |
| | | TCGACATAATGGTCTTCGCCATTTATGTCGCAATTATCATTGGGG |
| | | TCGGACTTTGGGTATCTCGTGATAAAAAAGGCACTCAGAAAAGT |
| | | ACGGAAGATTATTTCTTGGCGGGAAAATCTTTGCCTTGGTGGGC |
| | | TGTCGGTGCTTCGCTAATTGCTGCAAATATTTCTGCGGAACAATT |
| | | TATAGGAATGTCTGGTTCAGGCTATTCAATTGGCTTGGCTATCGC |
| | | ATCTTATGAATGGATGTCGGCAATAACATTGATTATTGTTGGTA |
| | | AGTACTTTCTACCTATTTTCATTGAAAAAGGAATCTATACCATTC |
| | | CTGAATTTGTTGAAAAACGCTTCAATAAAAAACTAAAAACAATT |
| | | TTGGCCGTTTTTTGGATTTCCTTGTACATTTTTGTAAACCTAACTT |
| | | CAGTACTGTATTTAGGTGGTTTGGCTCTCGAAACCATTTTGGGTA |
| | | TTCCGTTGATGTACTCAATTCTAGGTCTTGCGCTGTTTGCGTTGG |
| | | TGTACTCAATTTATGGTGGTTTATCGGCAGTAGTATGGACCGAT |
| | | GTCATCCAAGTGTTCTTCTTAGTTTTGGGTGGTTTTATGACTACC |
| | | TACATGGCAGTGAGCTTTATTGGTGGTACGGACGGTTGGTTCGC |
| | | TGGGGTGTCTAAAATGGTCGATGCAGCTCCTGGCCACTTTGAGA |
| | | TGATCTTGGATCAAAGTAATCCACAATACATGAACCTTCCTGGT |
| | | ATTGCCGTATTAATTGGTGGTCTTTGGGTAGCAAACTTATATTAC |
| | | TGGGGCTTTAACCAGTACATTATTCAAAGAACGCTTGCTGCAAA |
| | | ATCAGTATCGGAAGCTCAGAAAGGTATTGTTTTTGCAGCGTTTTT |
| | | GAAACTTATCGTTCCGTTTCTCGTGGTATTGCCAGGTATTGCCGC |
| | | TTACGTTATTACTTCGGACCCACAACTAATGGCAAGCCTTGGTG |
| | | ATATTGCAGCAACAAACCTTCCAAGTGCTGCTAATGCGGATAAA |
| | | GCATACCCTTGGCTAACTCAGTTCTTGCCTGTTGGTGTTAAAGGT |
| | | GTTGTTTTTGCGGCTCTTGCTGCTGCAATTGTTTCTTCACTAGCAT |
| | | CAATGCTTAACTCAACAGCCACTATCTTCACTATGGATATTTACA |
| | | AAGAGTATATCTCTCCTGACTCAGGTGACCACAAGTTGGTGAAT |
| | | GTTGGGCGTACTGCAGCTGTGGTGGCACTAATTATTGCTTGCCTA |
| | | ATTGCCCCAATGTTAGGTGGTATTGGCCAAGCATTCCAATACAT |
| | | CCAAGAATATACAGGTTTAGTTAGCCCTGGTATTTTGGCTGTATT |
| | | CTTACTTGGCTTATTCTGGAAGAAAACAACCAGTAAAGGGGCTA |
| | | TTATTGGTGTAGTAGCATCAATACCATTTGCCTTGTTCTTGAAAT |
| | | TTATGCCACTTTCCATGCCATTTATGGATCAAATGCTATACACAT |
| | | TATTGTTTACAATGGTTGTTATCGCATTTACAAGTTTGAGCACAT |
| | | CAATTAATGATGATGATCCTAAAGGTATTAGTGTTACATCATCG |
| | | ATGTTTGTAACAGATCGAAGCTTTAATATCGCTGCTTACGGCAT |
| | | AATGATTGTTTTGGCAGTGTTATATACATTGTTCTGGGTAAACGC |
| | | AGACGCAGAAATCACACTAATCATTTTCGGTGTAATGGCAGGCG |
| | | TAATCGGTACAATCCTACTAATCAGCTACGGTATCAAAAAACTA |
| | | ATCAAAGGTGGCGGTGGTGGCGGTCATCACCATCATCACCATGG |
| | | TGGCGGTGGCGGTGGCTCAGAGCAATACGCATTAGTAGGCGAC |
| | | ATCGGCGGTACTAACGCAAGATTGGCTCTCTGCGAGTTATCTAC |
| | | GGGCACGATTTCTGATATCGTCACTTATCCTGCAGCAGAATACG |
| | | AATCCCTCGAAGTAGTAATGCGCCAGTACTTGGATCGTCATGAT |
| | | ACCCCGATCTCTTCAGCGTGTATCGCCATTGCATGTCCAGTAACA |
| | | GGTGACTGGGTAAGTATGACTAATCACCATTGGGAATTCTCTAT |
| | | CCAGGAGCTAAAATCACAACTTAACCTACAAGTACTAGAAGTAA |
| | | TCAACGATTTTACGGCTGTGAGTTTTGCTATTCCAAGCTTGAAAC |
| | | CAGAGGACCGTGTCCAAATTGGGGGTGGTGAAGCGATCGCAAA |
| | | CAAGCCAATCGCTGTGTATGGTGCTGGTACCGGCCTGGGTGTTG |
| | | CGCAGCTGATCCACGGAGGCAACCTATGGCATTCGGTTCCGGGC |
| | | GAGGGCGGACATGTTGACTTAGCAGCGTGTACACCTGAAGAAG |
| | | ATGATCTTATTGTTTACTTGAGAAACAAGTTTGGTAGAGTCTCTG |
| | | CTGAGCGTTGTTTATCAGGCCAAGGTATCCGTAACATTTACGAC |
| | | TTCGTTGTTTCCCACCATGGTGCAGCACCTGAAGATTACACACC |
| | | AGCAATGATCACAGAAAAAGCTCTTAAAGCGGAGTGCAAAGAC |
| | | TGTGAACGCGCACTGTATCTTTTTTGTATCCTTATGGGCCGATTT |
| | | GGAGGCAATCTTGCGCTAACTAACGGCAGTTTCGGCGGTGTGTA |
| | | CATTGCCGGTGGTATCGTACCAAAAGTCCAGGAACTTTTTATCA |
| | | AGAGCGGCTTCCGTGTTGCTTTCGAAGACAAGGGTCGATTTAAA |
| | | GAATACCTGCAGTCAATCCCGGTTTTTCTAATCACTCATTCAGAA |
| | | CCTGGTCTTCTAGGTGCCGGTACATATATTCGCCAATCTCTCAAC |
| | | ATTAGTATCAACTAAGGCCCGCATGTAGTCAAAAGCCTCCGGTC |
| | | GGAGGCTTTTGACTCTTGGTAAGGAGAAGCTTATTTGAAAAAAG |
| | | TGTTTGACAGTTGAACACTCGTTGCCTATAATCGAAAGTCGAGT |
| | | TATCGAGATTTTCAGGAGCTAAGGAAGCTCATATGTCGGAACAG |
| | | TATGCCTTGGTAGGGGATATCGGGGGAACCAACGCGCGTTTAGC |
| | | TCTTTGTGAGTTATCGACAGGAACGATCAGCGATATCGTGACTT |
| | | ACCCTGCTGCCGAATATGAATCGCTGGAAGTCGTCATGCGTCAA |
| | | TACCTTGATCGTCACGACACTCCGATCTCATCGGCCTGTATTGCG |
| | | ATTGCTTGTCCGGTAACGGGGGATTGGGTTTCCATGACTAACCA |
| | | CCACTGGGAGTTCTCTATCCAGGAACTAAAATCCCAACTTAACC |
| | | TTCAGGTTTTGGAAGTCATTAACGATTTCACTGCGGTTTCCTTTG |
| | | CTATTCCAAGTTTAAAACCTGAGGACAGAGTTCAGATCGGTGGC |
| | | GGTGAAGCGATAGCCAATAAGCCGATAGCCGTATACGGAGCGG |
| | | GAACCGGGCTTGGTGTCGCTCAGCTGATTCATGGCGGAAACCTA |
| | | TGGCATAGCGTACCAGGAGAAGGTGGGCATGTTGACCTGGCGG |
| | | CTTGTACCCCTGAAGAAGACGACTTGATTGTTTACCTACGTAAC |
| | | AAGTTCGGTCGTGTGTCTGCGGAACGCTGTTTGTCTGGCCAAGG |
| | | TATCCGAAATATATACGACTTTGTTGTGTCACACCATGGTGCCGC |
| | | ACCTGAAGACTACACCCCGGCAATGATCACTGAGAAAGCACTCA |
| | | AAGCGGAATGCAAGGATTGTGAGCGCGCACTCTATCTGTTTTGC |
| | | ATTTTGATGGGGCGTTTTGGCGGTAACCTGGCACTGACTAATGG |
| | | ATCATTTGGTGGTGTGTATATTGCTGGCGGTATTGTTCCTAAAGT |
| | | TCAAGAACTATTCATCAAGTCCGGCTTTCGAGTGGCTTTTGAAG |
| | | ATAAAGGCCGATTTAAAGAGTACCTTCAATCCATCCCTGTGTTCT |
| | | TAATCACTCATTCCGAACCTGGCTTGCTGGGCGCGGGCACCTAC |
| | | ATCAGACAGTCATTAAATATCTCTATAAATTAATATTTAACTCTA |
| | | ACCCGAATTCATAAATTGGATTCGGGATTCTGCTTGCCATCTTCA |
| | | TTTTTGTAATCAATTCTCCAGTTATAACCTGTACTTTCAAAGT |
| 3 | ΔmgsA1 | TCATGGCTTCTTTGCTCTTATCAATAATGAAAATACCCACCAACG |
| | (±100 bp) | ATGGCTGAGTGAACACCTGCTCAGTACTCCCTTTTACTTTTAGAA |
| | | GAAGACTGTTATGAATACGACAACTCGAATCATACCTGCGAGGA |
| | | CACTCGTCGCTTTCGGAGATTATGAAAAATATTTGAGTAACCGG |
| | | ACCTAGCCTTTTCCTCAGATTCTGAATAAGAACATCGTTTATAGG |
| | | AAATGAATGAAATGGAAACAAAAAAGCAGAGTAAGAATACTCT |
| | | GCTTTTAGGTATATGAAG |
| 4 | ΔmgsA2 | CAACATTCAGTGTCAAGCCTATTGATTGATCGATTAAAAAAGTG |
| | (±100 bp) | TTTCATTAACCAATACACCAAAAATAACTTTATCATTTTGAATCG |
| | | GAAATAACTTGATGCAAAAAACAACTCGTACCATGCCTGCAAGG |
| | | ACACTCGTCGCTTTCGGAGACTATGAAGCCTATCTGAGCGAGCG |
| | | CACATAATCTGTGCCCGATATTTTAGATAAAACAAAGGCGATAC |
| | | CGAGTATCGCCTTTGTTATTTCAAACGGTGTCTATTTTATTTAGC |
| | | GCGAAGTAATGATTGGGT |
| 5 | ΔmaeB | ATTTTTTAAGGATATTGCTCGCAATCTTACCTATGAAGATTGACT |
| | (±100 bp) | TCTATCCATTAGAATTTATTCCCCCTTACCTAAAAAATAAAAAG |
| | | AGAAACGCCCACATGTCTGAAGACAAAGCAGTAACTCCCGAAA |
| | | GGACACTCGTCGCTTTCGGAATTGAAATGCCTGAAAACTACATG |
| | | GCAGAGTAAGACAAAGATTTAAATGACATAAAAAAATGGGACG |
| | | CGATGGCGTCCCATTTTTTTATGTCATTTTTAGTATTAGGCGCTA |
| | | AATAACCGCTTGGTGTATTCA |
| 6 | ΔmaeA:pC75- | TGAGAGAGAATCACGCGACTTTGAAACCCTCTGGATGACTTCTT |
| | At_NADP-ME2* | TTGAGTATAAATTTCCACCAGACGTGAATTTTATTGCAACATTAC |
| | (±100 bp) | CTTGATGTGCAAAGCTTATTTGAAAAAAGTGTTTGACAAGGTGC |
| | | CCGAAACCGGTTATAATCCTCGTTCGAGTTATCGAGATTTTCAG |
| | | GAGCTAAGGAAGTCATATGGGTTCAACGCCAACTGATCTGCCAG |
| | | GCGAAGATGTCGCTGATAACCGTTCAGGCGTAGGCGGCGGAATC |
| | | TCTGATGTATACGGTGAGGACAGCGCGACGCTTGACCAGCTTGT |
| | | TACCCCTTGGGTGACAAGCGTAGCCTCTGGCTACACGTTAATGC |
| | | GTGATCCACGTTACAACAAAGGCCTAGCATTCACGGACAAAGA |
| | | GCGAGACGCACACTACTTAACTGGACTCCTACCACCTGTTATAC |
| | | TATCACAAGATGTCCAAGAAAGAAAGGTTATGCATAACCTACGA |
| | | CAGTATACAGTTCCACTTCAGCGATACATGGCACTAATGGACTT |
| | | ACAAGAACGCAACGAGCGCCTTTTTTACAAGCTGCTAATCGACA |
| | | ACGTAGAAGAACTTCTTCCGGTAGTCTACACGCCTACAGTAGGC |
| | | GAAGCATGTCAGAAATATGGTTCTATTTTCCGTAAACCACAGGG |
| | | TCTTTATATCAGTCTAAACGAAAAAGGAAAAATCCTTGAAGTAT |
| | | TAAAAAACTGGCCACAAAGAGGCATCCAAGTTATCGTGGTAACT |
| | | GACGGGGAACGCATTCTTGGTTTGGGCGATCTTGGTTGTCAAGG |
| | | TATGGGTATCCCTGTCGGCAAGCTCTCACTTTACACCGCGTTAGG |
| | | CGGTATTAGACCATCAGCCTGCCTCCCTATTACAATTGACGTCG |
| | | GAACAAACAACGAAAAACTATTAAATGACGAATTTTATATCGGC |
| | | CTTAAACAGCGTCGCGCTACTGGTCAGGAGTACGCAGAATTCCT |
| | | TCATGAGTTCATGTGTGCAGTTAAACAGAATTACGGTGAAAAAG |
| | | TCCTAGTGCAGTTTGAAGATTTCGCGAACCATAACGCCTTCGAT |
| | | CTACTTTCAAAGTATTCGGACAGTCACTTGGTTTTTAATGATGAC |
| | | ATTCAGGGTACAGCGTCTGTAGTTTTGGCTGGCTTGATCGCTGCA |
| | | CAAAAAGTACTAGGTAAAAAGCTAGCTGATCACACCTTTTTATT |
| | | TCTGGGTGCAGGTGAAGCTGGTACAGGTATTGCTGAATTGATCG |
| | | CCCTCAAGATCTCCAAAGAGACTGGCGCACCGATTACGGAGACG |
| | | CGTAAGAAGATCTGGCTCGTTGATTCTAAGGGTCTGATCGTATC |
| | | TTCTCGCAAAGAATCACTGCAACATTTTAAGCAACCGTGGGCTC |
| | | ATGAACATAAACCGGTGAAGGACCTTATCGGCGCAGTCAACGC |
| | | AATCAAACCGACTGTCTTAATCGGCACAAGTGGTGTAGGCCAAA |
| | | CATTTACTAAAGAGGTGGTGGAAGCAATGGCAACTAACAACGA |
| | | AAAACCATTAATCTTGGCACTATCAAATCCAACTAGTCAAGCGG |
| | | AGTGTACTGCGGAACAAGCATACACATGGACCAAAGGTAGAGC |
| | | GATCTTTGGTAGCGGTTCACCTTTTGACCCAGTAGTTTACGATGG |
| | | CAAAACGTACCTTCCGGGTCAGGCTAACAATTGTTATATCTTTCC |
| | | TGGTCTGGGCTTGGGGCTAATCATGTCCGGCGCAATTCGTGTTA |
| | | GAGACGATATGCTTTTGGCGGCGAGTGAGGCATTAGCAGCTCAA |
| | | GTAACTGAAGAGCACTATGCTAACGGACTGATTTATCCACCGTT |
| | | TAGTAACATCCGTGAAATTTCCGCGAACATCGCTGCCTGTGTTG |
| | | CAGCTAAGACATACGATCTTGGTCTGGCATCTAACCTACCTCGT |
| | | GCTAAAGATCTCGTTAAATTCGCAGAGTCGTCTATGTATAGCCC |
| | | CGTTTACCGTAACTACCGCTAAATTTAACCGCAAAAGAAAAACT |
| | | CGCCTAATAATTAGCCCCTAACACATAGGGGCTTTTGTTTTTTAA |
| | | ACGTTTTTTTATCATCCTAACCGTATTTTGGCA |
| 7 | Δmdh: pC100- | GAATGAAGGGATCAAAAAAGCCGACCCTAAGATCGGCTTTTTAT |
| | Cg_Mdh | AAACGTATAAACGCTTAAACGGGCCCCTGAAGTCAGCCCCATAC |
| | (±100 bp) | GATATAAGTTGTAAGCTTATTTGAAAAAAGTGTTTGACAGTTGA |
| | | ACACTCGTTGCCTATAATCGAAAGTCGAGTTATCGAGATTTTCA |
| | | GGAGAGGAAGCTCATATGAATTCACCGCAAAACGTCTCTACGAA |
| | | AAAGGTCACAGTCACCGGCGCAGCTGGTCAAATCTCTTATTCAC |
| | | TTTTATGGCGCATCGCCAACGGTGAAGTATTCGGCACTGACACG |
| | | CCAGTAGAACTGAAACTTCTAGAAATCCCGCAAGCTCTTGGCGG |
| | | GGCAGAGGGTGTTGCTATGGAACTTCTAGATTCGGCCTTCCCCC |
| | | TCCTACGAAACATCACGATCACTGCGGATGCCAACGAGGCATTC |
| | | GACGGCGCTAATGCTGCATTTTTGGTAGGTGCGAAACCTCGCGG |
| | | TAAAGGCGAAGAGAGAGCAGACTTACTGGCTAACAACGGCAAA |
| | | ATTTTTGGTCCTCAAGGTAAAGCTATCAACGACAACGCCGCAGA |
| | | TGACATCCGTGTGCTAGTTGTTGGAAACCCAGCGAACACCAACG |
| | | CGTTGATAGCTTCAGCTGCGGCACCAGATGTTCCAGCATCAAGA |
| | | TTCAACGCAATGATGCGCCTTGATCATAACCGTGCGATCAGTCA |
| | | GCTAGCAACTAAGCTTGGCCGTGGTTCTGCAGAATTTAACAACA |
| | | TCGTGGTATGGGGAAATCATTCCGCAACACAATTTCCAGACATC |
| | | ACGTACGCAACAGTTGGTGGAGAAAAGGTCACTGACTTAGTTGA |
| | | TCACGATTGGTACGTAGAAGAGTTTATTCCTCGTGTGGCTAACC |
| | | GTGGCGCTGAAATCATTGAGGTACGTGGTAAATCATCTGCAGCT |
| | | TCTGCAGCATCCTCTGCGATTGATCATATGAGAGATTGGGTACA |
| | | AGGTACAGAGGCGTGGAGCAGTGCGGCAATTCCTAGCACTGGT |
| | | GCATACGGCATTCCGGAAGGCATCTTTGTTGGTCTGCCAACAGT |
| | | AAGTCGAAACGGTGAATGGGAAATCGTTGAAGGCCTAGAGATT |
| | | TCAGATTTTCAGAGAGCACGTATCGACGCGAATGCTCAGGAATT |
| | | ACAGGCTGAACGCGAGGCAGTGCGCGACTTGCTCTAAGTAGTTC |
| | | TCCTTGAGAGTATTTTTTTATAAATGTATTTGTAGGGTAGTTTTT |
| | | AACCAACCAAGTAAAGCTATAGGAATCACAACTTGATTACAATC |
| | | AATT |
| 8 | ΔfumC::pC100- | CGCAGTATCGAGAGTAACAGCAGAAACACCACAAGAAGTGAAA |
| | Ro_fumR | GATCTGGTTGAGCACGTACGTGTACCAGTAGGTGCGGGCCAAGC |
| | (±100 bp) | TGTCGATCACTAACTGAAATAAAGAACAAAATAGAAAGCTGGG |
| | | CTTAATTGTAGGGATATAGCTCACTTGCTCCAAGCCGATGCCTGT |
| | | ATTCATGCATCGGCTTTTTTAAGCTTATTTGAAAAAAGTGTTTGA |
| | | CAGTTGAACACTCGTTGCCTATAATCGAAAGTCGAGTTATCGAG |
| | | ATTTTCAGGAGAGGAAGCTCATATGAACAACTCACCACGTTTGT |
| | | TTTCTTCAGCCAGTGCTGCACTACAAAAATTTCGCGCGGAGCGT |
| | | GATACATTCGGTGATCTCCAAGTGCCAGCTGACCGATACTGGGG |
| | | CGCACAAACTCAAAGATCTCTTCAAAACTTCGACATCGGAGGTC |
| | | CGACTGAACGAATGCCAGAGCCACTAATTAGAGCATTCGGTGTA |
| | | TTAAAAAAGGCCGCAGCAACGGTCAACATGACGTACGGTCTGG |
| | | ATCCTAAGGTTGGCGAAGCAATTCAAAAGGCAGCCGATGAAGTT |
| | | ATTGATGGTTCACTTATCGATCATTTTCCACTTGTTGTCTGGCAA |
| | | ACAGGTTCTGGAACACAGACAAAAATGAACGTGAACGAAGTAA |
| | | TCTCAAACCGCGCGATAGAGCTCTTAGGCGGTGAACTTGGCTCA |
| | | AAAGCTCCGGTTCATCCTAACGACCACGTAAACATGAGTCAATC |
| | | TTCAAACGACACGTTCCCGACAGCTATGCATGTGGCAGCGGTTG |
| | | TAGAGATTCATGGTCGTCTCATTCCAGCTCTAACGACGTTGCGC |
| | | GACGCGCTGCAGGCAAAATCCGCTGAATTTGAACACATCATCAA |
| | | AATCGGACGCACTCATTTGCAGGATGCGACTCCTCTAACCCTAG |
| | | GCCAGGAATTTTCGGGTTACACTCAACAGCTTACATATGGGATC |
| | | GCTAGAGTACAGGGCACGCTAGAGCGCCTTTATAATCTCGCACA |
| | | AGGCGGTACTGCAGTCGGCACTGGTTTGAACACGCGTAAAGGCT |
| | | TTGATGCTAAAGTTGCAGAAGCAATTGCAAGCATCACGGGCCTG |
| | | CCTTTTAAAACCGCGCCAAACAAATTCGAAGCACTAGCAGCGCA |
| | | CGACGCATTAGTCGAGGCTCATGGTGCTTTGAACACCGTGGCAT |
| | | GTTCTCTTATGAAGATCGCGAATGATATCCGTTACCTTGGCTCCG |
| | | GTCCTCGTTGTGGTCTAGGTGAACTTTCGTTACCCGAGAACGAA |
| | | CCGGGTTCAAGTATCATGCCAGGCAAGGTTAACCCTACACAATG |
| | | CGAGGCAATGACTATGGTTTGTGCTCAGGTTATGGGTAACAACA |
| | | CAGCGATCAGTGTAGCTGGTAGTAACGGCCAGTTCGAACTTAAC |
| | | GTATTTAAACCGGTAATGATCAAGAATTTAATCCAATCAATCAG |
| | | ACTAATTAGCGACGCGTCTATCTCTTTTACTAAAAACTGTGTAGT |
| | | CGGCATTGAGGCTAATGAAAAAAAGATTAGCTCTATCATGAATG |
| | | AATCCCTGATGCTTGTTACTGCCCTGAACCCACATATTGGCTACG |
| | | ACAAGGCTGCAAAATGTGCTAAAAAGGCGCACAAAGAAGGGAC |
| | | AACCCTAAAAGAAGCAGCACTTTCTTTAGGTTATCTGACATCAG |
| | | AAGAGTTTGATCAGTGGGTACGTCCGGAAGATATGATCAGCGCC |
| | | AAAGACTAAGTCATCAAAATCCTCGTAAATCACCTATGGTTGAT |
| | | AAGAGACTTAGGATTGAGAACCGAGGAGCACGTCATGACCAGA |
| | | GACTTTCGTATTGAAACCGACA |
| 9 | ΔfrdBCD::pC100- | TCTCAGCCTAAAGCGCGTCTATACGGTGAAGCTGCAGAAAAAGC |
| | Ka_frd (±100 bp) | CGCAGCAGAAGAAGCAGCGAAGAACGCAGAGGAGCAAGCATA |
| | | AGACACTGTTATAAAAGTCAAAAGCCTCCGACCGGAGGCTTTTG |
| | | ACTGGGCCCTGAAGTCAGCCCCATACGATATAAGTTGTAAGCTT |
| | | ATTTGAAAAAAGTGTTTGACAGTTGAACACTCGTTGCCTATAAT |
| | | CGAAAGTCGAGTTATCGAGATTTTCAGGAGAGGAAGCTCATATG |
| | | ACCAGTAACGAACGAATTCTGCAGCCATTTACTTTACCGAATGG |
| | | GACTGAACTGAAAAACCGTTTGCTAATGGCGCCAATGACCACCT |
| | | GCACCGGTTATTTCGATGGTACCGTCACCAGCGAGCTCGTTGAA |
| | | TACTATCGCGCCCGCGCCGGCAGCATCGGGACTATCATCGTTGA |
| | | ATGCTGCTTTATTGATGATTATGGTCTGGCCTTCCCGGGCGCTAT |
| | | TGGCATTGATAACGATGAAAAAATCGCCGGACTGGCGAAAATC |
| | | GCCGAAGCGATAAAAGCGGAAGGCTCGAAAGCGATTCTGCAGA |
| | | TTTACCACGGCGGCCGCATGGTTGACCCACAGTTGATTGGTGGT |
| | | CGCCAGCCGGTGGCGCCGAGCGCCATTGCCGCGCCGCGCGACG |
| | | GCGCCGCCATGCCGCGCGCCCTCAGTGGTGAAGAAGTCGAAGG |
| | | CATGATTGCCAAATTTGGCGACGGCGTACGCCGCGCCATTCTCG |
| | | CCGGTTTCGACGGCGTCGAAATCCATGGCGCCAACACCTATCTT |
| | | ATTCAACAGTTCTATTCGCCGAACTCCAACCAGCGTGACGATGA |
| | | GTGGGGCGGCAGCCGTGACAACCGCGCCAGATTCCCGCTGGCTG |
| | | TACTGGACATCACGCATAAAATGGCCCGTCAGTACGCTGACGAT |
| | | GCCTTTATTATCGGCTATCGCTTCTCGCCGGAAGAGATGGAAGT |
| | | CCCGGGGATCCGCTTCGACGACACCATGTATCTGCTGGAAAAAC |
| | | TAGCCGCCCGCGGCGTCGATTACCTGCACTTCTCGGTTGGCGCG |
| | | ACGCTGCGCCCGTCTATCGTAGATACCAGCGATCCGACGCCGCT |
| | | GATTGAAAAATACTGCGCCATGCGTTCTGAAACGCTGGCGCAGG |
| | | TGCCGGTGATGGGCGTCGGCGGGGTGGTTAACGCCGCTGATGCC |
| | | GAACAGGGCCTCGATCACGGTTATGATCTGATGGCCGTCGGCCG |
| | | CGCCTGCATCGCCTATCCGGATTGGGCTTCACGTATCGCCGCCG |
| | | GCGAAGAGCTGGAACTCTTTATCGACAGCACCCAGCGCGAGGC |
| | | GCTGCATATTCCGGAACCGCTGTGGCGTTTCTCGCTGGTCGAAG |
| | | CGATGATCCGCGACATGAGCATGGGCGACGCGAAATTTAAACC |
| | | GGGGATGTTTGTCGAAACCGTTCAGGACGATGCCAACGAACTGG |
| | | TCATTAACGTCAGCCTCGAAAACGATCGCATTGCGGATATTGAG |
| | | CTGGCCGCAAGCCCAGTACAGAGCGTTGAATTTACCACCAGCTT |
| | | CGAAGAGATCCGCGAACGTATTCTGACCGCCAACACGCCGCACG |
| | | TCGACGCTATCTCCGGCGCGACTAGCCAGAGCGAAGCGGTGAA |
| | | AAAAGCGGTGTCCAAAGCGATGCTGAAATCTAGCAAAGCGCTG |
| | | GCTGCGGAAGAGGGCGGCAACGATGCCGCGCCGAAAAGCTATG |
| | | ACGTGGTGGTGGTAGGGAGCGGCGGCGCCGGTCTGGCCGCGGC |
| | | GATTCAGGCCCACGACGAAGGCGCCAGCGTGCTGATCGTTGAAA |
| | | AAATGCCGACTATTGGCGGCAACACCATCAAAGCTTCCGCCGGG |
| | | ATGAACGCCGCGGAAACCCGCTTCCAGCGCGTCAAAGGTATTCA |
| | | GGACAGTAAAGAGTTGTTCTATCAGGAAACCCTGAAGGGCGGC |
| | | CACAACAAAAACAACCCGCAGCTGCTGCGCCGTTTCGTTGAAAA |
| | | CGCGCCGCAAGCCATTGAATGGTTGGCGGACCGTGGGATTATGC |
| | | TTAACGATATCACCACTACCGGCGGGATGAGTATCGACCGTACC |
| | | CACCGTCCGCGCGATGGATCGGCGGTTGGCGGCTACCTGATTAG |
| | | CGGTCTGGTGCGTAATATCACCAAACGCGGTATCGATGTACTGC |
| | | TGGATACCTCGGTAGAAGAGATCCTGATGCGCCACGACGAAGTG |
| | | AGCGGCGTGCGTCTTATCAACGATGAACAAGAGATCATCGACGT |
| | | GCAGACCAAAAGCATCATCGTGGCAACCGGCGGCTTCAGCGCC |
| | | AACAGCGCGATGGTGGTGAAATATCGTCCTGACCTCGAAGGCTT |
| | | CGTGACCACCAACCATAAAGGGGCGACCGGTAGCGGTATTGCG |
| | | CTGCTGGAGCGCATTGGTGCCGGCACCGTGGACATGGGCGAAAT |
| | | TCAAATTCACCCGACCGTCGAGCAGCAGACCTCGTACCTGATTT |
| | | CCGAATCGATTCGCGGCGGCGGCGCTATTCTCGTTAACCAGCAG |
| | | GGGAACCGCTTCTTCAACGAGATGGAGACCCGCGATAAAGTCTC |
| | | TGCGGCGATTATCGCGCTGCCGGAACACTATGCTTACATCGTCTT |
| | | CGACGAGTATGTGCGGGCGAAAAACAAAGCCGCCGACGAGTAT |
| | | ATCGCCAAAGGCTTCGTCACTAGCGCCAGCTCGCCGCGTGAACT |
| | | GGCGGAGAAGCTGGGGATGGATTACCATGCGTTCCTCGCTACCC |
| | | TGGAATGCTACAACGGCGCGGTGGAAAAACAGCACGATGAAGC |
| | | GTTTGGCCGCACAACCGCGCTGCGCGCGCCGATTAACGAAGGTC |
| | | CGTTCCACGCCATTCGTATTGCCCCAGGGGTGCACCACACTATG |
| | | GGCGGCGTGACCATTAATACCGATGGCGAAGTGCTGAATGTGGC |
| | | GCAGCAGCCGATTCGCGGCGCCTACGCCGCGGGTGAAGTGGTTG |
| | | GCGGTATCCATGGCGGTAACCGCATCGGCGGTAACGCGGTCGCG |
| | | GATATCATCATCTTCGGTACCCTCGCGGGCCATCAGGCGGCGAA |
| | | ACGTGCCAGAGGATAATAGGTAATTGCTGATGCCGGGCTGGCTT |
| | | AGCCGCCCGGCTTATCCCTTCTAAGGAGAGCCCATGAAAAAAGC |
| | | TGGCTTTATGCCAGCTTTTTTTGTTTTT |
| 10 | ΔsdhABCD | TATTCTCGGTGATTATCGGTCGTATCTGTTAAATAAATGTTAACT |
| | (±100 bp) | TTTGTACATTTGGACGCTGAAATTGGTTATAACAATAAATGCTC |
| | | AATGGAGCTGAGTGAGCAAGCCCGTGAAAGAAAGAAAGTCAAG |
| | | GACACTCGTCGCTTTCGGAATCAAGTCGATGCTGGTAAATCGCT |
| | | CGGTGTAATTTACTCGTAGATGAACCAGTTGCAGTGTTGTGTAA |
| | | AGTAAACCCCATCACTGCGCTGCAACTGCATAACTCGAGTAGAC |
| | | GAATATTAAAATTGCCGATT |
| 11 | ΔpdhR::pflbp- | AGGCATGGTTTCAACCATGCCTTTTTGTATACAAAAAATAATTA |
| | aceEF_pflbp- | CTTTTGTTGAAAATTAACTACATTTTTGTGTGTACAGCGAGGGAA |
| | lpdA* (±100 bp) | GTGGCCGATATAAGTTGAGCATTTATTGATTTAGTTTAAGGTTTG |
| | | CAAAAAAATTTTAGTTTAACCTGCAATTGTTGTTTCAAAAGGTA |
| | | GACGTTTAAAGCAGTGACAATATTTATGGTTTTGTAACCCTAAA |
| | | AAGTGATTTTAAGACTATAAATATTGTTATTCATAAGAGTGTACT |
| | | GTTCCCCTTTATTTCTGGGGATAGCTGGCTCAACGGCGAAGCAG |
| | | TACGTATTTTTTTCTACTAAAAAGGTAGGTATGTCATGTCTGATA |
| | | TGAAGCATGACGTAGATGCACTGGAAACTCAAGAATGGCTACA |
| | | AGCACTTGAGTCAGTTGTACGTGAAGAAGGCGTAGAGCGTGCTC |
| | | AGTTCCTACTTGAGCAAGTTCTAGATAAAGCACGTCTAGACGGT |
| | | GTTGACATGCCAACTGGAATCACCACGAACTACATCAACACGAT |
| | | TCCAGCAGATCAAGAACCAGCTTACCCAGGTGACACAACACTTG |
| | | AGCGTCGTATTCGTTCCATCATTCGCTGGAACGCAATCATGATC |
| | | GTTCTACGTGCATCGAAGAAAGACCTAGAACTAGGCGGCCACAT |
| | | GGCGTCTTTCCAGTCTTCTGCTGCATTCTACGAAACATGTTTCAA |
| | | CCATTTCTTCCGTGCTCCAAACGAGAAGGATGGTGGCGACCTAG |
| | | TTTACTACCAAGGTCACATTTCTCCAGGTATCTACTCTCGTGCAT |
| | | TCGTTGAAGGCCGTCTAACTGAAGAGCAGCTAGACAACTTCCGT |
| | | CAAGAAGTAGATGGTAAAGGTATCCCGTCATACCCACACCCTAA |
| | | ACTAATGCCTGAATTCTGGCAGTTCCCAACCGTATCTATGGGTCT |
| | | AGGTCCGATCTCTGCGATCTACCAAGCTCGTTTCCTTAAGTACCT |
| | | TGAAGGCCGCGGCATGAAAGATACTTCTGAGCAGCGCGTTTACG |
| | | CGTTCCTGGGTGACGGTGAGATGGATGAGCCAGAATCACGTGGT |
| | | GCTATCTCTTTCGCTGCGCGTGAGAAGCTAGACAACCTATGTTTC |
| | | CTAATCAACTGTAACCTTCAGCGTCTAGACGGCCCTGTAATGGG |
| | | TAACGGTAAGATCATTCAAGAACTTGAAGGTCTGTTCAAAGGTG |
| | | CTGGCTGGAACGTAGTTAAAGTAATCTGGGGTAACAACTGGGAT |
| | | TCTCTACTAGCTAAAGATACAACTGGTAAGCTTCTACAGCTTAT |
| | | GAACGAAACAATCGATGGCGATTACCAAACGTTCAAAGCGAAA |
| | | GACGGTGCATACGTACGTGAGCACTTCTTCGGTAAGTACCCAGA |
| | | AACAGCTGCACTAGTTGCTGACATGACTGATGACGAAATCTTCG |
| | | CACTTAAGCGTGGTGGTCACGAGTCTTCTAAGCTATACGCTGCA |
| | | TACAAAAACGCGGCAGACACTAAAGGCCGTCCAACGGTTATCCT |
| | | AGCTAAGACTGTTAAAGGTTACGGCATGGGTGAAGCGGCTGAA |
| | | GGTAAGAACATCGCGCACCAAGTTAAGAAGATGGACATGACTC |
| | | ACGTTCTACACCTACGTGATCGTCTAGGTCTACAAGATCTTCTAA |
| | | CTGACGAAGCTGTGAAAGAGCTTCCGTACCTGAAACTTGAAGAA |
| | | GGTTCAAAAGAGTACGAATACCTACACGCTCGTCGTAAAGCGCT |
| | | ACACGGTTACACGCCTCAGCGTCTACCGAACTTCACTCAAGAAC |
| | | TGATCGTTCCTGAACTAGAAGAATTCAAGCCTCTACTAGAAGAG |
| | | CAGAAGCGTGACATCTCTTCTACTATGGCATTCGTACGTTCACTG |
| | | AACGTTCTGCTTAAGAACAAGAACATTGGTAAGAACATCGTTCC |
| | | TATCATTGCTGACGAAGCACGTACATTCGGTATGGAAGGTCTAT |
| | | TCCGTCAAATCGGTATTTACAACCCGCACGGCCAGACTTACACA |
| | | CCAGAAGACCGTGGCGTTGTTTCTTACTACAAAGAAGCGACTTC |
| | | AGGTCAGGTTCTACAAGAAGGTATCAACGAGCTAGGTGCTATGT |
| | | CTTCATGGGTTGCAGCAGCAACTTCATACTCGACAAACGACCTA |
| | | CCAATGATTCCGTTCTACATCTACTACTCTATGTTCGGTTTCCAA |
| | | CGTGTTGGCGACATGGCGTGGATGGCTGGTGACCAACAAGCACG |
| | | TGGTTTCCTACTAGGTGCAACTGCTGGTCGTACGACACTGAACG |
| | | GTGAAGGTCTGCAGCACGAAGATGGTCACTCGCACATCATGGCG |
| | | GGTACAGTTCCTAACTGTATCTCTTACGATCCGACATTCGCTTAC |
| | | GAAGTAGCGGTAATCATGCAAGACGGTATCCGTCGCATGTACGG |
| | | TGAGCAAGAGAACGTGTTCTACTACCTAACGCTAATGAACGAGA |
| | | ACTACGCAATGCCAGCAATGCCAGAAGGCGCTGAAGAAGGCAT |
| | | TCGTAAGGGTATCTACAAGCTAGAAACTTACACTGGTTCTAAAG |
| | | GTAAAGTTCAGCTAATGAGCTCTGGTACTATCATGAACGAAGTA |
| | | CGTAAAGCAGCTCAAATCCTTAGCGATGAGTACGGTGTTGCATC |
| | | TGACGTTTACTCTGTAACGTCATTCAACGAAGTAACTCGCGACG |
| | | GTCAAGCGGCAGAGCGTTACAACATGCTACACCCAGAAGCGGA |
| | | AGCGCAAGTACCATACATCCAAACAGTAATGGGTACTGAGCCA |
| | | GCTATCGCAGCAACTGACTACATGAAGAACTACGCAGAGCAGG |
| | | TTCGTGCATTCATCCCTGCTGAATCTTTCAAAGTTCTTGGTACTG |
| | | ATGGCTTCGGTCGCTCAGACAGCCGTGAAAACCTACGTCGTCAC |
| | | TTCGAAGTGAACGCAGGTTACGTAGTAGTAGCAGCGCTAACTGA |
| | | ACTAGCGAAACGTGGCGAAGTTGAGAAGTCTGTAATTGCTGAAG |
| | | CAATTAAGAAATTCGACATCGACACTGAAAAAACAAACCCGCT |
| | | ATACGCTTAATTAAGAAGGTAGATAAGAAATGGCAATCGAAATT |
| | | AATGTACCAGACATCGGTACGGATGAGGTTGAAGTTACTGAGAT |
| | | TCTTGTAAGCGTTGGCGACAAGGTTGAAGAAGAGCAGTCTCTGA |
| | | TCACTGTTGAAGGCGATAAAGCTTCTATGGAAGTTCCTGCTTCTC |
| | | AAGCGGGTATCGTTAAAGAAATCAAAGTTGCAGAAGGCGACAA |
| | | GGTTTCTACTGGTTCTCTAATCATGATTTTCGAAGCCGAGGGTGC |
| | | AGCTGAAGCTGCGCCTGCTCCTGCGGCGGAAGCAGCTCCAGCAG |
| | | CAGCTCCAGCTCCAGCAGCAGCGGCAGAACTGAAAGAAGTTCA |
| | | CGTACCAGATATCGGTGGTGACGAAGTTGAAGTTACTGAAATCA |
| | | TGGTAGCAATCGGCGACAGCATCGAAGAAGAGCAATCTCTGATC |
| | | ACTGTAGAAGGCGACAAAGCTTCTATGGAAGTTCCTGCACCATT |
| | | CGCAGGTACGCTTAAAGAAATCAAAGTAGCAGCAGGCGATAAA |
| | | GTATCGACTGGCTCTCTAATCATGGTATTCGAAGTAGCAGGTTC |
| | | AGGCGCTCCAGCAGCAGCTCCTGCGGCAGTAGAAGCACCAGCG |
| | | GCAGCAGCTCCAGCAGCATCTGCGGCAAAAGAAGTTAACGTTCC |
| | | AGATATCGGTGGCGACGAAGTAGAAGTTACTGAAATCATGGTTG |
| | | CAGTTGGCGATACAGTGGAAGAAGAGCAATCTCTAATCACTGTA |
| | | GAAGGCGACAAAGCTTCTATGGAAGTTCCTGCACCATTCGCTGG |
| | | TACTGTTAAAGAAATCAAGATTGCAGCTGGCGACAAAGTGTCTA |
| | | CTGGCTCACTAATCATGGTATTCGAAGTGGCGGGCGCAGCTCCA |
| | | GCTCCTGCAGCGGCTCCAGCTCAAGCGGCAGCACCTGCAGCAGC |
| | | GGCTCCTAAAGCAGAAGCTCCAGCGGCGGCAGCTCCAGCAGCG |
| | | ACAGGCGATTTCAAAGAGAACGACGAGTACGCTCACGCGTCTCC |
| | | AGTTGTTCGTCGTCTAGCTCGTGAGTTCGGCGTAAACCTTTCTAA |
| | | GGTTAAAGGTTCTGGTCGTAAGAGCCGTATCCTGAAAGAAGACG |
| | | TTCAGAACTACGTGAAAGAAGCGCTTAAGCGTCTTGAGTCTGGT |
| | | GCTGCAGCATCTGGCAAAGGCGACGGTGCAGCTCTTGGTCTACT |
| | | ACCATGGCCAAAAGTGGACTTCAGCAAGTTCGGTGAGACAGAA |
| | | GTTCAACCTCTATCTCGCATTAAGAAGATTTCTGGTGCTAACCTA |
| | | CACCGTAACTGGGTAATGATCCCGCACGTTACACAGTGGGATAA |
| | | CGCAGACATCACGGCTCTTGAAGCATTCCGTAAAGAGCAAAACG |
| | | CGATTGAAGCGAAGAAAGACACTGGCATGAAGATCACTCCACT |
| | | AGTGTTCATCATGAAAGCAGTTGCTAAAGCACTTGAAGCGTTCC |
| | | CGGCGTTCAACTCTTCTCTATCAGAAGACGGTGAGAGCCTAATT |
| | | CTGAAGAAATACGTGAACGTTGGTATCGCGGTAGATACGCCAAA |
| | | CGGTCTGGTTGTTCCTGTATTTAAAGACGTGAACAAGAAAGGCA |
| | | TCTACGAGCTATCTGAAGAGCTAATGGCTGTTTCTAAGAAAGCT |
| | | CGTGCTGGTAAGCTAACTGCGGCTGACATGCAAGGTGGCTGTTT |
| | | CACTATCTCTAGCCTTGGCGGCATTGGCGGTACTGCGTTTACTCC |
| | | AATCGTGAACGCGCCAGAAGTGGGTATCCTAGGTGTATCTAAGT |
| | | CAGAGATGAAACCGGTATGGAACGGTAAAGAGTTCGAACCGCG |
| | | TCTACAACTTCCACTGTCTCTATCATACGACCACCGTGTGATCGA |
| | | TGGTGCGGAAGGTGCGCGTTTCATCACTTACCTAAACTCATGTCT |
| | | ATCTGACATTCGTCGTCTAGTTCTTTAATAGAAGTTAGATGTATG |
| | | AGAGGCGGCAATTTAGCCGCCTCTGCTAATAATAATTACTGAAC |
| | | AATTTGGTTATCGCCCTTATTCACTATGGGGTTTTCACAAAGTTG |
| | | AGCATTTATTGATTTAGTTTAAGGTTTGCAAAAAAATTTTAGTTT |
| | | AACCTGCAATTGTTGTTTCAAAAGGTAGACGTTTAAAGCAGTGA |
| | | CAATATTTATGGTTTTGTAACCCTAAAAAGTGATTTTAAGACTAT |
| | | AAATATTGTTATTCATAAGAGTGTACTGTTCCCCTTTATTTCTGG |
| | | GGATAGCTGGCTCAACGGCGAAGCAGTACGTATTTTTTTCTACT |
| | | AAAAAGGTAGGTATGTCATGAGCAAAGAAATTAAAGCCCAAGT |
| | | TGTTGTACTTGGTTCTGGTCCTGCTGGTTACTCTGCAGCATTCCG |
| | | TTGTGCGGATTTAGGTCTTGAAACTGTACTAGTTGAACGTTACA |
| | | GCACTCTTGGCGGTGTGTGTCTGAACGTGGGTTGTATCCCATCTA |
| | | AAGCACTTCTTCACGTTTCTAAAGTAATTGAAGAAGCAAAAGCA |
| | | ATGGCTGATCACGGCGTTGTATTTGGCGAACCGCAAACTGACAT |
| | | CAACAAAATCCGCATCTGGAAAGAAAAAGTTGTTAACCAACTTA |
| | | CTGGCGGCCTAGGTGGCATGGCTAAGATGCGTAACGTAACTGTA |
| | | GTTAACGGTTACGGTAAGTTTACTGGTCCTAACTCTATCCTTGTA |
| | | GAAGGTGAAGGCGAGTCAACTGTTGTTAACTTCGACAACGCAAT |
| | | CGTTGCTGCTGGTTCTCGTCCAATCAAACTGCCATTTATCCCGCA |
| | | TGAAGACCCACGTATTTGGGATTCTACTGACGCTCTTGAACTAA |
| | | AAGAAGTGCCAGAAAAACTACTTATCATGGGTGGTGGTATCATC |
| | | GGTCTGGAAATGGGTACGGTTTACCACTCTCTAGGTTCTAAAGT |
| | | TGAAGTTGTTGAGATGTTTGATCAGGTAATCCCTGCAGCAGACA |
| | | AAGATATCGTTAAGGTTTACACCAAGCGTATCAAAGACAAGTTC |
| | | AAGCTAATGCTAGAAACGAAAGTAACAGCAGTTGAAGCGAAAG |
| | | AAGACGGTATCTACGTTTCAATGGAAGGTAAGAAAGCACCTGCT |
| | | GAAGCAGAGCGCTACGATGCTGTTCTTGTTGCTATCGGTCGTGT |
| | | ACCAAACGGCAAACTGATTGACGGTGAAAAAGCGGGTCTAGAA |
| | | ATCGACGAGCGCGGCTTCATCAACGTTGACAAGCAAATGCGTAC |
| | | TAACGTTCCTCACATCTTCGCGATCGGTGATATCGTTGGTCAACC |
| | | AATGCTTGCTCACAAAGGTGTGCATGAAGGTCACGTAGCAGCTG |
| | | AAGTTATCTCTGGTAAGAAGCACTACTTTGACCCTAAAGTTATC |
| | | CCTTCAATCGCATACACTAAACCAGAAGTGGCTTGGGTTGGTAA |
| | | GACTGAGAAAGAAGCGAAAGACGAAGGCATCAAGTACGAAGTG |
| | | GCAACATTCCCATGGGCTGCATCAGGTCGTGCAATCGCATCTGA |
| | | CTGTTCAGACGGTATGACTAAGCTAATCTTCGACAAAGAAACTC |
| | | ACCGCGTAATCGGTGGTGCTATCGTTGGTACTAACGGTGGTGAA |
| | | CTACTTGGCGAAATCGGTCTTGCGATCGAGATGGGTTGTGATGC |
| | | AGAAGATATCGCTCTGACTATCCACGCTCACCCAACTCTACACG |
| | | AGTCTGTTGGTCTAGCTGCTGAAGTATTTGAAGGTTCTATCACTG |
| | | ACCTTCCAAACAAAAAAGCAGTTAAGAAGAAGTAAGTTTCTTTA |
| | | CTGATTAAATAATAAAAACCGCTGAACATTCAGCGGTTTTTTTGT |
| | | TTTTAACAGCTAAGTTTCAAATACAAAAAAGCCTGGCAGAAGCC |
| | | AG |
| 12 | ΔaspA1::pC75- | CCAAAAAGGTGATCTAGTTACTATTTTTATGTGCTAGGTAAAGT |
| | dcuA (±100 bp) | ATGATGTGCCACATAAGTCAGGACGGATTTTCCAGCCCAAAATT |
| | | CTGATGAATGGAAGCTTATTTGAAAAAAGTGTTTGACAAGGTGC |
| | | CCGAAACCGGTTATAATCCTCGTTCGAGTTATCGAGATTTTCAG |
| | | GAGCTAAGGAAGTCATATGGTCATGGTCGAACTCTTCGTGGTTC |
| | | TGCTCTTTATCTATTTGGGAGCGAGAATCGGCGGTATCGGCATC |
| | | GGTTTTGCCGGTGGTGCGGGTGTCATTG |
| 13 | ΔdctA | ACCAAGATTAGGTTTTTTCTTTCGTGTTTTTTCACTCCGCTGCTAA |
| | (±100 bp) | CCTGCACGCCACTTTGAAGTGAACTCAGAGTGCATAACTATAAG |
| | | GAGTGTTCTCATGAATACCAAGAAACCGATGTCGCTTACCAGGA |
| | | CACTCGTCGCTTTCGGAGAAGAAGTGCATCTTAAACGCGCTGAC |
| | | GCTTAAACCGTTTACGGTTATCCTGTCTAAAACAAAGCCCCGAT |
| | | GGAAACATCGGGGCTTTGTTCATTGAGAAAGGTTTATTAAGCAA |
| | | TCTTAAAGGCTTTTAGTC |
| 14 | ΔlldH ΔdldH | TATCCGCATTGGCGTATATATCTTCAAGAGAGCTGTAACGTGCG |
| | (±100 bp) | CCCATCTCTATTGCCAGTGGGTTTTCGTAAGGATCATGGCAAAG |
| | | AATTTCCATGCCGTTGTCGCCGTTAGTACGCCATGGTTCTCTCTC |
| | | GAAATCATTGTGTTCTCTAATACCGTAATGCAACTTAGGTACAA |
| | | TGAATAGATAAATCTGCAATACGGGCTCTGTCATACCAAAATGC |
| | | CATTAACATAGGTTTTTTACGTCCCCACGTTATAATCAGATTGCT |
| | | TTGTCATCATCAACTGACTAGCATATCCCTTCTCTTCTTTCCTTGT |
| | | TGGCTTCTTTATTTATTAGGCGTTCTGGCGATGTCAGTAAAATGG |
| | | TGTTCTTTACTTGTATGTTGTGCGTTCGTGTTCGAAGCGCAGGGT |
| | | GCTGTCGATCTTGTTAAAGTCGACAAGTCGAAGCGTCGAATGTA |
| | | CTTGATGGAAGAAGGCGCTGTGATAAAGGAGTATCGAATTGCGC |
| | | TGGGTGCTAACCCCAAAGGTCATAAACAAGAAGAAGGAGACAA |
| | | TCGCACACCAGAAGGTGATTATACTCTGGATTACGTGATCTATG |
| | | ACTCGGCTTTTTACCGTTCCGTACATATCAGCTACCCCGATCCTA |
| | | TTGATACGCTTGAGGCCCATCGTCGCGGTGTCTACCCTGGTGGC |
| | | AACATTAAAATCCATGGCTTAAAAAATGGAGAAACCCAAGATC |
| | | CAAGCTTTATACAAAGTTTTGATTGGACCAATGGGTGTATCGCG |
| | | ATTACTAACGAAGAGATGGATGAGTTTATTCAATTGGTTAGAAG |
| | | GGGCACACCTATTACCATTGAGTGGTAGCAGTATATCCCCCTCA |
| | | TGATCTAAAACAGTCTTTTACCGAAGAAACCGTGTAGGGTAATC |
| | | ACTGAAGATGCCATCTAGAGGTGGCAGATGTTTGAGTTTATGTG |
| | | GGTTGTTGACGGTATAGCACATCACTTTATAGCCATGTTGTTGGA |
| | | GTTTTTTAATCTGACGGGAGCGCACCCAATTGATGTTTAAGTTAC |
| | | AGCTGTAAGCGTTGACTTCTTCAAGCAGCAATTGGTCTTTTCTGG |
| | | TGAGAAACTCACTCAATACACCTAAACGGTAGCCTCTGCAGTGC |
| | | TCAAAGAGCGCTCTAATCACATCATGACTAAAACTAGAAAGTAA |
| | | AATACTCTCTGAGGGCAGAGGGCTACCAGAAAGCACTTTCGCTA |
| | | ATAACTTTGCAACTGGCTCTGCGGGATGACGATCAACTTTAACT |
| | | TCGATGTTTAAATTGAGGTTGTGCTCTGCGGCCAGTTCAAGAAG |
| | | CTCTTCCAACGTCATGATGGTTTCACCCTTAAACTCATCACTAAA |
| | | CCAGCTACCAAAGTCTAAGCTTTTGAGTTCATCCAAAGTCAGTT |
| | | CATCTATTCGCCCTTGTCCGTTACTACAGCGGTTGACCGTGTGGT |
| | | CATGGCATACCACCAAAATGTTGTCTTTCGTCGGCTGAACATCG |
| | | ACTTCTACCCATTTCAAATCAAGGTTTATTGCAGCCAGAACGCTT |
| | | ATCTTTGTGTTTTCTGGGTAAGACCCCGCCACACCTCGGTGACCA |
| | | ATTAACAATGTATCCATGTTGTCGTTCTTCTCGTTTGGGTTGAAC |
| | | CCGATACTAATCGATTTTAAGTGGTTTGGTTTAGTGGGCATCAA |
| | | GCGAGGTCGATGCAACACCTTCATTTACAGCGGTATTCACAGGA |
| | | ATACAAATATGTTGGACTTTTACTCTATCGCAGCTCATTTTTAAA |
| | | GTAACGACTAATCTTGCAAGCGCAGAACAATACCCAGAAACGC |
| | | ACGGAGTTGAGCTGAGCATAAGCCTTTATGCATGTCGAAAAATT |
| | | TGATGATTGGATGGACTTGAATCCTGTATTTTGCAGTGATTTTTT |
| | | AGAATTATCAGGGAGAGAAAAATGGCGTACTAACGGCGACAAC |
| | | TTTGGTTACGTATTAGGCGAGCATGGTAGTCATTGTTTTACGCCG |
| | | AAGAGTTTAATTTCTATCGCAGGCCAACCGGCGGATTACTACTG |
| | | TGATACACACA |
| 15 | Δald | TTGGTGCCAAATATTACAAATCTATAACTATACTAGCTATTAATT |
| | (±100 bp) | CGATAATAACACCCTACAAATAGTTAAAATAACCATTATAGAAA |
| | | GAGGATACAGGATGGCGTACTAACGGCGACAACCCTGCAAAAG |
| | | TCGTCATTGTGGGCGGCGGTGTCGTTGGCGCAAACGCAGCTCGT |
| | | ATGGCTGTGGGCCTTCGTGCAGACGTCACTATTCTTGATCGTAAT |
| | | GTCGATACTCTGCGTAAACTGGACGAAGAATTCCAAGGCCGCGC |
| | | TAAAGTCGTTTATTCTACAGAAGACGCGATTGAGAAGCATGTTG |
| | | TAGAAGCTGACCTAGTAATTGGCGCAGTTCTTATCCCGGGTGCA |
| | | GCTGCACCTAAACTGGTGACAAAAGAACACATTGCGAAGATGA |
| | | AGCCAGGCGCAGCGGTAGTTGATGTAGCTATCGACCAAGGTGGT |
| | | TGCTTTGAAACATCGCACGCAACCACTCACGCAGAACCGACATA |
| | | CATCGTCGATGAAGTCGTTCACTACTGCGTAGCAAACATGCCAG |
| | | GTGCCGTTGCCCGTACTTCTACTTTTGCATTGAACAACGCAACGT |
| | | TACCTTACATTTTGAAACTGGCGAATAAAGGCTACCAAAAAGCC |
| | | CTTCTCGACGAGAAAGGTTTCCTTGAAGGACTAAACGTTATTCA |
| | | CGGCAAAGTGACTTGTAAAGAAGTTGCCGACAGCTTTGGTCTTG |
| | | AATATGTAGAAGCAGAGCAAGCGATTGCAATGTTCAACTAATGA |
| | | CAAAATAATTCATTTTTGTCACGTTTTGTTTACAAAAGCCCACTA |
| | | GCAGGTGGGCTTTTTCCTATAGAGCATTTAGATATAAATAATAA |
| | | CCATTACT |
| 16 | ΔpflB | TGTTATTCATAAGAGTGTACTGTTCCCCTTTATTTCTGGGGATAG |
| | (±100 bp) | CTGGCTCAACGGCGAAGCAGTACGTATTTTTTTCTACTAAAAAG |
| | | GTAGGTATGTCATGGCGTACTAACGGCGACAACCCTGACGCATA |
| | | CGGCCGTGGTCGTATCATCGGTGACTACCGTCGCGTTGCGCTTTA |
| | | CGGTATCGACTTCCTAATGAAGGACAAACTAGCTCAGTTCACTT |
| | | CTCTTCAAGAGAAATTTGAGAACGGCGAAGACCTTCACATGACT |
| | | ATGCAACTTCGTGAAGAAATTGCAGAGCAACACCGCGCTCTAGG |
| | | TCAGATCAAACAAATGGCTGCGAAATACGGTTTCGATATTTCTC |
| | | GCCCTGCTGAAACTGCACAAGAAGCTATCCAATGGACTTACTTC |
| | | GGCTACCTAGCTGCTGTTAAGTCTCAAAACGGTGCTGCAATGTC |
| | | TCTAGGTCGTACTTCTACATTCCTAGACGTGTACATCGAGCGTGA |
| | | TATCGCTGCAGGTAAGATCACTGAAGACCAAGCTCAAGAAATG |
| | | ATCGACCACTTCGTAATGAAACTACGTATGGTTCGTTTCCTACGT |
| | | ACTCCTGAGTACGATGAGCTATTCTCTGGCGACCCAATCTGGGC |
| | | AACAGAATCAATGGGTGGTATGGGTCTTGACGGTCGTACGCTAG |
| | | TAACGCGTTCTAACTTCCGTTTCCTAAACAGCCTATACACTATGG |
| | | GGCCTTCTCCAGAGCCAAACATCACTGTTCTTTGGTCTGAAGCA |
| | | CTTCCAGATGGCTTCAAACGTTTCTGTGCAAAAGTATCTATCGAT |
| | | ACTTCTTCTATCCAGTACGAAAACGACGATCTAATGCGTCCAGA |
| | | CATGGAATCAGACGATTACGCTATCGCTTGTTGTGTATCTCCAAT |
| | | GGTTGTTGGTAAGCAAATGCAGTTCTTCGGTGCTCGTGCGAACC |
| | | TTGCTAAAACTATGCTTTACACCATCAACGGC |
| 17 | ΔackA | GACCAAGATTAGTACGCACCCTTGCTGGTATCGACTATTCTTGTG |
| | (±100 bp) | ATTAATTTTTCATTCTTTAACTGATTTTGATCAGACGAATAACAG |
| | | GTAGTCATACATGTCTAAGCTAGTTTTAGTTTTAAACTGCAGGAC |
| | | ACTCGTCGCTTTCGGAGCTGAAGACACTGCACGCCTAGCAGGTC |
| | | TTTAATGCATAAACTGGCTAGCCAATTGGCTAGCCAGTTTTTCTT |
| | | CCAAAGAGCTTTCTTTAGGGGCTCAGCTCCTATTCTTTGAAGATC |
| | | ATGGAATAAGAATTG |
| 18 | ΔadhE | TAAGTCGTTTGTTTGTTAAGTGGCTGATAAGCAGCAAAAAGAGA |
| | (±100 bp) | TAAGCAATAGCCTTACTAAAAAGTTTTTAATATTTGTTAATTTTA |
| | | GGAGAATCCCTATGCCTGTAACTAATATGGCTGAACTAGATAGG |
| | | ACACTCGTCGCTTTCGGAAAAGCTAAAAAAGAAAAAGCAGACG |
| | | CGTAAGTAAGCGTTGTTTTGAACAAGGTTTTCGCTAGCACGAAA |
| | | CCTTAATCGGGAAAAGTTAAAAGCCCCAGTCGAATGACTGGGGC |
| | | TTTTTTTATCTTCTTTG |
| 19 | lldH KO Sequence | ATGGCGTACTAACGGCGACAACTTTGGTTACGTATTAGGCGAGC |
| | | ATGGTAGTCATTGTTTTACGCCGAAGAGTTTAATTTCTATCGCAG |
| | | GCCAACCGGCGGATTACTACTGTGATACACACAATATCAAACGA |
| | | ATTGATGCGGATGAGCTGTTGGAGGCGGTGAAACAAGCCGGCT |
| | | ATGAAATATTTCGTCGCAAGAACAATACCGTACACGGCATTGCC |
| | | GCGAGTGTATTTCGGATAGTGCAGGCAGTTATCATTAACGAGCG |
| | | CTCGGTGTTGCCTGTCGGAACGATGATAAAAGGGCAATATGGCG |
| | | TGAATAAAGTCGTACTCAGTTTGCCAACGGTTGTGGGTAAAAAT |
| | | GGCGCAGACTCGGTTCTTACTCATCCATTTAGTGGTGAAGAGCT |
| | | AAAAACGTTAAGAGCCATCAGTGAGAACTTACGTTCAATCGTAA |
| | | AGAACGTTGCATCGTCAACCAGTTTAGAGTGTTAG |
| 20 | dldH KO | TTAGTTGATCAGTTCATTGCCTGAAACTTGCCCAGAGAAGAAGG |
| | Sequence | CTTCTACGTTATTTAGTGTCACCGACGCGATGTTGTTGAGCGCTT |
| | | CGTGAGTTAAGAAAGCTTGATGACCCGTGAACAGCACGTTATGG |
| | | CATGCCGACAGTCGGCGGAAAACATCGTCTACGATAATGTCGTT |
| | | TGATTTATCCTGGAAGAACAACTCTTTTTCATTGTCGTACACATC |
| | | CAAGCCTAGTGAGCCGATTCTGCCTTGTTTTAGCGCTTCGATTGC |
| | | TGCTACAGAATCCAATAGCTCTCCACGACTCGTATTAATGATCA |
| | | TCACCCCATCTTTCATTTTCGAAAATGAGTCAGCATTGAGGAGG |
| | | TGGTAGTTTTCTTTACTCATCGGGCAATGCAAAGTAATAATATCC |
| | | GCATTGGCGTATATATCTTCAAGAGAGCTGTAACGTGCGCCCAT |
| | | CTCTATTGCCAGTGGGTTTTCGTAAGGATCATGGCAAAGAATTT |
| | | CCATGCCGTTGTCGCCGTTAGTACGCCAT |
| 21 | alD KO Sequence | ATGGCGTACTAACGGCGACAACCCTGCAAAAGTCGTCATTGTGG |
| | | GCGGCGGTGTCGTTGGCGCAAACGCAGCTCGTATGGCTGTGGGC |
| | | CTTCGTGCAGACGTCACTATTCTTGATCGTAATGTCGATACTCTG |
| | | CGTAAACTGGACGAAGAATTCCAAGGCCGCGCTAAAGTCGTTTA |
| | | TTCTACAGAAGACGCGATTGAGAAGCATGTTGTAGAAGCTGACC |
| | | TAGTAATTGGCGCAGTTCTTATCCCGGGTGCAGCTGCACCTAAA |
| | | CTGGTGACAAAAGAACACATTGCGAAGATGAAGCCAGGCGCAG |
| | | CGGTAGTTGATGTAGCTATCGACCAAGGTGGTTGCTTTGAAACA |
| | | TCGCACGCAACCACTCACGCAGAACCGACATACATCGTCGATGA |
| | | AGTCGTTCACTACTGCGTAGCAAACATGCCAGGTGCCGTTGCCC |
| | | GTACTTCTACTTTTGCATTGAACAACGCAACGTTACCTTACATTT |
| | | TGAAACTGGCGAATAAAGGCTACCAAAAAGCCCTTCTCGACGA |
| | | GAAAGGTTTCCTTGAAGGACTAAACGTTATTCACGGCAAAGTGA |
| | | CTTGTAAAGAAGTTGCCGACAGCTTTGGTCTTGAATATGTAGAA |
| | | GCAGAGCAAGCGATTGCAATGTTCAACTAA |
| 22 | pflB KO Sequence | ATGGCGTACTAACGGCGACAACCCTGACGCATACGGCCGTGGTC |
| | | GTATCATCGGTGACTACCGTCGCGTTGCGCTTTACGGTATCGACT |
| | | TCCTAATGAAGGACAAACTAGCTCAGTTCACTTCTCTTCAAGAG |
| | | AAATTTGAGAACGGCGAAGACCTTCACATGACTATGCAACTTCG |
| | | TGAAGAAATTGCAGAGCAACACCGCGCTCTAGGTCAGATCAAA |
| | | CAAATGGCTGCGAAATACGGTTTCGATATTTCTCGCCCTGCTGA |
| | | AACTGCACAAGAAGCTATCCAATGGACTTACTTCGGCTACCTAG |
| | | CTGCTGTTAAGTCTCAAAACGGTGCTGCAATGTCTCTAGGTCGT |
| | | ACTTCTACATTCCTAGACGTGTACATCGAGCGTGATATCGCTGC |
| | | AGGTAAGATCACTGAAGACCAAGCTCAAGAAATGATCGACCAC |
| | | TTCGTAATGAAACTACGTATGGTTCGTTTCCTACGTACTCCTGAG |
| | | TACGATGAGCTATTCTCTGGCGACCCAATCTGGGCAACAGAATC |
| | | AATGGGTGGTATGGGTCTTGACGGTCGTACGCTAGTAACGCGTT |
| | | CTAACTTCCGTTTCCTAAACAGCCTATACACTATGGGGCCTTCTC |
| | | CAGAGCCAAACATCACTGTTCTTTGGTCTGAAGCACTTCCAGAT |
| | | GGCTTCAAACGTTTCTGTGCAAAAGTATCTATCGATACTTCTTCT |
| | | ATCCAGTACGAAAACGACGATCTAATGCGTCCAGACATGGAATC |
| | | AGACGATTACGC |
| 23 | pC100-aceEF | AGTCAAAAGCCTCCGACCGGAGGCTTTTGACTAAGTTGAGCATT |
| | pC100-lpdA | TAAAGCTTATTTGAAAAAAGTGTTTGACAGTTGAACACTCGTTG |
| | | CCTATAATCGAAAGTCGAGTTATCGAGATTTTCAGGAGAGGAAG |
| | | CTCATATGTCTGATATGAAGCATGACGTAGATGCACTGGAAACT |
| | | CAAGAATGGCTACAAGCACTTGAGTCAGTTGTACGTGAAGAAG |
| | | GCGTAGAGCGTGCTCAGTTCCTACTTGAGCAAGTTCTAGATAAA |
| | | GCACGTCTAGACGGTGTTGACATGCCAACTGGAATCACCACGAA |
| | | CTACATCAACACGATTCCAGCAGATCAAGAACCAGCTTACCCAG |
| | | GTGACACAACACTTGAGCGTCGTATTCGTTCCATCATTCGCTGG |
| | | AACGCAATCATGATCGTTCTACGTGCATCGAAGAAAGACCTAGA |
| | | ACTAGGCGGCCACATGGCGTCTTTCCAGTCTTCTGCTGCATTCTA |
| | | CGAAACATGTTTCAACCATTTCTTCCGTGCTCCAAACGAGAAGG |
| | | ATGGTGGCGACCTAGTTTACTACCAAGGTCACATTTCTCCAGGT |
| | | ATCTACTCTCGTGCATTCGTTGAAGGCCGTCTAACTGAAGAGCA |
| | | GCTAGACAACTTCCGTCAAGAAGTAGATGGTAAAGGTATCCCGT |
| | | CATACCCACACCCTAAACTAATGCCTGAATTCTGGCAGTTCCCA |
| | | ACCGTATCTATGGGTCTAGGTCCGATCTCTGCGATCTACCAAGCT |
| | | CGTTTCCTTAAGTACCTTGAAGGCCGCGGCATGAAAGATACTTC |
| | | TGAGCAGCGCGTTTACGCGTTCCTGGGTGACGGTGAGATGGATG |
| | | AGCCAGAATCACGTGGTGCTATCTCTTTCGCTGCGCGTGAGAAG |
| | | CTAGACAACCTATGTTTCCTAATCAACTGTAACCTTCAGCGTCTA |
| | | GACGGCCCTGTAATGGGTAACGGTAAGATCATTCAAGAACTTGA |
| | | AGGTCTGTTCAAAGGTGCTGGCTGGAACGTAGTTAAAGTAATCT |
| | | GGGGTAACAACTGGGATTCTCTACTAGCTAAAGATACAACTGGT |
| | | AAGCTTCTACAGCTTATGAACGAAACAATCGATGGCGATTACCA |
| | | AACGTTCAAAGCGAAAGACGGTGCATACGTACGTGAGCACTTCT |
| | | TCGGTAAGTACCCAGAAACAGCTGCACTAGTTGCTGACATGACT |
| | | GATGACGAAATCTTCGCACTTAAGCGTGGTGGTCACGAGTCTTC |
| | | TAAGCTATACGCTGCATACAAAAACGCGGCAGACACTAAAGGC |
| | | CGTCCAACGGTTATCCTAGCTAAGACTGTTAAAGGTTACGGCAT |
| | | GGGTGAAGCGGCTGAAGGTAAGAACATCGCGCACCAAGTTAAG |
| | | AAGATGGACATGACTCACGTTCTACACCTACGTGATCGTCTAGG |
| | | TCTACAAGATCTTCTAACTGACGAAGCTGTGAAAGAGCTTCCGT |
| | | ACCTGAAACTTGAAGAAGGTTCAAAAGAGTACGAATACCTACA |
| | | CGCTCGTCGTAAAGCGCTACACGGTTACACGCCTCAGCGTCTAC |
| | | CGAACTTCACTCAAGAACTGATCGTTCCTGAACTAGAAGAATTC |
| | | AAGCCTCTACTAGAAGAGCAGAAGCGTGACATCTCTTCTACTAT |
| | | GGCATTCGTACGTTCACTGAACGTTCTGCTTAAGAACAAGAACA |
| | | TTGGTAAGAACATCGTTCCTATCATTGCTGACGAAGCACGTACA |
| | | TTCGGTATGGAAGGTCTATTCCGTCAAATCGGTATTTACAACCC |
| | | GCACGGCCAGACTTACACACCAGAAGACCGTGGCGTTGTTTCTT |
| | | ACTACAAAGAAGCGACTTCAGGTCAGGTTCTACAAGAAGGTATC |
| | | AACGAGCTAGGTGCTATGTCTTCATGGGTTGCAGCAGCAACTTC |
| | | ATACTCGACAAACGACCTACCAATGATTCCGTTCTACATCTACT |
| | | ACTCTATGTTCGGTTTCCAACGTGTTGGCGACATGGCGTGGATG |
| | | GCTGGTGACCAACAAGCACGTGGTTTCCTACTAGGTGCAACTGC |
| | | TGGTCGTACGACACTGAACGGTGAAGGTCTGCAGCACGAAGAT |
| | | GGTCACTCGCACATCATGGCGGGTACAGTTCCTAACTGTATCTCT |
| | | TACGATCCGACATTCGCTTACGAAGTAGCGGTAATCATGCAAGA |
| | | CGGTATCCGTCGCATGTACGGTGAGCAAGAGAACGTGTTCTACT |
| | | ACCTAACGCTAATGAACGAGAACTACGCAATGCCAGCAATGCC |
| | | AGAAGGCGCTGAAGAAGGCATTCGTAAGGGTATCTACAAGCTA |
| | | GAAACTTACACTGGTTCTAAAGGTAAAGTTCAGCTAATGAGCTC |
| | | TGGTACTATCATGAACGAAGTACGTAAAGCAGCTCAAATCCTTA |
| | | GCGATGAGTACGGTGTTGCATCTGACGTTTACTCTGTAACGTCAT |
| | | TCAACGAAGTAACTCGCGACGGTCAAGCGGCAGAGCGTTACAA |
| | | CATGCTACACCCAGAAGCGGAAGCGCAAGTACCATACATCCAA |
| | | ACAGTAATGGGTACTGAGCCAGCTATCGCAGCAACTGACTACAT |
| | | GAAGAACTACGCAGAGCAGGTTCGTGCATTCATCCCTGCTGAAT |
| | | CTTTCAAAGTTCTTGGTACTGATGGCTTCGGTCGCTCAGACAGCC |
| | | GTGAAAACCTACGTCGTCACTTCGAAGTGAACGCAGGTTACGTA |
| | | GTAGTAGCAGCGCTAACTGAACTAGCGAAACGTGGCGAAGTTG |
| | | AGAAGTCTGTAATTGCTGAAGCAATTAAGAAATTCGACATCGAC |
| | | ACTGAAAAAACAAACCCGCTATACGCTTAATTAAGAAGGTAGAT |
| | | AAGAAATGGCAATCGAAATTAATGTACCAGACATCGGTACGGA |
| | | TGAGGTTGAAGTTACTGAGATTCTTGTAAGCGTTGGCGACAAGG |
| | | TTGAAGAAGAGCAGTCTCTGATCACTGTTGAAGGCGATAAAGCT |
| | | TCTATGGAAGTTCCTGCTTCTCAAGCGGGTATCGTTAAAGAAAT |
| | | CAAAGTTGCAGAAGGCGACAAGGTTTCTACTGGTTCTCTAATCA |
| | | TGATTTTCGAAGCCGAGGGTGCAGCTGAAGCTGCGCCTGCTCCT |
| | | GCGGCGGAAGCAGCTCCAGCAGCAGCTCCAGCTCCAGCAGCAG |
| | | CGGCAGAACTGAAAGAAGTTCACGTACCAGATATCGGTGGTGA |
| | | CGAAGTTGAAGTTACTGAAATCATGGTAGCAATCGGCGACAGCA |
| | | TCGAAGAAGAGCAATCTCTGATCACTGTAGAAGGCGACAAAGC |
| | | TTCTATGGAAGTTCCTGCACCATTCGCAGGTACGCTTAAAGAAA |
| | | TCAAAGTAGCAGCAGGCGATAAAGTATCGACTGGCTCTCTAATC |
| | | ATGGTATTCGAAGTAGCAGGTTCAGGCGCTCCAGCAGCAGCTCC |
| | | TGCGGCAGTAGAAGCACCAGCGGCAGCAGCTCCAGCAGCATCT |
| | | GCGGCAAAAGAAGTTAACGTTCCAGATATCGGTGGCGACGAAG |
| | | TAGAAGTTACTGAAATCATGGTTGCAGTTGGCGATACAGTGGAA |
| | | GAAGAGCAATCTCTAATCACTGTAGAAGGCGACAAAGCTTCTAT |
| | | GGAAGTTCCTGCACCATTCGCTGGTACTGTTAAAGAAATCAAGA |
| | | TTGCAGCTGGCGACAAAGTGTCTACTGGCTCACTAATCATGGTA |
| | | TTCGAAGTGGCGGGCGCAGCTCCAGCTCCTGCAGCGGCTCCAGC |
| | | TCAAGCGGCAGCACCTGCAGCAGCGGCTCCTAAAGCAGAAGCT |
| | | CCAGCGGCGGCAGCTCCAGCAGCGACAGGCGATTTCAAAGAGA |
| | | ACGACGAGTACGCTCACGCGTCTCCAGTTGTTCGTCGTCTAGCTC |
| | | GTGAGTTCGGCGTAAACCTTTCTAAGGTTAAAGGTTCTGGTCGT |
| | | AAGAGCCGTATCCTGAAAGAAGACGTTCAGAACTACGTGAAAG |
| | | AAGCGCTTAAGCGTCTTGAGTCTGGTGCTGCAGCATCTGGCAAA |
| | | GGCGACGGTGCAGCTCTTGGTCTACTACCATGGCCAAAAGTGGA |
| | | CTTCAGCAAGTTCGGTGAGACAGAAGTTCAACCTCTATCTCGCA |
| | | TTAAGAAGATTTCTGGTGCTAACCTACACCGTAACTGGGTAATG |
| | | ATCCCGCACGTTACACAGTGGGATAACGCAGACATCACGGCTCT |
| | | TGAAGCATTCCGTAAAGAGCAAAACGCGATTGAAGCGAAGAAA |
| | | GACACTGGCATGAAGATCACTCCACTAGTGTTCATCATGAAAGC |
| | | AGTTGCTAAAGCACTTGAAGCGTTCCCGGCGTTCAACTCTTCTCT |
| | | ATCAGAAGACGGTGAGAGCCTAATTCTGAAGAAATACGTGAAC |
| | | GTTGGTATCGCGGTAGATACGCCAAACGGTCTGGTTGTTCCTGT |
| | | ATTTAAAGACGTGAACAAGAAAGGCATCTACGAGCTATCTGAA |
| | | GAGCTAATGGCTGTTTCTAAGAAAGCTCGTGCTGGTAAGCTAAC |
| | | TGCGGCTGACATGCAAGGTGGCTGTTTCACTATCTCTAGCCTTGG |
| | | CGGCATTGGCGGTACTGCGTTTACTCCAATCGTGAACGCGCCAG |
| | | AAGTGGGTATCCTAGGTGTATCTAAGTCAGAGATGAAACCGGTA |
| | | TGGAACGGTAAAGAGTTCGAACCGCGTCTACAACTTCCACTGTC |
| | | TCTATCATACGACCACCGTGTGATCGATGGTGCGGAAGGTGCGC |
| | | GTTTCATCACTTACCTAAACTCATGTCTATCTGACATTCGTCGTC |
| | | TAGTTCTTTAATAGAAGTTCTAGCATAACCCCTTGGGGCCTCTAA |
| | | ACGGGTCTTGAGGGGTTTTTTGGCTTAGCCACAAGCTTATTTGAA |
| | | AAAAGTGTTTGACAGTTGAACACTCGTTGCCTATAATCGAAAGT |
| | | CGAGTTATCGAGATTTTCAGGAGAGGAAGCTCATATGAGCAAAG |
| | | AAATTAAAGCCCAAGTTGTTGTACTTGGTTCTGGTCCTGCTGGTT |
| | | ACTCTGCAGCATTCCGTTGTGCGGATTTAGGTCTTGAAACTGTAC |
| | | TAGTTGAACGTTACAGCACTCTTGGCGGTGTGTGTCTGAACGTG |
| | | GGTTGTATCCCATCTAAAGCACTTCTTCACGTTTCTAAAGTAATT |
| | | GAAGAAGCAAAAGCAATGGCTGATCACGGCGTTGTATTTGGCG |
| | | AACCGCAAACTGACATCAACAAAATCCGCATCTGGAAAGAAAA |
| | | AGTTGTTAACCAACTTACTGGCGGCCTAGGTGGCATGGCTAAGA |
| | | TGCGTAACGTAACTGTAGTTAACGGTTACGGTAAGTTTACTGGT |
| | | CCTAACTCTATCCTTGTAGAAGGTGAAGGCGAGTCAACTGTTGT |
| | | TAACTTCGACAACGCAATCGTTGCTGCTGGTTCTCGTCCAATCAA |
| | | ACTGCCATTTATCCCGCATGAAGACCCACGTATTTGGGATTCTAC |
| | | TGACGCTCTTGAACTAAAAGAAGTGCCAGAAAAACTACTTATCA |
| | | TGGGTGGTGGTATCATCGGTCTGGAAATGGGTACGGTTTACCAC |
| | | TCTCTAGGTTCTAAAGTTGAAGTTGTTGAGATGTTTGATCAGGTA |
| | | ATCCCTGCAGCAGACAAAGATATCGTTAAGGTTTACACCAAGCG |
| | | TATCAAAGACAAGTTCAAGCTAATGCTAGAAACGAAAGTAACA |
| | | GCAGTTGAAGCGAAAGAAGACGGTATCTACGTTTCAATGGAAG |
| | | GTAAGAAAGCACCTGCTGAAGCAGAGCGCTACGATGCTGTTCTT |
| | | GTTGCTATCGGTCGTGTACCAAACGGCAAACTGATTGACGGTGA |
| | | AAAAGCGGGTCTAGAAATCGACGAGCGCGGCTTCATCAACGTTG |
| | | ACAAGCAAATGCGTACTAACGTTCCTCACATCTTCGCGATCGGT |
| | | GATATCGTTGGTCAACCAATGCTTGCTCACAAAGGTGTGCATGA |
| | | AGGTCACGTAGCAGCTGAAGTTATCTCTGGTAAGAAGCACTACT |
| | | TTGACCCTAAAGTTATCCCTTCAATCGCATACACTGAGCCAGAA |
| | | GTGGCTTGGGTTGGTAAGACTGAGAAAGAAGCGAAAGACGAAG |
| | | GCATCAAGTACGAAGTGGCAACATTCCCATGGGCTGCATCAGGT |
| | | CGTGCAATCGCATCTGACTGTTCAGACGGTATGACTAAGCTAAT |
| | | CTTCGACAAAGAAACTCACCGCGTAATCGGTGGTGCTATCGTTG |
| | | GTACTAACGGTGGTGAACTACTTGGCGAAATCGGTCTTGCGATC |
| | | GAGATGGGTTGTGATGCAGAAGATATCGCTCTGACTATCCACGC |
| | | TCACCCAACTCTACACGAGTCTGTTGGTCTAGCTGCTGAAGTATT |
| | | TGAAGGTTCTATCACTGACCTTCCAAACAAAAAAGCAGTTAAGA |
| | | AGAAGTAA |
| 24 | lpdA E354K | ATGAGCAAAGAAATTAAAGCCCAAGTTGTTGTACTTGGTTCTGG |
| | | TCCTGCTGGTTACTCTGCAGCATTCCGTTGTGCGGATTTAGGTCT |
| | | TGAAACTGTACTAGTTGAACGTTACAGCACTCTTGGCGGTGTGT |
| | | GTCTGAACGTGGGTTGTATCCCATCTAAAGCACTTCTTCACGTTT |
| | | CTAAAGTAATTGAAGAAGCAAAAGCAATGGCTGATCACGGCGT |
| | | TGTATTTGGCGAACCGCAAACTGACATCAACAAAATCCGCATCT |
| | | GGAAAGAAAAAGTTGTTAACCAACTTACTGGCGGCCTAGGTGGC |
| | | ATGGCTAAGATGCGTAACGTAACTGTAGTTAACGGTTACGGTAA |
| | | GTTTACTGGTCCTAACTCTATCCTTGTAGAAGGTGAAGGCGAGT |
| | | CAACTGTTGTTAACTTCGACAACGCAATCGTTGCTGCTGGTTCTC |
| | | GTCCAATCAAACTGCCATTTATCCCGCATGAAGACCCACGTATT |
| | | TGGGATTCTACTGACGCTCTTGAACTAAAAGAAGTGCCAGAAAA |
| | | ACTACTTATCATGGGTGGTGGTATCATCGGTCTGGAAATGGGTA |
| | | CGGTTTACCACTCTCTAGGTTCTAAAGTTGAAGTTGTTGAGATGT |
| | | TTGATCAGGTAATCCCTGCAGCAGACAAAGATATCGTTAAGGTT |
| | | TACACCAAGCGTATCAAAGACAAGTTCAAGCTAATGCTAGAAAC |
| | | GAAAGTAACAGCAGTTGAAGCGAAAGAAGACGGTATCTACGTT |
| | | TCAATGGAAGGTAAGAAAGCACCTGCTGAAGCAGAGCGCTACG |
| | | ATGCTGTTCTTGTTGCTATCGGTCGTGTACCAAACGGCAAACTG |
| | | ATTGACGGTGAAAAAGCGGGTCTAGAAATCGACGAGCGCGGCT |
| | | TCATCAACGTTGACAAGCAAATGCGTACTAACGTTCCTCACATC |
| | | TTCGCGATCGGTGATATCGTTGGTCAACCAATGCTTGCTCACAA |
| | | AGGTGTGCATGAAGGTCACGTAGCAGCTGAAGTTATCTCTGGTA |
| | | AGAAGCACTACTTTGACCCTAAAGTTATCCCTTCAGTTGTTTACA |
| | | CTGAGCCAGAAGTGGTATGGGTTGGTAAGACTGAGAAAGAAGC |
| | | GAAAGACGAAGGCATCAAGTACGAAGTGGCAACATTCCCATGG |
| | | GCTGCATCAGGTCGTGCAATCGCATCTGACTGTTCAGACGGTAT |
| | | GACTAAGCTAATCTTCGACAAAGAAACTCACCGCGTAATCGGTG |
| | | GTGCTATCGTTGGTACTAACGGTGGTGAACTACTTGGCGAAATC |
| | | GGTCTTGCGATCGAGATGGGTTGTGATGCAGAAGATATCGCTCT |
| | | GACTATCCACGCTCACCCAACTCTACACGAGTCTGTTGGTCTAG |
| | | CTGCTGAAGTATTTGAAGGTTCTATCACTGACCTTCCAAACAAA |
| | | AAAGCAGTTAAGAAGAAGTAA |
| 25 | ptsI KO Sequence | ATGATTTCAGGCATCCTAGCATCTCCTGGTAGGACACTCGTCGCT |
| | | TTCGGAGTTGAAAAGTTCATTGCTGAGAAAGTTCAGTAA |
| 26 | pC75-EcExuT | GGGCCTTAATGTTGCGGTGCGGGATCGTTATGTGTTTCCTGCGCC |
| | | ACCTCAATCGCCGGTTTGTTCTGCAACACGGTCCAGATAACCAG |
| | | CGCACCTAACAGGTCGAACACTGCCAGAACTGCGAACAGCGGG |
| | | CTGAAGCCGATGGTGTCAGCCAGTGCACCGACAACCAGCGCAA |
| | | ACAGCGTACTTGCCAGCCATGCGGACATCCCGGTTAAACCGTTT |
| | | GCCGTTGCCACTTCGTTACGACCAAACACGTCAGAAGAGAGCGT |
| | | AATCAGCGCGCCAGACAGTGCCTGGTGGGCAAAACCACCGATA |
| | | CACAGCAGCATAATTGCGACATACGGGTTGGTGAACAGACCGAT |
| | | CATACCCGGGCCAATCATCAGCACAGCACCCAGCGTTACGACCA |
| | | TCTTACGGGAAACGATCAGGTTCACACCAAACCAACGCTGGAAC |
| | | AGCGGCGGCAGGTAACCACCGAGGATACAACCGAGGTCAGCAA |
| | | ACAGCATCGGCATCCAGGCGAACATCGCGATCTCTTTCAGGTTA |
| | | AAGCCGTAAACTTTAAACATGAACAGCGGGATCCACGCGTTAAA |
| | | AGTACCCCAGGCCGGTTCTGCCAGGAAACGCGGCAGCGCGATA |
| | | CCCCAGAACTGACGGTTACGCAGGATCTGACCAACGGACATTTT |
| | | CTTCGCCGTGCTCACCTGGTGCTGGGCTTCCTGACCATTAATAAT |
| | | ATAGTCGCGTTCTTCATCGGTCAGATGCTTCTGGTCGCGCGGATG |
| | | TTTATAGAAAATCAGCCATGCCATCGCCCAGATAAAGCTCAATG |
| | | CACCGGAGATGATAAATGCCATCTGCCAGCTGTGCATTACGATT |
| | | GCCCATACCACCAGCGGCGGCGCAATCATCGCACCAATAGAAG |
| | | AACCTACGTTAAAGTAACCTACTGCGATGGAACGCTCTTTCGCC |
| | | GGGAACCATTCGGAGCTGGCTTTCAGACCCGCCGGGATCATCGC |
| | | TGCTTCCGCGGCACCGACTGCACCACGAGCAATCGCCAGGCCAC |
| | | CCCAACTACCGGCCAGCGCGGTTGCACCACAGAACACGGCCCAC |
| | | AGTACAGCAAACATTGCATAACCGATTTTCGTACCCAGTACATC |
| | | CAGTACATAGCCCGCTACCGGTTGCATGACCGTATAAGCAGCAG |
| | | AATAGGCTGCGATGATATAGGAATACTGTTGGGTGGAGATGTTT |
| | | AACTCTTCCATCAGAGTTGGCGCAGCTGCCGCCACAGTGTTACG |
| | | CGTCAGGTAACCAAGCACGGTGCCAAGCGTCACCAGTGCGATCA |
| | | TATACCAACGTAACCCTTTAATTTTACGCATATGACTTCCTTAGC |
| | | TCCTGAAAATCTCGATAACTCGAACGAGGATTATAACCGGTTTC |
| | | GGGCACCTTGTCAAACACTTTTTTCAAATAAGCTT |
| 27 | pC75-VnGLK- | AAGCTTATTTGAAAAAAGTGTTTGACAAGGTGCCCGAAACCGGT |
| | VpSGLT | TATAATCCTCGTTCGAGTTATCGAGATTTTCAGGAGCTAAGGAA |
| | | GTCATATGTCAGAGCAATACGCATTAGTAGGCGACATCGGCGGT |
| | | ACTAACGCAAGATTGGCTCTCTGCGAGTTATCTACGGGCACGAT |
| | | TTCTGATATCGTCACTTATCCTGCAGCAGAATACGAATCCCTCGA |
| | | AGTAGTAATGCGCCAGTACTTGGATCGTCATGATACCCCGATCT |
| | | CTTCAGCGTGTATCGCCATTGCATGTCCAGTAACAGGTGACTGG |
| | | GTAAGTATGACTAATCACCATTGGGAATTCTCTATCCAGGAGCT |
| | | AAAATCACAACTTAACCTACAAGTACTAGAAGTAATCAACGATT |
| | | TTACGGCTGTGAGTTTTGCTATTCCAAGCTTGAAACCAGAGGAC |
| | | CGTGTCCAAATTGGGGGTGGTGAAGCGATCGCAAACAAGCCAA |
| | | TCGCTGTGTATGGTGCTGGTACCGGCCTGGGTGTTGCGCAGCTG |
| | | ATCCACGGAGGCAACCTATGGCATTCGGTTCCGGGCGAGGGCGG |
| | | ACATGTTGACTTAGCAGCGTGTACACCTGAAGAAGATGATCTTA |
| | | TTGTTTACTTGAGAAACAAGTTTGGTAGAGTCTCTGCTGAGCGTT |
| | | GTTTATCAGGCCAAGGTATCCGTAACATTTACGACTTCGTTGTTT |
| | | CCCACCATGGTGCAGCACCTGAAGATTACACACCAGCAATGATC |
| | | ACAGAAAAAGCTCTTAAAGCGGAGTGCAAAGACTGTGAACGCG |
| | | CACTGTATCTTTTTTGTATCCTTATGGGCCGATTTGGAGGCAATC |
| | | TTGCGCTAACTAACGGCAGTTTCGGCGGTGTGTACATTGCCGGT |
| | | GGTATCGTACCAAAAGTCCAGGAACTTTTTATCAAGAGCGGCTT |
| | | CCGTGTTGCTTTCGAAGACAAGGGTCGATTTAAAGAATACCTGC |
| | | AGTCAATCCCGGTTTTTCTAATCACTCATTCAGAACCTGGTCTTC |
| | | TAGGTGCCGGTACATATATTCGCCAATCTCTCAACATTAGTATCA |
| | | ACTAA |
| 28 | pC100-CgMDH | TTAGAGCAAGTCGCGCACTGCCTCGCGTTCAGCCTGTAATTCCT |
| | | GAGCATTCGCGTCGATACGTGCTCTCTGAAAATCTGAAATCTCT |
| | | AGGCCTTCAACGATTTCCCATTCACCGTTTCGACTTACTGTTGGC |
| | | AGACCAACAAAGATGCCTTCCGGAATGCCGTATGCACCAGTGCT |
| | | AGGAATTGCCGCACTGCTCCACGCCTCTGTACCTTGTACCCAATC |
| | | TCTCATATGATCAATCGCAGAGGATGCTGCAGAAGCTGCAGATG |
| | | ATTTACCACGTACCTCAATGATTTCAGCGCCACGGTTAGCCACA |
| | | CGAGGAATAAACTCTTCTACGTACCAATCGTGATCAACTAAGTC |
| | | AGTGACCTTTTCTCCACCAACTGTTGCGTACGTGATGTCTGGAAA |
| | | TTGTGTTGCGGAATGATTTCCCCATACCACGATGTTGTTAAATTC |
| | | TGCAGAACCACGGCCAAGCTTAGTTGCTAGCTGACTGATCGCAC |
| | | GGTTATGATCAAGGCGCATCATTGCGTTGAATCTTGATGCTGGA |
| | | ACATCTGGTGCCGCAGCTGAAGCTATCAACGCGTTGGTGTTCGC |
| | | TGGGTTTCCAACAACTAGCACACGGATGTCATCTGCGGCGTTGT |
| | | CGTTGATAGCTTTACCTTGAGGACCAAAAATTTTGCCGTTGTTAG |
| | | CCAGTAAGTCTGCTCTCTCTTCGCCTTTACCGCGAGGTTTCGCAC |
| | | CTACCAAAAATGCAGCATTAGCGCCGTCGAATGCCTCGTTGGCA |
| | | TCCGCAGTGATCGTGATGTTTCGTAGGAGGGGGAAGGCCGAATC |
| | | TAGAAGTTCCATAGCAACACCCTCTGCCCCGCCAAGAGCTTGCG |
| | | GGATTTCTAGAAGTTTCAGTTCTACTGGCGTGTCAGTGCCGAAT |
| | | ACTTCACCGTTGGCGATGCGCCATAAAAGTGAATAAGAGATTTG |
| | | ACCAGCTGCGCCGGTGACTGTGACCTTTTTCGTAGAGACGTTTTG |
| | | CGGTGAATTCATATGAGCTTCCTCTCCTGAAAATCTCGATAACTC |
| | | GACTTTCGATTATAGGCAACGAGTGTTCAACTGTCAAACACTTTT |
| | | TTCAAATAAGCTT |
| 29 | pC100-AsVnPCK | CGATATAAGTTGTAAGCTTATTTGAAAAAAGTGTTTGACAGTTG |
| | hybrid-2 | AACACTCGTTGCCTATAATCGAAAGTCGAGTTATCGAGATTTTC |
| | | AGGAGAGGAAGCTCATATGACTGACCTAAACAAACTCGTCAAG |
| | | GAACTTAATGACCTAGGCCTTACAGATGTTAAGGAAATTGTATA |
| | | TAACCCCAGTTACGAGCAACTTTTCGAGGAGGAAACTAAACCTG |
| | | GCTTGGAGGGTTTTGATAAAGGGACGCTAACCACTCTTGGCGCA |
| | | GTTGCCGTCGATACGGGGATTTTTACAGGACGTTCACCGAAGGA |
| | | TAAATATATCGTTTGTGATGAAACTACGAAGGACACAGTATGGT |
| | | GGAACAGCGAAGCTGCGAAAAACGATAACAAACCAATGACGCA |
| | | AGAGACTTGGAAGAGTTTGAGAGAATTAGTGGCTAAACAACTTT |
| | | CTGGTAAAAGACTATTCGTGGTAGAGGGTTACTGCGGCGCAAGT |
| | | GAAAAACACCGCATCGGTGTGCGTATGGTCACTGAAGTGGCATG |
| | | GCAGGCGCATTTTGTAAAAAACATGTTTATCCGACCAACAGATG |
| | | AAGAGCTTAAAAATTTCAAGGCGGATTTTACCGTGCTAAACGGT |
| | | GCTAAATGTACTAACCCAAACTGGAAAGAACAGGGCCTCAACA |
| | | GTGAAAACTTTGTCGCTTTTAATATTACCGAAGGTATTCAGCTTA |
| | | TCGGCGGCACTTGGTACGGCGGTGAAATGAAAAAGGGTATGTTT |
| | | TCAATGATGAACTACTTCCTGCCATTAAAAGGTGTAGCTTCTATG |
| | | CATTGTTCGGCAAACGTAGGAAAAGACGGTGACGTAGCTATTTT |
| | | CTTCGGCCTATCTGGTACGGGTAAAACAACGCTTTCGACCGATC |
| | | CTAAACGCCAATTAATCGGTGATGACGAGCACGGTTGGGATGAA |
| | | AGCGGCGTATTTAACTTCGAAGGTGGCTGTTACGCGAAGACCAT |
| | | CAAACTATCGAAAGAAGCCGAGCCTGATATCTACAACGCTATCC |
| | | GCCGCGATGCGCTGCTAGAAAACGTAACGGTACGTAACGATGGT |
| | | TCTATCGACTTTGATGATGGTTCTAAGACAGAGAACACTCGTGT |
| | | TTCTTACCCGCTTTACCACATTGAAAACATCGTTAAGCCAGTATC |
| | | GAAAGGCGGTCATGCGAATAAAGTGATCTTCCTGTCTGCTGACG |
| | | CGTTTGGTGTGCTTCCTCCAGTATCGAAACTGACACCAGAGCAA |
| | | ACCAAGTACCACTTCCTGTCTGGCTTTACTGCCAAACTAGCAGG |
| | | TACAGAGCGCGGTATCACTGAGCCGACGCCAACTTTCTCTGCGT |
| | | GTTTTGGCGCTGCGTTCCTAACGCTTCACCCAACTAAGTACGCA |
| | | GAAGTACTGGTTAAACGTATGGAAGCAGCAGGTGCAGAAGCTT |
| | | ACCTGGTTAACACTGGCTGGAATGGTAGCGGTAAACGTATCTCA |
| | | ATTCAGGATACACGCGGCATCATTGATGCGATCCTAGATGGCTC |
| | | AATTGAAGATGCACCAACTAAGCATATCCCAATCTTCAACCTTG |
| | | AAGTTCCTACTTCACTACCAGGTGTTGATCCAAGCATTCTTGATC |
| | | CACGTGATACTTACGTTGATCCACTGCAATGGGAAAGCAAAGCG |
| | | AAAGATCTAGCAGAACGCTTTATCAATAACTTTGATAAATACAC |
| | | AGACAACGCAGAAGGTAAATCTCTGGTTGCTGCTGGTCCACAGC |
| | | TTGACTAA |
| 30 | PN96_RS22390 | TTACGACCGAAGGACCAAGTCGTTTTGGTAAAGAGAGCGAAAA |
| | KO Sequence | CGTGAATAACGCTCAGACAGTCCTGCTTCACGCTCGACATTCGG |
| | | TTGATAAACGCGATGAATTTCCGGCTTCTTACATACTTCATCCAC |
| | | CGATTTTCCTGACGCCAGGCAAGCAAGTCGAGCCGCCCCCAACG |
| | | CCCCACCCGTTTCACCACCTTTGTGAGTAATCGTTGGCAAGTGA |
| | | AGAATATCTGCAAGTAACTGCGCCCAGAACGAGCTACGTGCACC |
| | | ACCACCAACCAATGAGCACTGTTCGATGTGCGTACCGCTTTCTT |
| | | GCAACACTCGCAAACCATCAGCAATACCAAAGCTCACCCCTTCG |
| | | ATCACCGCATAGCCAAGTAAGGCGCGATTGGTGGAATGGGTCAT |
| | | ACTGTGGAACATACCCGTCGCTTCCGGATCGTTATGTGGCGTAC |
| | | GCTCACCAGACAGATACGGTAAGAAAATTGGCGCTTTTGCTTTC |
| | | TCTTCTTCACTAAGCTGCTCAATTTCAGCCAGCAACTCCACTTCC |
| | | GTTGTGCCTACTATGCGGCAGAACCATTGAAGGCAGCTGGCAGC |
| | | ACTTAGCATGACACTCATCTGATGCCATCGTTGTGGCAAAACAT |
| | | GACAGAAGGCGTGTACGGCAGACTCAGGTGCGGGGCGATATTT |
| | | CTCGTTGACCACAAACAGTACGCCAGAAGTGCCAAGGGATATA |
| | | AACGCATCACCCGGATTGACCGCACCAACACCGATTGCGCTTAC |
| | | GGCATTATCACCACCGCCACCAGCGACGATGACAGATGGACTTA |
| | | GTCCCCATTTGTGGGCCATTTCAGCGGATAAAGAAGCCGATACT |
| | | TCACAGCCCTCGACCAGTTCAGGCATTTGTTCCCGCGTTAGCTGG |
| | | CATTTGTCGAGCAGAGAATCTGACCAGTCACGCTTAGCGACATC |
| | | CAACCATAACGTGCCTGCAGAATCCGACATGTCGGAAACTTTTT |
| | | TGCCCGTCATTTTGAAGCGAAGATAATCTTTCGGCAGCAGCACT |
| | | GTGCTAATGCGGGAAAAGTTTTCAGGTTCATGCTTGCGAACCCA |
| | | AATTAACTTGGGAGCCGTAAATCCTGGCATCGCAAGGTTACCAG |
| | | CAATTTCATGCAGATCCGGAGCAGCCGCTTCAAGTTCGGTGCAC |
| | | TCTTCGTGACAACGTGTGTCGTTCCACAAAATAGCTGGACGAAT |
| | | CACATCATTGGCCTCATCCAGAAGCACTGCGCCATGCATCTGTC |
| | | CAGACAAACCAATGGCTTTAATTGCTCCCCAATGCTCAGTGCAT |
| | | TTCTCACGCAGCGCCCCGATCAAATAGTTCGTTGCTTCCCACCAC |
| | | TCTTGCGGTGCCTGTTCCGACCAATGAAGATGCGGGCGTTGAAT |
| | | CGTCAGTGGTGCGCTGTGTGAAGCGACCGTTTCACCGTGTTCAT |
| | | CAATCACCAACGCTTTCACTTCTGAGGTGCCGAGATCTATACCT |
| | | AGATACATGCCCATTCCTTATTGAATCATTGAGTAAGCTGCTGC |
| | | AACTTTTTCTCGCAGCAATGTTTCAAAATCCGCGCGACTAGCTA |
| | | GTTCACCAAACAAAGCGACATCTTGCGCGTAAGCGGCGATTGGG |
| | | TCTTCAGAATCAAACATGCTGTGCACGGCATTTTCATCAAGAAT |
| | | CCCATCCTGATACTCATAAGGCAGTAAACCTTTGTGCCACTGGT |
| | | GCATAAAGACAAAGAACAGCGCAGGCAGCATCGCAGTCGCATC |
| | | TGGTGTAACACCTCGCTGATAGCATTCAATCATCGTCGGCGTAA |
| | | TCATGGCCGGGATTTTAGAAAACCCATCCGCAGCGACACGCTGG |
| | | TTGGTGTCTTTAATGTATGGGTTGGTAAAGCGCTCTAACACCACC |
| | | TCAAGATAGGTCGGCAGATCGATACCATTGTCACCAAGGCAAGG |
| | | AATCACATCCTGACTGACGTAATCGTGCGCAATCTTGTAGATAA |
| | | AATCCGTCAAAGTACTTTCGTGAATAAATGACTGGCCAACTAAT |
| | | GTTCCGGCCCAAGCGATACAGCTGTGCGAAGCATTCAAGATTCG |
| | | GATTTTCGCCTCTTCGTAAGGAATAACCGACTCCACCATTTCCAC |
| | | GCCTACATTTTCCAGTGCAGGTCGAACGTCTTTGAAACTGTCTTC |
| | | AATTACCCACTGAATGAAAGTTTCACCCATCACTGGCGCGTTAT |
| | | CTTCAATACCTGTTTGTGCTTTAATCCGATATGGCAGGTCTTCAG |
| | | CGGGACGAGGTGTAATACGATCCACCATGGTGTTAGGACTTGTC |
| | | GTATTTTCCTTCACCCATTGCAACACGTCAGCCTTACCAGTCAGA |
| | | GTCAGGAACTCCATTAGACCATCTCGGAATCGCTCACCATTGTG |
| | | ACGCACGTTGTCACAGTTCAGCAGTGTCACTGGTCCAGCACTAT |
| | | CCGCCATACGGCGCTCAAGGATTTTCGTCACCGTGCCGTAAATT |
| | | GTTTGGTGGCCACCGCTCAGATCAGCTTTTAGGACTGCGTTATCA |
| | | ACATCCAGTGAGTGATCGGTTCTTAAGTAGTACCCGCCTTCTGTG |
| | | ACCGTAAAGGCGATCACTTTTGTCTGCGCTTTTGCACCTTCTTCA |
| | | ATCAGCGGCGCCAAATCTTCCTGCCACGGGAGCAACGTCTGAAT |
| | | AGATTTGATCACCTCATATTCACGCTCCCCTGCTGGACTGACCGT |
| | | CTCTAACACATACTCACCATTCTGCGCACGGAGAGTTTCAACGG |
| | | TTGATTCTGCATCATTGCGAATGTTACCTGCCGCGATATGCCAGC |
| | | TTTGGTCACCCGTTTCTAGCAATTTGTGCAGGTACCAGGCTTGGT |
| | | GCGCACGGTGAAATGAACCCAGACCGATGTGAAGCCACGTATA |
| | | TTGGTTTTTCATTATATTTCCTCGTTTTTGTTTGATGGCTTAACTC |
| | | TAATTGATCAAATTACCAAATAAAGAGCGATGGATCACAAATGA |
| | | ACAAAAGACCGATAATTGGTTTTTTGTTCATTTGTGAGTAATTGA |
| | | TTGCTATTGCACTCACGCGAAGTTAGAGAGAAGAGTGGCTAGAG |
| | | ACGATAGTTTTAAAACCGATAGGACACTCGTCGCTTTCGGAGCA |
| | | ACAGCGAGATGGTTATTAACGCAATAAGCGAATTTTCAGTTTAA |
| | | GCCGTAGATCTCTTCAGGTGTCGCCCGCGCCCCGTTAAACCAGA |
| | | CTTTCAATTGTCCGCATAACGCTATCAATTTGCGGCTCAATTGAA |
| | | AGTCTGCATCAAGACAACAACGTTTTCTCATTAGAAACACGAGA |
| | | AGTTAATGTGTTTAATTAATACGGTAGAAACGTTTAGCCAAATT |
| | | AGTGTGTTAGCCTTTCTATCAACGCTTGTTGATATTCAGGTAATT |
| | | TCGAACGAGAGACGGATTGAAATGCTGATGGCGTTAGGTAAGTC |
| | | ATGCCTGCATGTAGCGCACTCGCTTTCATTGGTAAGAACACATC |
| | | TTCTAGTTTAACGCCTATTCTGCCCGATTCCCCGTAGGTACTTGC |
| | | TCCACCAGCCGCAGTCGTCACCAACAGCATTTCTTTTCCCTGCCA |
| | | TAATCCAGGTCCTACACTCCCCCAAACATCATTCATGTAAGCTTT |
| | | CATCATCGGAGTAATATTGAACCAATGGGTTGGAAACATAAATA |
| | | CAATGCGATCTTGTTCTCTCGTTAGCTCACGCTCCTTTTGTGCAT |
| | | CGATGGCGCGAGTATCAAAACCATAAATAGTCTCTAGGTTTCGG |
| | | ACTGTGACTCCTTCAACACTTTCAGCCGCTTGCTGCATCCCCTTG |
| | | GTCATCACTGACCTTTCTGGATAGGGATGTGAGACAATAACCAG |
| | | CGTTTTTATATCCGTTTTATGAACCTGCTCTGCAGCGGACACAAC |
| | | ATTGATGCATGTAAGCCACAGAACTTGCAAGGCGAGAAAAAAT |
| | | CCAGCCTGCTTAATGACGCTATTCCATTTCATTTGCGCTGTTCCT |
| | | AAAATTCTATCTTTCCACAGTTGTTGTAAAAAAAATTAAGCCCA |
| | | GCGAGCTGAGCTTAATTTTATTGATGCCAAGATTTTATTGACCAG |
| | | CAACGTATTGCTCGTCAGTCACTTTCTCTTTCCAGATAACATTTT |
| | | TGCCATCTTTCGATGCTGTAACAACAAGGTGTGTCATTGGTGCAT |
| | | CTGGGGTCGCGCCGTGCCAGTGATCAATACCCGGTGGGCACCAA |
| | | ACGCTCTCGTTTTCGCTAAAGCGAATCACTTTACCATCTCGGGTA |
| | | CCAGTAAGTGCCACACCAGAAGTGACAATCATATGCTGGCCTGC |
| | | AGGATGCATGTGCCACGCGGTACGAGCGCCTTTTTCGAATGTCA |
| | | CATAAGCAGCGCTGTAATTCGCGACGTCATTTTCAGGGAATAGC |
| | | ATGTCCACTTTTACATTTCCTGTGAACAAATCTTCCGGTCCGTTA |
| | | AAGGATTTAAGATCTTGTGCGCGATACGCTTGCTGGCTGCCTGC |
| | | CGCATCGTCAGCAGCATTGGTAGTTGAAACCATTGCCGTCGATG |
| | | AAACCATCCCTGCCAGTAACGTTAATATGAGTTTATTTTTCATAA |
| | | TCGAATTCCCTGTTATTTATTTAGGACCGAGTCCAGAACTTGTTG |
| | | TGCGTTGTTAGCCACGTCTTCACCAACTTGCTTTTCAATGATGGT |
| | | GATGAATTCTTTTAGCTCATCAACGGTTACATCGTTATTCATACT |
| | | AATGCTGTAATGAGCTGCCAATTGGCTGTTTACACCTGGTATATT |
| | | GGATAGAGTCGCAATCGTAGCGACTTCACGTTGTTTCCAAGTCA |
| | | GCACATCACGAGCAAAAATGTCACCAAACAAATGAGATTTCAG |
| | | GTATTCATCAATTTGAGGAGAGAAATCAAATAACGGCCCTTTTA |
| | | CTTCAACACCGATGAGTTTAGTTTGGTTTTCAGCACCAAATTCAA |
| | | GGCTGGTTTTATCTGTCGGTAGTGGCGATGACTCTGGACCAACG |
| | | ACGTCTTCGATTCCATTGCTCTTTCTTTCTTCAAGAACCTGCATA |
| | | AATGCAGCCAATGCATTCAGGCTACGAGGGAAACCAGCATATG |
| | | CGTATAGCTGACCCAGCACTTCCTTGGTTTCATTAATGGTTAAAC |
| | | CTGCGTCCAAGCCTTCGTTCAAGCTTACTTTTAGCTTCTCGATTT |
| | | CACCACTGGCGGCAAACGCTGCGATCGGAATAATCGCCTTCTCT |
| | | TTAGTATCAAAACCTTCTGCCGCCGTTGCGGTATTCATAGTCAAT |
| | | ACCGCTAGCAAACTCGCCACAAAAATGGACCATTTACTGAAATA |
| | | TCTGCCTATATGAAACATTTGTGTCTCCCTATTCCCTTTGCATTTT |
| | | TGAGTCTTCAATCGCACTCTCTAAATATAAAGTTATCGCAAAATT |
| | | TGCATACTACTTTGCCTAGAAGAGTTTAATTATTGCCTAATCTTC |
| | | TATTAAGCAAACTCCACTAGAATTGAGATATAAATAAAAACATG |
| | | ATGTTACATTTGGAAACAATAATAACGCGACCATTTACTTGACT |
| | | ATTAAAATGTAAAAACTACTCAATAAACAATTAATCGTTATTCG |
| | | TACTAGCTAGTCATTTAAATGTAGAAGTAAACATCGTCTATTTA |
| | | ATTAAATAGTAATAATCAGGCGTAATTTCATTTTTATCTCGGCCC |
| | | GCTTATTAACGTAGAAATGCCTGTTAATCTTTTTGATTAACAGGC |
| | | ATTTATTTACAAAATCCAATGTATTATTCAATAACCTCTATAGTG |
| | | ACTTTCATTGAACCACCAGAAGTAAAGCGCGATAAATCGCCGTC |
| | | AACTTTCCCGAGGTTAATCAATCCAGATGCATAGCCAAAGTTAT |
| | | TATAAAACACGACTAAGTTCCCCCAAGGTGCGTAGTAAGAAATG |
| | | TCTCCCGCTTTGGAACTCACGCCAGCTGGCGCCCCTTCCTTGGTT |
| | | AACTTCGATGGTAGGTAGGCTATTTTTTCAGTACTGGCGTAATCT |
| | | TCCAACTCTAAAGTTAATGGTAGTTGGGCGATAAAATCTTGTGT |
| | | GCTCGGACTATCATTTAACGTCGCAGTGACGGTGCGATCATCAA |
| | | ATACAAAACGAATCTTCATTGTACTCTCCCCGGTAATAACTCCG |
| | | CTCTCGGTCACGTTGCCGGAAGCGAAACAAATTTGGCTAATAAG |
| | | TAATGCGGGCAACACTGTAAGCGTGGATAAAATGGTTTTGTGCC |
| | | ACTTCTTTAACAACATAGTAGTGATTCCCTTTTAGTTAAGTGACA |
| | | GTT |
| 31 | pC25-sbtA | GTGAGCGAAAACAGCGTTAAAATCGCACATTTAACATTCATTCA |
| | | GTCTACTATAACCCGTATGGGGCAAAATTCTTTTCTGCTAAAGG |
| | | GGTGGTGTATTACACTAATTGCCGCTCTATTTGCGTTATCTCCGA |
| | | GTTCATCTAACAAGTCGTTTGCTCTTATCTCTTACATGCCATTAT |
| | | TTTTATTCTGGTTGATGGATGCTTATTATCTTTCGCTCGAACGTTC |
| | | ATACATCCAGCTTTATAACCTAGTGGCAAAGAATGAGGTTAAAA |
| | | GTGATGCACTTACCTTAGATTTAGAACAAGTAGGCGGTGGAGAG |
| | | AGTTTGTTCAATACGGCTTTTTCCAAAACTCTGTTGGTGTTTTAT |
| | | GGAAGTGTGATTTCTATAGTTTTATTTTTGATGTTTGGAGTTTTG |
| | | GGCTAATGAGTAGTGAAAGCTGTGATTATTTAATATCCCTATCTT |
| | | TTGCTGGTGAAGATAGAGAGTATGTCGATGCTGTTGCCTCTGGG |
| | | TTAAAGAATAATGGTATATCAGTTTTTTACGACAAGTATGAGCA |
| | | AGCCTCTCTCTGGGGTAAGGATCTTTACCAGCACCTAACAGACG |
| | | TTTATCAAAATAGATCCCGCTACGTTGTAATGTTTATTTCCGAGC |
| | | ATTATGCTAAAAAACTTTGGACCTCACACGAAAAACGAATGGCT |
| | | CAGGCGAGAGCATTTAAAGAAAGTAGGGAGTACATATTACCCG |
| | | TAAAGTTTGATGACACTATTATTCCTGATATTCCAGACACAACTG |
| | | GTTATTTGGATGCCCGAGAATTTGTGCCAGATGAGATAGTTAAG |
| | | TTCATTAAACAAAAACTGCAAGATGACGGGATAACAATAACAA |
| | | AACAAAAGTCACTGACCAATGAGTTATTAAAATCGACAGCAGA |
| | | AGAGCTAGTAAATTTAATTAGAGAAGAACTAATCCAGCACGAA |
| | | CAGAAAACCGAGAGCCTTAATAGTCCAATTCGGTCCCTTCATTC |
| | | TGAAGCTGAGTTTTCCAAACTTTGGGAGGCGCGGACTAAGAGAC |
| | | AACATCAATTAAAAAAGTGGTTATTAAATAGTTTTTCTAACAAA |
| | | TATCAATCGAAATCCATATTAATAAAAAATGAATTAATACAAAG |
| | | GCTTAATTTAAATGAAAGAGATGTTTATTTCGATGCAGTGTACG |
| | | AAAACCCTGTAAACCCGCTAGGTATATCAGAAGTTGCGGCAGAC |
| | | CTAGAAAAATTAAGTATGATGCTGGATACAAACGAATGAGCCAT |
| | | TTGAAGTCAATAAATAAAATTCCTTTCAAAGAGTTACTTATATGT |
| | | GGAAGTAGAGTTTATGAAACGCAAAAAATTATTGAATACAAAG |
| | | ATATAGAGCCAATTGTAATTGCGAATGGGATAAAGCCAAGAAT |
| | | ATGGCTAACGGTATTAGTTGAAAATGGCGATTCGTTCGCTTTGG |
| | | TAGATGACTCCAGAGCGAAGCATGAATCCGTTATTTGCAATGTT |
| | | ACAACTAGTAACGTGGAAATTTATGTAGATGACCATTTTATCCTT |
| | | AAAGGCACCAGATCGAGAACGGAGCGATTTCATATACATCACCT |
| | | AGACTTAAGAAGCTTAGGTTTTAGTGTATACGGTTCGGATGAAT |
| | | CTGGCTTGTATGCGAATGGAGTATCTTTAAATTCTATCTCAGCTA |
| | | GAGGCGGTCGTTGCCTTATCAAGTTAGGTTAATTTACGCTTGCTG |
| | | GGTATCCATTCGGCGGCTTTCGTTCGATGGCCGCCACGAAACGG |
| | | TAAGACGTTTTCTTTTGCAGGATAGACCTTAGGTTGGCTGATGTG |
| | | ACCATACATTTCGCCAAACTAAAAATGTTCGTCACGCAAAGCTT |
| | | TCTAATTCCTTCGGGCTGAACTCACGACGGTGTGGTGACAATGG |
| | | CGCGTTTCTCGATGATTTTAAACCTAGACTAACGACGGCCATTG |
| | | GGTAATCAAACCAACGCTACTTGGCTCTTCAGAAAAAAGAGCAG |
| | | GGAAGTCATGAAGTTAGCTCCACAAGTCGTCAGTAAACATTCTA |
| | | GTGAATCTAAGTTTTAGTGACCGCGTAACTTAATGTCAATGATA |
| | | AATCAACAAGATTCAGGTAGATGTGGGATCTGAATATGGCGTGT |
| | | ATTCTCAGTCTGGTTTTTTCAGCTATATTTATCTTGAATATCACAT |
| | | TTAACAAGATGGGCTAGACAATGAAACTAGCATATTACGTAAGC |
| | | GATATAGCTCAAGATAATAGCGACCACGAGGTCCATAATAGTGG |
| | | TGACCATGAAGTCCATAACTCGGGTTGTCCTCATTTTGATAGAAT |
| | | CCAATCCAAGACTTATTTAGGCTTATTTGATAATTGTGCTGAAGC |
| | | AGTCATTGAAGCAAAGAAAAAATACGACCAGTCAAATGGTTGTT |
| | | ATTGGTGTTGTAACGAATGCCACATGTCACACTAGTTAGGAACG |
| | | GGAACTTTCTACAAACAAATCGCGCACGCACATTCTGGTAGTAA |
| | | GCTCCCAATAGAAATGAAGGCGACACTTGAGAAACACCAAGAG |
| | | CCATACGCAGCAGGAACTTGGTGCAAGTGACGCCGCCAATAACT |
| | | CCGGATTAAAGTTAAAGCGCTTTGAGTTTATAAGAAGCAGCAAA |
| | | CAACATTTCACTGAACGCCAGTAAGTCGGACAGTCAGAAATGTT |
| | | GAGAGCGTATCTACACAATGAGAACTCTAGAAAATCAGAGCGT |
| | | AAGAAAGTACGGCTCAATTAGAATGCTCAGGCGAATTAGTAGC |
| | | AGAACTTCAAGAACGCCAACTTGAACTGATAAACCGAGCGAATT |
| | | TTAGTTCAAGGATATAGTTGCTGTAGGCAAGCAATAGAAAATCA |
| | | ATCCAGCTATTCGGTTACTTGAATGAGCCTTAACGCTGTCCATCG |
| | | AACAATGTGAGCAAGCAGACTGGGTTTATTCGCGGCAATGTGAA |
| | | GATCCTTCCCCTATTTTGTCTAATAGCATAAAAGAAAAAGCCAC |
| | | CTATCACGCGCGGCGTAATAGGTGGCTTTGTAATTCAACGGGGC |
| | | CCCTGAAGTCAGCCCCATACGATATAAGTTGTAAGCTTATTTGA |
| | | AAAAAGTGTTTGACAGTTGAACACTCGTTGCCTATAATCGAAAG |
| | | TCGAGTTATCGAGATTTTCAGGAGCTAAGGAAGCTCATATGGAT |
| | | TTTTTGTCCGATTTTTTGACTAAATTTGGTTCACAGTTACAGTCC |
| | | CCGACGCTCGGCTTTTTAATTGGCGGTATTGTCATCGCAGCCTTC |
| | | GGTAGCCGACTTACAATCCCAGATGCAGTATACAAGTTCATCGT |
| | | TTTTATGCTGTTAATCAAAGTCGGTCTTTCAGGCGGTATCGCAAT |
| | | TCGTAATACCAACATCACGGAGATGCTACTCCCTGCGTTATTTGC |
| | | TGTACTAATGGGCATCCTAATCGTATTTATCGGGCGTTTTACCTT |
| | | AGCAAAACTGCCAGGTATCAGAACGGTAGACGCAGTGGCAACT |
| | | GCAGGCCTATTCGGCGCAGTGAGTGGTTCGACCCTTGCTGCTGG |
| | | AATCACGGTCATGGAAGGCCAAGGTGTTTTCTACGAACCGTGGG |
| | | CAGCGGCACTTTATCCTTTTATGGACATTCCCGCCCTGGTGACAG |
| | | CGATTGTTGTAGCTTCACTTTATAAGTCAAAACAGCGCGAGGTT |
| | | GAAGCAGATGACTTCAGCAAACAACCAGTAGCAGCTGGTGAAT |
| | | ACTCTGGTGAGCCTGTTTACCCAACAACGCGTCAAGAATACCTG |
| | | GGTCAAAAACGTGGTAAGGCTACTAACAGAGTTGAAATTTGGCC |
| | | AATTGTTAAGGAATCACTACAGGGTTCTGCCCTATCAGCATTGC |
| | | TTCTCGGACTTGCTCTAGGTTTGCTTACTCGACCAGAAAGTGTCT |
| | | TTGAGAGTTTCTACGAGCCACTCTTCCGTGGTTTTCTTTCTATCTT |
| | | GATGCTGGTAATGGGGATGGAAGCTTGGTCTAGACTTGGCGAAC |
| | | TGCGCAAAGTTGCTCAATGGTACGCTGTCTATGCGTTTATTGCGC |
| | | CGCTACTCCATGGATTTATTGCATTCGGTCTAGGCATGATCGCAC |
| | | ACTATGTAACAGGCTTCAGTCCTGGTGGTGTTGCCCTATTAGCA |
| | | ATTATCGCGGCGTCTTCAAGCGACATCTCTGGCCCGCCTACTTTA |
| | | CGCGCTGGAATTCCGTCGGCTAACCCTTCTGCTTACATCGGTAGT |
| | | TCTACTGCAATCGGTACACCAGTAGCGATCGCAATCGGCATACC |
| | | ACTTTTTATCGGCCTTGCGCAAGCAACAATGGGCGGCTAAGGCC |
| | | CGCATGTAGTCAAAAGCCTCCGGTCGGAGGCTTTTGACTCTTGG |
| | | TAAGGAGTAGTGTTATTCCACGGTAACTGCTTTTGCCAGGTTAC |
| | | GACAAGATGAATAAGCAAGATGCCGAGCCTACGTGCATGGTGT |
| | | CGAACGTAACAAAAGAATCAGTGTAAGAACTTAATCGGCGTTTA |
| | | AAGCTGATGACTGGTTTTTAAAAAATTGGACGTCGTGGTGAAGT |
| | | TGAATTCATGGTCCAGGCAAGAAAGCGGATAAAACCTTTAAAG |
| | | AAGCAAAAGCCAAATACGGTGTAATTAAGCTGACATCAGCACT |
| | | GTTAATCTTTTAACCCTTTAGTGCGCATTATGTATATTATGTTAA |
| | | ATAAAGCATAACGCTACGCCAAATCTTTACTCGTTTAGCCTCCTA |
| | | TCCCTCTCTTATTTCAATTTCATCTAGGGGTTTGAGGCTTTCATTA |
| | | AACATAATGGCTATTTACCATAAAATACTTAATATAGATCTTAA |
| | | TAATCTATCGATTGTTAAGATCTATATTAAGGCTAATCATTTTCT |
| | | ACAAACACTTGAATGGCTTCATAGATGTCTTGTCTACGGACTGG |
| | | TTTGGAAATGAAGCTGTCCATACCGGCTTCTAAGCATTTGTCAC |
| | | GGTCACTTTCTAAAGCGTGTGCTGTTAATGCGATGATTGGAATC |
| | | GTGATGCCTTTTTCTCGGATCAAGCGCGTTGCGGTAATGCCGTCC |
| | | ATGACTGGCATCGATACATCCATAAGTATAATGTCGATTGAGTC |
| | | GCTGTTATCTTCCAGAAATGCCAGTGCTTCTTCGCCATGGCTTGA |
| | | GATGTGAACGTTATGTCCTAACTTGTTGAGAATTAATTTAATCAC |
| | | GAGCTGGTTTGAATGTGTATCTTCAACCACAAGTAAATTTAACG |
| | | CTTTGACTGGGCTGTTTGCTTCGATTTTTACAGGACGATTCTGAT |
| | | GAAGCCTAGTCAATACCGGAATCTTTATTGTGAATGACGAGCCC |
| | | ACGTGTTCTTTACTATCAACTATGATGGTTCCGCGCATCATCTCT |
| | | ACTAAATGTTTGGTGATAGCCAGGCCTAATCCTGTTCCTCCGAA |
| | | ACGTCTGGTTATCGATCTGTCGCCTTGATGAAAAGCCGTGAATA |
| | | AACGAGCTTGTTTGGCTTTTGATATACCAATGCCCGTGTCAGAG |
| | | ACGGAAATTATTAGCTCGTCTTTCTGTTCCAAAACCGAAACGCT |
| | | CACTGTACCGCTGTCGGTGAATTTAATCGCGTTACCGATAAGAT |
| | | TAAATAAAACCTGAGATAACCTGGTCGAATCAATCCAATACTGT |
| | | TTGTCTTGCGGTATGCGGCAATCAAGATTGAAGTTGAGGTTTTTG |
| | | CTAATAGCTATTTGTTTTTGCTGGGAAATGACAAAAGTCACAGC |
| | | GTCACTTAAATTGGTCCACTGTTCATGTAACTGAAAGCTACCCG |
| | | ACTCGATTTTAGAGAGATCCAGGATGTCACTTATGATGGCGAGA |
| | | AGCAGTTCCGCAGAGCTCTCCATTTGACACAGCGCTTCGAACTG |
| | | CTCGTTGGATAATGTTGATTGCCTTAATATATCGAGCATACCCAG |
| | | AACGGAATTAAGCGGCGTTCGGATTTCATGGCTCATCATGGCCA |
| | | GAAACTCGGACTTAGATTGGTTTGCGAGTTCAGCTTCTTCGCGTG |
| | | CTTTAGCTAGCTCTGTTGTACGTACATCAACGATACGTTGTAACT |
| | | TCTCTTTGGTCTCGATGTCTATTACCGCTCGCTCTATTAGAGGGC |
| | | GAAAGCGTCGAAGTGTTTCTTTGTTTTCAATACTGAAGTGCCCTT |
| | | TTTGTGCTCCAATTAATAGGATTATATTTTGAGTTACTTCAGAGC |
| | | GAATGCCAGTCAGAATGACAGAGTTCACGTGGTTCTTGACGAAT |
| | | ATATTTAAGTTAGAGAACTCGCCCAACCTTAATGGTTCGAACAG |
| | | TAATACACATTCACCATTTAGCGCTCGCTCCATGGTGTTACCGTG |
| | | TAACCAATTAACCGTGTCGAATACGCTGTTCGTTGATATTAACGT |
| | | TTTGAAAGATTGCCTGTTATCGTCACGAGTAATAACGATAAAGT |
| | | CTTCAAAGTTAATGTATTTTTTTAAGACCGTATTTAAGCCAGAGA |
| | | AGATTTCATTGCGGTTTTTGGCTTCGCTCATGGCTGATATTGCCG |
| | | AAAGAATGGCTTTGTTCTCGTCAGCGAGCAATTTTTCTCTATGCT |
| | | TGGCTTGTTGTAACTCAACCAATGCTTCCTGCAAAGCTTCTTGTT |
| | | GGTAGGTCTCTAAATATTTATCTTTCATAAGGCTTATGGAATATC |
| | | GCTGATGAGATCATTAGGTTCCCATGTCCATTTTCGCCACCAATA |
| | | AATCTTCCTTGTTCGCCAAAGGTAAATGGGCATATAAATGGCAG |
| | | GTGTTCCAGTTCTTGGTTGATTTGTTCACAAACGCTGTTCATGTC |
| | | TTGCTTCAAGTGCAACATGGGCC |
| 32 | ppc KO Sequence | TTAACCAGTGTTACGCATACCTGCCGCGATACCTCCGAAAGCGA |
| | | CGAGTGTCCTGCTTTTGAGTGCGGCGTATTTCTCGTTCAT |
| 33 | pC100-CgSucE | AAGCTTATTTGAAAAAAGTGTTTGACAGTTGAACACTCGTTGCC |
| | | TATAATCGAAAGTCGAGTTATCGAGATTTTCAGGAGAGGAAGCT |
| | | CATATGAGTTTTCTTGTAGAAAACCAACTATTGGCGCTCGTCGTT |
| | | ATTATGACAGTCGGTCTTTTGCTGGGCCGAATTAAGATCTTTGGT |
| | | TTTAGACTGGGTGTAGCTGCTGTGCTTTTTGTTGGTTTAGCATTA |
| | | AGCACGATCGAACCAGATATCTCAGTTCCGTCGCTCATTTACGTT |
| | | GTGGGTCTATCCCTTTTTGTATACACCATCGGTCTAGAAGCTGGT |
| | | CCAGGCTTTTTCACTTCCATGAAAACTACAGGCTTACGCAACAA |
| | | CGCTTTAACGCTCGGCGCAATCATAGCTACGACAGCCTTAGCAT |
| | | GGGCTCTAATCACTGTCTTAAACATCGATGCAGCTTCTGGCGCG |
| | | GGTATGCTTACGGGTGCGTTAACTAATACACCTGCAATGGCGGC |
| | | GGTAGTTGATGCGCTTCCGTCTCTTATCGATGATACCGGCCAGCT |
| | | GCATCTGATTGCAGAACTTCCGGTCGTAGCGTATAGTCTTGCAT |
| | | ATCCACTAGGTGTATTGATTGTCATTCTTAGTATTGCAATTTTTT |
| | | CTAGTGTATTCAAAGTGGATCATAACAAGGAAGCGGAGGAAGC |
| | | TGGCGTAGCAGTTCAAGAACTTAAAGGTAGACGTATCCGTGTTA |
| | | CTGTCGCAGACCTGCCTGCACTTGAGAACATCCCAGAACTCCTT |
| | | AACCTGCATGTAATCGTTTCGCGCGTTGAGCGTGATGGGGAACA |
| | | GTTTATCCCATTGTACGGTGAACACGCTAGAATCGGTGACGTTC |
| | | TTACAGTAGTCGGTGCCGACGAGGAGCTAAACCGAGCAGAAAA |
| | | AGCAATCGGTGAGCTTATCGACGGCGATCCATACTCAAACGTTG |
| | | AACTGGACTACAGACGTATTTTTGTATCAAATACTGCGGTAGTG |
| | | GGTACGCCGCTCTCTAAACTGCAACCTCTATTTAAAGATATGCT |
| | | AATTACACGTATCCGCCGCGGTGACACCGACCTAGTTGCTTCAT |
| | | CTGATATGACACTTCAACTAGGCGATCGTGTTCGCGTTGTAGCA |
| | | CCAGCTGAGAAACTGCGTGAAGCGACACAATTACTGGGAGACA |
| | | GCTACAAGAAACTATCAGACTTCAACCTCTTACCACTCGCAGCA |
| | | GGCCTAATGATCGGTGTGCTTGTGGGAATGGTTGAATTCCCACT |
| | | TCCGGGAGGCTCCTCACTCAAATTGGGCAACGCAGGAGGCCCTT |
| | | TAGTTGTGGCACTATTGCTAGGCATGATTAACCGTACTGGCAAA |
| | | TTCGTGTGGCAAATTCCTTACGGTGCTAATTTGGCTTTACGCCAG |
| | | CTAGGCATCACCTTGTTTCTAGCTGCCATTGGTACGTCTGCGGGT |
| | | GCAGGTTTTAGAAGCGCTATCTCAGACCCTCAAAGTCTAACTAT |
| | | CATCGGCTTTGGGGCGCTTCTAACACTTTTCATCTCTATCACGGT |
| | | CCTGTTCGTAGGCCATAAGTTAATGAAGATCCCGTTCGGTGAGA |
| | | CAGCCGGTATCCTTGCAGGCACTCAGACGCACCCAGCAGTCTTG |
| | | TCATATGTATCTGATGCTTCACGTAACGAACTACCCGCAATGGG |
| | | ATACACTTCTGTATATCCTTTGGCAATGATCGCTAAAATCCTTGC |
| | | AGCCCAGACACTACTTTTTTTACTCATTTAA |
| 34 | btsS with P311R | ATGGAACTGGTTATTTCTCTATTACAGCAAATGTGTGTTTACTTA |
| | mutation | GTACTGGCGTACATGCTGAGTAAAACACCCATTATATTGCCTCT |
| | | GTTGAGCATCTCTTCTCGCTTAAGCCACCGCTTAATTTGTTACGT |
| | | ACTTTTCTCGGGCTTCTGTATTCTCGGCACCTATTTTGGTCTGCAT |
| | | ATTAACGATGCAATTGCCAATACTCGTGCTATCGGTGCAGTCAT |
| | | GGGCGGGCTATTTGGTGGCCCCGTGGTTGGTTTCGCGGTAGGAC |
| | | TTACTGGTGGTATTCACCGCTACACACTCGGTGGTTTTACCGATC |
| | | TGGCTTGTGCAATCTCAACCACGGCTGAAGGGGTTATCGGTGGC |
| | | CTGCTTCACGTATACCTAATCAAACGCAACAAAGGTGCCCTGCT |
| | | ATTTAACCCAAGCGTCGTGTTCAGCGTGACTTTTGTGGCTGAAGT |
| | | TGTACAAATGATCTTACTGCTCGCGGTCGCAAAACCGTTCGACC |
| | | AGGCTTATGAACTGGTATCAGCGATTGCTGCTCCGATGATCATT |
| | | GCCAACTCGTTTGGTGCTGCTCTGTTCATGAGCATTCTTCAAGAC |
| | | CGAAAAACCATTTTTGAAAAGTACTCGGCAACCTTTTCACGCCG |
| | | AGCGTTAACCATTGCCGACCGCTCAGTGGGCATTTTAAGTAACG |
| | | GTTTTAACACTGAAAACGCAGAAAAAATCGCCCGTATTATTTAT |
| | | GAAGAGACAAAAGTAGGCGCGGTCGCGATTACCGATCAGGAGA |
| | | AAATCCTCGCCTTTGTTGGTATCGGTGATGATCACCACAAACCC |
| | | AACACCCCGATTTCATCGCAAAGTACCTTAGACTCGATGGAAAA |
| | | GAACGACATTATTTATCTGGATGGCTCCGAGCGCCCTTATCAGT |
| | | GCTCCATTGCGAAAGACTGTAAACTGGGTTCAGCGCTTATTATT |
| | | CGACTTAGAGCGGGCAAGGAAGTAATCGGTACTATCAAACTCTA |
| | | CGAGCCGAAGCGAAAACTGTTTTCGACGGCAAACATGTCGATGG |
| | | CAGAAGGGATTGCACAACTGTTATCCAGCCAGATTTTATATGGA |
| | | GATTATCAGCAACAACAAACCTTATTAGCTCAGGCAGAAATCAA |
| | | ATTGCTGCACGCGCAGGTTAATCCACATTTCTTGTTTAATGCGCT |
| | | CAATACCATCAGCGCGATTACACGACGTGACCCGGATAAAGCTC |
| | | GAGAACTGATCCAAAACTTATCGCACTTCTTCCGCAGTAACTTA |
| | | AAACAGAACATCAACACGGTAACGCTAAAAGAAGAGCTTGCTC |
| | | ATGTGAATTCTTACCTGAGTATTGAAAAGGCTCGTTTTACTGACC |
| | | GACTCGAAGTCGAGATAGATATTGAACCAGAATTACTGGACATC |
| | | AAGTTACCAAGCTTTACACTTCAGCCTTTGGTAGAAAATGCCAT |
| | | TAAACACGGCGTCTCTAATATGCTGGAAGGCGGCAAGGTGAGA |
| | | ATATACAGCCAAGCGCATCCACAAGGTCATCTGATTACCGTGGA |
| | | AGACAATGCAGGCAGTTTTGAGCCACCTAAAGACAACCACTCTG |
| | | GTTTAGGTATGGAGATTGTCGACAAACGTCTCACAAATCAGTTT |
| | | GGGCGTGATTCAGCGCTAAAAATTGCGTGCGAGAAGCATCAATT |
| | | TACTAAAATGAGTTTTATTATTCCCACAACTTCGTAA |
| 35 | C. glutamicum | ACCCACAACCAATAAATCCGGTTGCCATTCTTTGATCTGTTTTTC |
| | PYC in DNS | GATGTCGTCCCAGTTTGGAATACCATCATTGGCTTTAAAAGCCTT |
| | | GAGCGGAGAGGCGGTACCGGTAATTTCCTGGCCAATCGCGCTGC |
| | | CGATGCTTTTGGTTCCAAAATCAAAAGCCATAATTGTACGTGAC |
| | | ATAAGGTGTCTCAAATCTCAATCTAGGGGAACAGTCTGCCTTAT |
| | | CAGGCATGTCCTGCATCAGAAGAAAGCTGACTCGGATCGATCCC |
| | | CAGTTTTTCTACTGCTTTTTTCCAACGGTCTGTAATTGGAGTATC |
| | | AAAGATGATTTCTGGCGTAGCTTCTATGGTCAGCCAAGAGTTTT |
| | | CAACTAACTCATTTTCCAGCTGCCCTGCGCTCCAACCTGCGTAGC |
| | | CCAAAGCAACCAAATAGTCGCTTGGCTCAGCTTCGGTCCCCAAC |
| | | ACAGTCAGAATGTCTCGCGAGGTTGTCACTGCAAGCTCATCAGT |
| | | CATCTGAATACTGGATTCATAGTAATCTTTCGGTTTATGCAGAAT |
| | | AAAGCCACGATCTTCTGAGATCGGGCCGCCGTTATATACAGGCC |
| | | GGTCAAGGCTTGCTTCGAACAAACGCGGGTGTACAGGTTGTACT |
| | | TTGACTTGCTTAAGCATGTTACCCACGGTGATATCGACTGGCGC |
| | | ATTGATTATCAACCCCATTGCGCCTTCTTCGTTGTGCTCGCAGAC |
| | | GTAGATCACGCTATTCTGGAAATAGGGATCTTTCATTCCAGGCA |
| | | TGGCAACTAAAAAATGGTTTGTGAGGTTCATATTCATACTGATC |
| | | CTTAGGACTCAGAAGTGCACTTAAGCAAATTGGCAATATATGCA |
| | | TGAATTTGCTTAAGTTTGCATTTAGCTCTCTACTATAACTTTACT |
| | | ATATATTTAGGGTCTGTTGACCTTTCGCGCTGATTTTTGCAGCTG |
| | | TTTGTGGGTTCTTTATACAAGGCAGAGGCTTTGAAATGTAGTTG |
| | | GCCTACCTGATAAGCCGATAACGCAGTAGAAAGTATCCACAAAC |
| | | GCTGCCCGAAGGGTTCGGCTAAAAGCGCTTTACTCTTTGTTGAG |
| | | AGGTATTTTGCTTAGAATGACTAGGCTATAAACCTCTCGCCGCG |
| | | ATTAAAACGCTTTTATCTCGAACAAAAATTAACCACGCAAGATA |
| | | AACAGACCCTATTTATTCGCCTTTTACTCGGCGTTCAATGGCATC |
| | | CATTAGTTTGCCCGTAATTGAGATATCAAATGCTGCTTCGATTTC |
| | | ACGAATACAGGTCGGGCTGGTTACGTTGATTTCTGTCAGCTTATC |
| | | GCCAATCACATCAAGACCCACAAAAATAAGACCTTTTTCTTTCA |
| | | GAGTTGGTGCTACGGTTTGGGCAATTTTAATGTCTGTTTCACTTA |
| | | GAGGACGAGCTTCACCCGTGCCGCCCGCCGCAAGGTTACCGCGC |
| | | GTTTCGCCTTTCGCCGGAATACGTGCCAGACAGTAAGGCATTGG |
| | | CTCGCCGTCAACCACGAGAATGCGCTTGTCACCATTGCTGATAT |
| | | CAGGCACGAATGTTTGTGCCATCGCGTAATGCTGACCATGGTTA |
| | | GTCAGCGTTTCGATGATCACTGATACGTTTGGATCGTTCTCATTT |
| | | ACGCGGAAAATAGACGCACCGCCCATACCGTCAAGAGGCTTTA |
| | | AGATCACATCGCCGTGTTCTTCACGGAATGCTTTAATCTTTTCAG |
| | | CTTTACGGGTAACGATAGTGGTTGGAGTCAATTCAGGGAACCAT |
| | | GCTGTAAACAACTTCTCATTGCAGTCACGAAGGCTCTGAGGTTT |
| | | GTTGACGATCAGTGTGCCTTGCTCTTCTGCACGCTCAAGGATGTA |
| | | CGTTGCGTAGATGTATTCAGTGTCAAACGGAGGATCTTTACGCA |
| | | TCAGAACCGCGTCTAACTCAGATAGCTCGATAGTTTGCTCTGATT |
| | | TGAATTCGTACCAGCCGTTTGGATCTTCTTTTAGCTCAACCACTT |
| | | TGGTGTCGGCAATGGCCACACCTTGGTCGAGGTGAAGATCATTC |
| | | ATTTCCATGTAGTGGATTTCGTAGCCGCGGCGTTGCGCTTCAAGC |
| | | ATCATGGCAAAGCTAGAGTCTTTCTTGATGTTAATGGACGAAAT |
| | | TGGGTCCATTACAATACCGAGTTTGATCATTATTTTTCTCCGTTT |
| | | TAACCAAGATCGCCGAAACGAACTTGTAAAGCTGTAATTGCGGT |
| | | AAGAGCCGCTGTTTCGGTGCGAAGTACACGCGGACCGAGAAGC |
| | | GTCTCTTCAAATTGGTACTCGCGCGTCATATCGATTTCTTCAGCT |
| | | GACAAACCGCCTTCAGGGCCAATCAGCAGGCGCACTTTTTCAAC |
| | | TGGTGTAGGCAGGGTGTTGATCGAGTATTTGGCACGAGGGTGAA |
| | | GGTTGAGCTTAAGTCCATCGTACTCTTCTTTGCTCCACTCTTCCA |
| | | AGCTCATGATTGGGCGAATTTCTGGAACGATGTTACGTCCACAC |
| | | TGCTCGCAAGCACTGATAGCAATCTTCTGCCATTGGGCCAGTTTC |
| | | TTCTCAAATCGTTTTTGATCGAGCTTTACGCCACAACGTTCAGAA |
| | | ATAAGGGGAGTGATGGTATTTACTCCGAGTTCGACTGACTTCTG |
| | | AATGGTGAACTCCATCTTGTCGCCTCGTGAAATCACCTGTCCTAG |
| | | GTGAAGATCCAAAGGGGATTCAATGCTGTTCTCTACGCGCTCAG |
| | | AGATGTCTACGAGGACATTCTTTTTGCTGACTTCTGCGATAACTG |
| | | CGGGAAACTCTGCACCACTACCGTCAAATAGGAGAACTTCCTGA |
| | | CCTTCCTTCATACGAAGTACGCGGCCAATATGGCCAGCGGCGTC |
| | | GTCACTTAAAGCGAGTGTACCAAGTTGGTGAATGGTTTCTGGAT |
| | | GATAAATTCGAGGGATACGCATGAGATTTCCAGCAAGAGAATA |
| | | AACTTAGTTCCTAACATGGATGCTTTCCATCGAAAAAACAAGGG |
| | | GCAAGAGGCGTTTCAATTGGAGTAAATGAAACGCCTCAAACAG |
| | | AAAGATTAGCGATTGTCGCGATTGGTGAGGATTTTAGTTACGGG |
| | | GCCTTGACAATTAATCATCGGCTCGTATAATGTGTGGAATTGTG |
| | | AGCGGATAACAATTTCACAAAGGAGGTGCGGCCGCATGTCGACT |
| | | CACACATCTTCAACGCTTCCAGCATTCAAAAAGATCTTGGTAGC |
| | | AAACCGCGGCGAAATCGCGGTCCGTGCTTTCCGTGCAGCACTCG |
| | | AAACCGGTGCAGCCACGGTAGCTATTTACCCCCGTGAAGATCGG |
| | | GGATCATTCCACCGCTCTTTTGCTTCTGAAGCTGTCCGCATTGGT |
| | | ACCGAAGGCTCACCAGTCAAGGCGTACCTGGACATCGATGAAAT |
| | | TATCGGTGCAGCTAAAAAAGTTAAAGCAGATGCCATTTACCCGG |
| | | GATACGGCTTCCTGTCTGAAAATGCCCAGCTTGCCCGCGAGTGT |
| | | GCGGAAAACGGCATTACTTTTATTGGCCCAACCCCAGAGGTTCT |
| | | TGATCTCACCGGTGATAAGTCTCGCGCGGTAACCGCCGCGAAGA |
| | | AGGCTGGTCTGCCAGTTTTGGCGGAATCCACCCCGAGCAAAAAC |
| | | ATCGATGAGATCGTTAAAAGCGCTGAAGGCCAGACTTACCCCAT |
| | | CTTTGTGAAGGCAGTTGCCGGTGGTGGCGGACGCGGTATGCGTT |
| | | TTGTTGCTTCACCTGATGAGCTTCGCAAATTAGCAACAGAAGCA |
| | | TCTCGTGAAGCTGAAGCGGCTTTCGGCGATGGCGCGGTATATGT |
| | | CGAACGTGCTGTGATTAACCCTCAGCATATTGAAGTGCAGATCC |
| | | TTGGCGATCACACTGGAGAAGTTGTACACCTTTATGAACGTGAC |
| | | TGCTCACTGCAGCGTCGTCACCAAAAAGTTGTCGAAATTGCGCC |
| | | AGCACAGCATTTGGATCCAGAACTGCGTGATCGCATTTGTGCGG |
| | | ATGCAGTAAAGTTCTGCCGCTCCATTGGTTACCAGGGCGCGGGA |
| | | ACCGTGGAATTCTTGGTCGATGAAAAGGGCAACCACGTCTTCAT |
| | | CGAAATGAACCCACGTATCCAGGTTGAGCACACCGTGACTGAAG |
| | | AAGTCACCGAGGTGGACCTGGTGAAGGCGCAGATGCGCTTGGCT |
| | | GCTGGTGCAACCTTGAAGGAATTGGGTCTGACCCAAGATAAGAT |
| | | CAAGACCCACGGTGCAGCACTGCAGTGCCGCATCACCACGGAA |
| | | GATCCAAACAACGGCTTCCGCCCAGATACCGGAACTATCACCGC |
| | | GTACCGCTCACCAGGCGGAGCTGGCGTTCGTCTTGACGGTGCAG |
| | | CTCAGCTCGGTGGCGAAATCACCGCACACTTTGACTCCATGCTG |
| | | GTGAAAATGACCTGCCGTGGTTCCGACTTTGAAACTGCTGTTGC |
| | | TCGTGCACAGCGCGCGTTGGCTGAGTTCACCGTGTCTGGTGTTG |
| | | CAACCAACATTGGTTTCTTGCGTGCGTTGCTGCGGGAAGAGGAC |
| | | TTCACTTCCAAGCGCATCGCCACCGGATTCATTGCCGATCACCC |
| | | GCACCTCCTTCAGGCTCCACCTGCTGATGATGAGCAGGGACGCA |
| | | TCCTGGATTACTTGGCAGATGTCACCGTGAACAAGCCTCATGGT |
| | | GTGCGTCCAAAGGATGTTGCAGCTCCTATCGATAAGCTGCCTAA |
| | | CATCAAGGATCTGCCACTGCCACGCGGTTCCCGTGACCGCCTGA |
| | | AGCAGCTTGGCCCAGCCGCGTTTGCTCGTGATCTCCGTGAGCAG |
| | | GACGCACTGGCAGTTACTGATACCACCTTCCGCGATGCACACCA |
| | | GTCTTTGCTTGCGACCCGAGTCCGCTCATTCGCACTGAAGCCTGC |
| | | GGCAGAGGCCGTCGCAAAGCTGACTCCTGAGCTTTTGTCCGTGG |
| | | AGGCCTGGGGCGGCGCGACCTACGATGTGGCGATGCGTTTCCTC |
| | | TTTGAGGATCCGTGGGACAGGCTCGACGAGCTGCGCGAGGCGAT |
| | | GCCGAATGTAAACATTCAGATGCTGCTTCGCGGCCGCAACACCG |
| | | TGGGATACACCCCGTACCCAGACTCCGTCTGCCGCGCGTTTGTT |
| | | AAGGAAGCTGCCAGCTCCGGCGTGGACATCTTCCGCATCTTCGA |
| | | CGCGCTTAACGACGTCTCCCAGATGCGTCCAGCAATCGACGCAG |
| | | TCCTGGAGACCAACACCGCGGTAGCCGAGGTGGCTATGGCTTAT |
| | | TCTGGTGATCTCTCTGATCCAAATGAAAAGCTCTACACCCTGGA |
| | | TTACTACCTAAAGATGGCAGAGGAGATCGTCAAGTCTGGCGCTC |
| | | ACATCTTGGCCATTAAGGATATGGCTGGTCTGCTTCGCCCAGCT |
| | | GCGGTAACCAAGCTGGTCACCGCACTGCGCCGTGAATTCGATCT |
| | | GCCAGTGCACGTGCACACCCACGACACTGCGGGTGGCCAGCTGG |
| | | CAACCTACTTTGCTGCAGCTCAAGCTGGTGCAGATGCTGTTGAC |
| | | GGTGCTTCCGCACCACTGTCTGGCACCACCTCCCAGCCATCCCTG |
| | | TCTGCCATTGTTGCTGCATTCGCGCACACCCGTCGCGATACCGGT |
| | | TTGAGCCTCGAGGCTGTTTCTGACCTCGAGCCGTACTGGGAAGC |
| | | AGTGCGCGGACTGTACCTGCCATTTGAGTCTGGAACCCCAGGCC |
| | | CAACCGGTCGCGTCTACCGCCACGAAATCCCAGGCGGACAGTTG |
| | | TCCAACCTGCGTGCACAGGCCACCGCACTGGGCCTTGCGGATCG |
| | | TTTCGAACTCATCGAAGACAACTACGCAGCCGTTAATGAGATGC |
| | | TGGGACGCCCAACCAAGGTCACCCCATCCTCCAAGGTTGTTGGC |
| | | GACCTCGCACTCCACCTCGTTGGTGCGGGTGTGGATCCAGCAGA |
| | | CTTTGCTGCCGATCCACAAAAGTACGACATCCCAGACTCTGTCA |
| | | TCGCGTTCCTGCGCGGCGAGCTTGGTAACCCTCCAGGTGGCTGG |
| | | CCAGAGCCACTGCGCACCCGCGCACTGGAAGGCCGCTCCGAAG |
| | | GCAAGGCACCTCTGACGGAAGTTCCTGAGGAAGAGCAGGCGCA |
| | | CCTCGACGCTGATGATTCCAAGGAACGTCGCAATAGCCTCAACC |
| | | GCCTGCTGTTCCCGAAGCCAACCGAAGAGTTCCTCGAGCACCGT |
| | | CGCCGCTTCGGCAACACCTCTGCGCTGGATGATCGTGAATTCTTC |
| | | TACGGCCTGGTCGAAGGCCGCGAGACTTTGATCCGCCTGCCAGA |
| | | TGTGCGCACCCCACTGCTTGTTCGCCTGGATGCGATCTCTGAGCC |
| | | AGACGATAAGGGTATGCGCAATGTTGTGGCCAACGTCAACGGCC |
| | | AGATCCGCCCAATGCGTGTGCGTGACCGCTCCGTTGAGTCTGTC |
| | | ACCGCAACCGCAGAAAAGGCAGATTCCTCCAACAAGGGCCATG |
| | | TTGCTGCACCATTCGCTGGTGTTGTCACCGTGACTGTTGCTGAAG |
| | | GTGATGAGGTCAAGGCTGGAGATGCAGTCGCAATCATCGAGGCT |
| | | ATGAAGATGGAAGCAACAATCACTGCTTCTGTTGACGGCAAAAT |
| | | CGATCGCGTTGTGGTTCCTGCTGCAACGAAGGTGGAAGGTGGCG |
| | | ACTTGATCGTCGTCGTTTCCTAACCGGCAGTTAAAGTCTTTAAAA |
| | | AGTATGACTTTATCCATTCTATCACTCAGATATTAGCTTTCGTAA |
| | | TAAGCAAGTTAATAATAACAAAAGAAATGTTGGTCAATATTTCC |
| | | TATTGAGACTTAATTGAACTGAGGATTAGGAAAGCTGGACACCA |
| | | GTGAAGCTAAGAGGCTGACGACATTTTTGACACGAATACACGGC |
| | | TTGTTTGCGCAGCACTTTGTTGTGACGGCGAATAGAAAGGGGAT |
| | | ATGTCGTGCAGCGACATCGGTATTCGAAGGTTTTGCCTTGAACG |
| | | GAAGAGACTTCGAAACTATGTGTGGTTTTGGCCGGAACGTTAAA |
| | | TACGGATTCCATTACGGTTTGCCACTCTTTGCCATGAGGACGGA |
| | | CGCGACCGTAAACCTGATATGTGATTAAGTGAGCGACCTCATGC |
| | | GGTATCACTTCTTGCAAGAACGCTTGGGTGTTTTCTTTAAATAAA |
| | | ACCGGGTTAAGGCGAATCTCATTGAGCTGAAGGTACGCTTTTCC |
| | | TGCTGCTTTACCTCGGACTTTAAAGGTCAGGCTGGGAATAGGGA |
| | | ATGAACGCTTAAAAGCATGTTCAGCTAATGCGATATAGTGCGCC |
| | | ATCACCTGTTTTGCTTTGTAGCTTAGTTCAATGTCCATTCGTTCCT |
| | | CTCAAGTTCAATAAAAAGCCCCCGCAGTTGCGAGGGCCGGATCA |
| | | TAGCATCACTGAATTAAGGTGTGTGTTTTTTCTTAATCGTTGCAT |
| | | GGTAGGCGTGCCAAGTACCGTAGCCGAGAATAGGCATAGTAAA |
| | | CAGCATACCGATTCCGTAGGTGGCGAAACCAATTAAGATACCCC |
| | | CACAGATGATTGCCGCCCACACGATCATTGCTGGAATGTTGGAT |
| | | TTGACCGCATTAAAGCTGGTGAATACCGCCGTCATCATGTCAAC |
| | | TCTGCGCTCCATCATTAATGGAATAGAGAAGGCAGAAATGCTGA |
| | | ATACCACGCTTGCTAGTACAAAACCAATGAGTGACCCAATCACT |
| | | AAGAAGGGCAAGAACTCTGTCAGCGGTGCACCTTGAACGGAAG |
| | | GGTACAATGCATGGAGAAGTGCGGCGATTCGCATCCAGAAAAT |
| | | CATACAAACCGCAAGCAGAACCGCAAATGCCCATTGAGAGCTT |
| | | GAGTTACGACCTATCGCTTTCATTGAGTGGAACAGGCGAGCTTT |
| | | GTGGCCGCGCTCTCGTTCCCAACTGGCATCATACAAACCTAGGG |
| | | CTAAAAATGGCCCAATAAGCATATAGACGATCAAACTCGGCATC |
| | | ACGACAAGGTGCGTCCCTTGCCATTGCACAAGCAATACGATGCC |
| | | AATCGCTGCTGCCATGAAGCACAGTCCATAAAAGGCACTAATGA |
| | | GTGGCATACGCACGAAGTCATGAAGCCCTAGCGCGAGCCAGTG |
| | | GAAAGGCGCGCTAAGATTGACCGTGTTGCACGGAATGGTTCTAG |
| | | CGTATTCGTTATCAGGGACTTCCTTTCGTTTACTATGGAAGTCGG |
| | | ATGGATTGACTGTTCGAGGCATAACTCCTCCTTGGCTTATTCCTA |
| | | CACAGTTGTCTGTCAAATGACTTGTACCCATCGGAGTAAATAAT |
| | | TGTTCATTCTAACGCGTTCAAATACTCGATAATTCCGCGACAATG |
| | | TTTGACTGGAAAATAATTATTTATCAAAACATTACGCGTTAATTT |
| | | CAGGCGTTTTGACTACGCTATTCCTGACAGTTTGCTTACTTGGAT |
| | | TGGTACTAACCCCTATAAGTTTTAAAAGCATAAAGGATGTTTAG |
| | | CTAACGTTTTCCCCAAGGTTGCTGTCGTCACACTAGCTTACATCC |
| | | ACGTTGTTACGGAAACGCAATTTAACAATATTTTAACTATTGGC |
| | | GGAGTTGGGGGAATTTACAACTATTTGCTTAAAATTTGTTCTTAG |
| | | TGGGTGGGGCTGGAGGATAGAGAAGGAGGGGAGTCGGGGGAA |
| | | ACAAAAAGCCCCCACCATAAGGCGAGGGCTTTGGAAATTCAGA |
| | | GCAAGCTCGATTACTTGATGCCAGCGAATTCGCGAAGAAGTGCT |
| | | GCTTTATCAGTCGCTTCCCATGGGAACTCTTCACGACCAAAGTG |
| | | GCCGTATGCTGCTGTCTTCTTGTAGATTGGCTGCAGAAGGTTAA |
| | | GCATTTCTTGCAGACCGTATGGACGTAGGTCGAAGAACTGACGT |
| | | ACTGCTTCGATGATGATGTCGTGAGATACTTTCTCAGTACCAAA |
| | | CGTTTCCACCATGATTGACGTTGGATCTGCCACACCGATAGCGT |
| | | AAGAAAGTTGGATTTCACAACGGTCAGCCATACCAGCTGCAACG |
| | | ATGTTTTTCGCTACGTAACGCGCTGCGTATGCTGCACTACGGTCA |
| | | ACTTTTGATGGATCTTTACCTGAGAACGCACCACCACCGTGACG |
| | | AGCCGCACCGCCGTAAGTATCTACGATAATCTTACGACCAGTTA |
| | | GACCACAGTCACCCATTGGACCACCGATTACGAAACGGCCAGTT |
| | | GGGTTGATGAAGAAGTTAGTCTCTTTGTTGATCCACTCTGAAGG |
| | | TAGAACTGGCTTGATGATCTCTTCCATTACCGCTTCACGCAGTTC |
| | | AGGAGTTGAGATTGAATCACAGTGCTGAGTTGATAGAACAACA |
| | | GCATCGATACCTACGATCTTACCTTGGTCGTATTGGAACGTTACC |
| | | TGAGATTTCGCATCTGGACGTAACCAAGGAAGTGTACCGTTCTT |
| | | ACGTACTTCTGCTTGTTTTTCAACAAGACGGTGAGAGTAAGTAA |
| | | TTGGAGCTGGCATTAAAACTTCAGTTTCGTTACATGCGTAACCG |
| | | AACATGATGCCTTGGTCACCGGCGCCTTGCTCTTTTGGATCCGCT |
| | | TTATCAACACCTTGGTTGATATCTGGTGACTGTTTACCAATTGTG |
| | | TTTAGTACTGCACAAGAGTTCGCATCAAAACCCATATCTGAGTG |
| | | TACGTAGCCGATTTCACGAACGGTTTCACGAGTTAGCTCTTCAAT |
| | | GTCAACCCATGCTGAAGTCGTGATTTCACCACCCACCATTACCA |
| | | TACCGGTTTTAACGTAAGTCTCACAT |
| 36 | pC100-AsVnPCK | GAATATCAGTGCCTTTAAAGCTGGCACTAAATTCATTTGCTATCG |
| | hybrid-1 | CACCAGGGGCACAGAGTAGGCTCCCTGCCAATAACCAAGCACTT |
| | | TTGCTTAACCAATGTTTCACGTATTTCTCCCTGCACTTACGCTGA |
| | | TAGCCTAAGTATTACAATTGGATATAGATTTCGTACTGCTGGCCT |
| | | TCACGCTCAACAGTCAGATTCAGCTCTTGCGGATCGTTCATGATT |
| | | CGCATCAATTCAGCTGAAGAAGAAGGATCACTCAAGTCGATCCC |
| | | ATTAAGTTGTACTGCAATATCACCAGACTCTAAGCCCACATCAT |
| | | TAAATAATGCAGCGTCCCGACCCGGAGCAACTCGGTAACCCACA |
| | | ATGTCATCACCATTCTTAACCGGTGAAATGCTGATGTATTTGAA |
| | | AACACTTTTCGGATCTTGAGCAATTTCCTCTCGGATTTGCTCCAA |
| | | TTTGTCACCCGCTACAGACTCTGGTTGTGCTGCCTGAGCACGTCT |
| | | TAATTGAGGCGATTCAGATAACTTGTTGTATTCAATCCCCTGCAT |
| | | CATCAGGGTTTCGTCACGGCCTTGGTTTTCGATGATCACACGGTC |
| | | CACCAATACGGCTTTCAGTGTCGCACGTGTGCCTTCGATTTCTTC |
| | | ACCGATACCATACGTTGACTGTTTACCTTTATTAGCAATCACCGC |
| | | TAAGCTGGTTTGCACCTGATTACTGGCCACTGCCCCCACGAGAG |
| | | TTAAGTTTAGACGAGTTTTAGGCGCATCTTTGACAACTGGTTGTT |
| | | CGATGATGACAGGTTTTTGTTCAGAGTATTGACCGAATAAGTGA |
| | | GCATTTTGAAGGGCATTGAAATCAATCGCGTCTTTGTTATTGCCA |
| | | GCAATAACGTTACCTGCTGAAGGCCGCCATGGGCTGATATCAGC |
| | | CGTTTGTGGGATCGCTAACCACACTATCTTTCCGAGCAGCCACC |
| | | CGGCTAGCGCGATAAATAAACAAGTAAACAGCAAACTAAGCTT |
| | | GGTTTGAATCGCGTAGTTATTGGATTTCATCTGAGCCAGTAGCG |
| | | GGCTACCAGATAATCCAGCACTCAGCTCTATTCGCTTCACTAAA |
| | | TTGATTCCTTTCTTTCGATGTAGGTCGAACTGGCTATTAAAAAAA |
| | | CATCGACTCTATCTTTCTAAAATGACAGTAAAATTACTAATAAA |
| | | CAAATAGCAGTGAACTATATCACAACAGATATGGCATACCTTTT |
| | | TCTTCTTCAGTTTTTGCCAACTCCCTTGAAAAAATTAGACCTTGG |
| | | CTCCATCTGTAGGTGAATAATCCATCACTCACTGCAGTTAATGG |
| | | CAGTGGTTAGATAGCGTAGTGTTCTATGAGTTCCGTTAATGAAG |
| | | CCGTTAGGCTAGATAAATGGCTTTGGGCCGCCAGATTTTATAAA |
| | | ACCCGCTCCATCGCTCGTAACATGGTCGATGGCGGTAAAGTCCA |
| | | CTATAATGGCCAACGAAGCAAACCGAGTAAAATCGTTGAAGTG |
| | | GGCGCCGAAATTAAACTTCGTCAAGGCAATGAAGAGAAAACGG |
| | | TGATCATTGAGATAATCTCCGATCAGCGTAAGGGCGCACCAATC |
| | | GCACAGACTCTGTATCGCGAAACTAATGAAAGTATAGCGAAAC |
| | | GTGAGGAACACGCGACACAACGTAAACTTAATGCCCACAACCC |
| | | AAGTCCGGAACGCCGCCCAGATAAGAAGCAGCGCCGAGACATC |
| | | ATTAAGTTCAAACACCAGTAAAAAGCCGGAATACCAACGAGGA |
| | | AATCACTCCATGGCAAATAATGTTTTAAACCGCTACCTATTTGA |
| | | AGACCTTTCAGTTCGTGGCGAGTTGGTACAATTGGACGAAGCGT |
| | | ACCAGCGTATTATCTCCAGCAAGGAATATCCGGCGGCAGTACAA |
| | | AAACTGTTAGGCGAGCTACTCGTATCAACCACACTTTTAACCGC |
| | | GACGCTTAAGTTCGAAGGCTCAATCACTATTCAGATTCAAGGCG |
| | | ACGGCCCGGTTTCTCTTGCTGTGATCAATGGTGACCACAAACAA |
| | | AGAATTCGTGGTGTTGCGCGCTGGGAAGGCGATATTGCCGATGA |
| | | GGCGAGCCTTCACGATATGATGGGTAAAGGTCATATGGTGATCA |
| | | CCATCGAACCTAAAGTAGGCGAGCGCTACCAAGGTGTTGTTGGC |
| | | TTAGAAGGTGACAATCTTGCAGAAGTTCTGGAAGGCTACTTCGC |
| | | TAACTCTGAGCAGCTAAAAACGCGTCTTTGGATCCGTACTGGTG |
| | | AATTCGAAGGTCAACCGCATGCTGCGGGTATGCTGATTCAGGTA |
| | | ATGCCGGATGGTACTGGCTCTCCTGATGACTTTGAACATTTGGA |
| | | GCAACTGACTGACACAGTAAAAGAGGAAGAGCTGTTCGGTTTA |
| | | GAAGCCAATGAACTACTTTACCGTCTTTACAACCAGGACAAAGT |
| | | ACGTCTTTACGAACCTCAACCAGTTTCATTCCAGTGCGGCTGTTC |
| | | TCGTGAACGCAGTGGTGCTGCGATCGTCACTGTTGAGAAAGCGG |
| | | AAATTTATGACATTTTAGCCGAGGTTGGATCAGTTTCGTTACATT |
| | | GTGATTACTGTGGCACAACTTATAGCTTCGATGAAGCTGAGGTT |
| | | ACTGCGCTCTACGAACAAGCTTCAACGGACAAAAAAACGCTGC |
| | | ATTAATTTTCATAGCTTACCAAGCACGTAATTCCCCCTTCAAAAG |
| | | GCCAGCTCAATGCTGGCCTTTTTGTGACCTAAGCCCCGTAAACC |
| | | CTAGCTTTTACCGTAATTTTAGTTACGGGGCCCTGAAGTCAGCCC |
| | | CATACGATATAAGTTGTAAGCTTATTTGAAAAAAGTGTTTGACA |
| | | GTTGAACACTCGTTGCCTATAATCGAAAGTCGAGTTATCGAGAT |
| | | TTTCAGGAGAGGAAGCTCATATGACTGACCTAAACAAACTCGTC |
| | | AAGGAACTTAATGACCTAGGCCTTACAGATGTTAAGGAAATTGT |
| | | ATATAACCCCAGTTACGAGCAACTTTTCGAGGAGGAAACTAAAC |
| | | CTGGCTTGGAGGGTTTTGATAAAGGGACGCTAACCACTCTTGGC |
| | | GCAGTTGCCGTCGATACGGGGATTTTTACAGGACGTTCACCGAA |
| | | GGATAAATATATCGTTTGTGATGAAACTACGAAGGACACAGTAT |
| | | GGTGGAACAGCGAAGCTGCGAAAAACGATAACAAACCAATGAC |
| | | GCAAGAGACTTGGAAGAGTTTGAGAGAATTAGTGGCTAAACAA |
| | | CTTTCTGGTAAAAGACTATTCGTGGTAGAGGGTTACTGCGGCGC |
| | | AAGTGAAAAACACCGCATCGGTGTGCGTATGGTCACTGAAGTGG |
| | | CATGGCAGGCGCATTTTGTAAAAAACATGTTTATCCGACCAACA |
| | | GATGAAGAGCTTAAAAATTTCAAGGCGGATTTTACCGTGCTAAA |
| | | CGGTGCTAAATGTACTAACCCAAACTGGAAAGAACAGGGCCTC |
| | | AACAGTGAAAACTTTGTCGCTTTTAATATTACCGAAGGTATTCA |
| | | GCTTATCGGCGGCACTTGGTACGGCGGTGAAATGAAAAAGGGT |
| | | ATGTTTTCAATGATGAACTACTTCCTGCCATTAAAAGGTGTAGCT |
| | | TCTATGCATTGTTCGGCAAACGTAGGAAAAGACGGTGACGTAGC |
| | | TATTTTCTTCGGCCTATCTGGTACGGGTAAAACAACGCTTTCGAC |
| | | CGATCCTAAACGCCAATTAATCGGTGATGACGAGCACGGTTGGG |
| | | ATGAAAGCGGCGTATTTAACTTTGAAGGCGGTTGTTACGCTAAA |
| | | ACCATCAACCTCTCTCAAGAGAACGAACCAGATATCTACGGCGC |
| | | AATCCGTCGTGACGCACTTTTAGAGAACGTCGTAGTTCGAGCAG |
| | | ACGGTTCCGTTGACTTTGACGACGGTTCAAAAACAGAGAATACT |
| | | CGTGTTTCATATCCGATTTACCACATCGACAACATCGTTCGTCCT |
| | | GTATCTAAAGCCGGCCATGCAACAAAAGTGATCTTCCTGTCTGC |
| | | TGACGCGTTTGGTGTGCTTCCTCCAGTATCGAAACTGACACCAG |
| | | AGCAAACCAAGTACCACTTCCTGTCTGGCTTTACTGCCAAACTA |
| | | GCAGGTACAGAGCGCGGTATCACTGAGCCGACGCCAACTTTCTC |
| | | TGCGTGTTTTGGCGCTGCGTTCCTAACGCTTCACCCAACTAAGTA |
| | | CGCAGAAGTACTGGTTAAACGTATGGAAGCAGCAGGTGCAGAA |
| | | GCTTACCTGGTTAACACTGGCTGGAATGGTAGCGGTAAACGTAT |
| | | CTCAATTCAGGATACACGCGGCATCATTGATGCGATCCTAGATG |
| | | GCTCAATTGAAGATGCACCAACTAAGCATATCCCAATCTTCAAC |
| | | CTTGAAGTTCCTACTTCACTACCAGGTGTTGATCCAAGCATTCTT |
| | | GATCCACGTGATACTTACGTTGATCCACTGCAATGGGAAAGCAA |
| | | AGCGAAAGATCTAGCAGAACGCTTTATCAATAACTTTGATAAAT |
| | | ACACAGACAACGCAGAAGGTAAATCTCTGGTTGCTGCTGGTCCA |
| | | CAGCTTGACTAATGTTGTGCGAAAACCCGGTCGAGAGTGACTTG |
| | | GTTACCAAAATATTCTACAAGCCCCTCTTTTGAGGGGCTTTTTAT |
| | | TGAATCCCACCCAAGATTGATACAAGCTAAAGCAGACTTCCATA |
| | | CTTTACGTAGGTTCTGGCAAGGATGAAAACAGCGCTTCGAATTC |
| | | TAATTCTGTTACTTGTATTACTGATTGCTATCCCTGCAACCGTTA |
| | | TTGCGCTGCTTACCACTTCCTACGCTAACCAAACCTGGGCCTTTA |
| | | TTTCAGAGCACCTGAACTTGCCTATTCAGGCAGAAAAGGTGCAC |
| | | TACGACTTTCCTTATCACCTCACCGTGCAAGGCATTCGAACGAA |
| | | TAGCGAAGATATATCCAGCATTGAGCAAGTCGATGTATGGCTAA |
| | | ACCCAGATGTGCGCCGCGATGGTAAGTGGATTGTCGATAGCTTA |
| | | CTTATCAATGGCGTGAGCTTAAAACAAGGACTGCCGGATTTCGC |
| | | CAATCTAGAATCTGTCCAGTTCCACCAAGTGGCGGTTAAAAATC |
| | | TCGATTACGCGAACAAACAGTTAGTGATCAATGGGCTGAATGTA |
| | | CAAATCCAATCACCAGACTGGCAAAACAACTCGTATGTACTGCC |
| | | TTATGGCGAGATTCAGTTGTCTGCTGCGCAAATCTACTGGAATG |
| | | GCGAAGCTTTCGATAATGTGTTGCTTGATATGGATTATCGACCA |
| | | GAAAACAGCACTCTCTATGGTACGTCGTTTAAATGGAGAGACAG |
| | | TCTGATCTCTGGTCAGGGTGAGCAATACCCTCAAGGCTGGTCTTT |
| | | GGTCAATGTCACCGTCGATAAGCTGAAAATGAACAATGCCCAGC |
| | | TGCAATCGCTGCTCGCTAAGCCATGGAATGCCATACCTTTCAAA |
| | | ATTAACCACATTAATAGTCTCGACCTGTTAAACGCTGACATTGA |
| | | ATGGGGCGACTGGCATTGGCAAAACCTGGAACTTTCGCTCGAGG |
| | | ATGCTTCACTTCCGCTATCACTTTGGCAGACACAAGCTCAGATCT |
| | | CTTTGCAAGCAGACAGCGTTAGCTTTCAGGAACAAACTGCAATA |
| | | GAGCCACGTTTAAGCGCGGTAATGACGCCTAGTCAGATTGAACT |
| | | GAAAGAACTCTACCTGGACTGGCAACAAGGCCGTGTTCAAATTT |
| | | CAGGACAGTTCCAACCAACCCAATGGCAAATAGACAATGCCTCA |
| | | ATTTATGGCTTGAAATGGGCAATGAAGCCTGACGAAAGTTCAGA |
| | | CTGGTGGCAAGCGGCAACAAAAGCACTAAACGAAGTGACGATA |
| | | AATCAGCTCGACATTGAACACAGCCAAATCATACAAATATCACG |
| | | ACAGCCATATTGGCAGGTTTCGGGCCTGAATTTGGAAGGTCGTC |
| | | AACTTGACCTCAAACGCCTAGGGACGCACTGGTCGGTGTGGAAT |
| | | GGGAACCTGGACGTTAGCGTCGTCAACGCTAGCTATGATCAGGT |
| | | GATTGCCTCTCACGCTGCGCTTTCAACTCAAAGTGACAATGGCC |
| | | TTTGGCAGTTGACTCGTTTATTTGCGCCATTAGAGCAAGGCTATA |
| | | TCGAAGGTTACGGGCAGATTGATGTCAGCACCACCAGCCAACCA |
| | | TGGACGCTGAACATCAATGCAGATGGCATTCCTCTGCGCCTCTTT |
| | | CACTCATACCTACCTAAAGTATTAACCGTAGAAGGCTTGTCCGA |
| | | TCTGAATCTTGATCTCCAAGGCCTGGCTGGCGATCATAATATGCT |
| | | GGCGTACAGCTTAACCGGAGATATCGAAGCCAACTTCCGTGACA |
| | | CGATGTTGCTCTCTCAAGCCGATCAGTCACTGAAGTCGATCACA |
| | | TTCTCTCCTGTCCGTCTGCAAGCGCGACGTGGTGAGGTTTCAATC |
| | | CAACCCATCACCATTTCGGGAAGCGACATTAAAGGCAAAATCAG |
| | | TGGTGCGTTTGATCTGGCTAACAACCCATTAAGTGGGCTTGAGT |
| | | ATCAGCTAGAAGAGCAGTGTGGCGTCATTAGCGGCGATATTTTC |
| | | AGCCATGAGCCGACGAAAAATGACTGTATCAAGCCCGCTGAGA |
| | | TCGAAGGACAGGAGTCAACAAAAGAGACTCAACAACCAGAGTT |
| | | AACAGCAGGAGAAGCGCAACCTACAGCTCCTATCGCGGAAATC |
| | | AATATGGAGCTGCAGGAAGAAGAGTTGGTTGAAGAGATAGTGG |
| | | AAGAAGATGAACTAACAATAAATGCTGTTGCCGACGAAGCACT |
| | | AACAGCTGAGTAATTGCGAATAATTAGGGATTAGCATGTAAGTC |
| | | CCGACAAAATCGACAGCTGGCGTGTCACCACTATAAATCACCAC |
| | | ATGAATCACGATTCGCGCTTTACGACCCGATTTCAGCCGATCCA |
| | | AGTCACCACTGATGCCATCAAGTGATGTGCTGGCGACAGGGTTT |
| | | TGCTCAACGGGTTGGCGATAACGAATGGAACTGTCCGCGAGAAC |
| | | AATATCACCGTGCAAGCCACGCTCTTTAAGTAGTAACCAGGTCA |
| | | TTCCCCACCCAGTCAATGTCGCCAAGGTAAAAGCAGAGCCAGCA |
| | | AACATGGTATTGTGAGGGTTAAGGTTAGGGTTAAGCTGGGCGCA |
| | | GCATTCAAATTGATAACCCGTGTATTGATTAATCTTAATCCCCAT |
| | | TTTGTCACTGATCGGGATTTGCTCTTCCCATCGCTTCTGGAGCTC |
| | | CGTACACCACTCAGGGCGGCGAAGAACATTAGCCATCGGGTCTA |
| | | AGGGCTTAACCATTTGTTGGTGGCGGACCGGGCCACGCTCATCA |
| | | GTCAGCTCACCACGGCGCTCAAAACCACTTTTCTCATAGAACGC |
| | | AATCGCGTCTTCACGGGCATTACATACCAAGCGTTTCGCACCTTC |
| | | CTGGCGCGCAAGTGACTCCAGTGCCACCAACACTAATGAACCCA |
| | | TGCCTTTGCCACGACGATTACTTTTTACCGCCATGTAACGAATCT |
| | | GACCGTCAT |
| 37 | aceE (unmodified) | MSDMKHDVDALETQEWLQALESVVREEGVERAQFLLEQVLDKAR |
| | | LDGVDMPTGITTNYINTIPADQEPAYPGDTTLERRIRSIIRWNAIMIV |
| | | LRASKKDLELGGHMASFQSSAAFYETCFNHFFRAPNEKDGGDLVY |
| | | YQGHISPGIYSRAFVEGRLTEEQLDNFRQEVDGKGIPSYPHPKLMPE |
| | | FWQFPTVSMGLGPISAIYQARFLKYLEGRGMKDTSEQRVYAFLGD |
| | | GEMDEPESRGAISFAAREKLDNLCFLINCNLQRLDGPVMGNGKIIQE |
| | | LEGLFKGAGWNVVKVIWGNNWDSLLAKDTTGKLLQLMNETIDGD |
| | | YQTFKAKDGAYVREHFFGKYPETAALVADMTDDEIFALKRGGHES |
| | | SKLYAAYKNAADTKGRPTVILAKTVKGYGMGEAAEGKNIAHQVK |
| | | KMDMTHVLHLRDRLGLQDLLTDEAVKELPYLKLEEGSKEYEYLH |
| | | ARRKALHGYTPQRLPNFTQELIVPELEEFKPLLEEQKRDISSTMAFV |
| | | RSLNVLLKNKNIGKNIVPIIADEARTFGMEGLFRQIGIYNPHGQTYTP |
| | | EDRGVVSYYKEATSGQVLQEGINELGAMSSWVAAATSYSTNDLPM |
| | | IPFYIYYSMFGFQRVGDMAWMAGDQQARGFLLGATAGRTTLNGE |
| | | GLQHEDGHSHIMAGTVPNCISYDPTFAYEVAVIMQDGIRRMYGEQ |
| | | ENVFYYLTLMNENYAMPAMPEGAEEGIRKGIYKLETYTGSKGKVQ |
| | | LMSSGTIMNEVRKAAQILSDEYGVASDVYSVTSFNEVTRDGQAAE |
| | | RYNMLHPEAEAQVPYIQTVMGTEPAIAATDYMKNYAEQVRAFIPA |
| | | ESFKVLGTDGFGRSDSRENLRRHFEVNAGYVVVAALTELAKRGEV |
| | | EKSVIAEAIKKEDIDTEKTNPLYA |
| 38 | aceF (unmodified) | MAIEINVPDIGTDEVEVTEILVSVGDKVEEEQSLITVEGDKASMEVP |
| | | ASQAGIVKEIKVAEGDKVSTGSLIMIFEAEGAAEAAPAPAAEAAPA |
| | | AAPAPAAAAELKEVHVPDIGGDEVEVTEIMVAIGDSIEEEQSLITVE |
| | | GDKASMEVPAPFAGTLKEIKVAAGDKVSTGSLIMVFEVAGSGAPA |
| | | AAPAAVEAPAAAAPAASAAKEVNVPDIGGDEVEVTEIMVAVGDTV |
| | | EEEQSLITVEGDKASMEVPAPFAGTVKEIKIAAGDKVSTGSLIMVFE |
| | | VAGAAPAPAAAPAQAAAPAAAAPKAEAPAAAAPAATGDFKENDE |
| | | YAHASPVVRRLAREFGVNLSKVKGSGRKSRILKEDVQNYVKEALK |
| | | RLESGAAASGKGDGAALGLLPWPKVDFSKFGETEVQPLSRIKKISG |
| | | ANLHRNWVMIPHVTQWDNADITALEAFRKEQNAIEAKKDTGMKIT |
| | | PLVFIMKAVAKALEAFPAFNSSLSEDGESLILKKYVNVGIAVDTPNG |
| | | LVVPVFKDVNKKGIYELSEELMAVSKKARAGKLTAADMQGGCFTI |
| | | SSLGGIGGTAFTPIVNAPEVGILGVSKSEMKPVWNGKEFEPRLQLPL |
| | | SLSYDHRVIDGAEGARFITYLNSCLSDIRRLVL |
| 39 | lpdA | MSKEIKAQVVVLGSGPAGYSAAFRCADLGLETVLVERYSTLGGVC |
| | | LNVGCIPSKALLHVSKVIEEAKAMADHGVVFGEPQTDINKIRIWKE |
| | | KVVNQLTGGLGGMAKMRNVTVVNGYGKFTGPNSILVEGEGESTV |
| | | VNFDNAIVAAGSRPIKLPFIPHEDPRIWDSTDALELKEVPEKLLIMG |
| | | GGIIGLEMGTVYHSLGSKVEVVEMFDQVIPAADKDIVKVYTKRIKD |
| | | KFKLMLETKVTAVEAKEDGIYVSMEGKKAPAEAERYDAVLVAIGR |
| | | VPNGKLIDGEKAGLEIDERGFINVDKQMRTNVPHIFAIGDIVGQPML |
| | | AHKGVHEGHVAAEVISGKKHYFDPKVIPSVVYTEPEVVWVGKTEK |
| | | EAKDEGIKYEVATFPWAASGRAIASDCSDGMTKLIFDKETHRVIGG |
| | | AIVGTNGGELLGEIGLAIEMGCDAEDIALTIHAHPTLHESVGLAAEV |
| | | FEGSITDLPNKKAVKKK |
| 40 | EcExuT | MRKIKGLRWYMIALVTLGTVLGYLTRNTVAAAAPTLMEELNISTQ |
| | | QYSYIIAAYSAAYTVMQPVAGYVLDVLGTKIGYAMFAVLWAVFC |
| | | GATALAGSWGGLAIARGAVGAAEAAMIPAGLKASSEWFPAKERSI |
| | | AVGYFNVGSSIGAMIAPPLVVWAIVMHSWQMAFIISGALSFIWAMA |
| | | WLIFYKHPRDQKHLTDEERDYIINGQEAQHQVSTAKKMSVGQILRN |
| | | RQFWGIALPRFLAEPAWGTFNAWIPLFMFKVYGFNLKEIAMFAWM |
| | | PMLFADLGCILGGYLPPLFQRWFGVNLIVSRKMVVTLGAVLMIGPG |
| | | MIGLFTNPYVAIMLLCIGGFAHQALSGALITLSSDVFGRNEVATANG |
| | | LTGMSAWLASTLFALVVGALADTIGFSPLFAVLAVFDLLGALVIWT |
| | | VLQNKPAIEVAQETHNDPAPQH |
| 41 | VpSGLT | MSNIEHGLSFIDIMVFAIYVAIIIGVGLWVSRDKKGTQKSTEDYFLA |
| | | GKSLPWWAVGASLIAANISAEQFIGMSGSGYSIGLAIASYEWMSAIT |
| | | LIIVGKYFLPIFIEKGIYTIPEFVEKRFNKKLKTILAVFWISLYIFVNLT |
| | | SVLYLGGLALETILGIPLMYSILGLALFALVYSIYGGLSAVVWTDVI |
| | | QVFFLVLGGFMTTYMAVSFIGGTDGWFAGVSKMVDAAPGHFEMIL |
| | | DQSNPQYMNLPGIAVLIGGLWVANLYYWGFNQYIIQRTLAAKSVS |
| | | EAQKGIVFAAFLKLIVPFLVVLPGIAAYVITSDPQLMASLGDIAATN |
| | | LPSAANADKAYPWLTQFLPVGVKGVVFAALAAAIVSSLASMLNST |
| | | ATIFTMDIYKEYISPDSGDHKLVNVGRTAAVVALIIACLIAPMLGGI |
| | | GQAFQYIQEYTGLVSPGILAVFLLGLFWKKTTSKGAIIGVVASIPFAL |
| | | FLKFMPLSMPFMDQMLYTLLFTMVVIAFTSLSTSINDDDPKGISVTS |
| | | SMFVTDRSFNIAAYGIMIVLAVLYTLFWVNADAEITLIIFGVMAGVI |
| | | GTILLISYGIKKLIKGGGGGGHHHHHH |
| 42 | rGLK | MSEQYALVGDIGGTNARLALCELSTGTISDIVTYPAAEYESLEVVM |
| | | RQYLDRHDTPISSACIAIACPVTGDWVSMTNHHWEFSIQELKSQLN |
| | | LQVLEVINDFTAVSFAIPSLKPEDRVQIGGGEAIANKPIAVYGAGTG |
| | | LGVAQLIHGGNLWHSVPGEGGHVDLAACTPEEDDLIVYLRNKFGR |
| | | VSAERCLSGQGIRNIYDFVVSHHGAAPEDYTPAMITEKALKAECKD |
| | | CERALYLFCILMGRFGGNLALTNGSFGGVYIAGGIVPKVQELFIKSG |
| | | FRVAFEDKGRFKEYLQSIPVFLITHSEPGLLGAGTYIRQSLNISIN |
| 43 | CgMDH | MNSPQNVSTKKVTVTGAAGQISYSLLWRIANGEVFGTDTPVELKLL |
| | | EIPQALGGAEGVAMELLDSAFPLLRNITITADANEAFDGANAAFLV |
| | | GAKPRGKGEERADLLANNGKIFGPQGKAINDNAADDIRVLVVGNP |
| | | ANTNALIASAAAPDVPASRFNAMMRLDHNRAISQLATKLGRGSAE |
| | | FNNIVVWGNHSATQFPDITYATVGGEKVTDLVDHDWYVEEFIPRV |
| | | ANRGAEIIEVRGKSSAASAASSAIDHMRDWVQGTEAWSSAAIPSTG |
| | | AYGIPEGIFVGLPTVSRNGEWEIVEGLEISDFQRARIDANAQELQAE |
| | | REAVRDLL |
| 44 | H1 PCK | MTDLNKLVKELNDLGLTDVKEIVYNPSYEQLFEEETKPGLEGFDKG |
| | | TLTTLGAVAVDTGIFTGRSPKDKYIVCDETTKDTVWWNSEAAKND |
| | | NKPMTQETWKSLRELVAKQLSGKRLFVVEGYCGASEKHRIGVRM |
| | | VTEVAWQAHFVKNMFIRPTDEELKNFKADFTVLNGAKCTNPNWK |
| | | EQGLNSENFVAFNITEGIQLIGGTWYGGEMKKGMFSMMNYFLPLK |
| | | GVASMHCSANVGKDGDVAIFFGLSGTGKTTLSTDPKRQLIGDDEH |
| | | GWDESGVFNFEGGCYAKTINLSQENEPDIYGAIRRDALLENVVVRA |
| | | DGSVDFDDGSKTENTRVSYPIYHIDNIVRPVSKAGHATKVIFLSADA |
| | | FGVLPPVSKLTPEQTKYHFLSGFTAKLAGTERGITEPTPTFSACFGA |
| | | AFLTLHPTKYAEVLVKRMEAAGAEAYLVNTGWNGSGKRISIQDTR |
| | | GIIDAILDGSIEDAPTKHIPIFNLEVPTSLPGVDPSILDPRDTYVDPLQ |
| | | WESKAKDLAERFINNFDKYTDNAEGKSLVAAGPQLD |
| 45 | H2 PCK | MTDLNKLVKELNDLGLTDVKEIVYNPSYEQLFEEETKPGLEGFDKG |
| | | TLTTLGAVAVDTGIFTGRSPKDKYIVCDETTKDTVWWNSEAAKND |
| | | NKPMTQETWKSLRELVAKQLSGKRLFVVEGYCGASEKHRIGVRM |
| | | VTEVAWQAHFVKNMFIRPTDEELKNFKADFTVLNGAKCTNPNWK |
| | | EQGLNSENFVAFNITEGIQLIGGTWYGGEMKKGMFSMMNYFLPLK |
| | | GVASMHCSANVGKDGDVAIFFGLSGTGKTTLSTDPKRQLIGDDEH |
| | | GWDESGVFNFEGGCYAKTIKLSKEAEPDIYNAIRRDALLENVTVRN |
| | | DGSIDFDDGSKTENTRVSYPLYHIENIVKPVSKGGHANKVIFLSADA |
| | | FGVLPPVSKLTPEQTKYHFLSGFTAKLAGTERGITEPTPTFSACFGA |
| | | AFLTLHPTKYAEVLVKRMEAAGAEAYLVNTGWNGSGKRISIQDTR |
| | | GIIDAILDGSIEDAPTKHIPIFNLEVPTSLPGVDPSILDPRDTYVDPLQ |
| | | WESKAKDLAERFINNFDKYTDNAEGKSLVAAGPQLD |
| 46 | sbtA | MDFLSDFLTKFGSQLQSPTLGFLIGGIVIAAFGSRLTIPDAVYKFIVF |
| | | MLLIKVGLSGGIAIRNTNITEMLLPALFAVLMGILIVFIGRFTLAKLP |
| | | GIRTVDAVATAGLFGAVSGSTLAAGITVMEGQGVFYEPWAAALYP |
| | | FMDIPALVTAIVVASLYKSKQREVEADDFSKQPVAAGEYSGEPVYP |
| | | TTRQEYLGQKRGKATNRVEIWPIVKESLQGSALSALLLGLALGLLT |
| | | RPESVFESFYEPLFRGFLSILMLVMGMEAWSRLGELRKVAQWYAV |
| | | YAFIAPLLHGFIAFGLGMIAHYVTGFSPGGVALLAIIAASSSDISGPPT |
| | | LRAGIPSANPSAYIGSSTAIGTPVAIAIGIPLFIGLAQATMGG |

1. A method of producing succinic acid, the method comprising:
a. culturing a plurality of non-naturally occurring Vibrio natriegens cells comprising one or more genetic disruptions, wherein the one or more genetic disruptions increase the production of succinate as compared to the production of succinate from a V. natriegens cell that does not comprise the one or more genetic disruptions; and
b. wherein under anaerobic conditions, the plurality of non-naturally occurring V. natriegens cells produce succinic acid from a glucose substrate at a rate of at least 0.48 g_{Succ} g_{CDW}⁻¹ h⁻¹.

2. The method of claim 1, wherein the plurality of V. natriegens cells comprise 6 or more genetic disruptions.

3. The method of claim 1, wherein the plurality of V. natriegens cells comprise a mutation to a native phosphoenolpyruvate carboxykinase (pck) gene, wherein the V. natriegens cells comprise a pck fusion comprising a V. natriegens pck sequence and an A. succinogenes pck sequence operatively linked to a constitutive promoter.

4. (canceled)

5. The method of claim 1, wherein the plurality of V. natriegens cells comprise a mutation to a native malate dehydrogenase (mdh) gene, wherein the mutation comprises a replacement of the native malate dehydrogenase (mdh) gene with an mdh gene from a C. glutamicum operatively linked to a constitutive promoter.

6-11. (canceled)

12. The method of claim 1, wherein the plurality of V. natriegens cells comprise a deletion of each of the lldH, dldH, alD, and pflB genes.

13. The method of claim 1, wherein the plurality of V. natriegens cells comprise a deletion of a ptsI gene.

14. The method of claim 1, wherein the plurality of V. natriegens cells comprise a native glucokinase gene (glk) operably linked to a constitutive synthetic promoter.

15. (canceled)

16. The method of claim 1, wherein the plurality of V. natriegens cells comprise a heterologous sodium-dependent glucose transporter protein (vsGLT) from Vibrio parahaemolyticus.

17. The method of claim 1, wherein the plurality of V. natriegens cells comprise a mutation to the native E1 and E2 subunits of a pyruvate dehydrogenase complex (aceEF) gene, wherein the mutation comprises a replacement of the native promoter of the aceEF gene with a synthetic promoter.

18. (canceled)

19. The method of claim 1, wherein the plurality of V. natriegens cells comprise a mutation to a native E3 subunit of a pyruvate dehydrogenase complex (lpdA) gene, wherein the mutation comprises an E354K mutation and a replacement of the native promoter of the lpdA gene with a synthetic promoter.

20. (canceled)

21. The method of claim 1, wherein the plurality of V. natriegens cells comprise a heterologous E. coli hexuronate transporter (exuT) gene, wherein the exuT gene is operably linked to a constitutive synthetic promoter.

22. The method of claim 1, wherein the plurality of V. natriegens cells comprise a deletion of a PN96_RS22390 gene.

23. The method of claim 1, wherein the plurality of V. natriegens cells comprise:
a. a deletion of each of the lldH, dldH, alD, and pflB genes; and
b. a pck fusion comprising a V. natriegens pck sequence and an A. succinogenes pck sequence operatively linked to a constitutive promoter.

24. The method of claim 1, wherein the plurality of V. natriegens cells comprise:
a. a deletion of each of the lldH, dldH, alD, and pflB genes;
b. a pck fusion comprising a V. natriegens pck sequence and an A. succinogenes pck sequence operatively linked to a constitutive promoter;
c. a mutation to the native E1 and E2 subunits of a pyruvate dehydrogenase complex (aceEF) gene;
d. a mutation to a native E3 subunit of a pyruvate dehydrogenase complex (lpdA) gene; and
e. a mutation to a native malate dehydrogenase (mdh) gene.

25. The method of claim 1, wherein the plurality of V. natriegens cells comprise:
a. a deletion of each of the lldH, dldH, alD, and pflB genes;
b. a pck fusion comprising a V. natriegens pck sequence and an A. succinogenes pck sequence operatively linked to a constitutive promoter;
c. a mutation to the native E1 and E2 subunits of a pyruvate dehydrogenase complex (aceEF) gene;
d. a mutation to a native E3 subunit of a pyruvate dehydrogenase complex (lpdA) gene;
e. a mutation to a native malate dehydrogenase (mdh) gene;
f. a native glucokinase gene (glk) operably linked to a constitutive synthetic promoter;
g. a deletion of a ptsI gene;
h. a heterologous sodium-dependent glucose transporter protein (vsGLT) from Vibrio parahaemolyticus; and
i. a heterologous E. coli hexuronate transporter (exuT) gene.

26. The method of claim 1, wherein the plurality of V. natriegens cells comprise:
a. a deletion of each of the lldH, dldH, alD, and pflB genes;
b. a pck fusion comprising a V. natriegens pck sequence and an A. succinogenes pck sequence operatively linked to a constitutive promoter;
C. a mutation to the native E1 and E2 subunits of a pyruvate dehydrogenase complex (aceEF) gene;
d. a mutation to a native E3 subunit of a pyruvate dehydrogenase complex (lpdA) gene;
e. a mutation to a native malate dehydrogenase (mdh) gene;
f. a native glucokinase gene (glk) operably linked to a constitutive synthetic promoter;
g. a deletion of a ptsI gene;
h. a heterologous sodium-dependent glucose transporter protein (vsGLT) from Vibrio parahaemolyticus;
i. a heterologous E. coli hexuronate transporter (exuT) gene; and
j. a deletion of a PN96_RS22390 gene.

27-29. (canceled)

30. A method of producing succinic acid, comprising culturing a population of genetically modified Vibrio natriegens cells in a two-phase cultivation for a period of greater than 24 hours, wherein the population of genetically modified V. natriegens cells comprise a deletion of each of the lldH, dldH, alD, and pflB genes.

31. The method of claim 30, wherein the two-phase cultivation is for a period of less than 7 days and/or wherein each phase of the two-phase cultivation takes place in the same vessel.

32. (canceled)

33. The method of claim 1, wherein the culturing does not comprise a concentration step.

34-55. (canceled)

56. The method of claim 30, wherein the population of genetically modified V. natriegens cells comprise:
a. a deletion of each of the lldH, dldH, alD, and pflB genes; and
b. a pck fusion comprising a V. natriegens pck sequence and an A. succinogenes pck sequence operatively linked to a constitutive promoter.

57-61. (canceled)

62. The method of claim 1, wherein the rate of succinic acid production is measured over a two hour period.

63. An engineered Vibrio natriegens cell comprising one or more genetic disruptions, wherein the one or more genetic disruptions increase the production of succinate as compared to the production of succinate from a V. natriegens cell that does not comprise the one or more genetic disruptions, wherein the V. natriegens cell produces succinic acid from a glucose substrate at a rate of at least 0.48 g_{Succ} g_{CDW}⁻¹ h⁻¹.

64-84. (canceled)

85. The engineered V. natriegens cell of claim 63, wherein the cell comprises:
a. a deletion of each of the lldH, dldH, alD, and pflB genes; and
b. a pck fusion comprising a V. natriegens pck sequence and an A. succinogenes pck sequence operatively linked to a constitutive promoter.

86-88. (canceled)

89. A bioreactor comprising the engineered V. natriegens cell of claim 63.

90. (canceled)
